(19) United States
(12) Patent Application Publication
(10) Pub. No.: US 2020/0253978 A1
(43) Pub. Date: Aug. 13, 2020

(54) HETEROCYCLICS AS INHIBITORS OF FIBROBLAST GROWTH FACTOR RECEPTOR

(71) Applicant: ADVENT PHARMA CO. LTD., Shanghai (CN)

(72) Inventors: Zhaoyin Wang, Shanghai (CN); Nanxin Li, Shanghai (CN)

(73) Assignee: ADVNET PHARMA CO. LTD., Shanghai (CN)

(21) Appl. No.: 16/857,764

(22) Filed: Apr. 24, 2020

Related U.S. Application Data
(63) Continuation of application No. PCT/CN2018/111690, filed on Oct. 24, 2018.
(60) Provisional application No. 62/707,180, filed on Oct. 24, 2017.

(51) Int. Cl. A61K 31/519; C07D 471/04; C07D 491/048; C07D 495/04; A61K 31/496; A61K 31/4365; A61K 31/437; A61K 31/4355

(52) U.S. Cl. CPC A61K 31/519; C07D 471/04; C07D 491/048; A61K 31/4355; A61K 31/496; A61K 31/4365; A61K 31/437; C07D 495/04

(57) ABSTRACT

This disclosure relates to heterocyclics as selective inhibitors of the fibroblast growth factor receptor 4 (FGFR-4), in particular relates to a compound of Formula I or a pharmaceutically acceptable salt thereof, and a pharmaceutical composition including the compound.

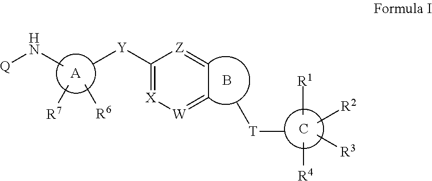

### CROSS REFERENCE TO RELATED APPLICATIONS

This application is a bypass continuation application of PCT Application Serial Number PCT/CN2018/111690 filed Oct. 24, 2018, which claims the benefit of U.S. Provisional Application Ser. No. 62/707,180, filed Oct. 24, 2017, all of which are incorporated herein by reference.

### TECHNICAL FIELD

The present invention relates to the field of medicinal chemistry, and in particular to inhibitors of FGFR-4.

### BACKGROUND

Fibroblast growth factor (FGF) receptor with tyrosine-protein kinase activity acts as cell-surface receptor for fibroblast growth factors and plays a role in the regulation of cell proliferation, differentiation and migration. Binding of fibroblast growth factors produces receptor dimerization, autophosphorylation and signal transduction. Fibroblast growth factor receptor (FGFR) family members 1-4 differ from one another in their ligand affinities and tissue distribution. Fibroblast growth factor receptor 4 (FGFR-4) is a protein that in humans is encoded by the FGFR-4 gene. FGFR-4 preferentially binds fibroblast growth factor 19 (FGF19) and has recently been associated with the progression of certain sarcomas, renal cell cancer, breast cancer, and liver cancer. Selective inhibition of FGFR-4 without inhibition of other isoforms of FGFR, including FGFR-1, may be desirable in order to avoid certain toxicities.

### SUMMARY OF THE INVENTION

The present invention describes inhibitors of FGFR-4. The present invention further describes pharmaceutical formulations that include an inhibitor of FGFR-4.

In one aspect, the invention features a compound of Formula I, or a pharmaceutically acceptable salt thereof:

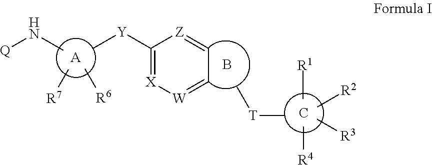

wherein each of R¹, R², R³ and R⁴ is, independently, halo, cyano, C₁₋₆ alkoxy, hydroxy, amino, C(O)NH₂, C(O)NHC₁₋₆alkyl, C(O)N(C₁₋₆alkyl)₂, C₁₋₆alkylsulfonyl, S(O)₂NH₂, S(O)₂NHC₁₋₆alkyl, NHC(O)NH₂, NHC(O)NHC₁₋₆alkyl, C₁₋₆alkyl, NHC(O)OC₁₋₆alkyl, C(O)—C₁₋₆alkyl, —C(O)C₁₋₆alkylamino, C₁₋₆heteroalkyl, heterocyclyl, or heterocyclylalkyl, wherein each of R¹, R², R³ and R⁴ is independently substituted with 0-5 occurrences of R¹⁰; Each of R¹, R², R³ and R⁴ together with the neighboring group can form a 5-12 membered carbocyclyl, 5-12 membered heterocyclyl;

each R¹° is, independently, selected from C₁₋₆-alkyl, C₁₋₆-alkoxy, halo, hydroxy, oxo, amino, cyano, cycloalkyl and heterocyclyl;

Q is a moiety capable of forming a covalent bond with a nucleophile and structures of exemplary Q are shown below:

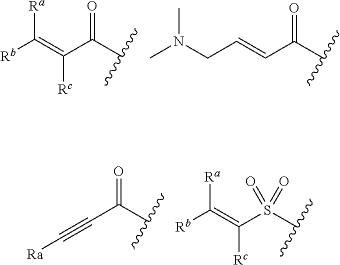

each R^{a}, R^{b}, and R^{c} is, independently, H, halogen, substituted or unsubstituted C₁₋₄alkyl, substituted or unsubstituted C₁₋₄cycloalkyl, or cyano;

ring A is a 5-10 membered aryl, 5-12 membered heteroaryl, 3-7 membered heterocyclyl or 3-12 membered cyclyloalkyl groups. Structures of exemplary ring A are shown below:

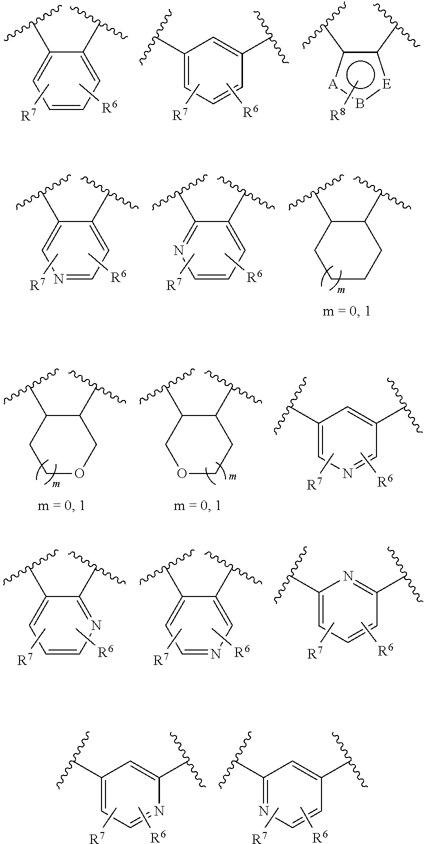

in which two of A, B, and E are N, and the other is C.

R⁶ and R⁷ are each independently selected from H, halogen, C₁₋₆alkyl, C₁₋₆halogenated alkyl, C₁₋₆cycloalkyl, C₁₋₆halogenated cycloalkyl, C₃₋₁₀heterocyclic ring and R⁶ and R⁷ can also be R⁸.

R⁸ is selected from H, halogen, C₁₋₆alkyl, C₁₋₆alkoxy, C₁₋₆halogenated alkyl, C₁₋₆cycloalkyl, C₁₋₆cycloalkoxy, C₁₋₆halogenated cycloalkyl, C₃₋₁₀heterocyclic ring, C₆₋₁₀aryl, C₃₋₁₀heteroaryl,

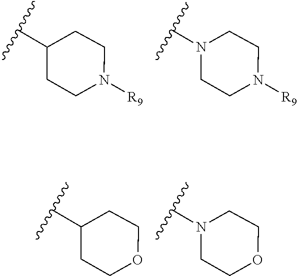

R⁹ is C₁₋₆-alkyl, C(O)R⁸, (C(O)N(R⁸)₂, C(S)R⁸, C(S)N(R⁸)₂, S(O)₂R⁸, S(O)₂N(R⁸)₂.

Y is NH, O, S, and CH₂ or absent.

Each X, W and Z is, independently, N or CR⁵. R⁵ is H, halogen, C₁₋₆alkyl, C₁₋₆halogenated alkyl.

Ring B is a 6-membered aryl, 5-membered heteroaryl, 5-7 membered heterocyclyl or 3-7 membered cyclyloalkyl groups which are unsubstituted or substituted with one or two R^{d}.

R^{d} is halogen, cyano, C₁₋₆alkyl; C₁₋₆halogenated alkyl, C₁₋₆alkoxy or R^{e}.

Structures of exemplary

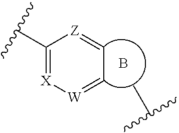

are shown below:

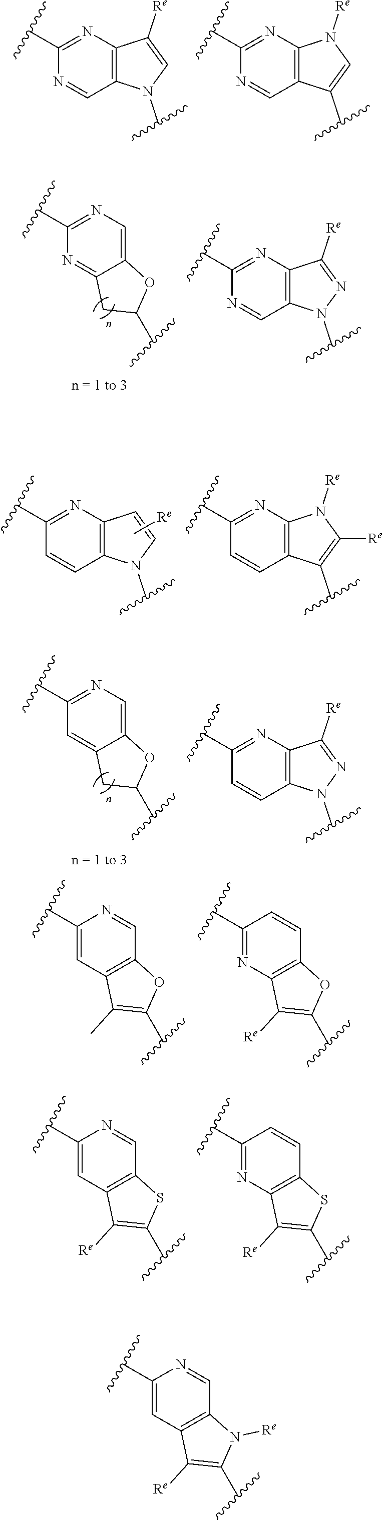

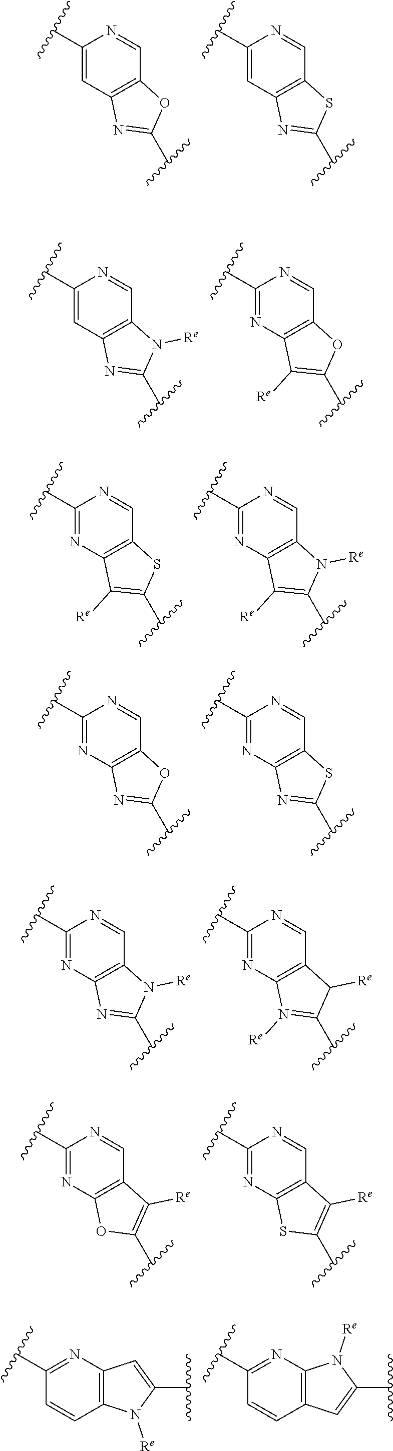

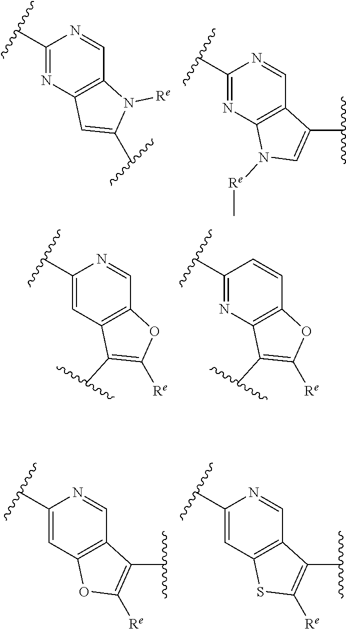

T is C(O), C(S), C(O)NR^{e}, C(S)NR^{e}, NR^{e}C(O)NR^{e}, NR^{e}C(S)NR^{e}, S(O)₂, S(O)₂NR^{e}, [C(R^{e})₂]_{q} or NR^{e}

and R^{e} is, independently, H, halogen, C₁₋₆alkyl and C₁₋₆halogenated alky, OH, OC₁₋₈alkyl, OC₁₋₈cycloalkyl, O-aryl, O-heteroaryl; alternatively, together with the carbon atom they are attached to, two R^{e} can form a 3 to 6-membered carbocyclic ring or heterocyclic ring in which one or more than one carbon atom can be replaced with a heteroatom such as O, S, S(O)₂ or NR^{e} with the proviso R^{e} is not halogen when R^{e} is on a nitrogen atom;

q is 1 to 3. or

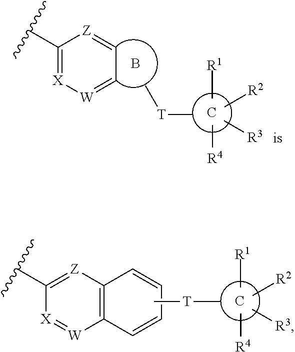

T is C(O), C(S), C(O)NR^{e}, C(S)NR^{e}, NR^{e}C(O)NR^{e}, NR^{e}C(S)NR^{e}, S(O)₂, S(O)₂NR^{e}, [C(R^{e})₂]_{q}, with the proviso that T is not a bond; Re is defined as above; q is 1 to 3.

Ring C is a 5-10 membered aryl or 5-12 membered heteroaryl.

Each of R¹, R², R³ and R⁴ is defined as above.

In some embodiments, ring A is unsubstituted, or independently substituted by R⁶ and R⁷, or independently substituted by R⁸; and R⁶, R⁷ and R⁸ are defined as above.

In another aspect, the invention features a compound or Formula II, or a pharmaceutically acceptable salt thereof:

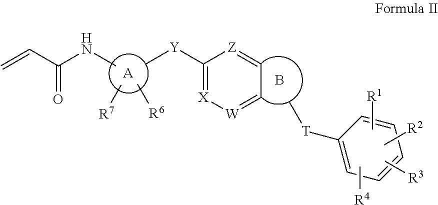

wherein Ring A, Ring , X, W, Z, T, R¹, R², R³, R⁴, R⁶ and R⁷ are defined as above.

In some embodiments, Ring A is phenyl.

In some embodiments, Ring A is cycloalkyl.

In some embodiments, Ring A is heterocyclyl.

In some embodiments, Ring A is heteroaryl.

In some embodiments, Ring A is cyclobutyl, cyclopentyl, cyclohexyl, pyrrolidinyl, piperidinyl, tetrahydrofuranyl, or tetrahydropyranyl.

In some embodiments, Ring A is

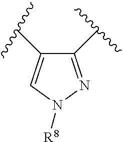

R⁸ is defined as above.

In some embodiments, a compound of Formula I is selected from the group consisting the compounds below, or a pharmaceutically acceptable salt thereof:

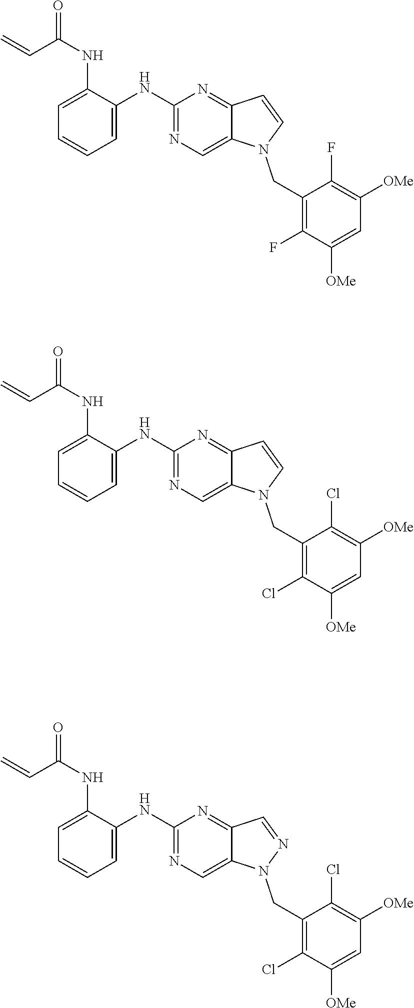 [SMILES: C=CC(=O)Nc1ccccc1Nc1ncc2c(ccn2Cc2c(F)c(OC)cc(OC)c2F)n1 ; C=CC(=O)Nc1ccccc1Nc1ncc2c(ccn2Cc2c(Cl)c(OC)cc(OC)c2Cl)n1 ; C=CC(=O)Nc1ccccc1Nc1ncc2c(cnn2Cc2c(Cl)c(OC)cc(OC)c2Cl)n1]

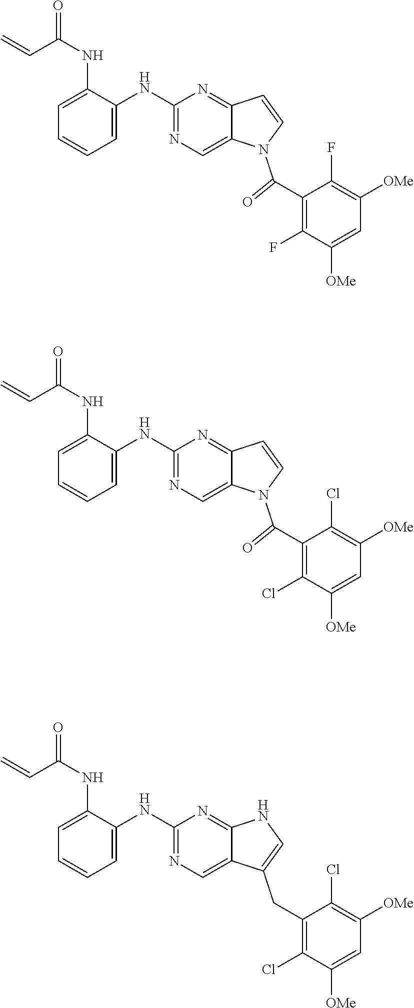 [SMILES: C=CC(=O)Nc1ccccc1Nc1ncc2c(ccn2C(=O)c2c(F)c(OC)cc(OC)c2F)n1 ; C=CC(=O)Nc1ccccc1Nc1ncc2c(ccn2C(=O)c2c(Cl)c(OC)cc(OC)c2Cl)n1 ; C=CC(=O)Nc1ccccc1Nc1ncc2c(Cc3c(Cl)c(OC)cc(OC)c3Cl)c[nH]c2n1]

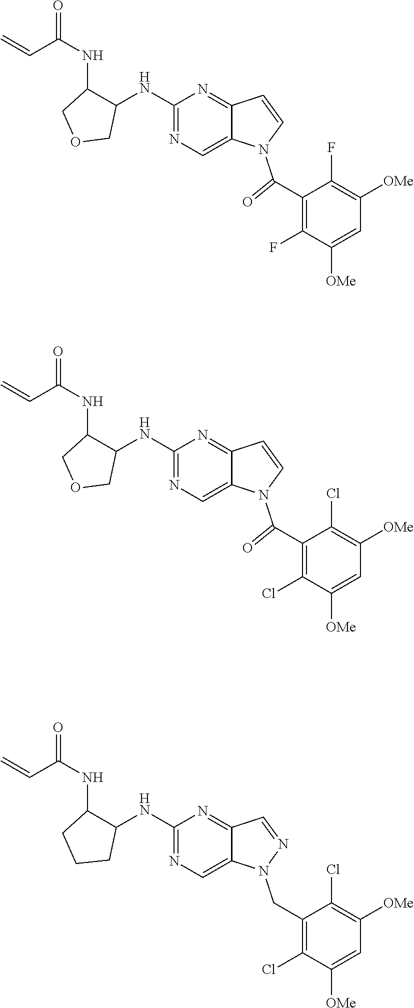 [SMILES: C=CC(=O)NC1COCC1Nc1ncc2c(ccn2C(=O)c2c(F)c(OC)cc(OC)c2F)n1 ; C=CC(=O)NC1COCC1Nc1ncc2c(ccn2C(=O)c2c(Cl)c(OC)cc(OC)c2Cl)n1 ; C=CC(=O)NC1CCCC1Nc1ncc2c(cnn2Cc2c(Cl)c(OC)cc(OC)c2Cl)n1]

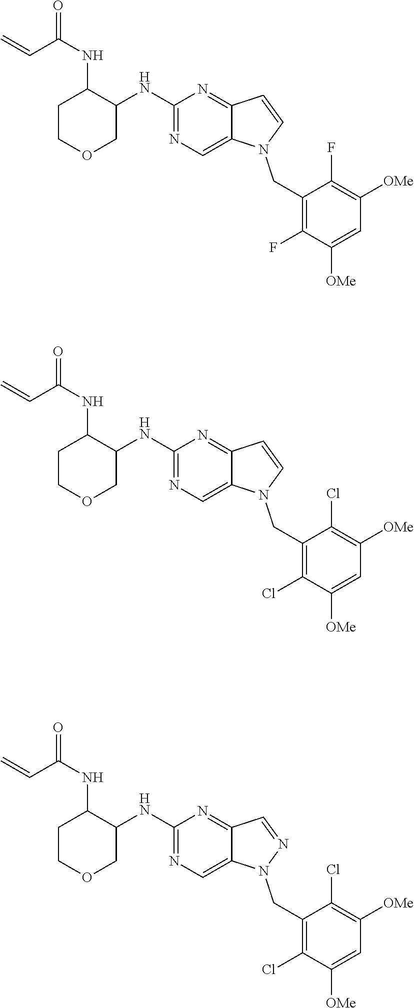 [SMILES: C=CC(=O)NC1CCOCC1Nc1ncc2c(ccn2Cc2c(F)c(OC)cc(OC)c2F)n1 ; C=CC(=O)NC1CCOCC1Nc1ncc2c(ccn2Cc2c(Cl)c(OC)cc(OC)c2Cl)n1 ; C=CC(=O)NC1CCOCC1Nc1ncc2c(cnn2Cc2c(Cl)c(OC)cc(OC)c2Cl)n1]

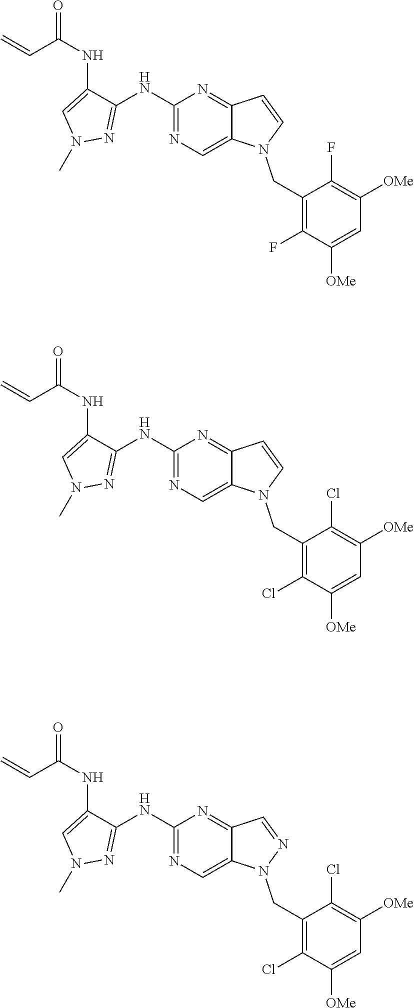 [SMILES: C=CC(=O)Nc1cn(C)nc1Nc1ncc2c(ccn2Cc2c(F)c(OC)cc(OC)c2F)n1 ; C=CC(=O)Nc1cn(C)nc1Nc1ncc2c(ccn2Cc2c(Cl)c(OC)cc(OC)c2Cl)n1 ; C=CC(=O)Nc1cn(C)nc1Nc1ncc2c(cnn2Cc2c(Cl)c(OC)cc(OC)c2Cl)n1]

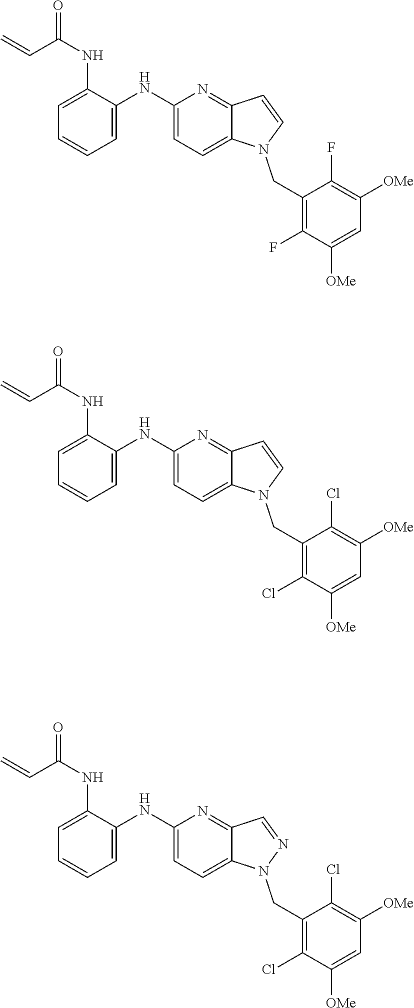 [SMILES: C=CC(=O)Nc1ccccc1Nc1ccc2c(ccn2Cc2c(F)c(OC)cc(OC)c2F)n1 ; C=CC(=O)Nc1ccccc1Nc1ccc2c(ccn2Cc2c(Cl)c(OC)cc(OC)c2Cl)n1 ; C=CC(=O)Nc1ccccc1Nc1ccc2c(cnn2Cc2c(Cl)c(OC)cc(OC)c2Cl)n1]

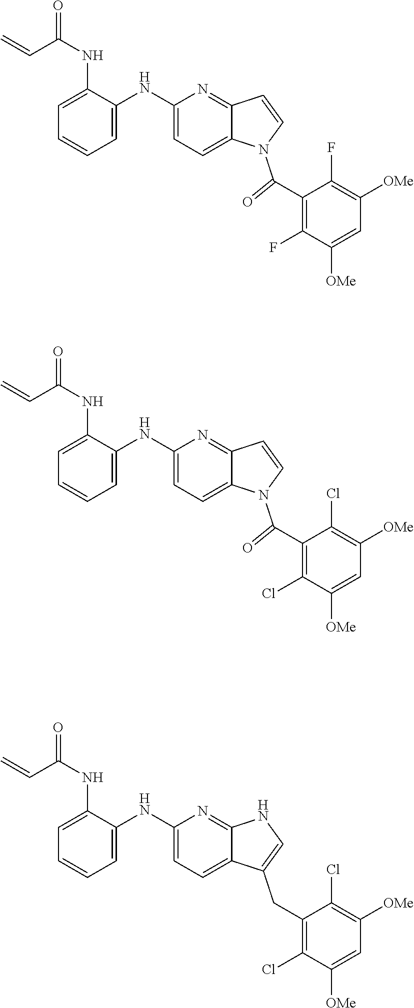 [SMILES: C=CC(=O)Nc1ccccc1Nc1ccc2c(ccn2C(=O)c2c(F)c(OC)cc(OC)c2F)n1 ; C=CC(=O)Nc1ccccc1Nc1ccc2c(ccn2C(=O)c2c(Cl)c(OC)cc(OC)c2Cl)n1 ; C=CC(=O)Nc1ccccc1Nc1ccc2c(Cc3c(Cl)c(OC)cc(OC)c3Cl)c[nH]c2n1]

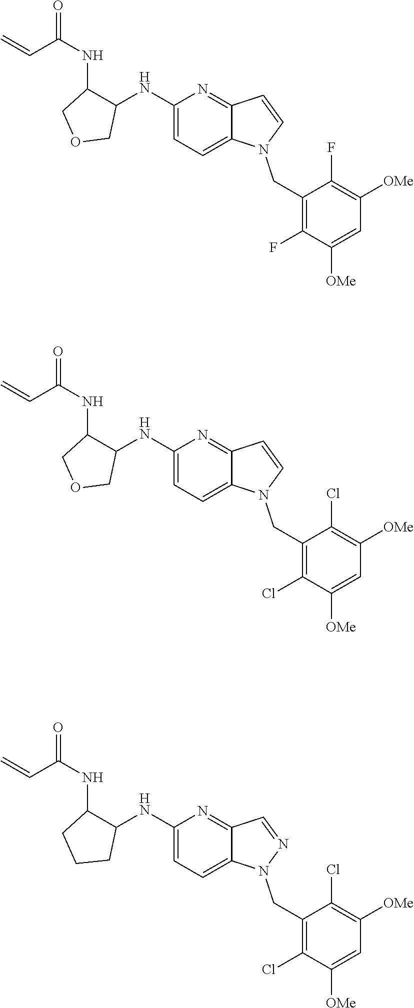 [SMILES: C=CC(=O)NC1COCC1Nc1ccc2c(ccn2Cc2c(F)c(OC)cc(OC)c2F)n1 ; C=CC(=O)NC1COCC1Nc1ccc2c(ccn2Cc2c(Cl)c(OC)cc(OC)c2Cl)n1 ; C=CC(=O)NC1CCCC1Nc1ccc2c(cnn2Cc2c(Cl)c(OC)cc(OC)c2Cl)n1]

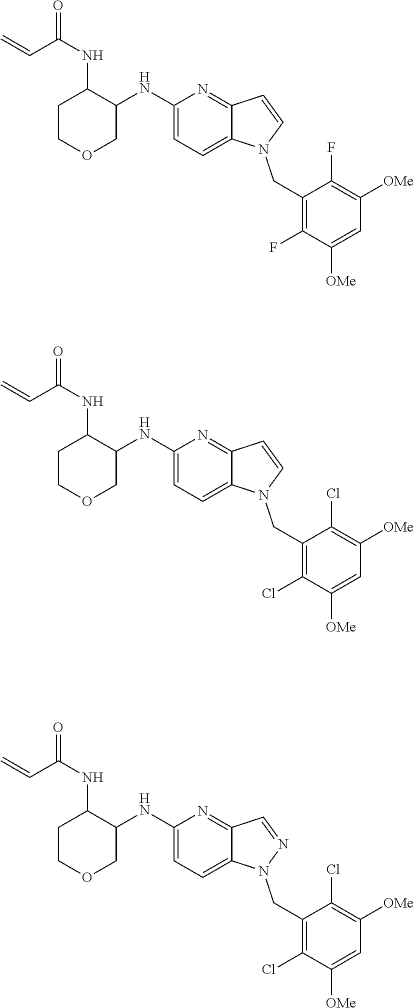 [SMILES: C=CC(=O)NC1CCOCC1Nc1ccc2c(ccn2Cc2c(F)c(OC)cc(OC)c2F)n1 ; C=CC(=O)NC1CCOCC1Nc1ccc2c(ccn2Cc2c(Cl)c(OC)cc(OC)c2Cl)n1 ; C=CC(=O)NC1CCOCC1Nc1ccc2c(cnn2Cc2c(Cl)c(OC)cc(OC)c2Cl)n1]

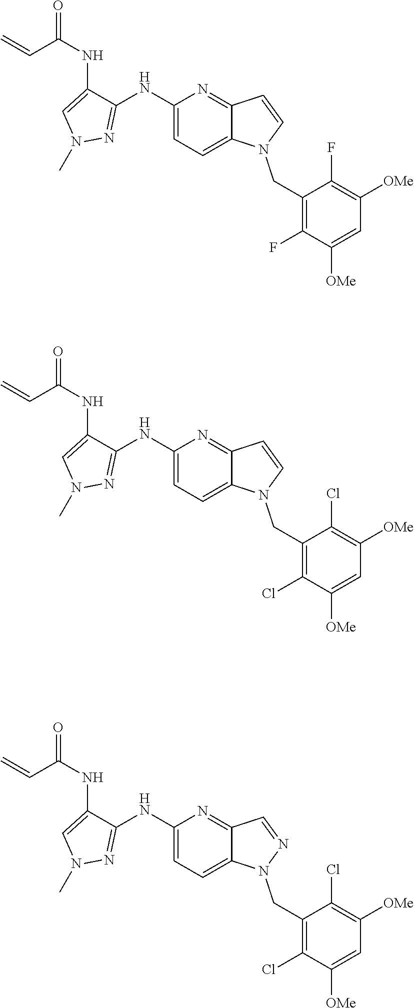 [SMILES: C=CC(=O)Nc1cn(C)nc1Nc1ccc2c(ccn2Cc2c(F)c(OC)cc(OC)c2F)n1 ; C=CC(=O)Nc1cn(C)nc1Nc1ccc2c(ccn2Cc2c(Cl)c(OC)cc(OC)c2Cl)n1 ; C=CC(=O)Nc1cn(C)nc1Nc1ccc2c(cnn2Cc2c(Cl)c(OC)cc(OC)c2Cl)n1]

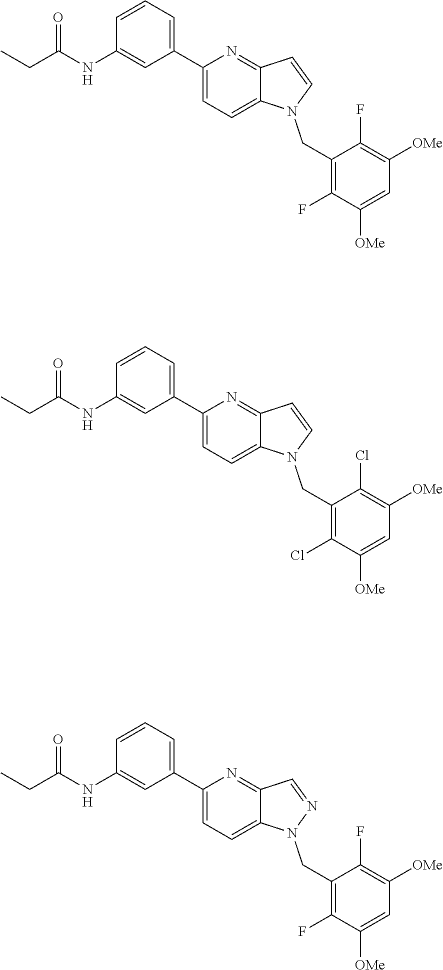 [SMILES: CCC(=O)Nc1cccc(-c2ccc3c(ccn3Cc3c(F)c(OC)cc(OC)c3F)n2)c1 ; CCC(=O)Nc1cccc(-c2ccc3c(ccn3Cc3c(Cl)c(OC)cc(OC)c3Cl)n2)c1 ; CCC(=O)Nc1cccc(-c2ccc3c(cnn3Cc3c(F)c(OC)cc(OC)c3F)n2)c1]

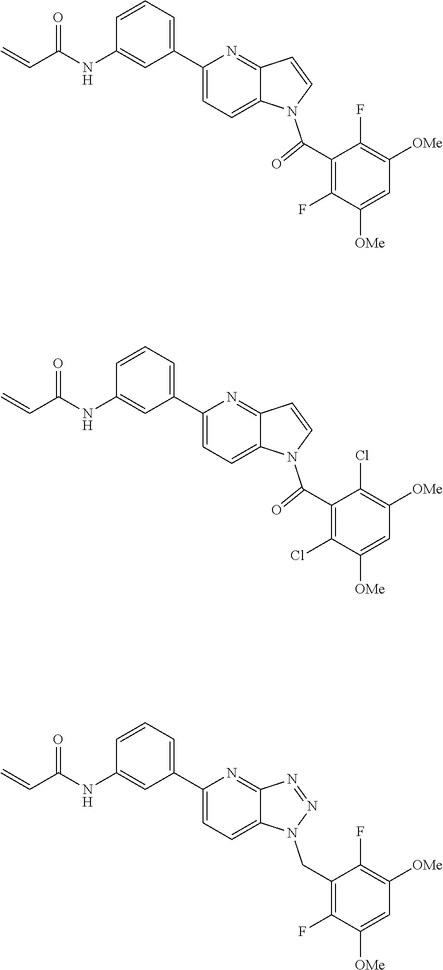 [SMILES: C=CC(=O)Nc1cccc(-c2ccc3c(ccn3C(=O)c3c(F)c(OC)cc(OC)c3F)n2)c1 ; C=CC(=O)Nc1cccc(-c2ccc3c(ccn3C(=O)c3c(Cl)c(OC)cc(OC)c3Cl)n2)c1 ; C=CC(=O)Nc1cccc(-c2ccc3c(nnn3Cc3c(F)c(OC)cc(OC)c3F)n2)c1]

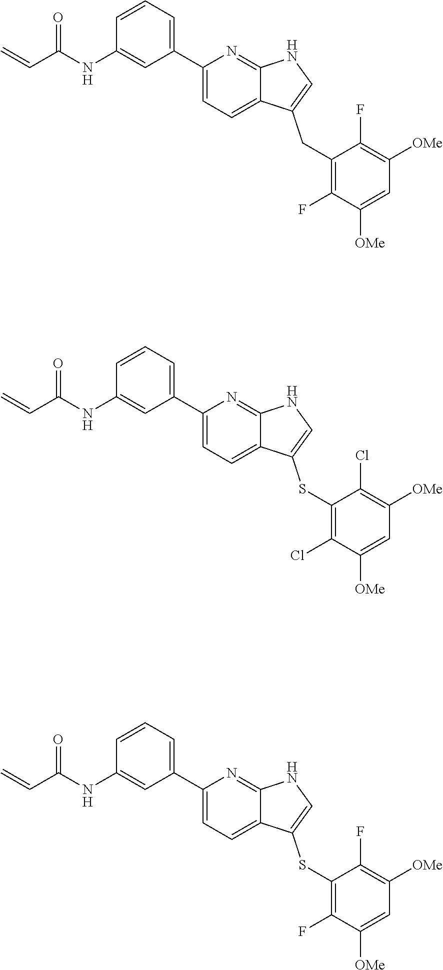 [SMILES: C=CC(=O)Nc1cccc(-c2ccc3c(Cc4c(F)c(OC)cc(OC)c4F)c[nH]c3n2)c1 ; C=CC(=O)Nc1cccc(-c2ccc3c(Sc4c(Cl)c(OC)cc(OC)c4Cl)c[nH]c3n2)c1 ; C=CC(=O)Nc1cccc(-c2ccc3c(Sc4c(F)c(OC)cc(OC)c4F)c[nH]c3n2)c1]

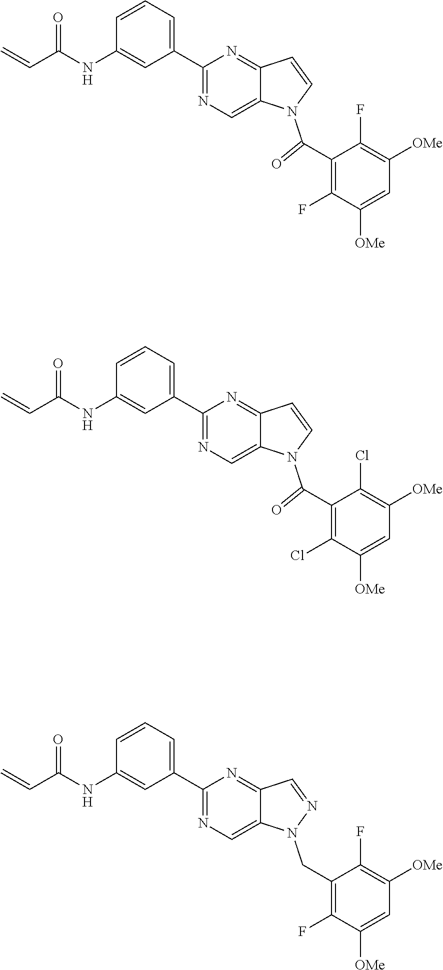 [SMILES: C=CC(=O)Nc1cccc(-c2ncc3c(ccn3C(=O)c3c(F)c(OC)cc(OC)c3F)n2)c1 ; C=CC(=O)Nc1cccc(-c2ncc3c(ccn3C(=O)c3c(Cl)c(OC)cc(OC)c3Cl)n2)c1 ; C=CC(=O)Nc1cccc(-c2ncc3c(cnn3Cc3c(F)c(OC)cc(OC)c3F)n2)c1]

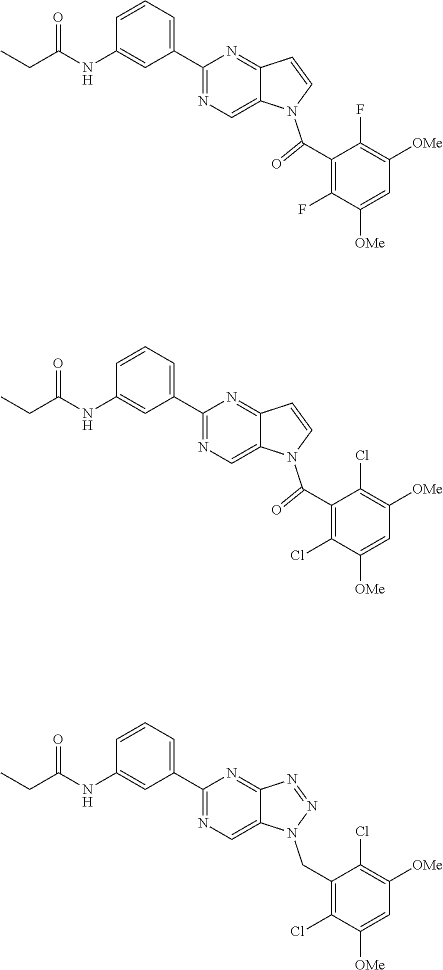 [SMILES: CCC(=O)Nc1cccc(-c2ncc3c(ccn3C(=O)c3c(F)c(OC)cc(OC)c3F)n2)c1 ; CCC(=O)Nc1cccc(-c2ncc3c(ccn3C(=O)c3c(Cl)c(OC)cc(OC)c3Cl)n2)c1 ; CCC(=O)Nc1cccc(-c2ncc3c(nnn3Cc3c(Cl)c(OC)cc(OC)c3Cl)n2)c1]

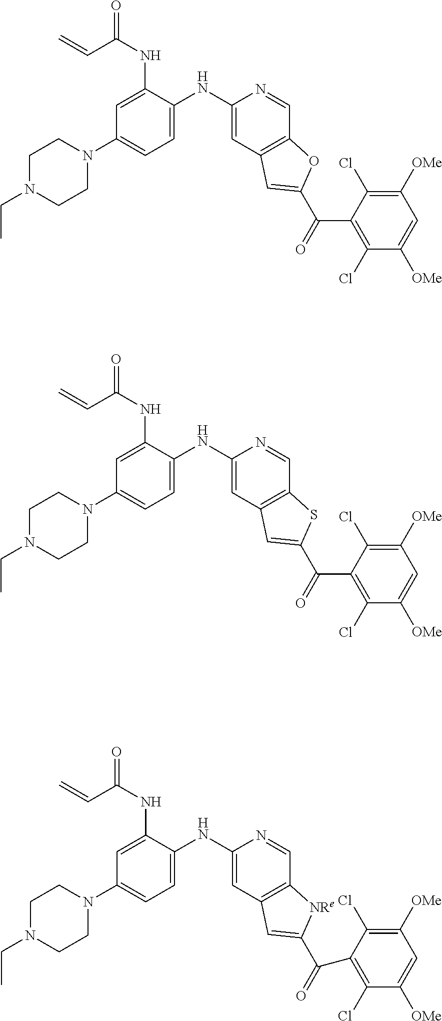

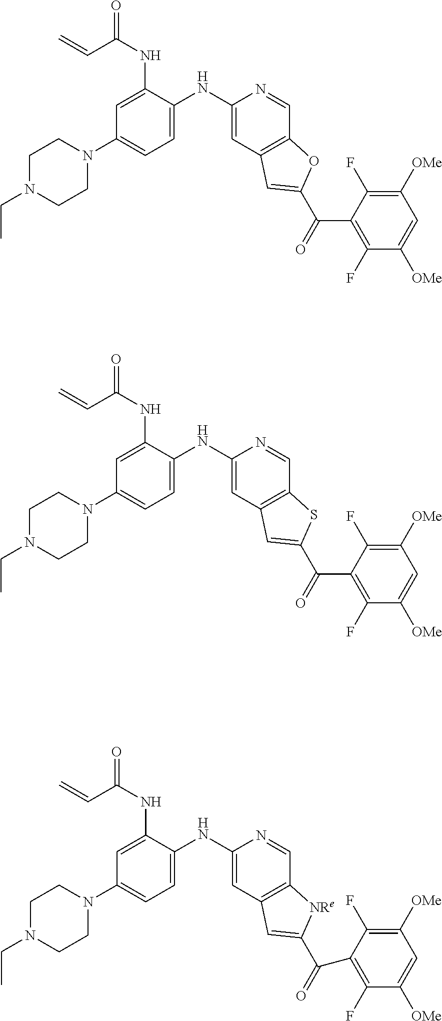

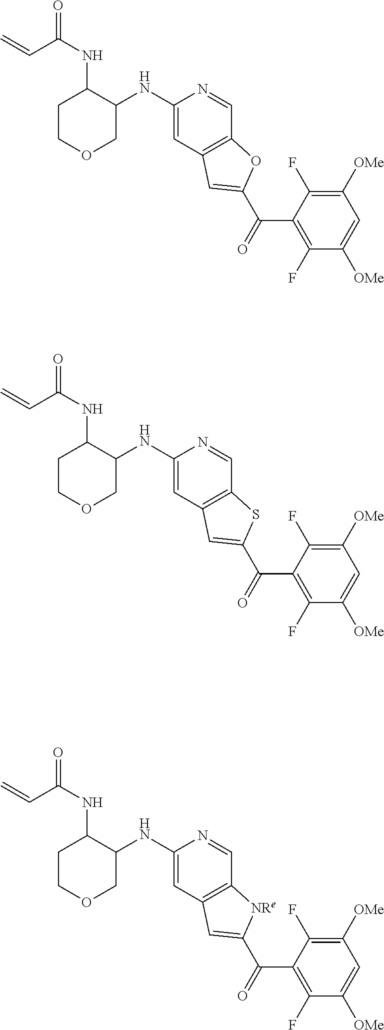

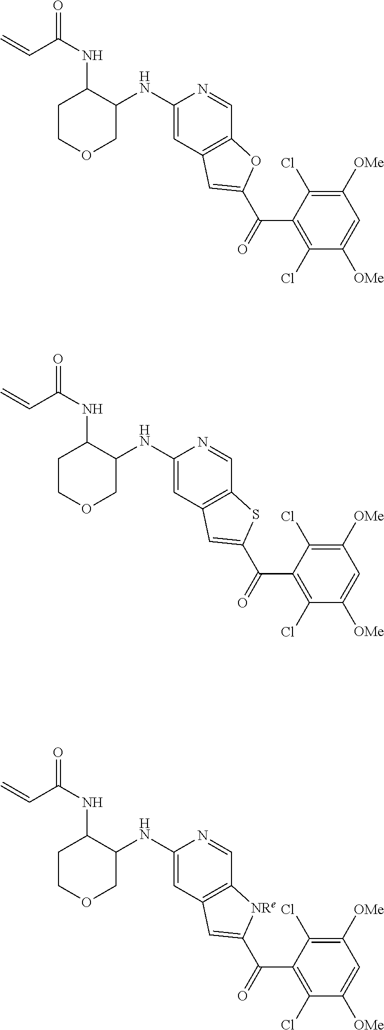

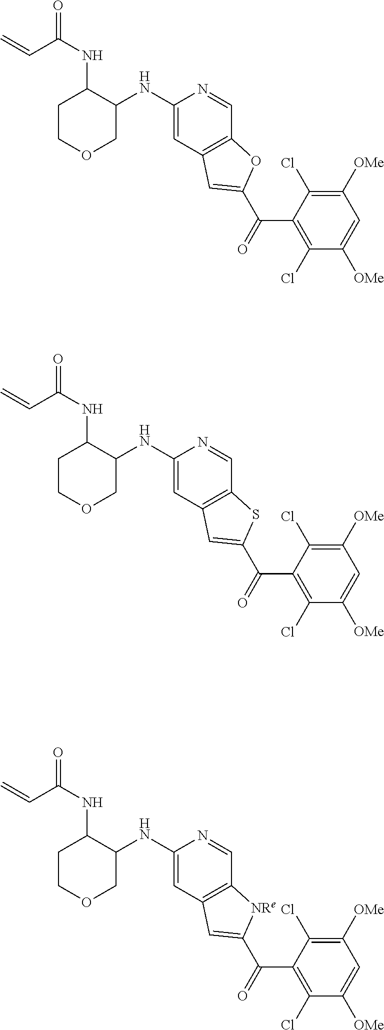

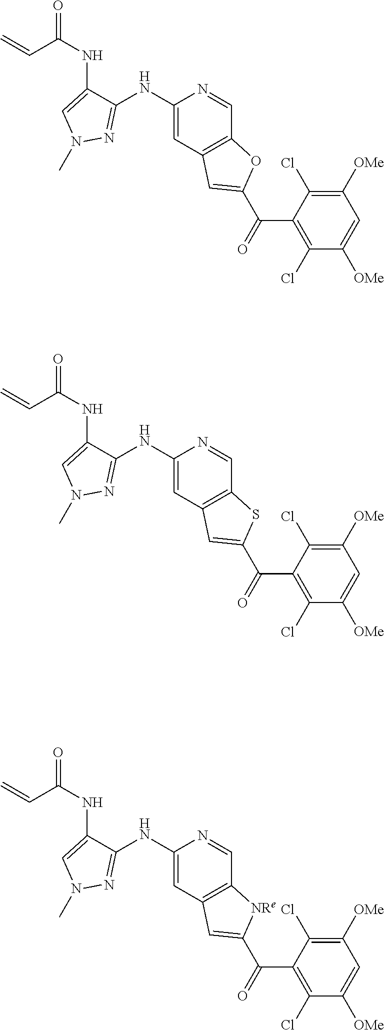

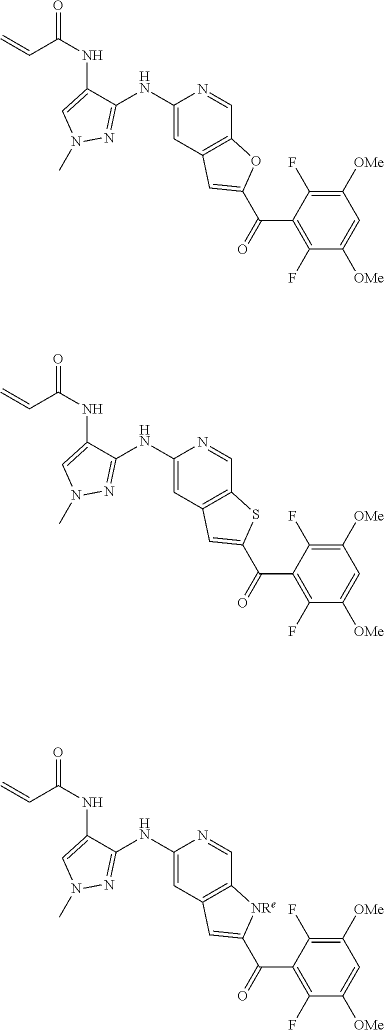

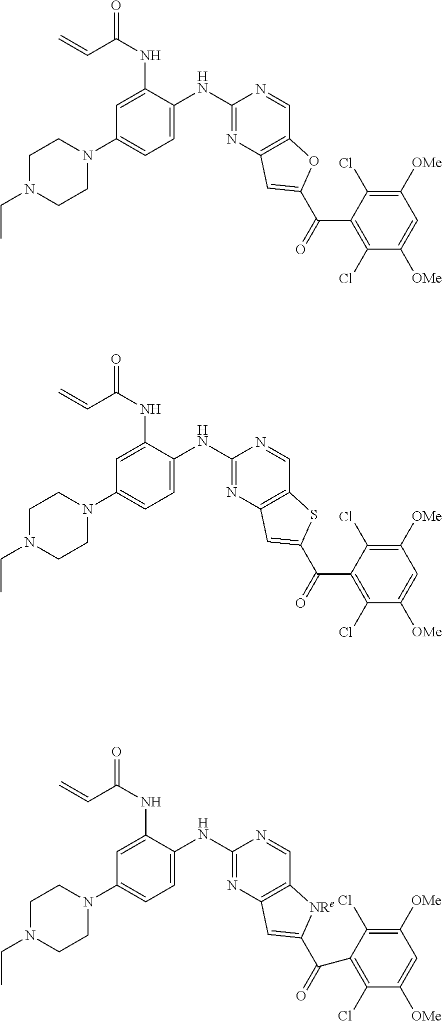

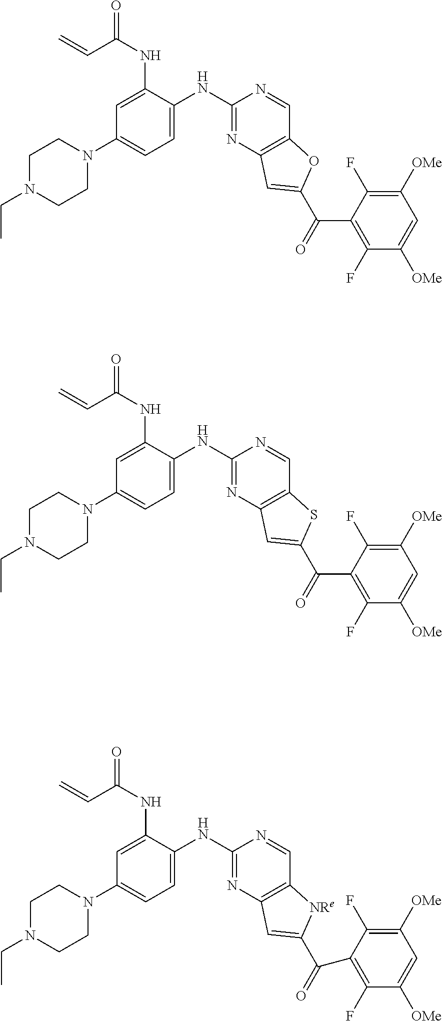

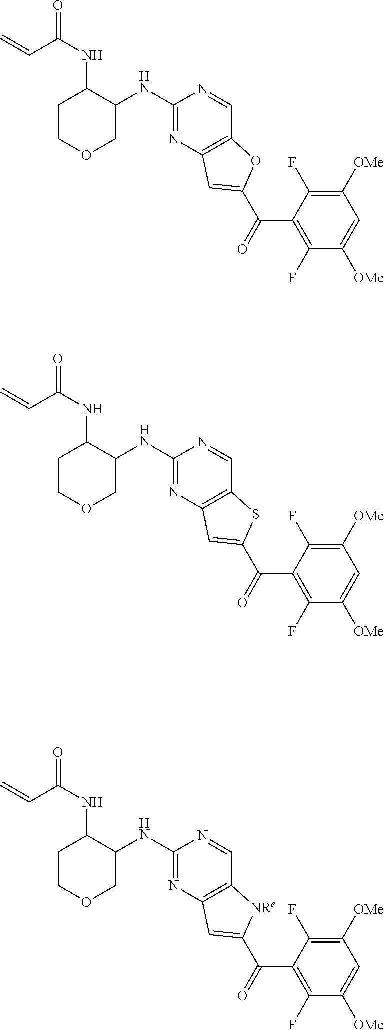

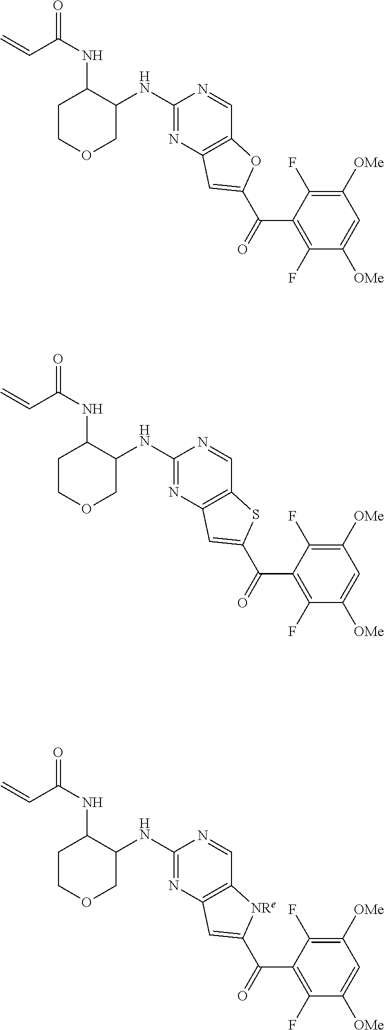

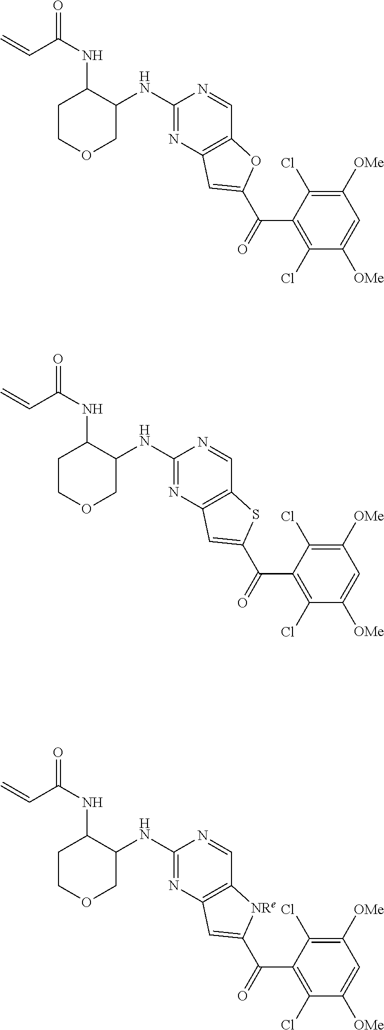

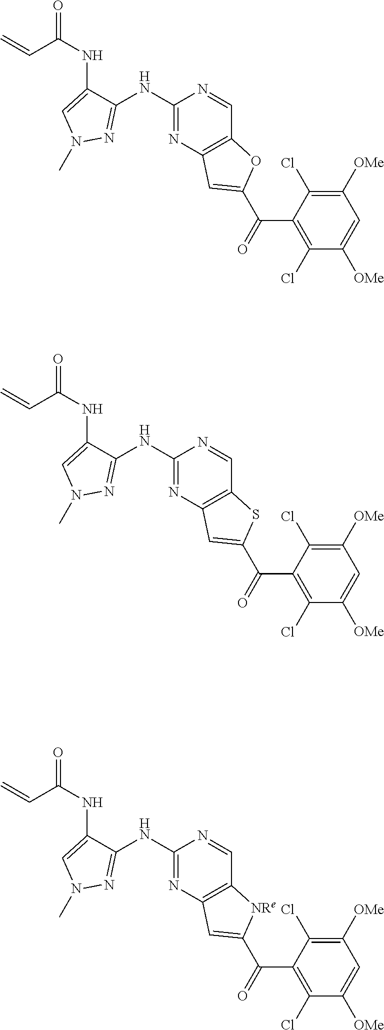

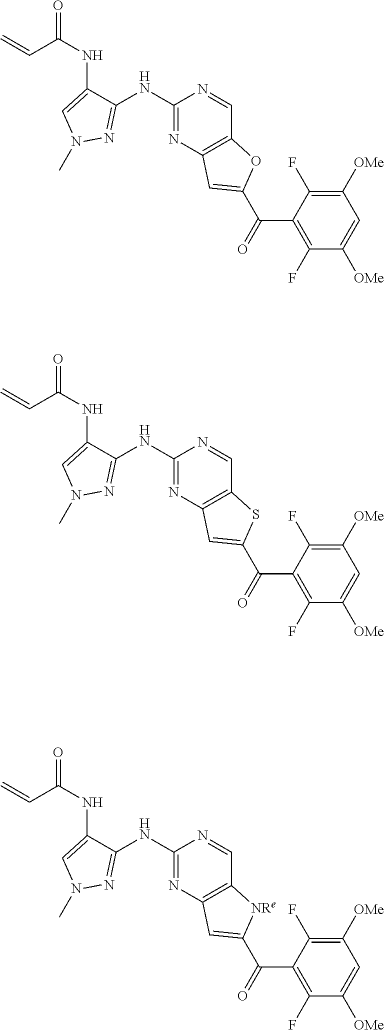

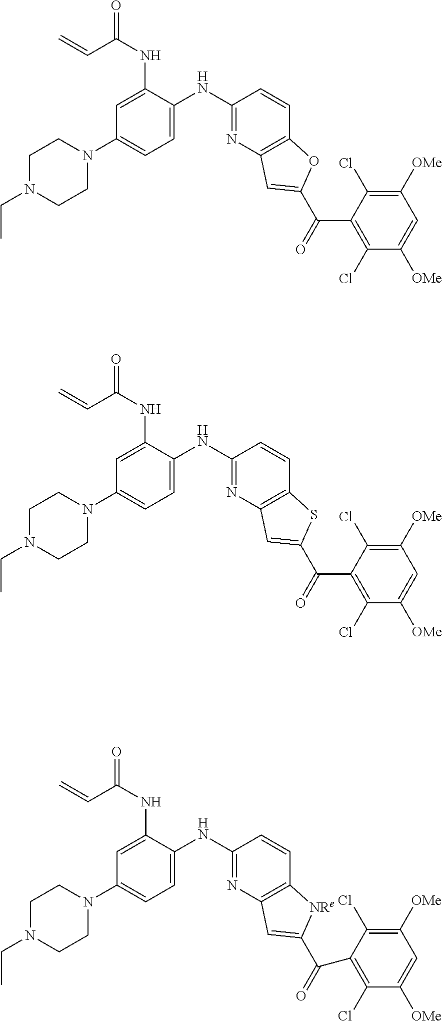

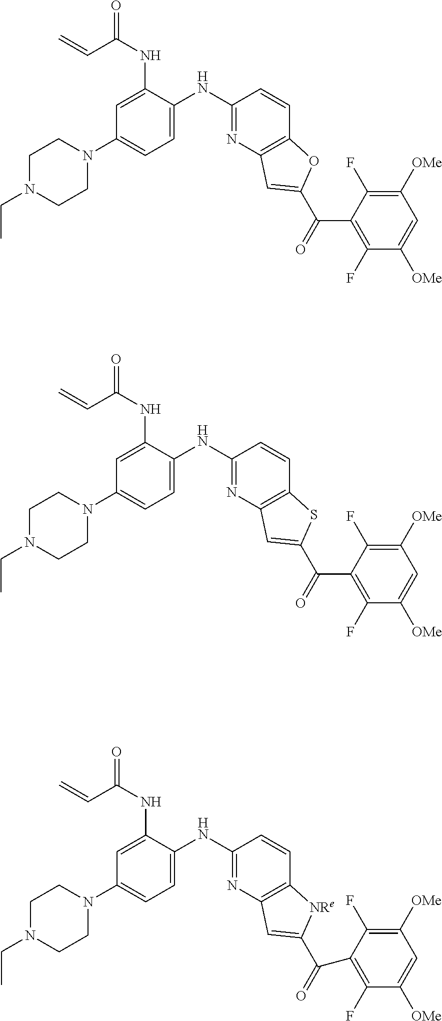

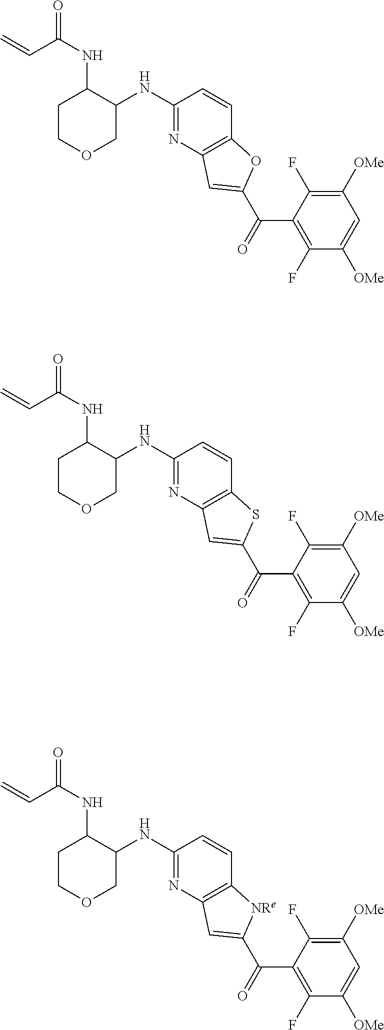

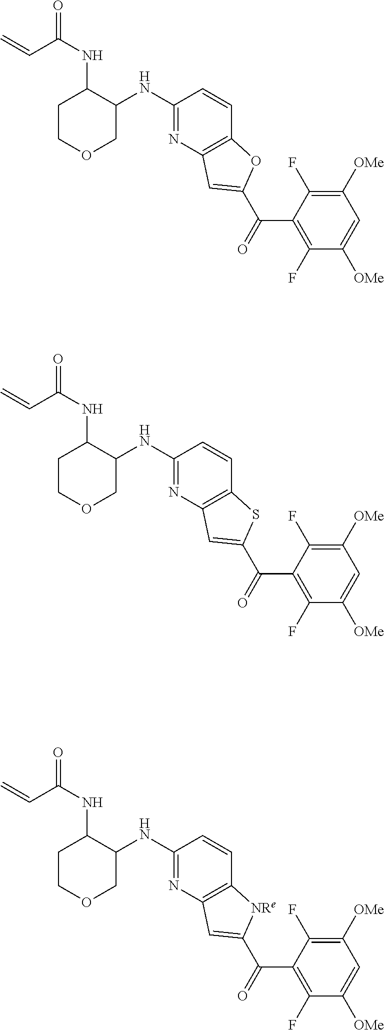

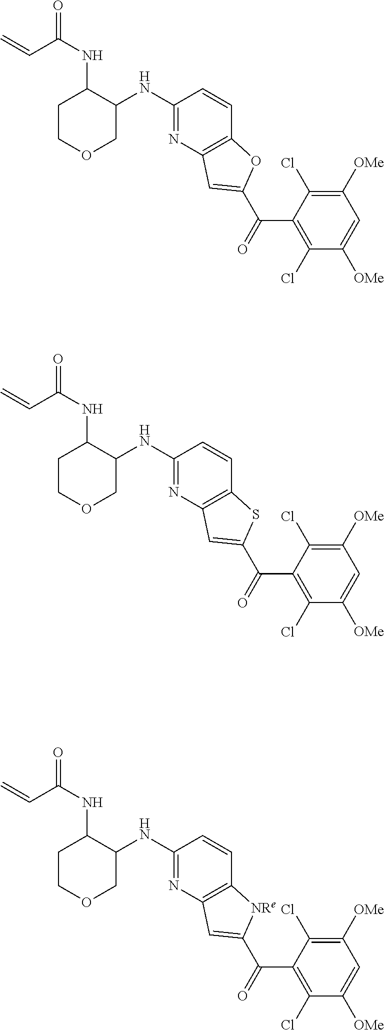

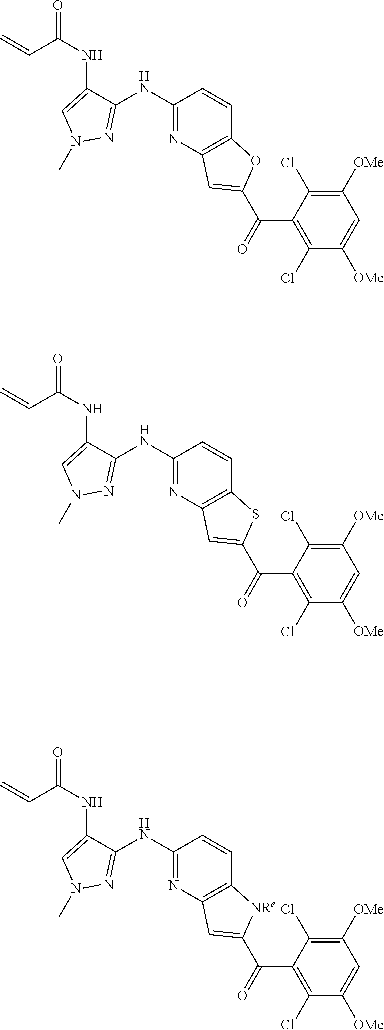

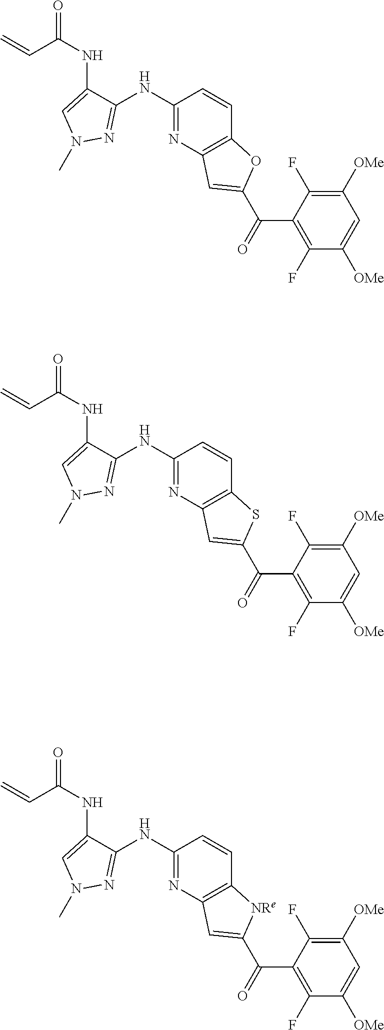

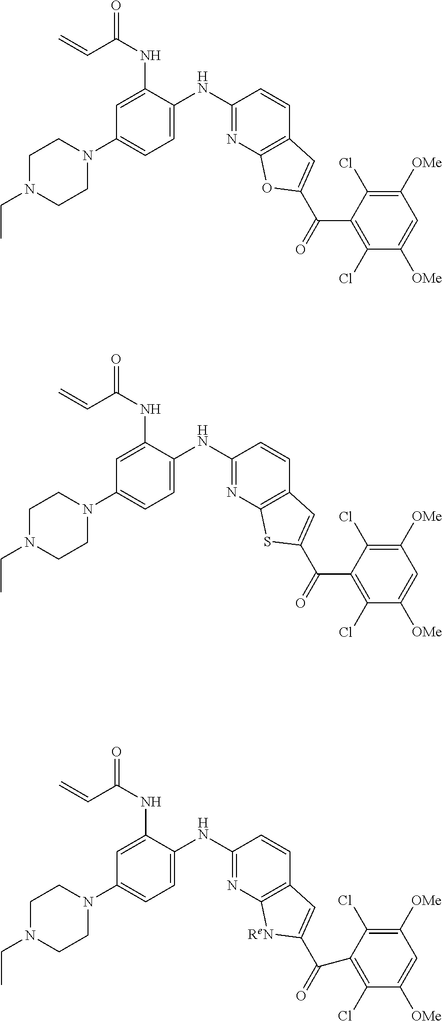

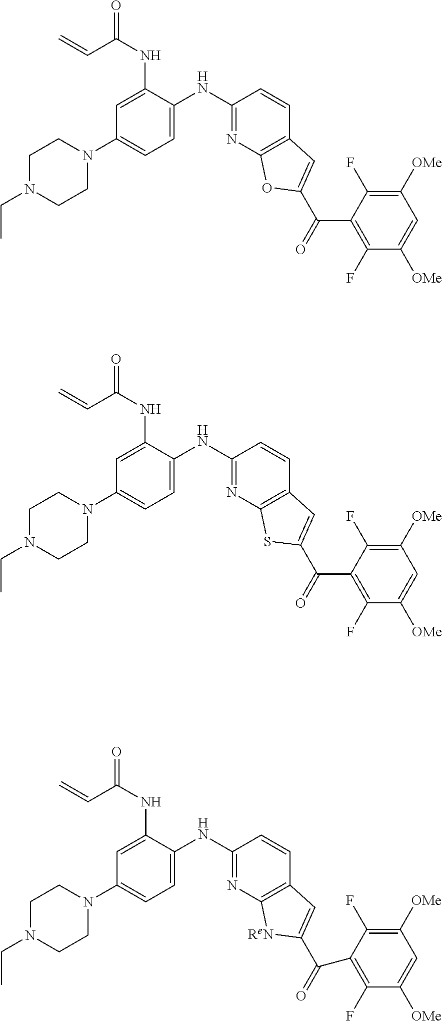

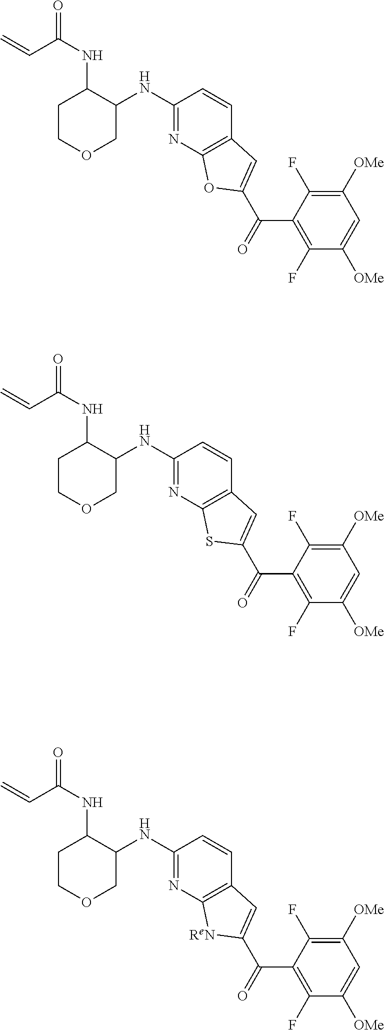

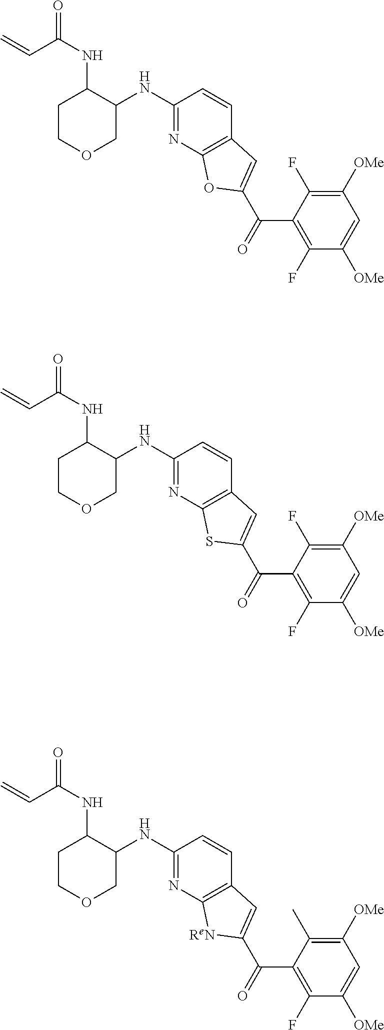

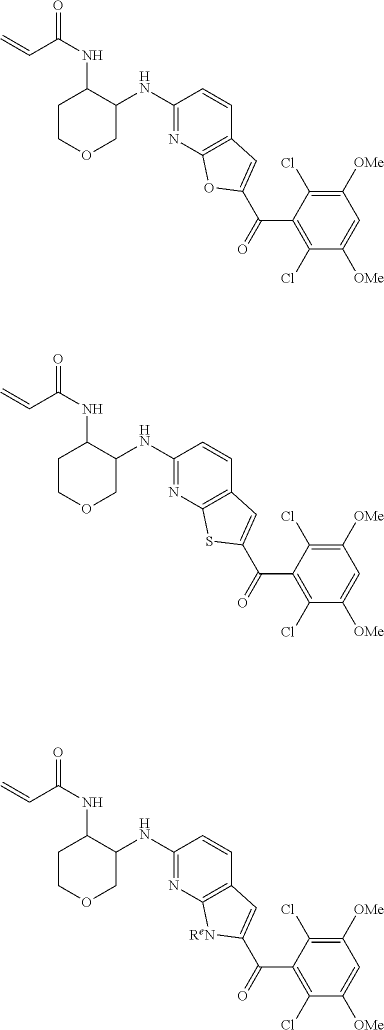

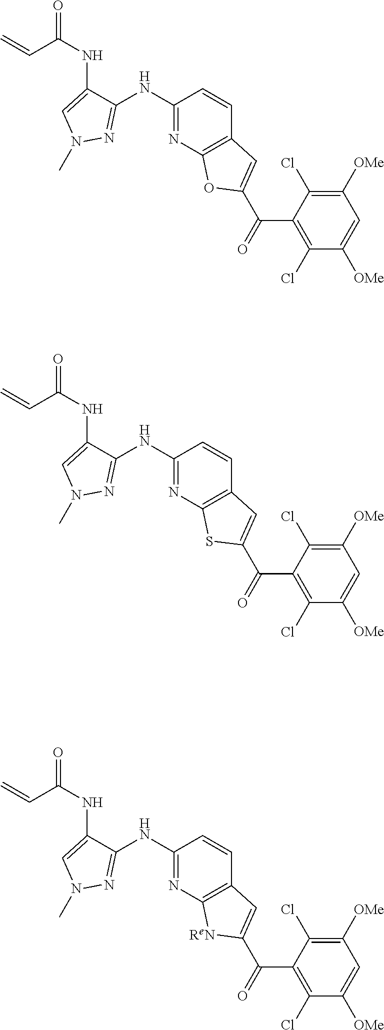

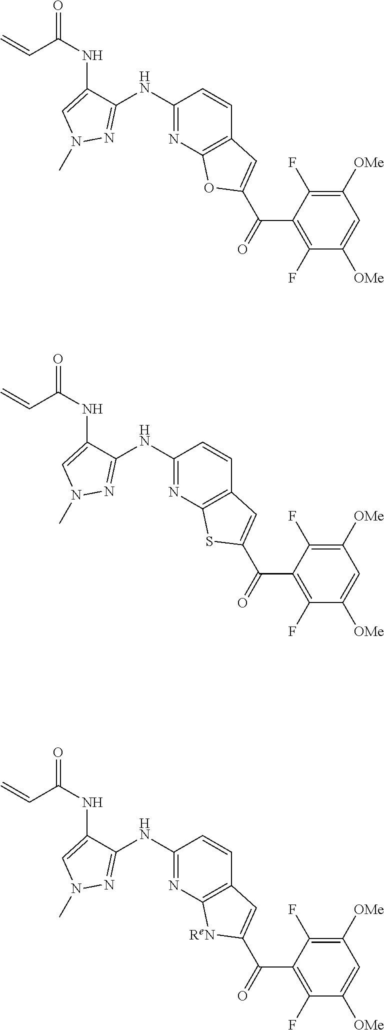

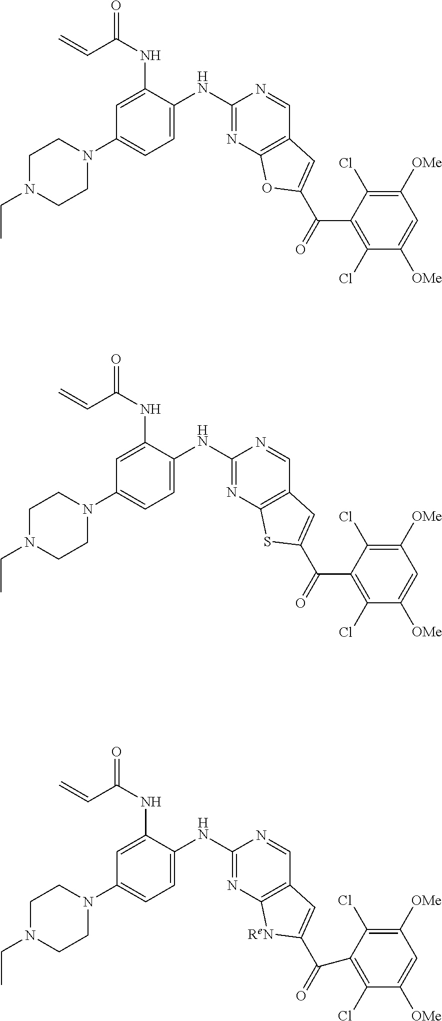

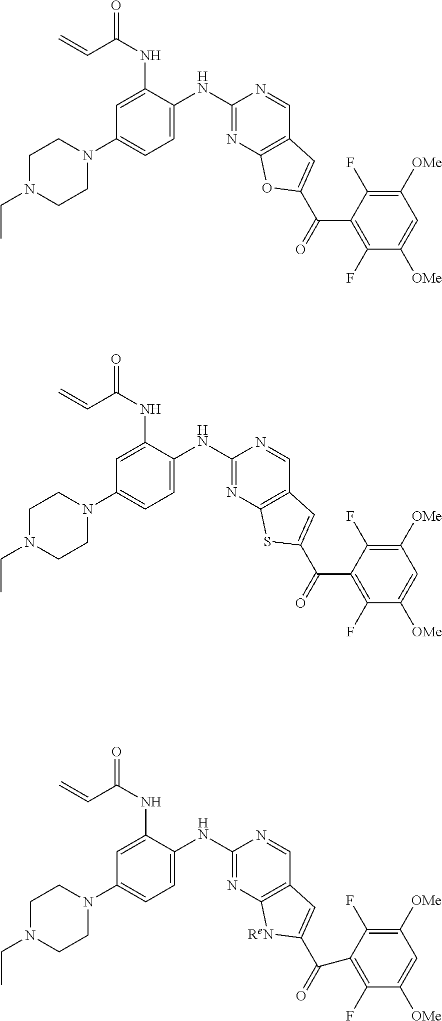

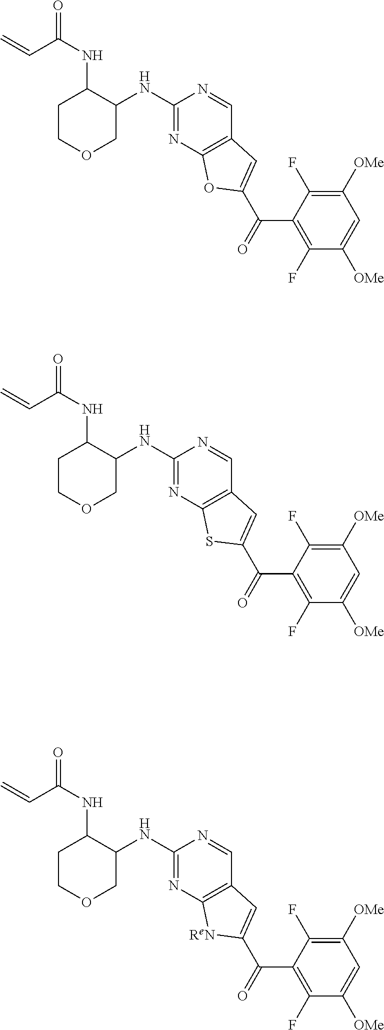

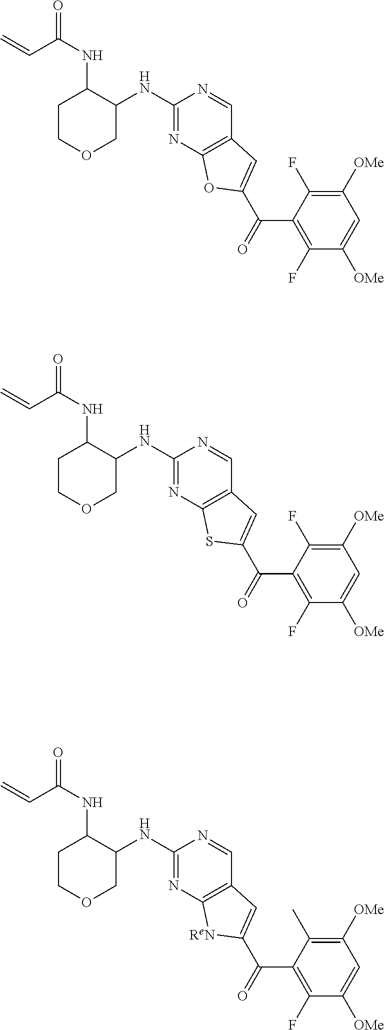

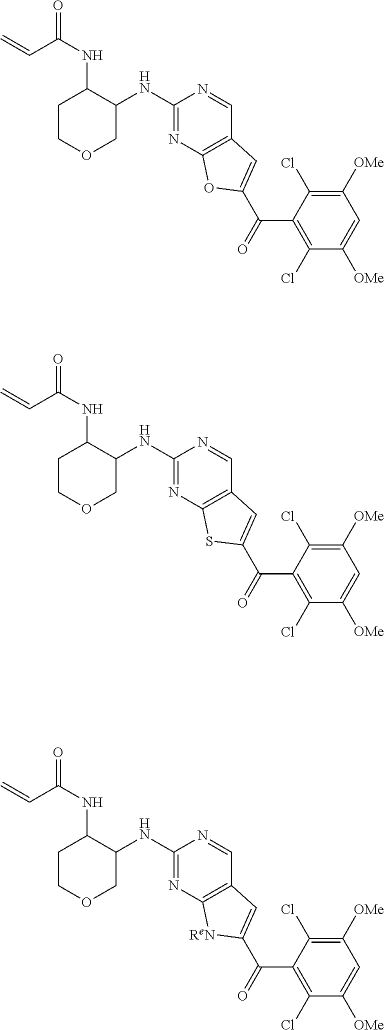

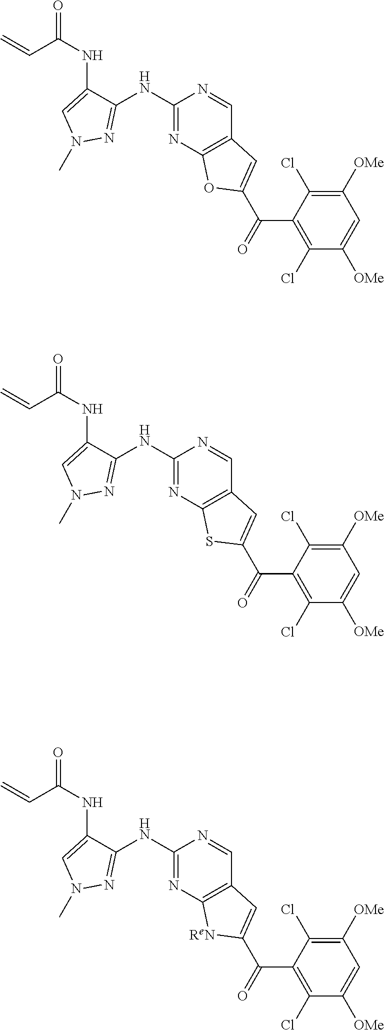

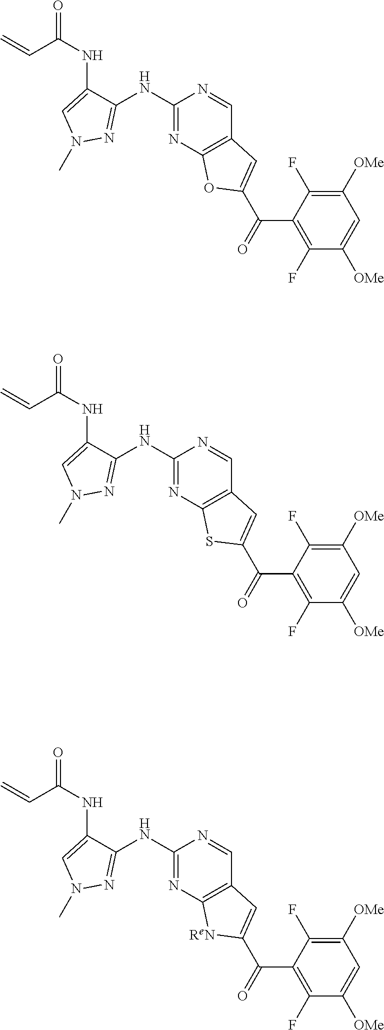

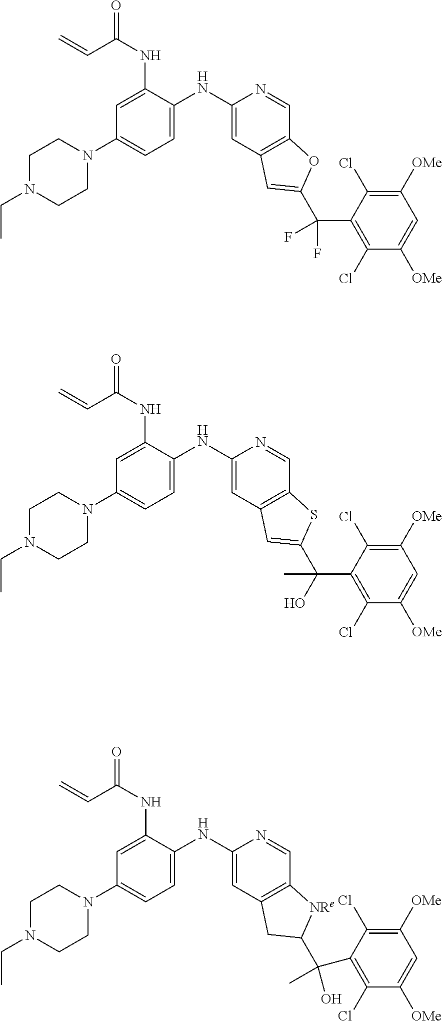

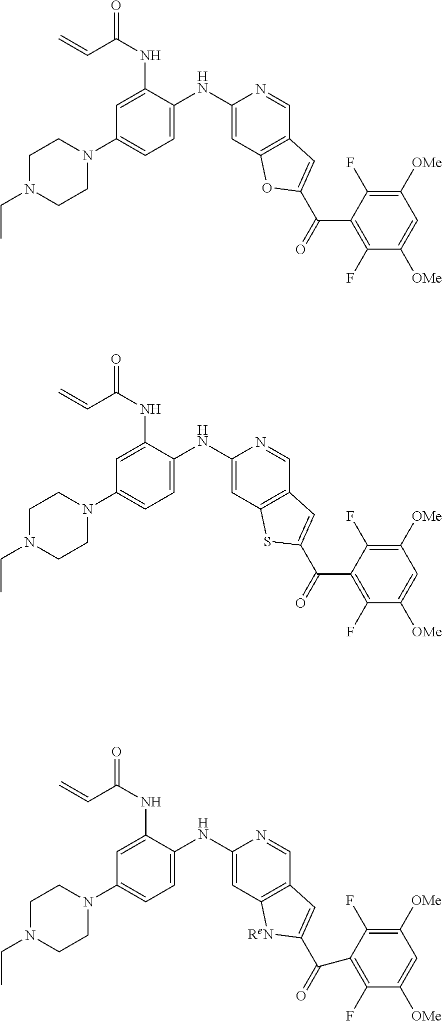

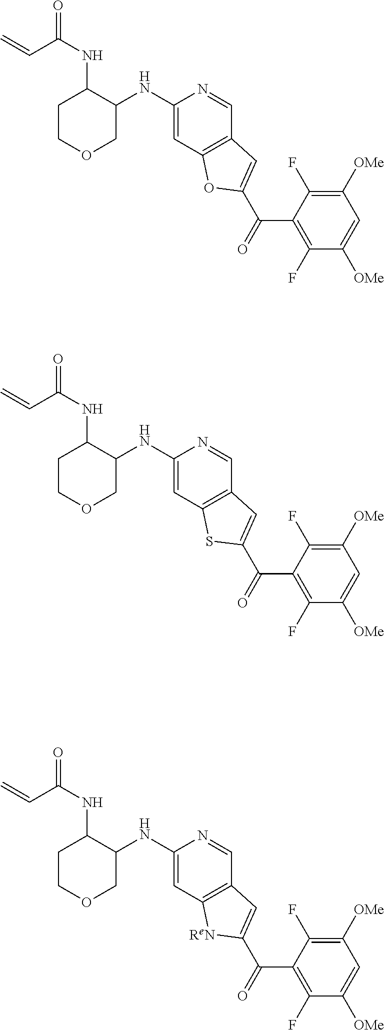

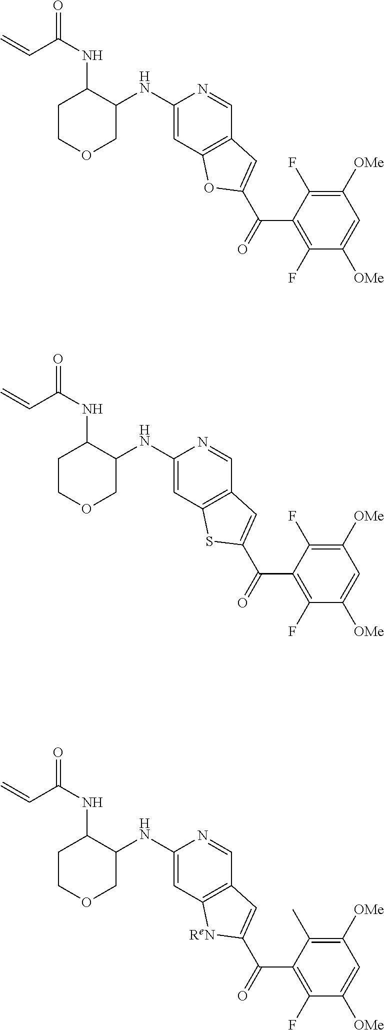

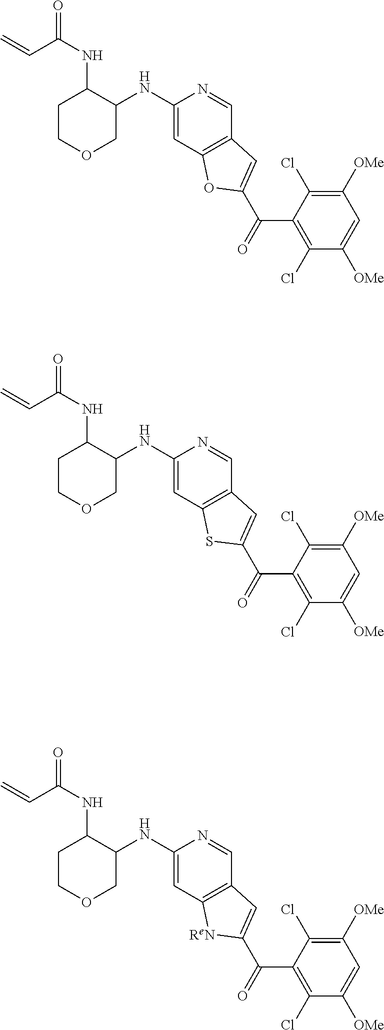

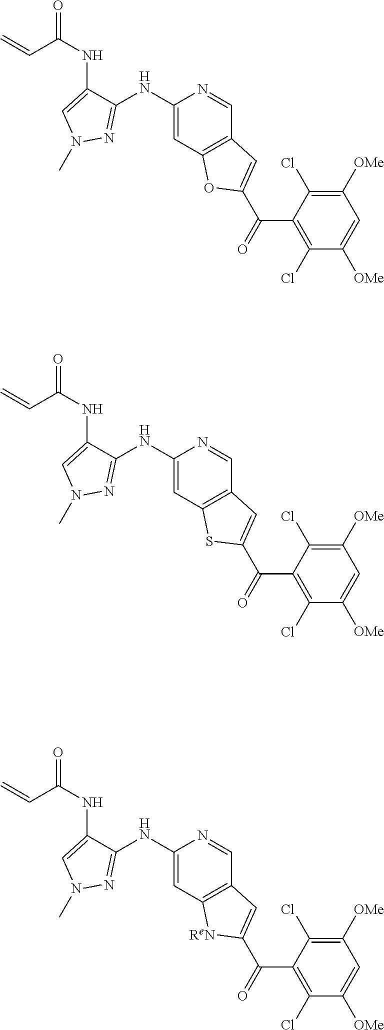

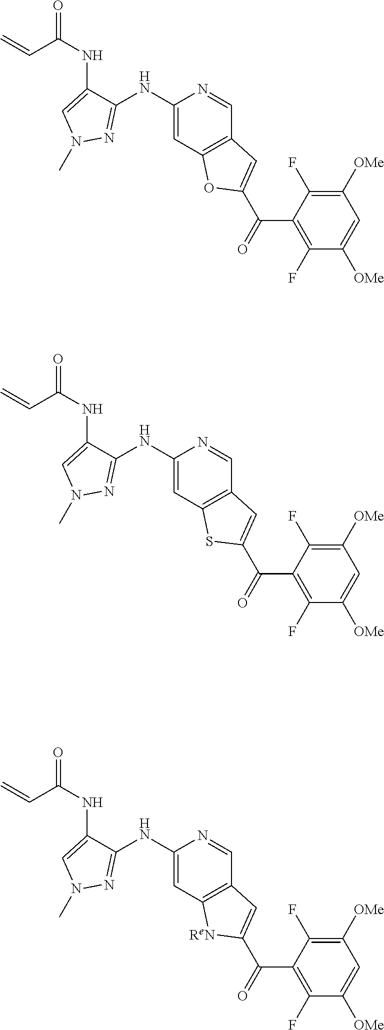

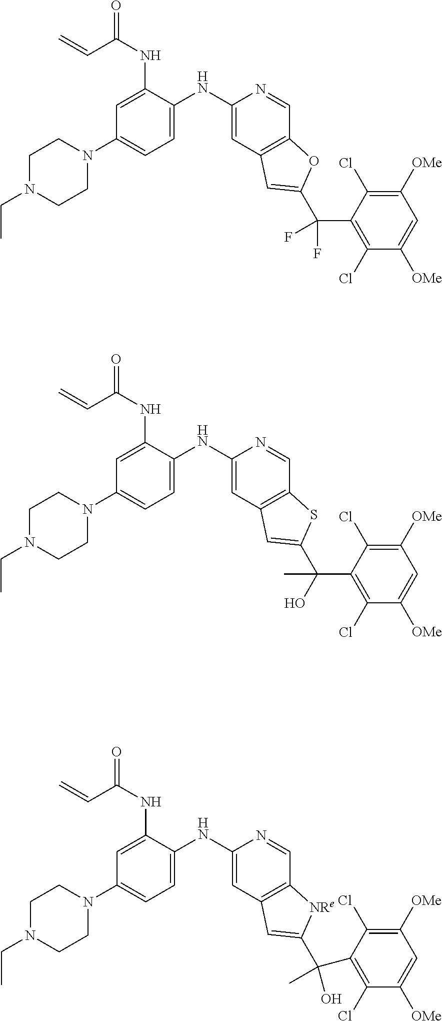

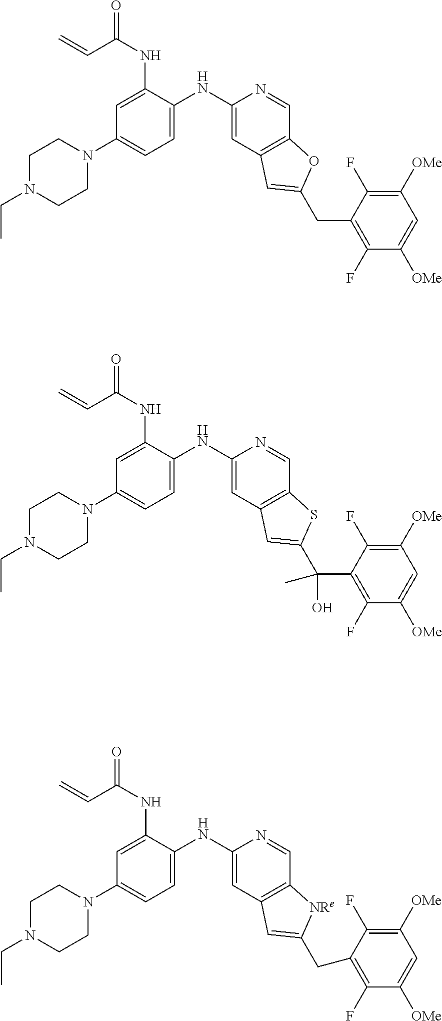

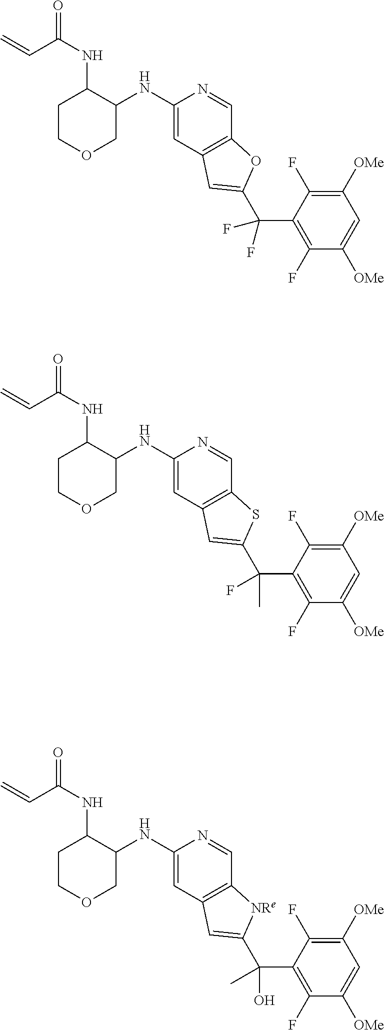

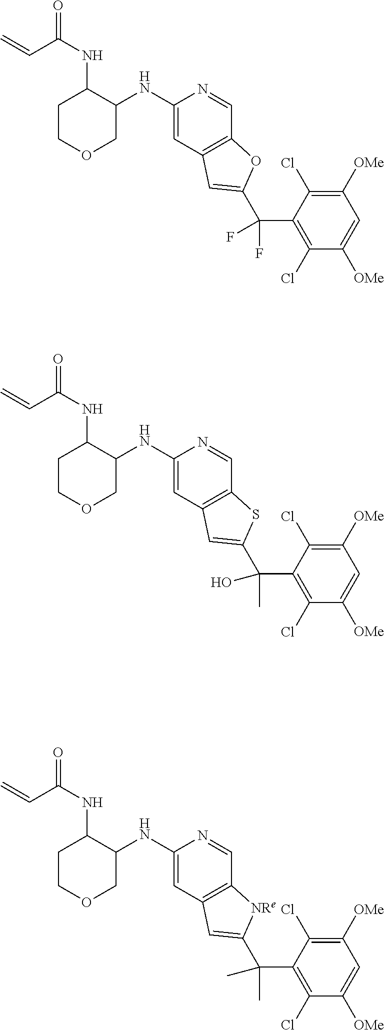

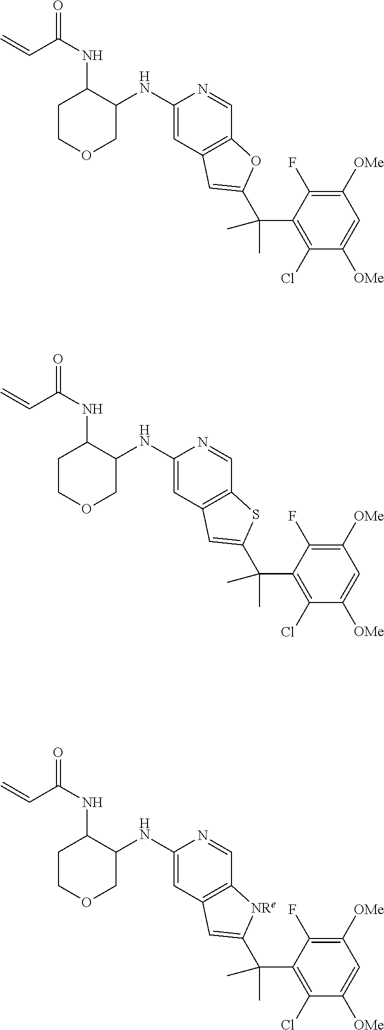

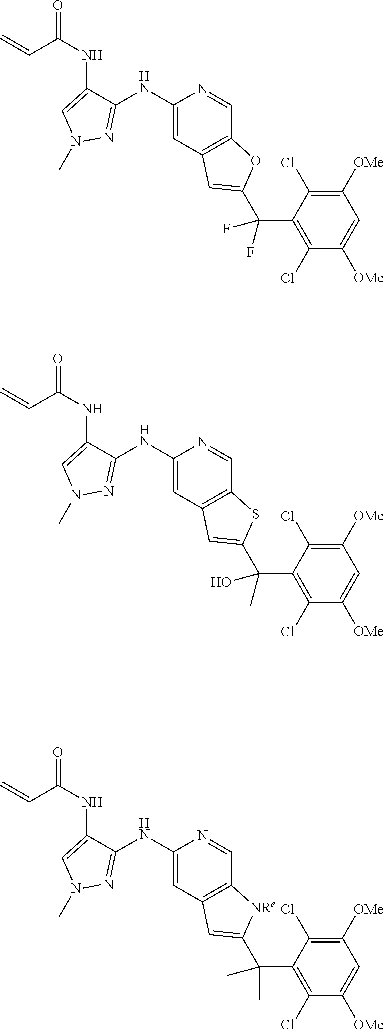

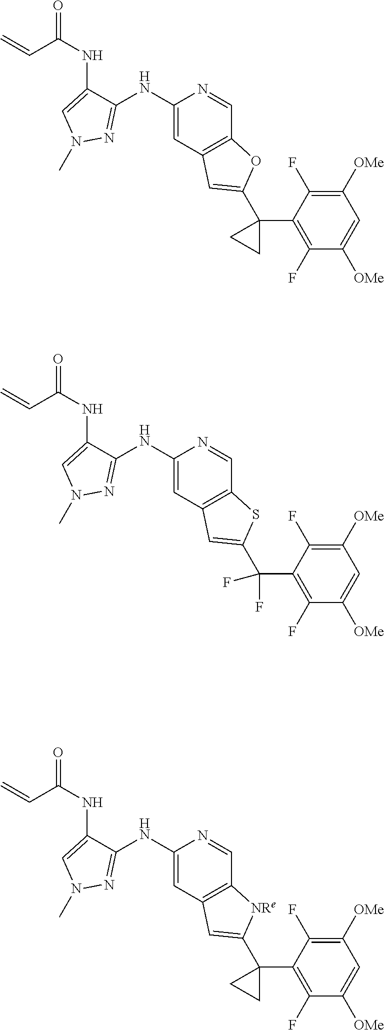

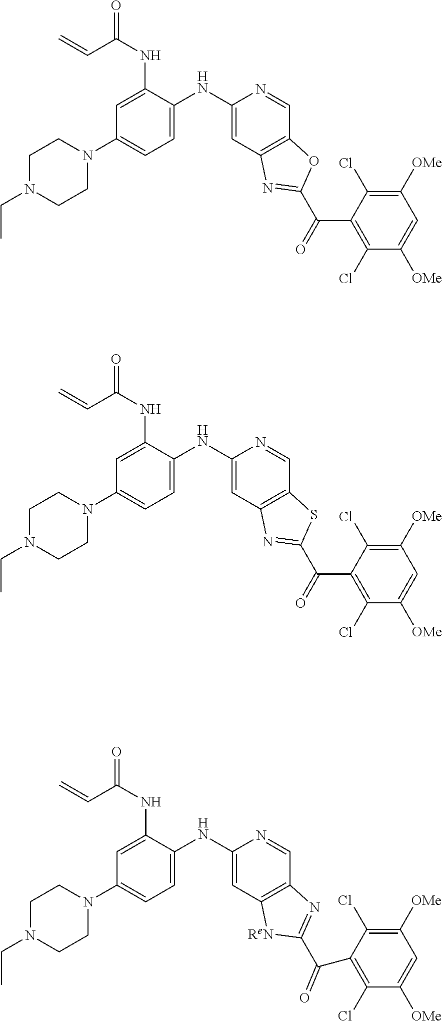

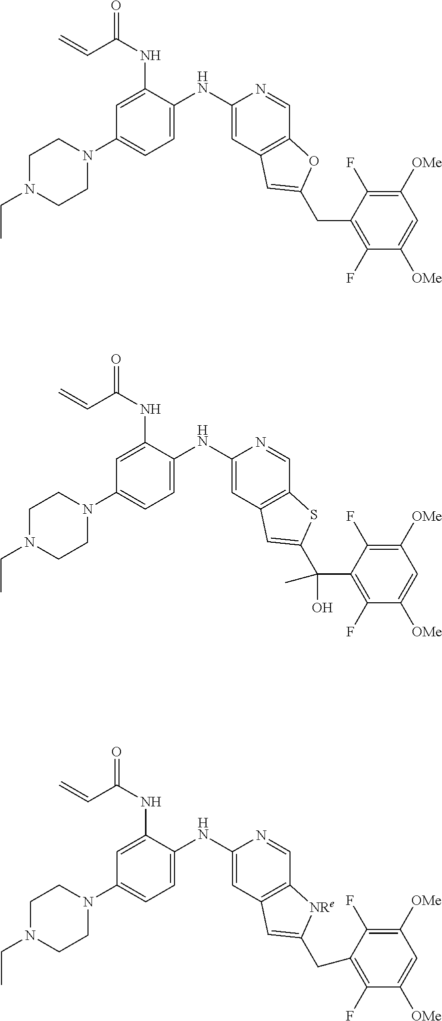

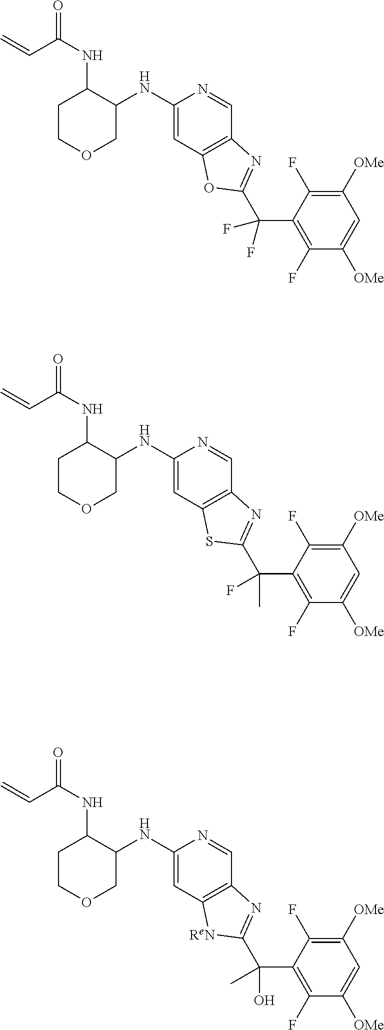

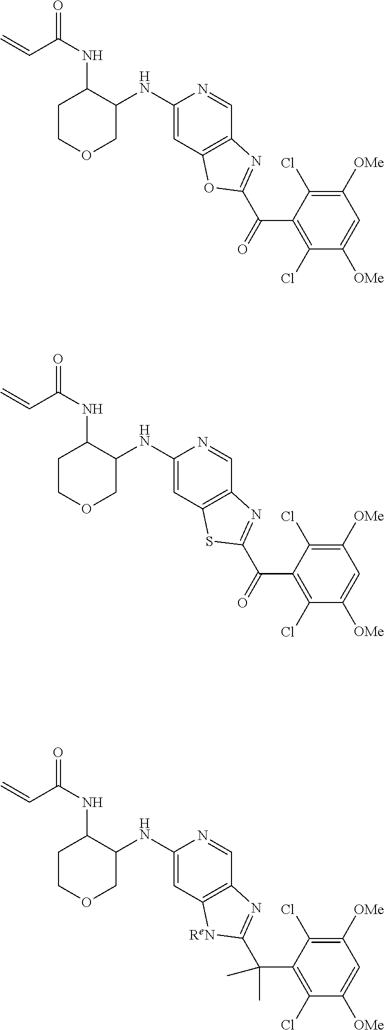

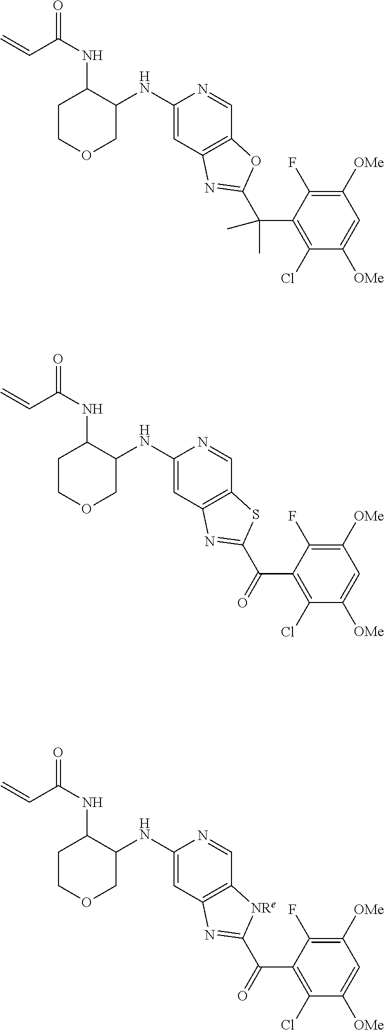

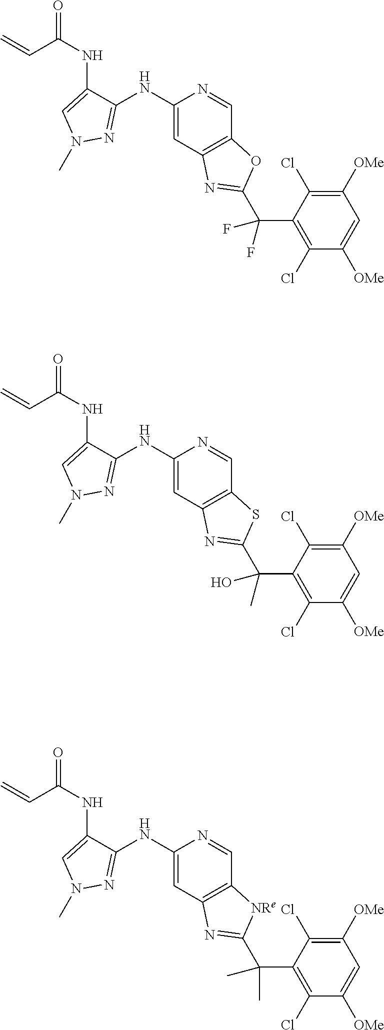

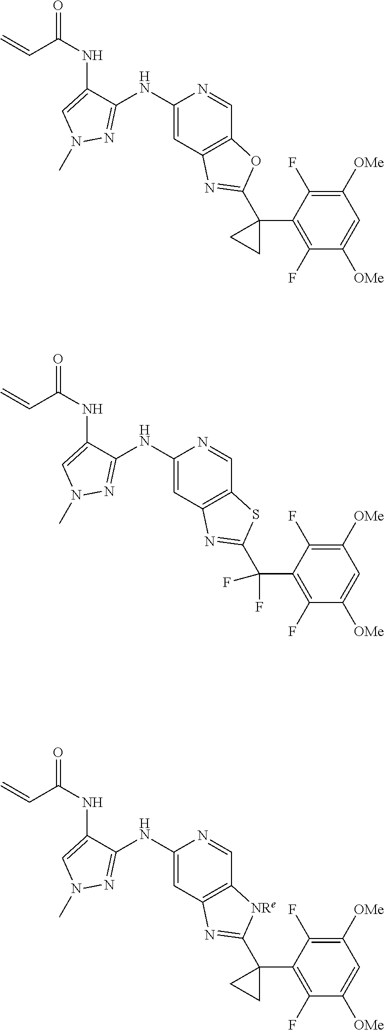

In certain embodiments, the FGFR-4 inhibitors of the invention inhibit FGFR-4 activity more potently than they inhibit FGFR-1 activity.

In another aspect, the invention features a pharmaceutical composition comprising a pharmaceutically acceptable carrier and a compound disclosed herein.

In another aspect the invention features a method for treating a condition mediated by FGFR-4, a condition characterized by overexpression of FGFR-4, a condition characterized by amplification of FGFR-4, a condition mediated by FGF19, a condition characterized by amplified FGF19, or a condition characterized by overexpression of FGF19, any of these methods comprising administering a therapeutically effective amount of a compound disclosed herein to a subject. In another aspect, the invention features a method of treating any of the following conditions by administering a therapeutically effective amount of a compound disclosed herein to a subject: hepatocellular carcinoma, breast cancer, ovarian cancer, lung cancer, liver cancer, a sarcoma, or hyperlipidemia.

The invention includes all possible combinations of the embodiments described above and below. It should be understood that, within the scope of the present invention, each technical feature of the present invention described above and in the following (as examples) may be combined with each other to form a new or preferred technical solution, which is not listed here due to space limitations.

Definitions

The term “alkyl,” by itself or as part of another substituent, means, unless otherwise stated, a straight (i.e. unbranched) or branched chain, or cyclic hydrocarbon radical, or combination thereof, which may be fully saturated, mono-or polyunsaturated and can include di- and multivalent radicals, having the number of carbon atoms designated (i.e. C₁₋₁₀ means one to ten carbons). Examples of saturated hydrocarbon radicals include, but are not limited to, groups such as methyl, ethyl, n-propyl, isopropyl, n-butyl, t-butyl, isobutyl, sec-butyl, cyclohexyl, (cyclohexyl)methyl, cyclopropylmethyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. Alkyl groups which are limited to hydrocarbon groups are termed “homoalkyl”. The alkyl is optionally substituted with one or more halogen atom(s).

The term “halogenated alkyl” means alkyl as defined above wherein one or more hydrogen atoms have been replaced by halogen atoms.

The term “alkylene” by itself or as part of another substituent means a divalent radical derived from an alkyl, as exemplified, but not limited, by —CH₂CH₂CH₂CH₂—, —CH₂CH═CHCH₂—, —CH₂ C≡H₂—, —CH₂CH₂CH(CH₂CH₂CH₃)CH₂—. Typically, an alkyl (or alkylene) group has from 1 to 24 carbon atoms, with those groups having 10 or fewer carbon atoms being preferred in the present invention. A “lower alkyl” or “lower alkylene” is a shorter chain alkyl or alkylene group, generally having eight or fewer carbon atoms. The alkylene is optionally substituted with one or more halogen atom(s).

The term “alkynyl” means carbon chains which contain at least one carbon-carbon triple bond, and which may be linear or branched or combinations thereof. Examples of alkynyl include ethynyl, propargyl, 3-methyl-1-pentynyl, 2-heptynyl and the like. The alkynyl is optionally substituted with one or more halogen atom(s).

The term “cycloalkyl” means mono- or bicyclic saturated carbocyclic rings, each of which has from 3 to 10 carbon atoms. A “fused analog” of cycloalkyl means a monocyclic ring fused to an aryl or heteroaryl group in which the point of attachment is on the non-aromatic portion. Examples of cycloalkyl and fused analogs thereof include cyclopropyl, cyclobutyl, cyclopentyl, cyclohexyl, cycloheptyl, tetrahydronaphthyl, decahydronaphthyl, indanyl, and the like. The cycloalkyl is optionally substituted with one or more halogen atom(s).

The term “alkoxy” means alkoxy groups of a straight or branched having the indicated number of carbon atoms. C₁₋₆alkoxy, for example, includes methoxy, ethoxy, propoxy, isopropoxy, and the like.

The term “heteroalkyl,” by itself or in combination with another term, means, unless otherwise stated, a stable straight or branched chain, or cyclic hydrocarbon radical, or combinations thereof, consisting of at least one carbon atoms and at least one heteroatom selected from the group consisting of O, N, P, Si and S, and wherein the nitrogen, phosphorus, and sulfur atoms may optionally be oxidized and the nitrogen heteroatom may optionally be quaternized. The heteroatom(s) O, N, P and S and Si may be placed at any interior position of the heteroalkyl group or at the position at which alkyl group is attached to the remainder of the molecule. Examples include, but are not limited to, —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O) ₂—CH₃, —CH═CH—O—CH₃, —Si(CH₃)₃, —CH₂—CH═N—OCH₃, —CH═CH—N(CH₃)—CH₃, —O—CH₃, —O—CH₂—CH ₃, and —CN. Up to two or three heteroatoms may be consecutive, such as, for example, —CH₂—NH—OCH₃ and —CH₂—O—Si(CH₃)₃. Similarly, the term “heteroalkylene” by itself or as part of another substituent means a divalent radical derived from heteroalkyl, as exemplified, but not limited by, —CH₂—CH₂—S —CH₂—CH₂— and —CH₂—S—CH₂—CH₂—NH—CH₂—. For heteroalkylene groups, heteroatoms can also occupy either or both of the chain termini (e.g., alkyleneoxo, alkylenedioxo, alkyleneamino, alkylenediamino, and the like). Still further, for alkylene and heteroalkylene linking groups, no orientation of the linking group is implied by the direction in which the formula of the linking group is written. For example, the formula —C(O)OR′— represents both—C(O)OR′—and —R′OC(O)—. As described above, heteroalkyl groups, as used herein, include those groups that are attached to the remainder of the molecule through a heteroatom, such as —C(O)R′, —C(O)NR′, —NR′R″, —OR′, —SR′, and/or —SO₂R′. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like.

The term “cycloalkoxy” means cycloalkyl as defined above bonded to an oxygen atom, such as cyclopropyloxy.

The term “halogenated alkoxy” means alkoxy as defined above wherein one or more hydrogen atoms have been replaced by halogen atoms.

The term “aryl” means mono- or bicyclic aromatic rings containing only carbon atoms. A “fused analog” of aryl means an aryl group fused to a monocyclic cycloalkyl or monocyclic heterocyclyl group in which the point of attachment is on the aromatic portion. Examples of aryl and fused analogs thereof include phenyl, naphthyl, indanyl, indenyl, tetrahydronaphthyl, 2,3-dihydrobenzofuranyl, dihydrobenzopyranyl, 1,4-benzodioxanyl, and the like.

The term “heteroaryl” means a mono- or bicyclic aromatic ring containing at least one heteroatom selected from N, O and S, with each ring containing 5 to 6 atoms. A “fused analog” of heteroaryl means a heteroaryl group fused to a monocyclic cycloalkyl or monocyclic heterocyclyl group in which the point of attachment is on the aromatic portion. Examples of heteroaryl include pyrrolyl, isoxazolyl, isothiazolyl, pyrazolyl, pyridyl, oxazolyl, oxadiazolyl, thiadiazolyl, thiazolyl, imidazolyl, triazolyl, tetrazolyl, furanyl, triazinyl, thienyl, pyrimidyl, pyridazinyl, pyrazinyl, benzoxazolyl, benzothiazolyl, benzimidazolyl, benzofuranyl, benzothiophenyl, furo(2,3-b)pyridyl, quinolyl, indolyl, isoquinolyl, and the like.

The alkyl groups, aryl groups and said heteroaryl groups referred to in the definitions are unsubstituted or are substituted by at least one substituent selected from the group consisting of substituents.

The substituents are selected from the group consisting of halogen atoms, alkyl groups having from 1 to 4 carbon atoms, alkoxy groups having from 1 to 4 carbon atoms, haloalkyl groups having from 1 to 4 carbon atoms, haloalkoxy groups having from 1 to 4 carbon atoms, cyano groups, alkynyl groups having from 2 to 6 carbon atoms, alkanoyl groups having from 1 to 5 carbon atoms, cycloalkyl groups having from 3 to 7 ring atoms, heteroaryl groups, aryl groups, aralkoxy groups having from 7 to 10 carbon atoms, arylcarbonyl groups, two adjacent-x groups are optionally joined together to form an alkylene or an alkenylene chain having 3 or 4 carbon atoms, aminocarbonyl groups, alkenyl groups having from 2 to 5 carbon atoms, alkylthio groups having from 1 to 4 carbon atoms, aminosulfinyl groups, aminosulfonyl groups, hydroxy groups, —SF₅, hydroxyalkyl groups having from 1 to 4 carbon atoms, nitro groups, amino groups, carboxy groups, alkoxycarbonyl groups having from 2 to 5 carbon atoms, alkoxyalkyl groups having from 1 to 4 carbon atoms, alkylsulfonyl groups having from 1 to 4 carbon atoms, alkanoylamino groups having from 1 to 4 carbon atoms, alkanoyl(alkyl)amino groups having from 1 to 6 carbon atoms, alkanoylaminoalkyl groups having from 1 to 6 carbon atoms in both the alkanoyl and alkyl part, alkanoyl(alkyl)aminoalkyl groups having from 1 to 6 carbon atoms in both the alkanoyl and each alkyl part, alkylsulfonylamino groups having from 1 to 4 carbon atoms, mono-or di-alkylaminocarbonyl groups having from 1 to 6 carbon atoms, mono-or di-alkylaminosulfinyl groups having from 1 to 6 carbon atoms, mono-or di alkylaminosulfonyl groups having from 1 to 6 carbon atoms, aminoalkyl groups having from 1 to 4 carbon atoms, mono-or di-alkylamino groups having from 1 to 6 carbon atoms, mono-or di-alkylaminoalkyl groups having from 1 to 6 carbon atoms in each alkyl part, aralkyl groups having from 7 to 10 carbon atoms, heteroarylalkyl groups having from 1 to 4 carbon atoms in the alkyl part, heteroarylalkoxy groups having from 1 to 4 carbon atoms in the alkoxy part and alkylsulfonylamino groups having from 1 to 4 carbon atoms;

The term “heterocyclyl” means mono- or bicyclic saturated rings containing at least one heteroatom selected from N, S and O, each of said ring having from 3 to 10 atoms in which the point of attachment may be carbon or nitrogen. A “fused analog” of heterocyclyl means a monocyclic heterocycle fused to an aryl or heteroaryl group in which the point of attachment is on the non-aromatic portion. Examples of “heterocyclyl” and fused analogs thereof include pyrrolidinyl, piperidinyl, piperazinyl, imidazolidinyl, 2,3-dihydrofuro(2,3-b)pyridyl, benzoxazinyl, tetrahydrohydroquinolinyl, tetrahydroisoquinolinyl, dihydroindolyl, and the like. The term also includes partially unsaturated monocyclic rings that are not aromatic, such as 2- or 4-pyridones attached through the nitrogen or N-substituted-(1H,3H)-pyrimidine-2,4-diones (N-substituted uracils).

The terms “halo” or “halogen,” by themselves or as part of another substituent, mean, unless otherwise stated, a fluorine, chlorine, bromine, or iodine atom. Additionally, terms such as “haloalkyl,” or “halogenated alkyl” are meant to include monohaloalkyl and polyhaloalkyl. For example, the term “halo(C₁-C₄)alkyl” is mean to include, but not be limited to, trifluoromethyl, 2,2,2-trifluoroethyl, 4-chlorobutyl, 3-bromopropyl, and the like.

A “prodrug” refers to an agent that is converted into the parent drug in vivo. Prodrugs are often useful because, in some situations, they may be easier to administer than the parent drug. They may, for instance, be bioavailable by oral administration whereas the parent is not. The prodrugs may also have improved solubility in pharmaceutical compositions over the parent drug. An example, without limitation, of a prodrug would be a compound of any of Formula I, which is administered as an ester (the “prodrug”) to facilitate transmittal across a cell membrane where water solubility is detrimental to mobility, but which then is metabolically hydrolyzed to the carboxylic acid, the active entity, once inside the cell where water-solubility is beneficial. A further example of a prodrug might be a short peptide (polyaminoacid) bonded to an acid group where the peptide is metabolized to reveal the active moiety.

Optical Isomers—Diastereomers—Geometric Isomers—Tautomers:

Compounds of Formula I may contain one or more asymmetric centers and may thus occur as racemates and racemic mixtures, single enantiomers, diastereomeric mixtures and individual diastereomers. The present invention is meant to comprehend all such isomeric forms of the compounds of Formula I.

Some of the compounds described herein contain olefinic double bonds, and unless specified otherwise, are meant to include both E and Z geometric isomers.

Some of the compounds of Formula I may contain one or more than one cyclic ring systems and may thus exist in cis- and trans- isomers. The present invention is meant to include all such cis- and trans- isomers.

Some of the compounds described herein may exist with different points of attachment of hydrogen, referred to as tautomers. Such an example may be a ketone and its enol form known as keto-enol tautomers. The individual tautomers as well as mixture thereof are encompassed with compounds of Formula I.

Compounds of the Formula I may be separated into diastereoisomeric pairs of enantiomers by, for example, fractional crystallization from a suitable solvent, for example MeOH or EtOAc or a mixture thereof. The pair of enantiomers thus obtained may be separated into individual stereoisomers by conventional means, for example by the use of an optically active amine or acid as a resolving agent or on a chiral HPLC column.

Alternatively, any enantiomer of a compound of the general Formula I may be obtained by stereospecific synthesis using optically pure starting materials or reagents of known configuration.

Stable Isotope-Labeled Analogs:

One or more than one of the protons in compounds of Formula I can be replaced with deuterium atom(s), thus providing deuterated analogs that may have improved pharmacological activities.

Salts and Formulations

The compounds described herein can be useful as the free base or as a salt.

The term “pharmaceutically acceptable salts” refers to salts prepared from pharmaceutically acceptable non-toxic bases or acids including inorganic or organic bases and inorganic or organic acids. Salts derived from inorganic bases include aluminum, ammonium, calcium, copper, ferric, ferrous, lithium, magnesium, manganic salts, manganous, potassium, sodium, zinc, and the like. Particularly preferred are the ammonium, calcium, magnesium, potassium, and sodium salts. Salts derived from pharmaceutically acceptable organic non-toxic bases include salts of primary, secondary, and tertiary amines, substituted amines including naturally occurring substituted amines, cyclic amines, and basic ion exchange resins, such as arginine, betaine, caffeine, choline, N,N′-dibenzylethylenediamine, diethylamine, 2-diethylaminoethanol, 2-dimethylaminoethanol, ethanolamine, ethylenediamine, N-ethyl-morpholine, N-ethylpiperidine, glucamine, glucosamine, histidine, hydramine, isopropylamine, lysine, methylglucamine, morpholine, piperazine, piperidine, polyamine resins, procaine, purines, theobromine, triethylamine, trimethylamine, tripropylamine, tromethamine, and the like.

When the compound of the present invention is alkaline, salts may be prepared from pharmaceutically acceptable non-toxic acids, including inorganic and organic acids. Such acids include acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid, and the like. Particularly preferred are citric, hydrobromic, hydrochloric, maleic, phosphoric, sulfuric, and tartaric acids.

It will be understood that, as used herein, references to the compounds of Formula I are meant to also include the pharmaceutically acceptable salts.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredients are mixed with water or an oil medium, for example peanut oil, liquid paraffin, or olive oil.

Aqueous suspensions contain the active material in admixture with excipients suitable for the manufacture of aqueous suspensions. Such excipients are suspending agents, for example sodium carboxymethyl-cellulose, methylcellulose, hydroxypropylmethy-cellulose, sodium alginate, polyvinyl-pyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethylene-oxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl, p-hydroxybenzoate, one or more coloring agents, one or more flavoring agents, and one or more sweetening agents, such as sucrose, saccharin or aspartame.

Oily suspensions may be formulated by suspending the active ingredient in a vegetable oil, for example arachis oil, olive oil, sesame oil or coconut oil, or in mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present.

The pharmaceutical compositions of the invention may also be in the form of an oil-in-water emulsions. The oily phase may be a vegetable oil, for example olive oil or arachis oil, or a mineral oil, for example liquid paraffin or mixtures of these. Suitable emulsifying agents may be naturally-occurring phosphatides, for example soy bean, lecithin, and esters or partial esters derived from fatty acids and hexitol anhydrides, for example sorbitan monooleate, and condensation products of the partial esters with ethylene oxide, for example polyoxyethylene sorbitan monooleate. The emulsions may also contain sweetening and flavouring agents.

Syrups and elixirs may be formulated with sweetening agents, for example glycerol, propylene glycol, sorbitol or sucrose. Such formulations may also contain a demulcent, a preservative and flavoring and coloring agents. The pharmaceutical compositions may be in the form of a sterile injectable aqueous or oleagenous suspension. This suspension may be formulated according to the known art using those suitable dispersing or wetting agents and suspending agents which have been mentioned above. The sterile injectable preparation may also be a sterile injectable solution or suspension in a non-toxic parenterally-acceptable diluent or solvent, for example as a solution in 1,3-butane diol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or diglycerides. In addition, fatty acids such as oleic acid find use in the preparation of injectables.

The compounds of the invention can also be administered intranasally or by inhalation, typically in the form of a dry powder (either alone, as a mixture, for example, in a dry blend with lactose, or as a mixed component particle, for example, mixed with phospholipids, such as phosphatidylcholine) from a dry powder inhaler or as an aerosol spray from a pressurised container, pump, spray, atomiser (preferably an I atomiser using electrohydrodynamics to produce a fine mist), or nebuliser, with or without the use of a suitable propellant, such as 1,1,1,2-tetrafluoroethane or 1,1,1,2,3,3,3-heptafluoropropane. For intranasal use, the powder may comprise a bioadhesive agent, for example, chitosan or cyclodextrin.

The pressurized container, pump, spray, atomizer, or nebulizer contains a solution or suspension of the compound(s) of the invention comprising, for example, ethanol, aqueous ethanol, or a suitable alternative agent for dispersing, solubilizing, or extending release of the active, a propellant(s) as solvent and an optional surfactant, such as sorbitan trioleate, oleic acid, or an oligolactic acid.

Prior to use in a dry powder or suspension formulation, the drug product is micronized to a size suitable for delivery by inhalation (typically less than 5 microns).

This may be achieved by any appropriate comminuting method, such as spiral jet milling, fluid bed jet milling, supercritical fluid processing to form nanoparticles, high pressure homogenization, or spray drying.

Capsules (made, for example, from gelatin or HPMC), blisters and cartridges for use in an inhaler or insufflator may be formulated to contain a powder mix of the compound of the invention, a suitable powder base such as lactose or starch and a performance modifier such as 1-leucine, mannitol, or magnesium stearate. The lactose may be anhydrous or in the form of the monohydrate, preferably the latter. Other suitable excipients include dextran, glucose, maltose, sorbitol, xylitol, fructose, sucrose and trehalose.

A suitable solution formulation for use in an atomizer using electrohydrodynamics to produce a fine mist may contain from 1 mg to 20 mg of the compound of the invention per actuation and the actuation volume may vary from 1 L to 100 L. A typical formulation may comprise a compound of Formula (A) propylene glycol, sterile water, ethanol and sodium chloride. Alternative solvents which may be used instead of propylene glycol include glycerol and polyethylene glycol.

Suitable flavors, such as menthol and levomenthol, or sweeteners, such as saccharin or saccharin sodium, may be added to those formulations of the invention intended for inhaled/intranasal administration.

Formulations for inhaled/intranasal administration may be formulated to be immediate and/or modified release using, for example, poly(DL-lactic-coglycolic acid (PGLA). Modified release formulations include delayed-, sustained-, pulsed-, controlled-, targeted and programmed release.

In the case of dry powder inhalers and aerosols, the dosage unit is determined by means of a valve which delivers a metered amount. Units in accordance with the invention are typically arranged to administer a metered dose or “puff” containing from 1 fig to 10 mg of the compound of Formula I. The overall daily dose will typically be in the range 1 lag to 10 mg which may be administered in a single dose or, more usually, as divided doses throughout the day.

Compounds of Formula I may also be administered in the form of suppositories for rectal administration of the drug. These compositions can be prepared by mixing the drug with a suitable non-irritating excipient which is solid at ordinary temperatures but liquid at the rectal temperature and will therefore melt in the rectum to release the drug. Such materials are cocoa butter and polyethylene glycols.

For topical use, creams, ointments, jellies, solutions or suspensions, etc., containing the compound of Formula I are employed. (For purposes of this application, topical application shall include mouth washes and gargles.)

Dosage levels of the order of from about 0.01 mg to about 140 mg/kg of body weight per day are useful in the treatment of the above-indicated conditions, or alternatively about 0.5 mg to about 7 g per patient per day. For example, a condition may be effectively treated by the administration of from about 0.01 to 50 mg of the compound per kilogram of body weight per day, or alternatively about 0.5 mg to about 3.5 g per patient per day, preferably 2.5 mg to 1 g per patient per day.

The amount of active ingredient that may be combined with the carrier materials to produce a single dosage form will vary depending upon the host treated and the particular mode of administration. For example, a formulation intended for the oral administration of humans may contain from 0.5 mg to 5 g of active agent compounded with an appropriate and convenient amount of carrier material which may vary from about 5 to about 95 percent of the total composition. Dosage unit forms will generally contain between from about 1 mg to about 500 mg of an active ingredient, typically 25 mg, 50 mg, 100 mg, 200 mg, 300 mg, 400 mg, 500 mg, 600 mg, 800 mg, or 1000 mg.

It will be understood, however, that the specific dose level for any particular patient will depend upon a variety of factors including the age, body weight, general health, sex, diet, time of administration, route of administration, rate of excretion, drug combination and the severity of the particular disease undergoing therapy.

Indications

Compounds of the present invention may be used to treat diseases with altered FGFR-4 and/or FGF19 status, such as hepatocellular carcinoma and other forms of cancer.

Combination and Targeted Therapy

Administration of the FGFR-4 inhibitors disclosed herein can be combined with other cancer treatments. For example, the inhibitors can be administered in combination with surgical treatments, radiation, or other therapeutic agents such as antibodies, other selective kinase inhibitors, or chemotherapeutics. The inhibitors may also be administered in combination with RNAi therapy, antisense therapy, or immunotherapies. The FGFR-4 inhibitors described herein may be combined with one, two, or more other therapeutic agents. In the examples outlined below, it is understood that “second therapeutic agent” also includes more than one therapeutic agent other than the FGFR-4 inhibitor. For instance, the compounds disclosed herein may be combined with an agent such as sorafenib, a PD-1 antibody or a PD-L1 antibody. A FGFR-4 inhibitor described herein may be administered with one, two, or more other therapeutic agents.

Synthesis

The compounds of the present invention can be prepared according to the following synthetic schemes:

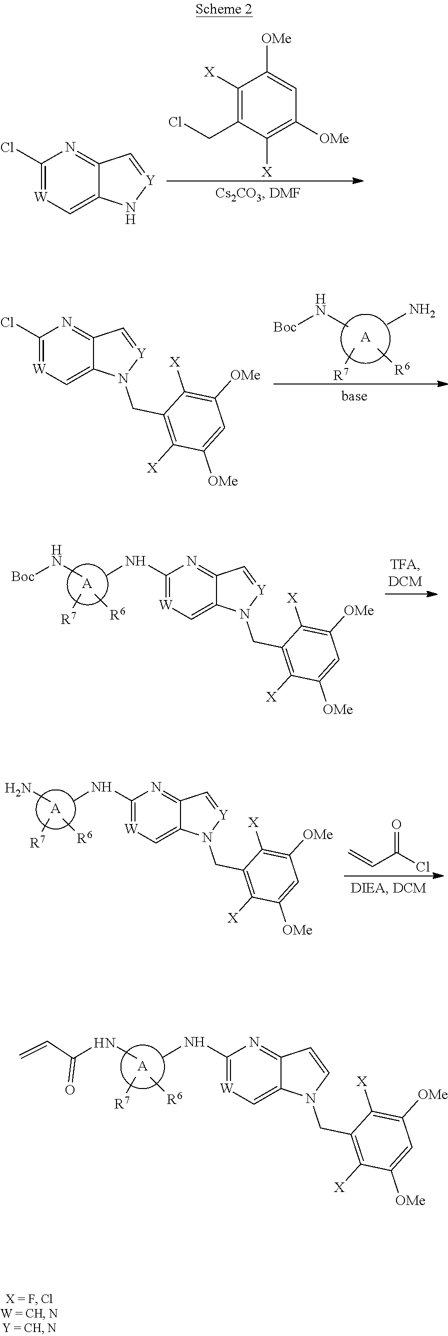

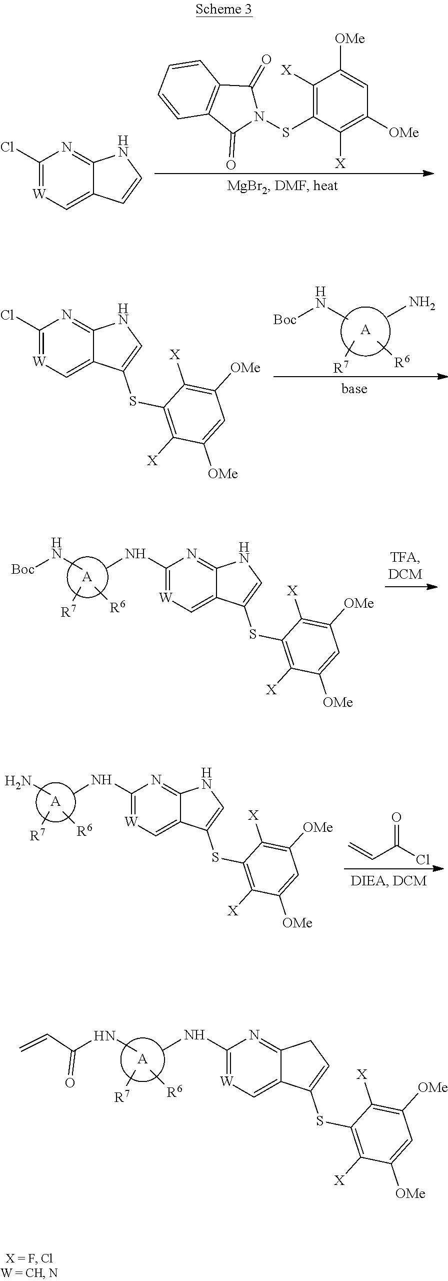

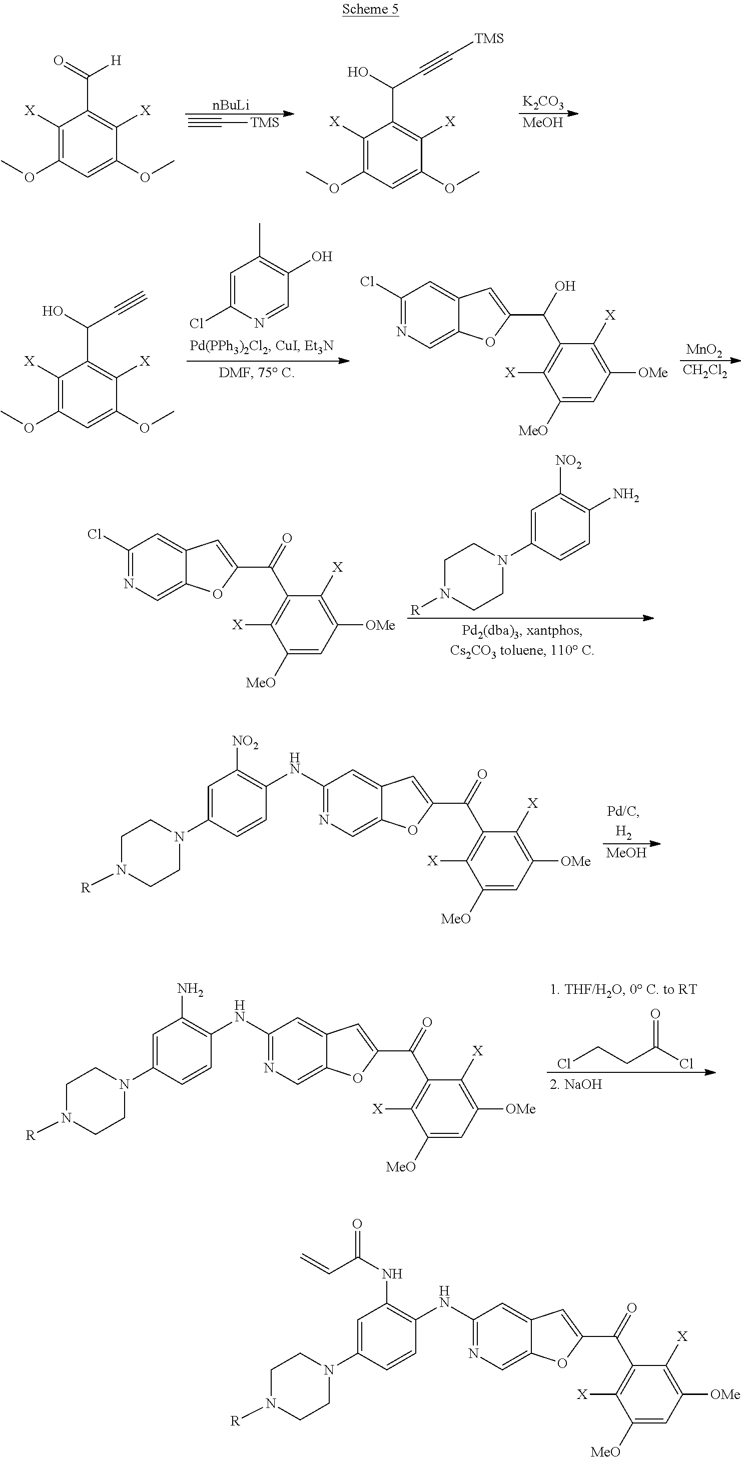

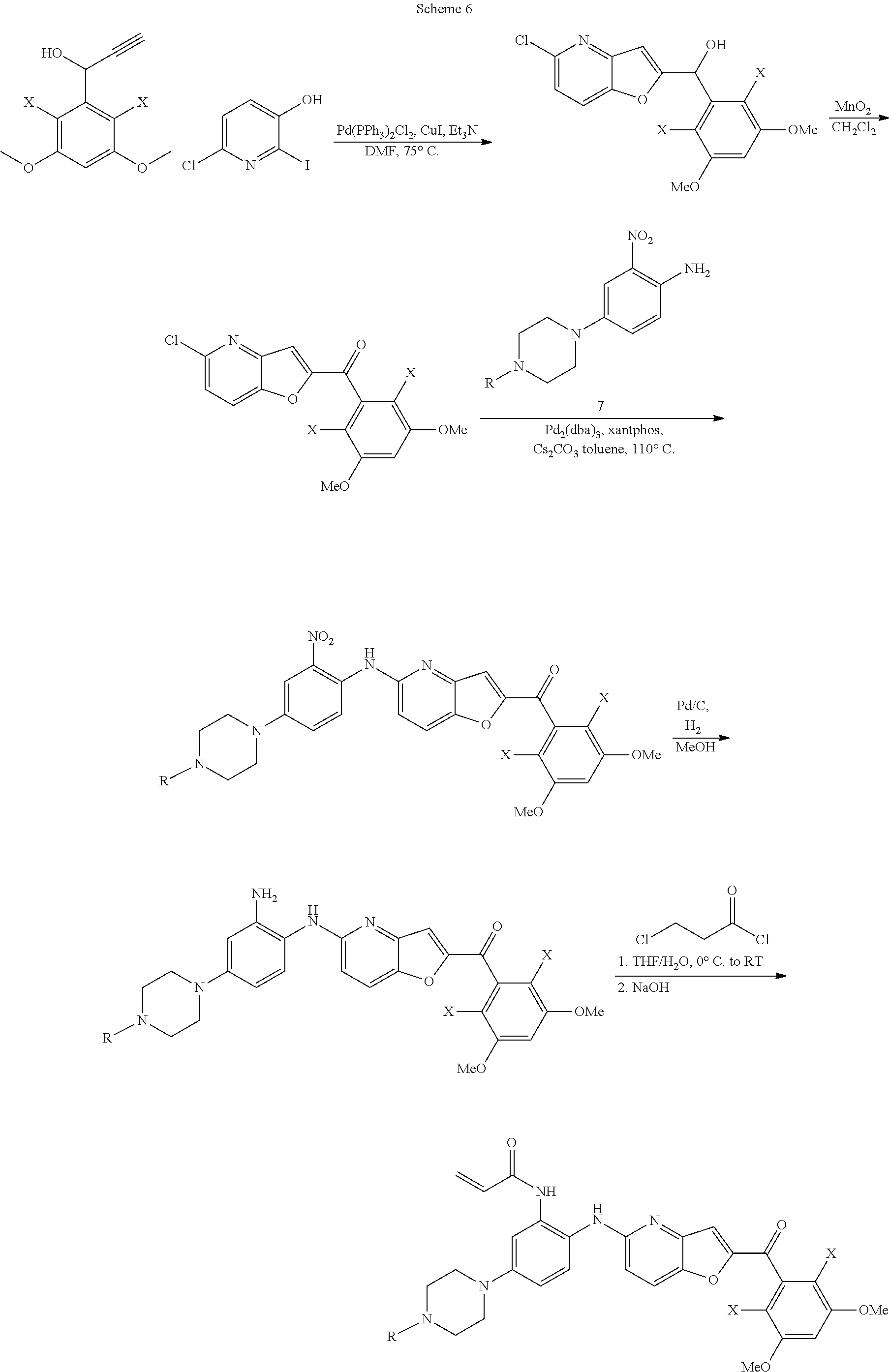

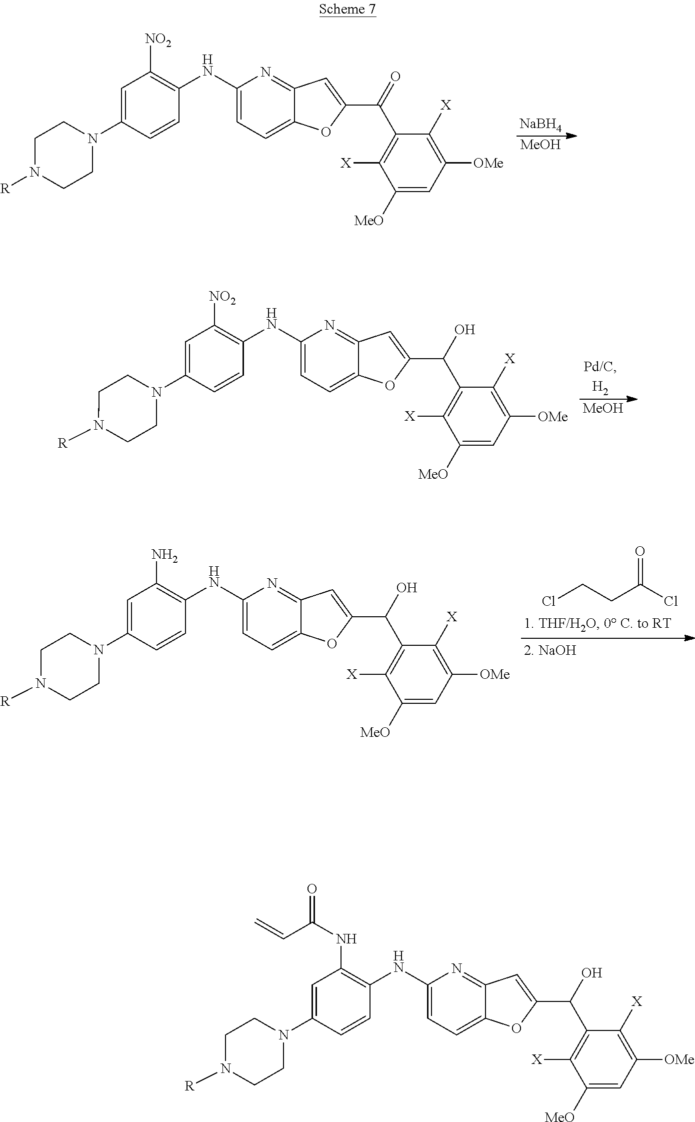

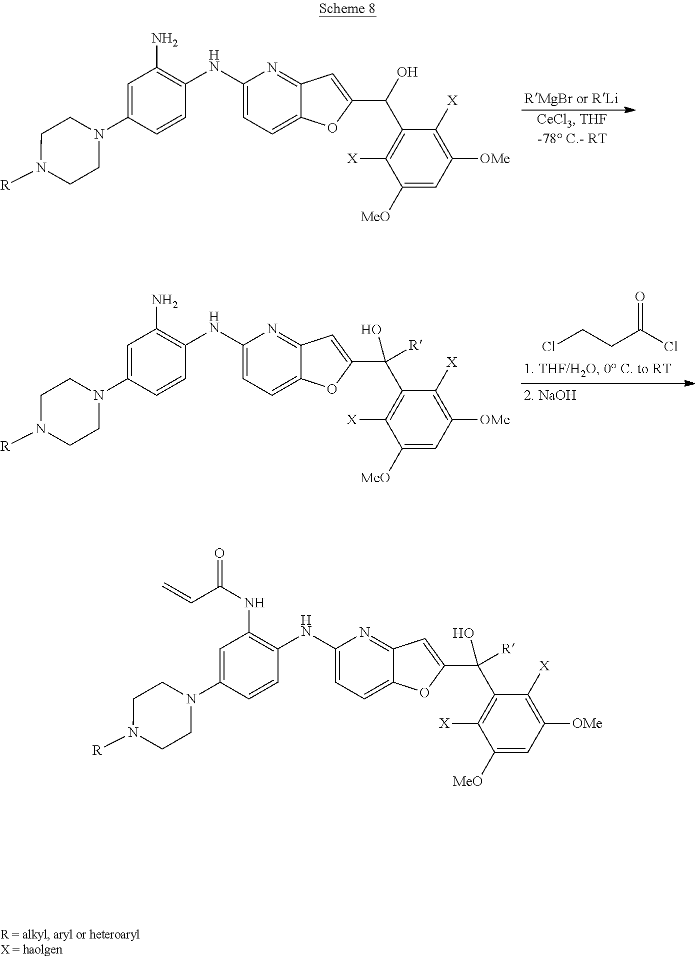

The following abbreviations have the meanings indicated. EA means ethyl acetate; DBU means 1,8-diazabicyclo[5.4.0]undec-7-ene; DIBAL means diisobutylaluminum hydride; DIEA means diisopropylethylamine; DMAP means N,N-dimethylaminopyridine; DME means 1,2-dimethoxyethane; DMF means N,N-dimethylformamide; dmpe means 1,2-bis(dimethylphosphino)ethane; DMSO means dimethylsulfoxide; dppb means 1,4-bis(diphenylphosphino)butane; dppe means 1,2-bis(diphenylphosphino)ethane; dppf means 1,1′-bis(diphenylphosphino)ferrocene; dppm means 1,1′-bis(diphenylphosphino)methane; DIAD means diisopropylazodicarboxylate; EDCI means 1-(3-dimethylaminopropyl)-3-ethylcarbodiimide; HATU means 2-(7-Aza-1H-benzotriazole-1-yl)-1,1,3,3-tetramethyluronium hexafluorophosphate; HMPA means hexamethylphosphorarnide; IPA means isopropyl alcohol; LDA means lithium diisopropylamide; LHMDS means lithium bis(hexamethyldisilylamide); LAH means lithium aluminum hydride; NCS means N-chlorosuccinimide; PE means petroleum ether; PyB OP means benzotriazol-1-yloxytripyrrolidinophosphonium hexafluorophosphate; TDA means tris(2-(2-methoxyethoxy)ethyl)amine; DCM means dichloromethame; TEA means triethylamine; TFA means trifluoroacetic acid; THF means tetrahydrofuran; NCS means N-chlorosuccinimide; NMM means N-methylmorpholine; NMP means N- methylpyrrolidine; PPh3 means triphenylphosphine, RT means room temperature; T3P means propylphosphonic anhydride.

HPLC-MS analyses were performed on Waters HPLC 2790 with Waters micromass ZQ 4000 (Model MAA050) as mass detector and Waters 2487 UV as detector. Column used was Phenomemex OOB-4605-E0 (5u-XB-C18-100A, 50×4.6 mm). The mobile phase consists eluent A (water, 0.05% TFA) and eluent B (CH3CN, 0.05% TFA), and the elution proceeded at 1 mL/min. The initial conditions were 90% A for 1 min, then 90% A to 10% A linearly decreased within 5 min, then from 10% A to 90% A within 1 min. The total run time is 7 minutes.

The present invention will be more readily understood by referring to the following examples which are given to illustrate the invention rather than to limit its scope. Unless otherwise indicated, percentages and parts are by weight and parts by weight.

Unless otherwise specified, materials and reagents used in the examples of the present invention are all commercially available products.

### EXAMPLE 1

### N-(2-((1-(2,6-difluoro-3,5-dimethoxybenzyl)-1H-pyrrolo[3,2-b]pyridin-5-yl)amino)-5-(4-ethylpiperazin-1-yl)phenyl)acrylamide

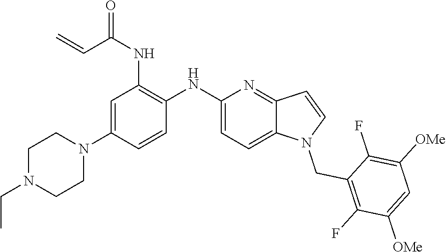 [SMILES: C=CC(=O)Nc1cc(N2CCN(CC)CC2)ccc1Nc1ccc2c(ccn2Cc2c(F)c(OC)cc(OC)c2F)n1]

### Step 1 tert-butyl (4-bromo-2-nitrophenyl)carbamate

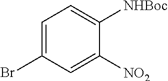 [SMILES: CC(C)(C)OC(=O)Nc1ccc(Br)cc1[N+](=O)[O-]]

The mixture of 4-bromo-2-nitroaniline (50 g), Boc2O (80 g) and DMAP (0.6 g) in THF (500 mL) was refluxed for 2 days. The reaction solution was cooled to room temperature and concentrated to dryness. The residue was purified by column to give 23 g of tert-butyl (4-bromo-2-nitrophenyl) carbamate as an orange solid in 32% yield.

### Step 2 tert-butyl (4-(4-ethylpiperazin-1-yl)-2-nitrophenyl)carbamate

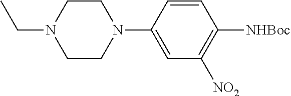 [SMILES: CCN1CCN(c2ccc(NC(=O)OC(C)(C)C)c([N+](=O)[O-])c2)CC1]

The mixture of tert-butyl-4-bromo-2-nitrophenylcarbamate (10.8 g), 1-ethylpiperazine (5.9 g), Pd₂(dba)₃ (3.2 g), Xantphos (4 g) and Cs₂CO₃ (22.2 g) in toluene (170 mL) was heated at 100° C. under N₂ for 5 h. The reaction mixture was cooled to room temperature and concentrated to dryness. The residue was purified by column to give 3.4 g of pure product as a brown solid. Yield: 28.6%

### Step 3 4-(4-ethylpiperazin-1-yl)-2-nitroaniline

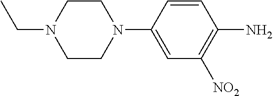 [SMILES: CCN1CCN(c2ccc(N)c([N+](=O)[O-])c2)CC1]

To a solution of tert-butyl (4-(4-ethylpiperazin-1-yl)-2-nitrophenyl) carbamate (3.4 g) in DCM (50 mL), TFA was added dropwise at 0-5° C. The resulting mixture was stirred at room temperature for 4 h. Concentrated to dryness. The residue was dissolved in DCM and basified with K₂CO₃ aq. The organic layer was separated and washed with brine. Dried over Na₂SO₄. Filtered and concentrated and the residue was purified by column to give 1.3 g of pure product as a brown solid. Yield:54.2%

### Step 4 2,6-difluoro-3,5-dimethoxybenzyl methanesulfonate

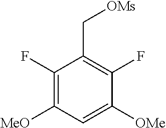 [SMILES: COc1cc(OC)c(F)c(COS(C)(=O)=O)c1F]

A solution of MsCl (0.64 g) in DCM (5 mL) was added drop wise to the solution of (2,6-difluoro-3,5- dimethoxyphenyl) methanol (1 g) and TEA (0.71 g) in DCM (5 mL) at 0-5° C. The mixture was stirred at room temperature for 20 min. Quenched with water and extracted with DCM. Dried over Na₂SO₄. Filtered and concentrated to give 1.2 g of pure product as a yellow solid. Yield: 92.3%.

### Step 5 5-bromo-1-(2,6-difluoro-3,5-dimethoxybenzyl)-1H-pyrrolo[3,2-b]pyridine

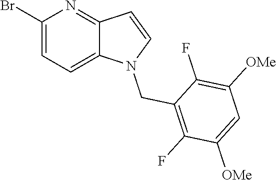 [SMILES: COc1cc(OC)c(F)c(Cn2ccc3nc(Br)ccc32)c1F]

NaH (85 mg) was added to the solution of 5-bromo-1H-pyrrolo[3,2-b] pyridine (356 mg) in DMF (15 mL) at room temperature under N2. The mixture was stirred at that temperature for 30 min. 2,6-difluoro-3,5-dimethoxybenzyl methanesulfonate (500 mg) was then added. The resulting mixture was stirred at room temperature for 2 h. Quenched with water (30 mL). The white precipitate was collected by filtration and washed with water. Dried to give 630 mg of pure product as a white solid. Yield: 92.9%.

### Step 6 1-(2,6-difluoro-3,5-dimethoxybenzyl)-N-(4-(4-ethylpiperazin-1-yl)-2-nitrophenyl)-1H-pyrrolo[3,2-b]pyridin-5-amine

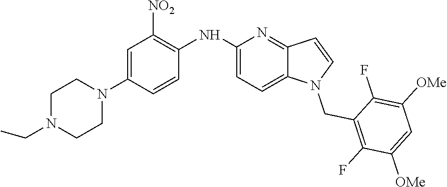 [SMILES: CCN1CCN(c2ccc(Nc3ccc4c(ccn4Cc4c(F)c(OC)cc(OC)c4F)n3)c([N+](=O)[O-])c2)CC1]

The degassed mixture of 4-(4-ethylpiperazin-1-yl)-2-nitroaniline (271 mg), 5-bromo-1-(2,6-difluoro-3,5- dimethoxybenzyl)-1H-pyrrolo[3,2-b] pyridine (500 mg), Pd₂(dba)₃ (198 mg), Xantphos (250 mg) and Cs₂CO₃ (704 mg) in toluene (20 mL) was heated at 100° C. under N₂ for 6 h. The salts were filtered off and washed with DCM. The filtrate was concentrated and the residue was purified by column to give 500 mg of product. Yield: 83.8%.

### Step 7 N¹-(1-(2,6-difluoro-3,5-dimethoxybenzyl)-1H-pyrrolo[3,2-b]pyridin-5-yl)-4-(4-ethylpiperazin-1-yl)benzene-1,2-diamine

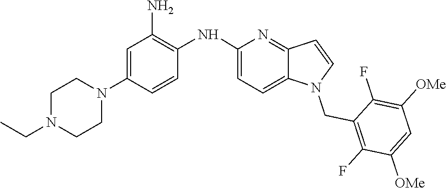 [SMILES: CCN1CCN(c2ccc(Nc3ccc4c(ccn4Cc4c(F)c(OC)cc(OC)c4F)n3)c(N)c2)CC1]

PtO₂ (20 mg) was added to the solution of 1-(2,6-difluoro-3,5-dimethoxybenzyl)-N-(4-(4-ethyl piperazin-1-yl)-2-nitrophenyl)-1H-pyrrolo[3,2-b] pyridin-5-amine (290 mg) and the mixture was hydrogenated under 1 atm of H₂ at room temperature for 4 h. Filtered and concentrated to give 250 mg of dark brown solid. Yield: 91.2%.

### Step 8 N-(2-((1-(2,6-difluoro-3,5-dimethoxybenzyl)-1H-pyrrolo[3,2-b] pyridin-5-yl)amino)-5-(4-ethylpiperazin-1-yl)phenyl)acrylamide

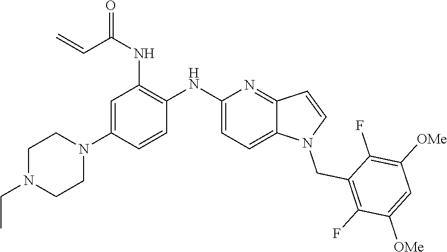 [SMILES: C=CC(=O)Nc1cc(N2CCN(CC)CC2)ccc1Nc1ccc2c(ccn2Cc2c(F)c(OC)cc(OC)c2F)n1]

To a solution of N¹-(1-(2,6-difluoro-3,5-dimethoxybenzyl)-1H-pyrrolo[3,2-b] pyridin-5-yl)-4-(4-ethyl piperazin-1-yl) benzene-1,2-diamine (390 mg) and acrylic acid (53.8 mg) in DCM was added DIPEA (193 mg) under N2. 1-Propanephosphoni acid anhydride (950 mg) was added in portions over 30 min. The resulting mixture was stirred at room temperature overnight. The mixture was treated with water and extracted with DCM. Dried over Na₂SO₄. Filtered and concentrated to give 214 mg of crude product. Purified by prepare HPlC to give 69 mg of pure product as a yellow solid. Yield: 16%. MS (ES+): 577.274 [M +1]⁺.

### EXAMPLE 2

### N-(2-((1-(2,6-difluoro-3,5-dimethoxybenzyl)-1H-pyrrolo[2,3-c]pyridin-5-yl)amino)-5-(4-ethylpiperazin-1-yl)phenyl)acrylamide

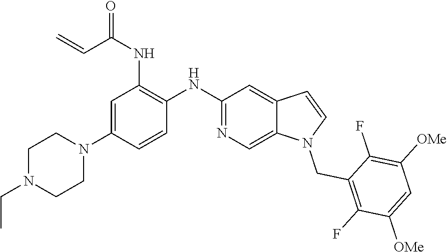 [SMILES: C=CC(=O)Nc1cc(N2CCN(CC)CC2)ccc1Nc1cc2ccn(Cc3c(F)c(OC)cc(OC)c3F)c2cn1]

The title compound was prepared by using the same method as described for Example 1 and 5-bromo-1H-pyrrolo[2,3-c]pyridine as starting material. MS (ES+) 577.274 [M+1]⁺. 564.75

### EXAMPLE 3

N-(2-((2-(2,6-difluoro-3,5-dimethoxybenzoyl)furo[2,3-c]pyridin-5-yl)amino)-5-(4-ethylpiperazin-1-yl)phenyl)acrylamide

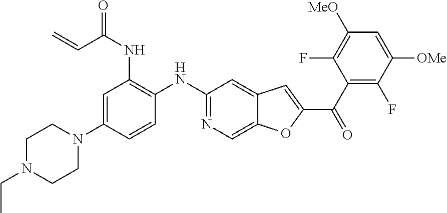 [SMILES: C=CC(=O)Nc1cc(N2CCN(CC)CC2)ccc1Nc1cc2cc(C(=O)c3c(F)c(OC)cc(OC)c3F)oc2cn1]

### Step 1 1-(2,6-difluoro-3,5-dimethoxyphenyl)-3-(trimethylsilyl)prop-2-yn-1-ol

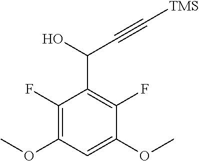 [SMILES: COc1cc(OC)c(F)c(C(O)C#C[Si](C)(C)C)c1F]

To a solution of trimethylsilylacetylene (3.28 g, 33.4 mmol) in THF (50 mL) at −78° C. was added nBuLi (1.6 M in hexane, 20 mL, 32 mmol). After stirring at 45 min at -78° C., 2,6-difluoro-3,5-dimethoxybenzaldehyde (5.0 g, 24.7 mmol) in THF (20 mL) was added to the reaction. The reaction was stirred for 30 min at −78° C., then warmed to 0° C. and stirred for 1 h. The ice bath was removed, and the reaction was stirred for 2 hrs at room temperature. The reaction mixture was quenched with saturated NH₄Cl, extracted with EtOAc (3×100 mL), dried over Na₂SO₄, filtered and concentrated to give 8.8 g of crude title compound which was used in the next step without further purification. HPLC retention time: 5.18 min.

### Step 2 1-(2,6-difluoro-3,5-dimethoxyphenyl)prop-2-yn-1-ol

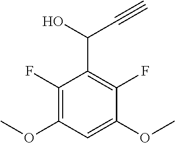

The product of Step 1 (8.8 g crude) and K₂CO₃ (10.25 g, 74.2 mmol) was stirred in MeOH (200 mL) at room temperature for 4 hrs. The solvent was removed by rotavap, the residue was partitioned between H₂O and EtOAc. The organic phase was washed with brine, dried with Na₂SO₄, filtered and concentrated. Residue obtained was loaded onto silica gel column with DCM and purified by column chromatography (eluent: EtOAc: Hexanes/0:100 to 20:80). 5.1 g of the title compound was obtained (yield: 91% over 2 steps). HPLC retention time: 3.42 min.

### Step 3 (5-chlorofuro[2,3-c]pyridin-2-yl)(2,6-difluoro-3,5-dimethoxyphenyl)methanol

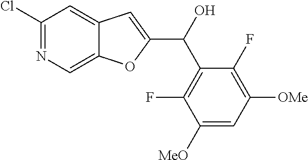 [SMILES: COc1cc(OC)c(F)c(C(O)c2cc3cc(Cl)ncc3o2)c1F]

To a solution of 6-chloro-4-iodopyridin-3-ol (1.0 g, 3.91 mmol) in DMF (5 mL) was added CuI (224 mg, 1.18 mmol) and Et₃N (790 mg, 7.82 mmol). Argon was bubbled through the mixture for 10 min and Pd(PPh₃)₂Cl₂ (275 mg, 0.39 mmol) was added. Argon was bubbled through the mixture again for 10 min. The mixture was stirred for 1 h, then a solution of the product of Step 2 (893 mg, 3.91 mmol) in DMF (4 mL) was added and the resulting mixture was stirred at 75° C. for 16 h under argon. After cooling to room temperature, the reaction mixture was partitioned between EtOAc and water. The aqueous phase was extracted with EtOAc (3×50 mL). The combined organic phases were washed with brine, dried with Na₂SO₄, filtered and concentrated. Residue obtained was loaded onto silica gel column with DCM and purified by column chromatography (eluent: EtOAc : Hexanes/0:100 to 30:70). 1.0 g of the title compound was obtained (yield: 72%). HPLC retention time: 4.28 min. MS: 355.53 [M+1]⁺.

### Step 4 (5-chlorofuro[2,3-c]pyridin-2-yl)(2,6-difluoro-3,5-dimethoxyphenyl)methanone

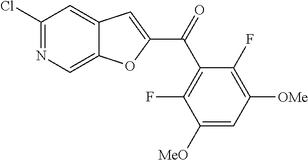 [SMILES: COc1cc(OC)c(F)c(C(=O)c2cc3cc(Cl)ncc3o2)c1F]

A mixture of the product of Step 3 (300 mg, 0.84 mmol) and MnO₂ (85%, 1.29 g, 12.6 mmol) in DCM (30 mL) was stirred at room temperature for 2 hrs. The mixture was filtered through a layer of Celite®. The filtrate was concentrated to give 300 mg of the title compound (yield: 100%) which was used in the next step without further purification. HPLC retention time: 5.15 min. MS: 354.28 [M+1]⁺.

### Step 5 (2,6-difluoro-3,5-dimethoxyphenyl)(5-((4-(4-ethylpiperazin-1-yl)-2-nitrophenyl)amino)furo[2,3-c]pyridin-2-yl)methanone

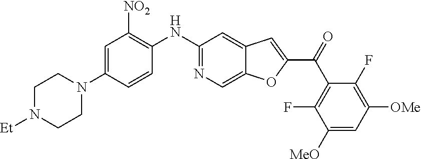 [SMILES: CCN1CCN(c2ccc(Nc3cc4cc(C(=O)c5c(F)c(OC)cc(OC)c5F)oc4cn3)c([N+](=O)[O-])c2)CC1]

To a solution of the product of Step 4 (120 mg, 0.34 mmol) and 4-(4-ethylpiperazin-1-yl)-2-nitroaniline (102 mg, 0.41 mmol) in toluene (10 mL) was added Xantphos (79 mg, 0.14 mmol), Cs₂CO₃ (221 mg, 0.68 mmol). Argon was bubbled through the mixture for 10 min and Pd₂(dba)₃ (63 mg, 0.069 mmol) was added. Argon was bubbled through the mixture again for 10 min. The reaction mixture was heated to 110° C. for 5.5 h under argon. After cooling to room temperature, the reaction mixture was partitioned between EtOAc and water. The aqueous phase was extracted with EtOAc (3×50 mL). The combined organic phases were washed with brine, dried with Na₂SO₄, filtered and concentrated. Residue obtained was loaded onto silica gel column with DCM and purified by column chromatography (eluent: MeOH:DCM/0:100 to 5:95). 149 mg of the title compound was obtained (yield: 77%). HPLC retention time: 4.37 min. MS: (ES+): 567.69 [M+1]⁺.

### Step 6 (5-((2-amino-4-(4-ethylpiperazin-1-yl)phenyl)amino)furo[2,3-c]pyridin-2-yl)(2,6-difluoro-3,5-dimethoxyphenyl)methanone

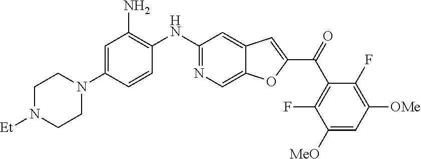 [SMILES: CCN1CCN(c2ccc(Nc3cc4cc(C(=O)c5c(F)c(OC)cc(OC)c5F)oc4cn3)c(N)c2)CC1]

Pd/C (10%, 10 mg) was added to a solution of the product of Step 5 (35 mg, 0.062 mmol) in MeOH (2.5 mL). The reaction mixture was stirred for 2 h under hydrogen (1 atm, balloon) at RT. The reaction mixture was filtered through a layer of Celite®. The filtrate was concentrated to afford 20 mg the crude title compound (yield: 60%) which was used in the next step without further purification. HPLC retention time: 3.29 min. MS (ES+): 536.80[M+1]⁺.

### Step 7 N-(2-((2-(2,6-difluoro-3,5-dimethoxybenzoyl)furo[2,3-c]pyridin-5-yl)amino)-5-(4-ethylpiperazin-1-yl)phenyl)acrylamide

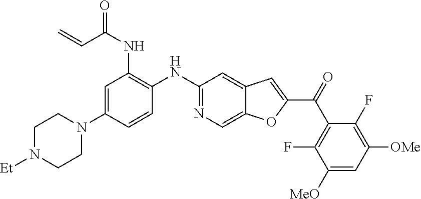 [SMILES: C=CC(=O)Nc1cc(N2CCN(CC)CC2)ccc1Nc1cc2cc(C(=O)c3c(F)c(OC)cc(OC)c3F)oc2cn1]

The product of Step 6 (20 mg, 0.037 mmol) was dissolved in a solution of THF (4 mL) and H₂O (0.4 mL). At 0° C., 3-chloropropionyl chloride (12.1 mg, 0.095 mmol) in THF (0.2 mL) was added dropwise to the reaction mixture. After stirring at 0° C. for 30 min, the reaction was warmed to room temperature and stirred for 2 h. Then NaOH (19 mg, 0.48 mmol) in H₂O (0.5 mL) was added the reaction, and the reaction was stirred overnight. The reaction mixture was partitioned between DCM and water. The aqueous phase was extracted with DCM (10 mL). The combined organic phases were washed with brine, dried with Na₂SO₄, filtered and concentrated. Residue obtained was purified by prepHPLC to give 1.7 mg of the title compound. HPLC retention time: 3.48 min. MS (ES+): 592.16 [M+1]⁺.

### EXAMPLE 4

### N-(2-((2-(2,6-difluoro-3,5-dimethoxybenzoyl)furo[3,2-b]pyridin-5-yl)amino)-5-(4-ethylpiperazin-1-yl)phenyl)acrylamide

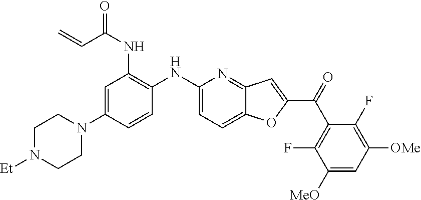 [SMILES: C=CC(=O)Nc1cc(N2CCN(CC)CC2)ccc1Nc1ccc2oc(C(=O)c3c(F)c(OC)cc(OC)c3F)cc2n1]

### Step 1 (5-chlorofuro[3,2-b]pyridin-2-yl)(2,6-difluoro-3,5-dimethoxyphenyl)methanol

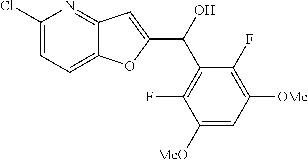 [SMILES: COc1cc(OC)c(F)c(C(O)c2cc3nc(Cl)ccc3o2)c1F]

The title compound was prepared from 1-(2,6-difluoro-3,5-dimethoxyphenyl)prop-2-yn-1-ol and 6-chloro-2-iodopyridin-3-ol following a procedure analogous to that described for Step 3 of Example 5.HPLC retention time: 4.39 min. MS: 356.79 [M+1]⁺.

### Step 2 (5-chlorofuro[3,2-b]pyridin-2-yl)(2,6-difluoro-3,5-dimethoxyphenyl)methanone

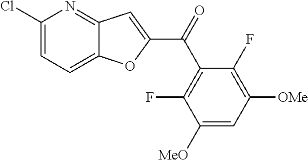 [SMILES: COc1cc(OC)c(F)c(C(=O)c2cc3nc(Cl)ccc3o2)c1F]

A mixture of the product of Step 1 (690 mg, 1.94 mmol) and MnO₂ (85%, 2.97 g, 29.1 mmol) in DCM (50 mL) was stirred at room temperature for 2 hrs. The mixture was filtered through a layer of Celite®. The filtrate was concentrated to give 680 mg of the title compound (yield: 98%) which was used in the next step without further purification. HPLC retention time: 5.20 min. MS (ES+): 354.97 [M+1]⁺.

### Step 3 (2,6-difluoro-3,5-dimethoxyphenyl)(5-((4-(4-ethylpiperazin-1-yl)-2-nitrophenyl)amino)furo[3,2-b]pyridin-2-yl)methanone

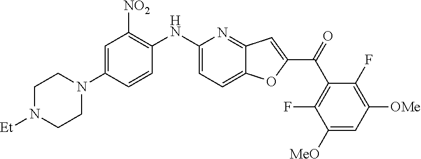 [SMILES: CCN1CCN(c2ccc(Nc3ccc4oc(C(=O)c5c(F)c(OC)cc(OC)c5F)cc4n3)c([N+](=O)[O-])c2)CC1]

To a solution of the product of Step 2 (35 mg, 0.099 mmol) and 4-(4-ethylpiperazin-1-yl)-2-nitroaniline (30 mg, 0.12 mmol) in toluene (3 mL) was added Xantphos (23 mg, 0.04 mmol), Cs₂CO₃ (65 mg, 0.20 mmol). Argon was bubbled through the mixture for 10 min and Pd₂(dba)₃ (63 mg, 0.02 mmol) was added. Argon was bubbled through the mixture again for 10 min. The reaction mixture was heated to 110° C. for 5.5 h. After cooling to room temperature, the reaction mixture was partitioned between EtOAc and water. The aqueous phase was extracted with EtOAc (3×15 mL). The combined organic phases were washed with brine, dried with Na₂SO₄, filtered and concentrated. Residue obtained was loaded onto silica gel column with DCM and purified by column chromatography (eluent: MeOH:DCM/0:100 to 5:95). 46 mg of compound the title compound was obtained (yield: 81%). HPLC retention time: 4.32 min. MS (ES+): 568.50 [M+1]⁺.

### Step 4 (5-((2-amino-4-(4-ethylpiperazin-1-yl)phenyl)amino)furo[3,2-b]pyridin-2-yl)(2,6-difluoro-3,5-dimethoxyphenyl)methanone

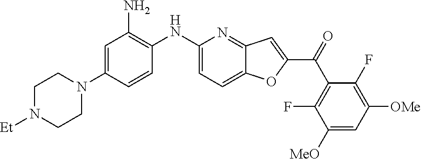 [SMILES: CCN1CCN(c2ccc(Nc3ccc4oc(C(=O)c5c(F)c(OC)cc(OC)c5F)cc4n3)c(N)c2)CC1]

Pd/C (10%, 11 mg) was added to a solution of the product of Step 3 (40 mg, 0.070 mmol) in MeOH (3 mL). The reaction mixture was stirred for 4 h under hydrogen (1 atm, balloon) at RT. The reaction mixture was filtered through a layer of Celite®. The filtrate was concentrated to afford 40 mg the crude title compound (yield: 100%) which was used in the next step without further purification. HPLC retention time: 3.23 min. MS (ES+): 538.64 [M+1]⁺.

### Step 5 N-(2-((2-(2,6-difluoro-3,5-dimethoxybenzoyl)furo[3,2-b]pyridin-5-yl)amino)-5-(4-ethylpiperazin-1-yl)phenyl)acrylamide

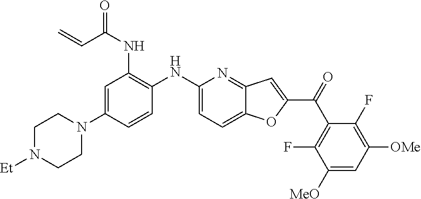 [SMILES: C=CC(=O)Nc1cc(N2CCN(CC)CC2)ccc1Nc1ccc2oc(C(=O)c3c(F)c(OC)cc(OC)c3F)cc2n1]

The product of Step 4 (25 mg, 0.047 mmol) was dissolved in a solution of THF (5 mL) and H₂O (0.5 mL). At 0° C., 3-chloropropionyl chloride (5.9 mg, 0.047 mmol) in THF (1 mL) was added dropwise to the reaction mixture. The reaction was warmed to room temperature and stirred for 10 min. Then NaOH (5.6 mg, 0.14 mmol) in H₂O (1.0 mL) was added the reaction, and the reaction was stirred at 65° C. for 4 h. The reaction mixture was cooled to room temperature and partitioned between DCM and water. The aqueous phase was extracted with DCM (10 mL). The combined organic phases were washed with brine, dried with Na₂SO₄, filtered and concentrated. Residue obtained was purified by preparative TLC to give 2.63 mg of the title compound. HPLC retention time: 3.59 min. MS (ES+): 592.54. [M+1]⁺.

### EXAMPLE 5

### (±)-N-(2-((2-((2,6-difluoro-3,5-dimethoxyphenyl)(hydroxy)methyl)furo[2,3-c]pyridin-5-yl)amino)-5-(4-ethylpiperazin-1-yl)phenyl)acrylamide

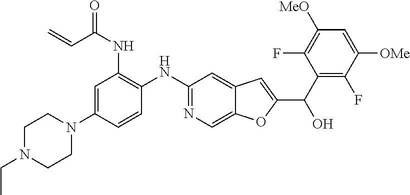 [SMILES: C=CC(=O)Nc1cc(N2CCN(CC)CC2)ccc1Nc1cc2cc(C(O)c3c(F)c(OC)cc(OC)c3F)oc2cn1]

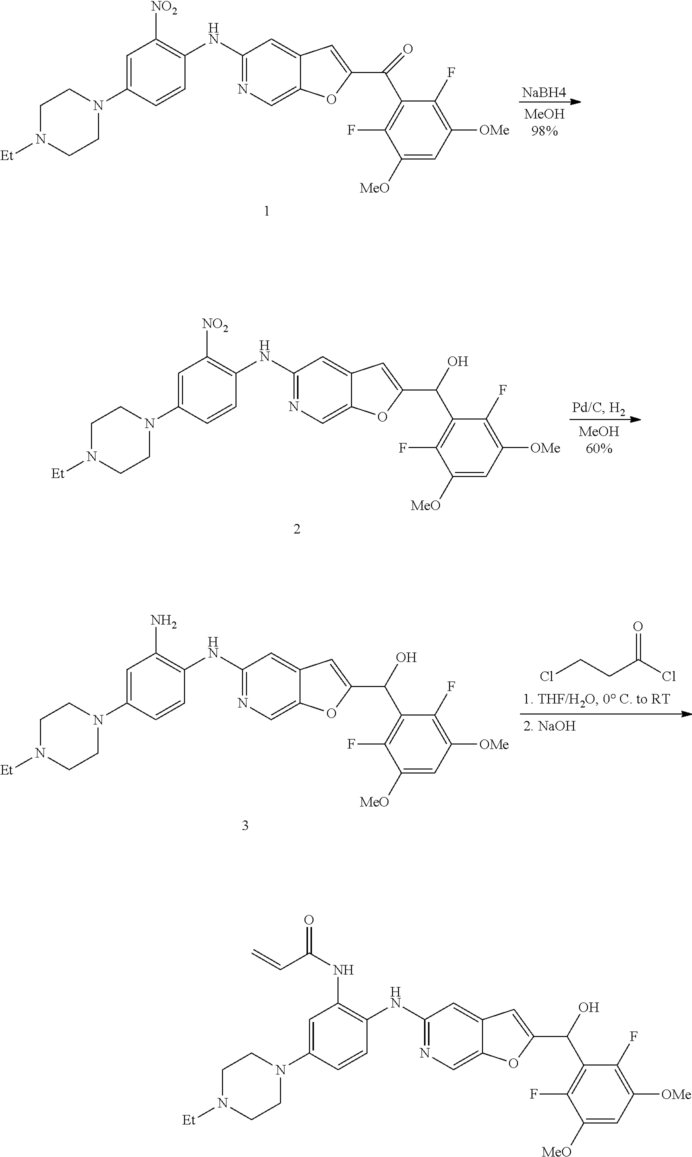

### Step 1 (2,6-difluoro-3,5-dimethoxyphenyl)(5-((4-(4-ethylpiperazin-1-yl)-2-nitrophenyl)amino)furo[2,3-c]pyridin-2-yl)methanol

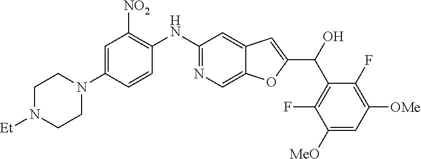 [SMILES: CCN1CCN(c2ccc(Nc3cc4cc(C(O)c5c(F)c(OC)cc(OC)c5F)oc4cn3)c([N+](=O)[O-])c2)CC1]

To a solution of the product of Step 5 of Example 5 (55 mg, 0.097 mmol) in MeOH (50 mL) at 0° C. was added NaBH₄ (11 mg, 0.29 mmol). After stirring at 15 min at 0° C., the reaction was warmed to room temperature and stirred for 2 h. NaBH₄ (11 mg, 0.29 mmol) was added to the reaction, and the reaction was stirred at room temperature for 1 h. The solvent was removed by rotavap, the residue was partitioned between H₂O and EtOAc. The organic phase was washed with brine, dried with Na₂SO₄, filtered and concentrated to give 50 mg of crude title compound (yield: 90%) which was used in the next step without further purification. MS (ES+): 570.03 [M+H]⁺.

### Step 2 (±)(5-((2-amino-4-(4-ethylpiperazin-1-yl)phenyl)amino)furo[2,3-c]pyridin-2-yl)(2,6-difluoro-3,5-dimethoxyphenyl)methanol

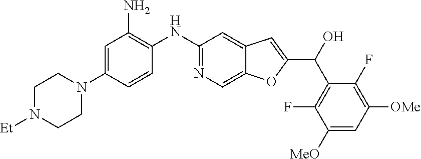 [SMILES: CCN1CCN(c2ccc(Nc3cc4cc(C(O)c5c(F)c(OC)cc(OC)c5F)oc4cn3)c(N)c2)CC1]

Pd/C (10%, 10 mg) was added to a solution of the product of Step 1 (50 mg, 0.087 mmol) in MeOH (3 mL). The reaction mixture was stirred for 1 h under hydrogen (1 atm, balloon) at RT. The reaction mixture was filtered through a layer of Celite®. The filtrate was concentrated to afford 40 mg crude title compound (yield: 75%) which was used in the next step without further purification. MS (ES+): 539.97 [M+H]⁺.

### Step 3 (±)-N-(2-((2-((2,6-difluoro-3,5-dimethoxyphenyl)(hydroxy)methyl)furo[2,3-c]pyridin-5-yl)amino)-5-(4-ethylpiperazin-1-yl)phenyl)acrylamide

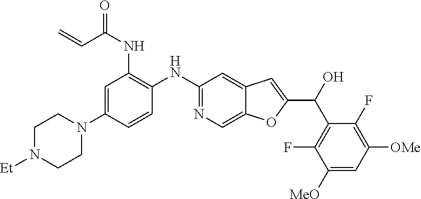 [SMILES: C=CC(=O)Nc1cc(N2CCN(CC)CC2)ccc1Nc1cc2cc(C(O)c3c(F)c(OC)cc(OC)c3F)oc2cn1]

The product of Step 2 (40 mg, 0.074 mmol) was dissolved in a solution of THF (4 mL) and H₂O (0.4 mL). At 0° C., 3-chloropropionyl chloride (18.8 mg, 0.148 mmol) in THF (0.2 mL) was added dropwise to the reaction mixture. After stirring at 0° C. for 30 min, the reaction was warmed to room temperature and stirred for 2 h. Then NaOH (18 mg, 0.45 mmol) in H₂O (0.5 mL) was added the reaction, and the reaction was stirred overnight. The reaction mixture was partitioned between DCM and water. The aqueous phase was extracted with DCM (10 mL). The combined organic phases were washed with brine, dried with Na₂SO₄, filtered and concentrated. Residue obtained was purified by prepHPLC to give 10 mg of the title compound

MS (ES+): 594.31 [M+1]⁺.

### EXAMPLE 6

### (±)-N-(2-((2-((2,6-difluoro-3-methoxyphenyl)(hydroxy)methyl)furo[3,2-b]pyridin-5-yl)amino)-5-(4-ethylpiperazin-1-yl)phenyl)acrylamide

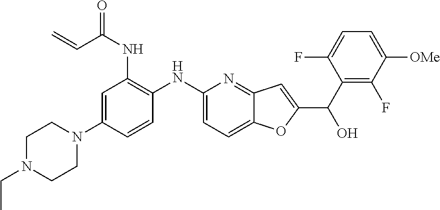 [SMILES: C=CC(=O)Nc1cc(N2CCN(CC)CC2)ccc1Nc1ccc2oc(C(O)c3c(F)ccc(OC)c3F)cc2n1]

Using the product of Step 3 of Example 6 as starting material, the title compound was prepared by the procedure analogous to that described for Example 7. MS (ES+): 564.20 [M+1]⁺.

### EXAMPLE 7

### (±)-N-(2-((2-(1-(2,6-difluoro-3,5-dimethoxyphenyl)-1-hydroxyethyl)furo[2,3-c]pyridin-5-yl)amino)-5-(4-ethylpiperazin-1-yl)phenyl)acrylamide

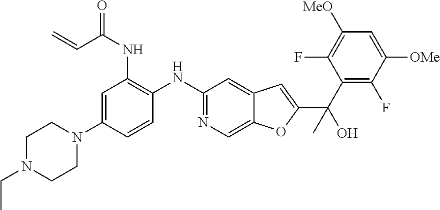 [SMILES: C=CC(=O)Nc1cc(N2CCN(CC)CC2)ccc1Nc1cc2cc(C(C)(O)c3c(F)c(OC)cc(OC)c3F)oc2cn1]

To a solution of Example 5 (0.1 g, 0.169 mmol) in THF (2 ml) at 5° C. was added MeMgBr (0.1m1, 3 M in THF, 0.3 mmol). After the reaction mixture was stirred for 30 minutes at 5° C., the reaction was quenched with water and extracted with EA . The organic layer was washed with brine and dried over Na₂SO₄. The solution was filtered and concentrated. The residue was purified by Pre-TLC (MeOH/DCM=10%) to give 10 mg of product as a gray solid. Yield: 18%. MS (ES+):608.2[M+1]⁺.

¹HNMR: (400 MHZ, CDCl₃)δ 1.23-1.29 (t, J=7.2 Hz, 3H), 2.05-2.10 (m, 4H), 2.69 (m, 2H), 3.42-3.45 (m, 4H), 3.49 (s, 3H), 3.86(s, 6H),5.63-5.65 (d, J=10 Hz, 1H), 6.16-6.348 (m, 4H), 6.46 (s, 1H), 6.62-8.68 (m,2H), 7.13-7.16(d, J=8.8, 1H), 8.18(s, 1H), 8.13(s, 1H),8.37(s,1H).

### EXAMPLE 8

### N-(2-((2-((2,6-difluoro-3,5-dimethoxyphenyl)difluoromethyl)furo[2,3-c]pyridin-5-yl)amino)-5-(4-ethylpiperazin-1-yl)phenyl)acrylamide

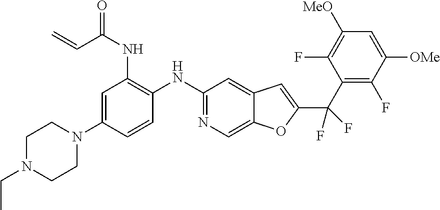 [SMILES: C=CC(=O)Nc1cc(N2CCN(CC)CC2)ccc1Nc1cc2cc(C(F)(F)c3c(F)c(OC)cc(OC)c3F)oc2cn1]

### Step 1 5-chloro-2-((2,6-difluoro-3,5-dimethoxyphenyl)difluoromethyl)furo[2,3-c]pyridine

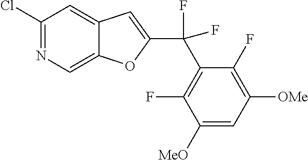 [SMILES: COc1cc(OC)c(F)c(C(F)(F)c2cc3cc(Cl)ncc3o2)c1F]

(5-Chlorofuro[2,3-c]pyridin-2-yl)(2,6-difluoro-3,5-dimethoxyphenyl)methanone (250 mg, 0.70 mmol) in Deoxo-Fluor (3.5 mL) was heated at 90° C. for 24 hrs. Then the reaction was cooled to RT and quenched with a small piece of ice. The reaction mixture was partitioned between EtOAc and sat. NaHCO₃. The aqueous phase was extracted with EtOAc (20 mL). The combined organic phases were washed with brine, dried with Na₂SO₄, filtered and concentrated. Residue obtained was loaded onto silica gel column with DCM and purified by column chromatography (eluent:Hexanes:DCM/100:0 to 50:50) to give 60 mg of compound 8. MS: [M+1]⁺: 376.72

### Step 2 N-(2-((2-((2,6-difluoro-3,5-dimethoxyphenyl)difluoromethyl)furo[2,3-c]pyridin-5-yl)amino)-5-(4-ethylpiperazin-1-yl)phenyl)acrylamide

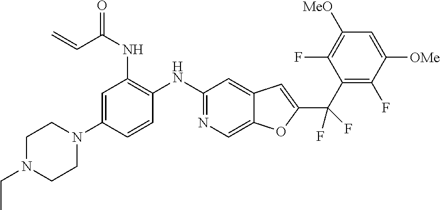 [SMILES: C=CC(=O)Nc1cc(N2CCN(CC)CC2)ccc1Nc1cc2cc(C(F)(F)c3c(F)c(OC)cc(OC)c3F)oc2cn1]

The title compound was prepared by using procedures analogous to those described in Example 3. MS: [M+H]⁺: 614.11

### EXAMPLE 9

### N-(2-((2-(2,6-dichloro-3,5-dimethoxybenzoyl)furo[2,3-c]pyridin-5-yl)amino)-5-(4-ethylpiperazin-1-yl)phenyl)acrylamide

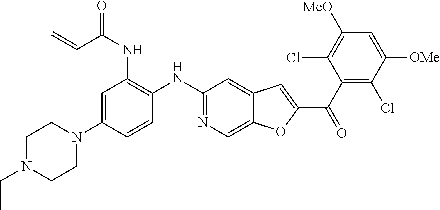 [SMILES: C=CC(=O)Nc1cc(N2CCN(CC)CC2)ccc1Nc1cc2cc(C(=O)c3c(Cl)c(OC)cc(OC)c3Cl)oc2cn1]

Using 2,6-chloro-3,5-dimethoxybenzaldehyde as starting material, the title compound was prepared by the procedure analogous to that described for Example 3. MS (ES+): 624.20 [M+1]⁺.

### EXAMPLE 10

### (±)-N-(3-((2-((2,6-difluoro-3,5-dimethoxyphenyl)(hydroxy)methyl)furo[2,3-c]pyridin-5-yl)amino)-1-methyl-1H-pyrazol-4-yl)acrylamide

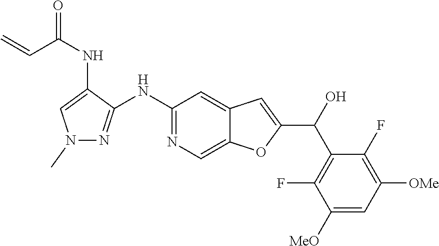 [SMILES: C=CC(=O)Nc1cn(C)nc1Nc1cc2cc(C(O)c3c(F)c(OC)cc(OC)c3F)oc2cn1]

### Step 1 (5-((4-amino-1-methyl-1H-pyrazol-3-yl)amino)furo[2,3-c]pyridin-2-yl)(2,6-difluoro-3,5-dimethoxyphenyl)methanol

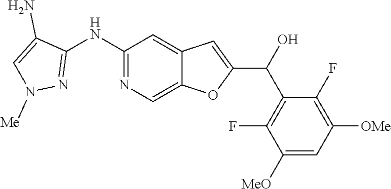 [SMILES: COc1cc(OC)c(F)c(C(O)c2cc3cc(Nc4nn(C)cc4N)ncc3o2)c1F]

To a solution of the product of Step 4 of Example 17 (31 mg, 0.067 mmol) in acetone (5 mL) at 0° C. was added NH₄Cl (37 mg, 0.67 mmol) in H₂O (0.5 mL) and Zn (26 mg, 0.40 mmol). The reaction mixture was stirred for 25 min at 0° C. The reaction mixture was filtered through a layer of Celite®. The filtrate was concentrated and partitioned between DCM and H₂O. The aqueous phase was extracted with DCM (20 mL×2). The combined organic layers were washed with brine, dried over Na₂SO₄ and concentrated to give 12 mg of the crude title compound which was used in the next step without purification. MS:M⁺: 431.59.

### Step 2 N-(3-((2-((2,6-difluoro-3,5-dimethoxyphenyl)(hydroxy)methyl)furo[2,3-c]pyridin-5-yl)amino)-1-methyl-1H-pyrazol-4-yl)acrylamide

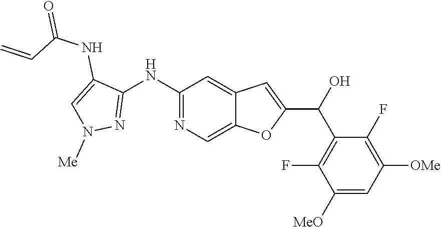 [SMILES: C=CC(=O)Nc1cn(C)nc1Nc1cc2cc(C(O)c3c(F)c(OC)cc(OC)c3F)oc2cn1]

The product of Step 1 (12 mg, 0.028 mmol) was dissolved in a solution of THF (2 mL) and H₂O (0.2 mL). At 0° C., 3-chloropropionyl chloride (7 mg, 0.055 mmol) was added dropwise to the reaction mixture. The reaction was warmed to room temperature and stirred for 45 min. NaOH (6.7 mg, 0.17 mmol) in H₂O (0.5 mL) was added the reaction, and the reaction was stirred overnight. The reaction mixture was partitioned between DCM and water. The aqueous phase was extracted with DCM (10 mL). The combined organic phases were washed with brine, dried with Na₂SO₄, filtered and concentrated. Residue obtained was purified by preparative TLC to give 2.85 mg of the title compound. MS: [M+H]⁺: 486.06.

### EXAMPLE 11

### N-(2-((2-(3,5-dimethoxybenzoyl)furo[2,3-c]pyridin-5-yl)amino)-5-(4-ethylpiperazin-1-yl)phenyl)acrylamide

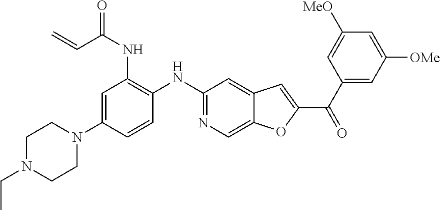 [SMILES: C=CC(=O)Nc1cc(N2CCN(CC)CC2)ccc1Nc1cc2cc(C(=O)c3cc(OC)cc(OC)c3)oc2cn1]

Using 3,5-dimethoxybenzaldehyde as starting material, the title compound was prepared by the procedure analogous to that described for Example 3. MS (ES+): 486.20 [M+1]⁺.

### EXAMPLE 12

### N-((3S,4S)-3-((2-(2,6-difluoro-3,5-dimethoxybenzoyl)furo[2,3-c]pyridin-5-yl)amino)tetrahydro-2H-pyran-4-yl)acrylamide

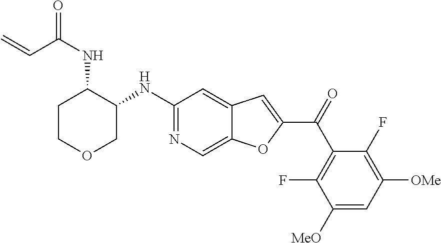 [SMILES: C=CC(=O)N[C@H]1CCOC[C@H]1Nc1cc2cc(C(=O)c3c(F)c(OC)cc(OC)c3F)oc2cn1]

### Step 1 (5-(((3S,4S)-4-azidotetrahydro-2H-pyran-3-yl)amino)furo[2,3-c]pyridin-2-yl)(2,6-difluoro-3,5-dimethoxyphenyl)methanone

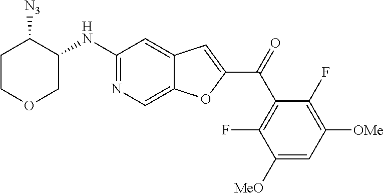 [SMILES: COc1cc(OC)c(F)c(C(=O)c2cc3cc(N[C@@H]4COCC[C@@H]4N=[N+]=[N-])ncc3o2)c1F]

To a solution of (35,45)-4-azidotetrahydro-2H-pyran-3-amine (284 mg, 2.0 mmol) and (5-(((3S,4S)-4-azidotetrahydro-2H-pyran-3-yl)amino)furo[2,3-c]pyridin-2-yl)(2,6-difluoro-3,5-dimethoxyphenyl)methanone (235 mg, 0.67 mmol) in toluene (15 mL) was added Xantphos (154 mg, 0.27 mmol), Cs₂CO₃ (868 mg, 2.66 mmol). Argon was bubbled through the mixture for 10 min and Pd₂(dba)₃ (122 mg, 0.13 mmol) was added. Argon was bubbled through the mixture again for 10 min. The reaction mixture was heated to 95° C. for 6 h under argon. After cooling to room temperature, the reaction mixture was partitioned between EtOAc and water. The aqueous phase was extracted with EtOAc (3×50 mL). The combined organic phases were washed with brine, dried with Na₂SO₄, filtered and concentrated. Residue obtained was loaded onto silica gel column with DCM and purified by column chromatography (eluent:MeOH:DCM/0:100 to 3:97). Then the collected compound title product was purified again by preparative TLC to give 41 mg of the title compound (yield: 13%). MS (ES+) M⁺: 459.57.

### Step 2 (5-(((3S,4S)-4-aminotetrahydro-2H-pyran-3-yl)amino)furo[2,3-c]pyridin-2-yl)(2,6-difluoro-3,5-dimethoxyphenyl)methanone

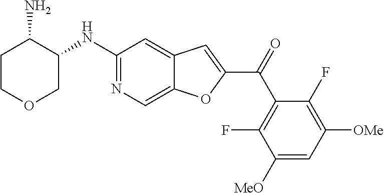 [SMILES: COc1cc(OC)c(F)c(C(=O)c2cc3cc(N[C@@H]4COCC[C@@H]4N)ncc3o2)c1F]

Pd/C (10%, 12 mg) was added to a solution of the product of Step 1 (48 mg, 0.104 mmol) in MeOH (4 mL) and THF (4 mL). The reaction mixture was stirred for 2.5 h under hydrogen (1 atm) at RT. The reaction mixture was filtered through a layer of Celite®. The filtrate was concentrated, then the crude product was purified by prep-HPLC to afford 12 mg the title compound (yield: 27%). MS:M⁺: 433.54.

### Step 3 N-((3S ,4S)-3-((2-(2,6-difluoro-3,5-dimethoxybenzoyl)furo[2,3-c]pyridin-5-yl)amino)tetrahydro-2H-pyran-4-yl)acrylamide

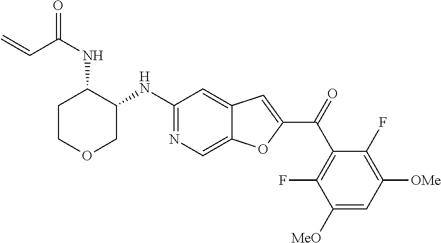 [SMILES: C=CC(=O)N[C@H]1CCOC[C@H]1Nc1cc2cc(C(=O)c3c(F)c(OC)cc(OC)c3F)oc2cn1]

The product of Step 2 (12 mg, 0.028 mmol) was dissolved in a solution of THF (2 mL) and H₂O (0.2 mL). At 0° C., 3-chloropropionyl chloride (7 mg, 0.055 mmol) in THF (0.11 mL) was added dropwise to the reaction mixture. The reaction was warmed to room temperature and stirred for 1 h. Another portion of 3-chloropropionyl chloride (21 mg, 0.165 mmol) in THF (0.3 mL) was added to the reaction mixture. After stirring for 3 h at RT, NaOH (68 mg, 1.70 mmol) in H₂O (1.0 mL) was added to the reaction mixture, and the reaction was stirred overnight. The reaction mixture was partitioned between DCM and water. The aqueous phase was extracted with DCM (10 mL). The combined organic phases were washed with brine, dried with Na₂SO₄, filtered and concentrated. Residue obtained was purified by preparative TLC twice to give 6 mg of the title compound. MS: 488.90 [M +1]⁺.

### EXAMPLE 13A and 13B

### N-(2-((2-((2,6-difluoro-3,5-dimethoxyphenyl)(hydroxy)methyl)furo[2,3-c]pyridin-5-yl)amino)-5-(4-ethylpiperazin-1-yl)phenyl)acrylamide

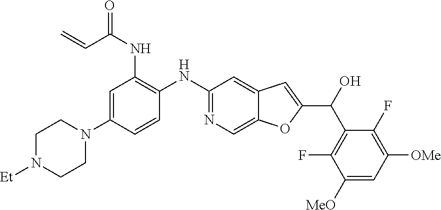 [SMILES: C=CC(=O)Nc1cc(N2CCN(CC)CC2)ccc1Nc1cc2cc(C(O)c3c(F)c(OC)cc(OC)c3F)oc2cn1]

(R and S enantiomers)

Example 5 was resolved on a Daicel AD-H column (4.6×250 mm, 5□) eluted with a mixture of 25% hexane/75% ethanol at a flow rate of 3 mL/min. Example 15A and Example 15B have 10 min and 16 min of retention time, respectively.

### EXAMPLE 14

### (±)N-((3S,4S)-3-((2-((2,6-difluoro-3,5-dimethoxyphenyl)(hydroxy)methyl)furo[2,3-c]pyridin-5-yl)amino)tetrahydro-2H-pyran-4-yl)acrylamide

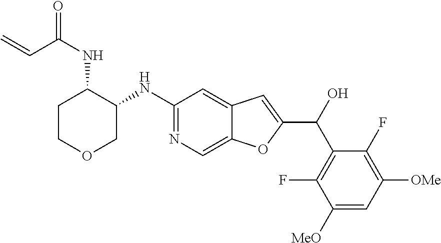 [SMILES: C=CC(=O)N[C@H]1CCOC[C@H]1Nc1cc2cc(C(O)c3c(F)c(OC)cc(OC)c3F)oc2cn1]

The title compound was prepared by the procedure analogous to that described in Step 1 for Example 5. MS (ES+) 490.2[M +1]⁺.

### EXAMPLE 15

### N-(3-((2-(2,6-difluoro-3,5-dimethoxybenzoyl)furo[2,3-c]pyridin-5-yl)amino)-1-methyl-1H-pyrazol-4-yl)acrylamide

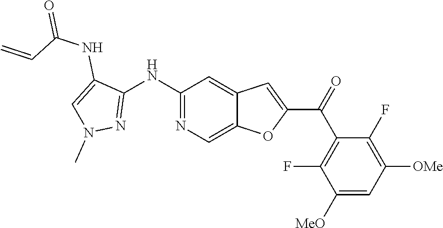 [SMILES: C=CC(=O)Nc1cn(C)nc1Nc1cc2cc(C(=O)c3c(F)c(OC)cc(OC)c3F)oc2cn1]

### Step 1 (2,6-difluoro-3,5-dimethoxyphenyl)(5-((1-methyl-4-nitro-1H-pyrazol-3-yl)amino)furo[2,3-c]pyridin-2-yl)methanone

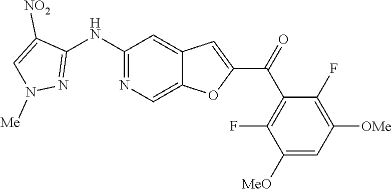 [SMILES: COc1cc(OC)c(F)c(C(=O)c2cc3cc(Nc4nn(C)cc4[N+](=O)[O-])ncc3o2)c1F]

To a solution of 1-methyl-4-nitro-1H-pyrazol-3-amine (80 mg, 0.56 mmol) and (5-chlorofuro[2,3-c]pyridin-2-yl)(2,6-difluoro-3,5-dimethoxyphenyl)methanone (200 mg, 0.56 mmol) in toluene (15 mL) was added Xantphos (131 mg, 0.22 mmol), Cs₂CO₃ (368 mg, 1.12 mmol). Argon was bubbled through the mixture for 10 min and Pd₂(dba)₃ (103 mg, 0.11 mmol) was added. Argon was bubbled through the mixture again for 10 min. The reaction mixture was heated to 110° C. for 6 h under argon. After cooling to room temperature, the reaction mixture was partitioned between EtOAc and water. The aqueous phase was extracted with EtOAc (3×50 mL). The combined organic phases were washed with brine, dried with Na₂SO₄, filtered and concentrated. Residue obtained was loaded onto silica gel column with DCM and purified by column chromatography (eluent:MeOH:DCM/0:100 to 3:97) to give 150 mg of the title compound (yield: 58%). MS:M⁺: 459.57.

### Step 2 tert-butyl (2-(2,6-difluoro-3,5-dimethoxybenzoyl)furo[2,3-c]pyridin-5-yl)(1-methyl-4-nitro-1H-pyrazol-3-yl)carbamate

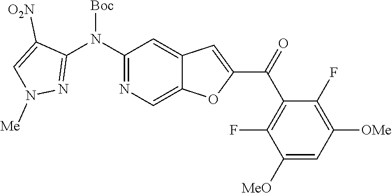 [SMILES: COc1cc(OC)c(F)c(C(=O)c2cc3cc(N(C(=O)OC(C)(C)C)c4nn(C)cc4[N+](=O)[O-])ncc3o2)c1F]

The product of Step 1 (22 mg, 0.028 mmol) was dissolved in a solution of THF (10 mL). (Boc)₂O (13 mg, 0.028 mmol) and DMAP (1.2 mg, 0.028 mmol) were added to the reaction, and the reaction was refluxed for 4 hrs. After cooling to RT, the solvent was removed by rotavap. Residue obtained was loaded onto silica gel column with DCM and purified by column chromatography (eluent:MeOH:DCM/0:100 to 5:95) to give 27 mg of the title compound.

### Step 3 tert-butyl (4-amino-1-methyl-1H-pyrazol-3-yl)(2-(2,6-difluoro-3,5-dimethoxybenzoyl)furo[2,3-c]pyridin-5-yl)carbamate

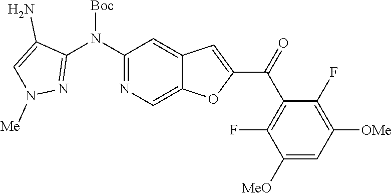 [SMILES: COc1cc(OC)c(F)c(C(=O)c2cc3cc(N(C(=O)OC(C)(C)C)c4nn(C)cc4N)ncc3o2)c1F]

Pd/C (10%, 10 mg) was added to a solution of the product of Step 2 (27 mg, 0.05 mmol) in MeOH (4 mL). The reaction mixture was stirred for 1 h under hydrogen (1 atm) at RT. The reaction mixture was filtered through a layer of Celite®. The filtrate was concentrated to afford 25 mg crude title compound which was used directly to the next step.

### Step 4 (5-((4-amino-1-methyl-1H-pyrazol-3-yl)amino)furo[2,3-c]pyridin-2-yl)(2,6-difluoro-3,5-dimethoxyphenyl)methanone

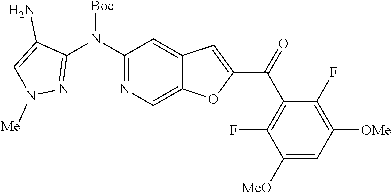 [SMILES: COc1cc(OC)c(F)c(C(=O)c2cc3cc(N(C(=O)OC(C)(C)C)c4nn(C)cc4N)ncc3o2)c1F]

To a solution of the product of Step 3 in DCM (3 mL) at 0° C. was added TFA (1 mL). Then the cold bath was removed and the reaction was stirred for 2 hours at RT. The reaction was concentrated under reduced pressure to give 11 mg crude title compound which was used directly to the next step.

### Step 5 N-(3-((2-(2,6-difluoro-3,5-dimethoxybenzoyl)furo[2,3 -c]pyridin-5-yl)amino)-1-methyl-1H-pyrazol-4-yl)acrylamide

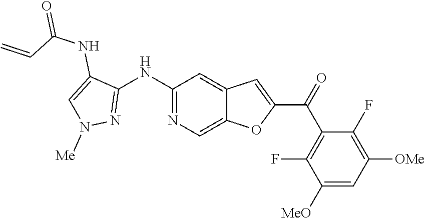 [SMILES: C=CC(=O)Nc1cn(C)nc1Nc1cc2cc(C(=O)c3c(F)c(OC)cc(OC)c3F)oc2cn1]

The product of Step 4 (11 mg, 0.026 mmol) was dissolved in a solution of THF (2 mL) and H₂O (0.2 mL). At 0° C., 3-chloropropionyl chloride (6.3 mg, 0.05 mmol) was added dropwise to the reaction mixture. The reaction was warmed to room temperature and stirred for 20 min. NaOH (6 mg, 0.15 mmol) in H₂O (0.5 mL) was added the reaction, and the reaction was stirred at 65° C. for 5 hrs. The reaction mixture was cooled to RT, and partitioned between DCM and water. The aqueous phase was extracted with DCM (10 mL). The combined organic phases were washed with brine, dried with Na₂SO₄, filtered and concentrated. Residue obtained was purified by column chromatography (eluent: MeOH:DCM/0:100 to 10:90) and then preparative TLC to give 0.76 mg of the title compound. MS: [M+H]⁺: 484.74.

### EXAMPLE 16

### N-(2-((2-(2,6-difluoro-3,5-dimethoxybenzoyl)thieno[2,3-c]pyridin-5-yl)amino)-5-(4-ethylpiperazin-1-yl)phenyl)acrylamide

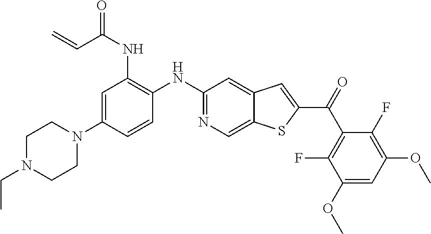 [SMILES: C=CC(=O)Nc1cc(N2CCN(CC)CC2)ccc1Nc1cc2cc(C(=O)c3c(F)c(OC)cc(OC)c3F)sc2cn1]

### Step 1 5-chlorothieno[2,3-c]pyridine-2-carboxylic acid

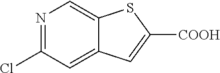 [SMILES: O=C(O)c1cc2cc(Cl)ncc2s1]

To a stirred solution of 2-chloro-5-fluoroisonicotinaldehyde (17 g, 0.1 mol) in DMF (300 ml), K₂CO₃ (44 g, 0.3 mol) and methyl 2-mercaptoacetate (34 g, 0.3 mol) were added. Then the mixture was heated to 90° C. and stirred for 3 h. The reaction mixture was poured into ice, then acidified with 1N HCl to pH=5-6. The resulting precipitate was collected by filtration and dried to give 20 g of the title product. Yield: 88.1%.

### Step 2 5-chloro-N-methoxy-N-methylthieno[2,3-c]pyridine-2-carboxamide

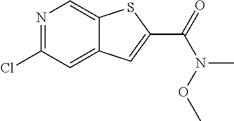 [SMILES: CON(C)C(=O)c1cc2cc(Cl)ncc2s1]

To a stirred solution of 5-chlorothieno[2,3-c]pyridine-2-carboxylic acid (4.92 g, 23.02 mmol) in DMF (50 ml) was added Et₃N (12.7 ml, 92.1 mmol) , HBTU (10.45 g , 27.64 mmol) and N,O-dimethylhydroxylamine hydrochloride (4.47 g , 46.06 mmol), respectively. Then the mixture was stirred overnight at RT. Water was added and the resulting precipitate was collected by filtration and dried to give 2.91 g of the title product. Yield: 49.1%.

### Step 3 5-chlorothieno[2,3-c]pyridine-2-carbaldehyde

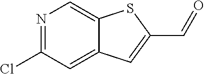 [SMILES: O=Cc1cc2cc(Cl)ncc2s1]

To a stirred solution of 5-chloro-N-methoxy-N-methylthieno[2,3-c]pyridine-2-carboxamide (2.9 g, 11.3 mmol) in THF (30 ml) was added LiA1H4 (0.857 g, 22.6 mmol) at 0° C. in portions. The mixture was stirred for 1 h at RT. Na₂SO₄.10H₂O and DCM were added to the reaction mixture at 0° C., and then stirred at RT for 1 h. The resulting precipitate was collected by filtration. The filtrate was concentrated and purified by column photography (PE:EA=9:1) to give 1 g of the title product as a white solid. Yield: 44.8%.

### Step 4 (5-chlorothieno[2,3-c]pyridin-2-yl)(2,6-difluoro-3,5-dimethoxyphenyl)methanol

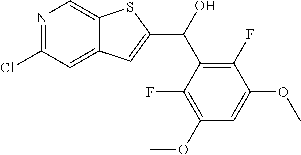 [SMILES: COc1cc(OC)c(F)c(C(O)c2cc3cc(Cl)ncc3s2)c1F]

To a stirred solution of 2,4-difluoro-3-iodo-1,5-dimethoxybenzene (1.39 g, 4.63 mmol) in THF (20 ml), isopropylmagnesium chloride (2 M in THF, 2.3 ml, 4.63 mmol) was added drop wise at 0° C. under Ar and stirred for 30 min. 5-chlorothieno[2,3-c]pyridine-2-carbaldehyde (0.91 g, 4.63 mmol) in THF (5 ml) was added while keeping the temperature below 5° C. After the addition was complete, the mixture was stirred at RT for 1 h. The solution was quenched with sat. NH₄Cl and extracted with EA. The organic layer was washed with brine and dried over Na₂SO₄. The solution was filtered, concentrated and purified by column photography (PE:EA=2:1) to give 730 mg of the title product as a white solid. Yield: 42.4%.

### Step 5 (2,6-difluoro-3,5-dimethoxyphenyl)(5-((4-(4-ethylpiperazin-1-yl)-2-nitrophenyl)amino)thieno[2,3-c]pyridin-2-yl)methanone

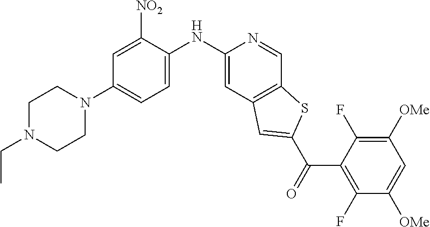 [SMILES: CCN1CCN(c2ccc(Nc3cc4cc(C(=O)c5c(F)c(OC)cc(OC)c5F)sc4cn3)c([N+](=O)[O-])c2)CC1]

A mixture of (5-chlorothieno[2,3-c]pyridin-2-yl)(2,6-difluoro-3,5-dimethoxyphenyl)methanol (730 mg, 1.96 mmol), 4-(4-ethylpiperazin-1-yl)-2-nitroaniline (590 mg, 2.36 mmol), Pd₂(dba)₃ (360 mg, 0.393 mmol), Xantphos (454 mg, 0.785 mmol) and Cs₂CO₃ (1.28 g, 3.93 mmol) in toluene (10 ml) was heated to 110° C. under argon and stirred overnight. The reaction mixture was cooled to room temperature, filtered and concentrated to dryness. The residue was purified by column photography (DCM:MeOH=95:5) to give 1.0 g of the title product as a dark purple solid. Yield: 86.8%.

### Step 6 (5-((2-amino-4-(4-ethylpiperazin-1-yl)phenyl)amino)thien[2,3-c]pyridin-2-yl)(2,6-difluoro-3,5-dimethoxyphenyl)methanone

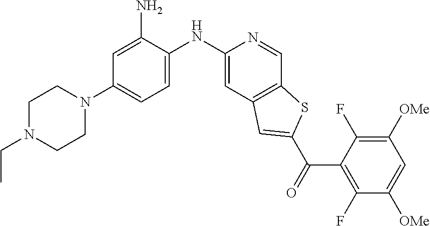 [SMILES: CCN1CCN(c2ccc(Nc3cc4cc(C(=O)c5c(F)c(OC)cc(OC)c5F)sc4cn3)c(N)c2)CC1]

To a stirred solution of (2,6-difluoro-3,5-dimethoxyphenyl)(5-((4-(4-ethylpiperazin-1-yl)-2-nitrophenyl)amino)thieno[2,3-c]pyridin-2-yl)methanone (500 mg, 0.857 mmol) in i-PrOH/H₂O (9 ml/3 ml), Fe (479 mg, 8.57 mmol) and NH₄Cl (459 mg, 8.57 mmol) were added. The reaction was stirred under reflux for 1 h, then cooled to room temperature and filtered. The solution was quenched with water and extracted with EA. The organic layer was washed with brine and dried over Na₂SO₄. The solution was filtered and concentrated to give 500 mg of the crude title compound which was used in the next step without further purification.

### Step 7 N-(2-((2-(2,6-difluoro-3,5-dimethoxybenzoyl)thieno [2,3-c]pyridin-5-yl)amino)-5-(4-ethylpiperazin-1-yl)phenyl)acrylamide

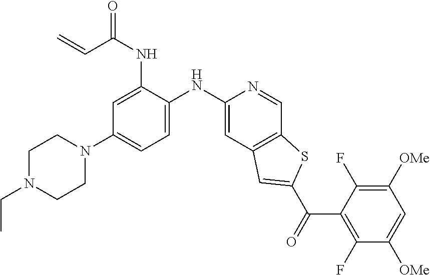 [SMILES: C=CC(=O)Nc1cc(N2CCN(CC)CC2)ccc1Nc1cc2cc(C(=O)c3c(F)c(OC)cc(OC)c3F)sc2cn1]

The product of Step 6 (500 mg crude, 0.968 mmol) was dissolved in DCM (6 ml). At RT, DIPEA (0.8 ml, 625.4 mg, 4.84 mmol), acrylic acid (349 mg, 4.84 mmol) and T3P (50% in DMF, 3 g, 4.84 mmol) were added to the reaction mixture, and then stirred overnight. The reaction was quenched with saturated aqueous sodium bicarbonate, and extracted with dichloromethane. The organic layer was washed with brine and dried over Na₂SO₄. The solution was filtered, concentrated and purified by column photography (DCM:MeOH=9:1) to get 14 mg of pure product. Yield: 2.7%. MS (ES+): 608.2 [M+1]⁺.

¹HNMR: (400 MHz, Methanol-d₄) δ 1.26-1.30 (t, J=16 Hz, 3H), 2.65-2.67 (q, 2H), 2.81(s, 4H), 3.28 (s, 4H), 3.95 (s, 6H), 5.71-5.73 (d, J=8 Hz, 1H), 6.33-6.37 (m, 2H), 6.91(s, 2H), 7.07-7.09 (t, J=8 Hz, 1H), 7.32-7.38 (m, 2H), 7.61(s, 1H), 8.77(s, 1H).

### EXAMPLE 17

### N-(2-((2-(2,6-difluoro-3,5-dimethoxybenzoyl)furo[2,3-c]pyridin-5-yl)amino)-3-methylphenyl)acrylamide

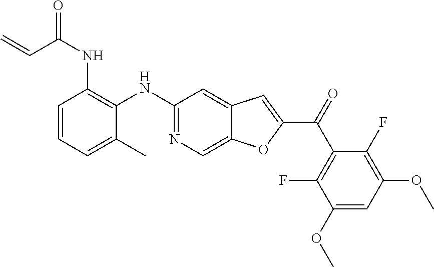 [SMILES: C=CC(=O)Nc1cccc(C)c1Nc1cc2cc(C(=O)c3c(F)c(OC)cc(OC)c3F)oc2cn1]

### Step 1 (2,6-difluoro-3,5-dimethoxyphenyl)(5-(2-methyl-6-nitrophenylamino)furo[2,3-c]pyridin-2-yl)methanone

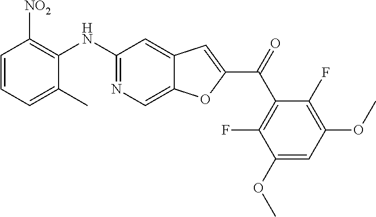 [SMILES: COc1cc(OC)c(F)c(C(=O)c2cc3cc(Nc4c(C)cccc4[N+](=O)[O-])ncc3o2)c1F]

A mixture of (5-chlorofuro[2,3-c]pyridin-2-yl)(2,6-difluoro-3,5-dimethoxyphenyl)methanone (0.500 g, 1.41 mmol), 2-methyl-6-nitroaniline (0.258 g, 1.70 mmol), Pd₂(dba)₃ (0.260 g, 0.284 mmol), X-phos (0.270 g, 0.567 mmol) and Cs₂CO₃ (0.922 g, 2.828 mmol) in toluene (10 ml) was heated at 115° C. under N₂ for 15 h. The reaction mixture was cooled to room temperature, filtered and concentrated to dryness. The residue was purified by column photography (EA/PE=1:2) to give 0.34 g of the title product as a yellow solid. Yield: 51.2%.

### Step 2 (5-(2-amino-6-methylphenylamino)furo[2,3-c]pyridin-2-yl)(2,6-difluoro-3,5-dimethoxyphenyl)methanone

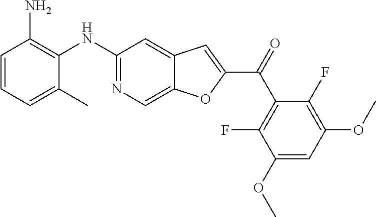 [SMILES: COc1cc(OC)c(F)c(C(=O)c2cc3cc(Nc4c(C)cccc4N)ncc3o2)c1F]

To a stirred solution of (2,6-difluoro-3,5-dimethoxyphenyl)(5-(2-methyl-6-nitrophenylamino)furo[2,3-c]pyridin-2-yl)methanone (240 mg, 0.51 mmol) in EtOH/H₂O (8 ml/2 ml), Fe (137 mg, 2.45 mmol) and NH₄Cl (144 mg, 2.69 mmol) were added. The resulting mixture was stirred at 85° C. for 2 h, then cooled to room temperature and filtered. The solution was quenched with water and extracted with DCM. The organic layer was washed with brine and dried over Na₂SO₄. The solution was filtered and concentrated to give 240 mg of crude title compound which was used in the next step without further purification. MS (ES+): 440 [M +1]⁺.

### Step 3 N-(2-(2-(2,6-difluoro-3,5-dimethoxybenzoyl)furo[2,3-c]pyridin-5-ylamino)-3-methylphenyl)acrylamide

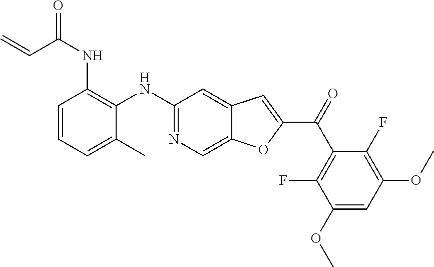 [SMILES: C=CC(=O)Nc1cccc(C)c1Nc1cc2cc(C(=O)c3c(F)c(OC)cc(OC)c3F)oc2cn1]

The product of Step 2 (240 mg, 0.55 mmol) and T3P (1.83 g. 2.87 mmol) (50% in DMF), DIEA (370 mg, 2.87 mmol) were dissolved in DCM (10 ml). At room temperature, acrylic acid (34.4 mg, 0.48 mmol) was added to the reaction mixture, and then stirred for 1 h. After the reaction was complete, the reaction mixture was concentrated and purified by pre-TLC (EA/PE=1:1) to get 60 mg of the title product as a yellow solid. Yield: 22.1%. MS (ES+): 494.7 [M+1]⁺.

¹HNMR Spectrum: (400 MHz, CDCl₃) δ 2.20 (s, 3H), 3.94 (s, 6H), 5.67-5.69 (d, J=8 Hz,1H), 5.97 (s, 1H), 6.10-6.16 (q, 1H), 6.28-6.30 (d, J=8 Hz, 1H), 6.34 (s, 1H), 6.79-6.83 (t, J=16 Hz, 1H), 7.07-7.09 (d, J=8 Hz, 1H), 7.21 (s, 1H), 7.30-7.33(d, J=12 Hz, 1H), 8.20 (s, 1H), 8.38-8.40(d, J=8 Hz, 1H), 8.66(s, 1H).

### EXAMPLE 18

### N-(5-(4-ethylpiperazin-1-yl)-2-((1-(4-methoxybenzyl)-1H-pyrrolo[2,3-c]pyridin-5-yl)amino)phenyl)acrylamide

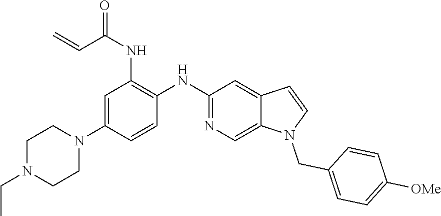 [SMILES: C=CC(=O)Nc1cc(N2CCN(CC)CC2)ccc1Nc1cc2ccn(Cc3ccc(OC)cc3)c2cn1]

### Step 1 5-chloro-1-(4-methoxybenzyl)-1H-pyrrolo[2,3-c]pyridine

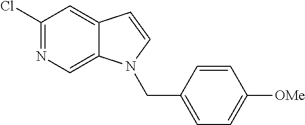 [SMILES: COc1ccc(Cn2ccc3cc(Cl)ncc32)cc1]

To a solution of 5-chloro-1H-pyrrolo[2,3-c]pyridine (0.61 g, 4.0 mmol) in DMF (6 ml) were added Cs₂CO₃ (2.62 g, 8 mmol) and PMBCl (0.63 g, 4.0 mmol). The resulting mixture was stirred at 40° C. for 2 h, then cooled to room temperature and quenched with water and stirred for 30 minutes, filtrated and washed with water. The solid was dried in vacuum to give 1.1 g of the title product as an off-white solid. Yield: 98%. MS (ES+): 273 [M +1]⁺

### Step 2 N-(4-(4-ethylpiperazin-1-yl)-2-nitrophenyl)-1-(4-methoxybenzyl)-1H-pyrrolo[2,3-c]pyridin-5-amine

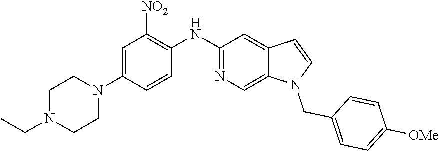 [SMILES: CCN1CCN(c2ccc(Nc3cc4ccn(Cc5ccc(OC)cc5)c4cn3)c([N+](=O)[O-])c2)CC1]

To a solution of the product of Stepl (1.8 g, 6.6 mmol) and 4-(4-ethylpiperazin-1-yl)-2-nitroaniline (1.5 g, 6.0 mmol) in toluene (25 ml) was added X-phos (1.4 g, 2.9 mmol), Cs₂CO₃ (3.9 g, 12 mmol). Argon was bubbled through the mixture for 10 min and Pd₂(dba)₃(1.1 g, 13.4 mmol) was added. Argon was bubbled through the mixture again for 10 min. The reaction mixture was heated to 115° C. and stirred overnight. After cooling to room temperature, the reaction was partitioned between EA and water. The aqueous phase was extracted with EA. The combined organic phases were washed with brine and dried over Na₂SO₄. The solution was filtered and concentrated. The residue was purified by column photography (MeOH/DCM=3%) to give 1.8 g of the title product as a dark brown solid. MS (ES+): 487 [M +1]⁺

### Step 3 4-(4-ethylpiperazin-1-yl)-N¹-(1-(4-methoxybenzyl)-1H-pyrrolo[2,3 -c]pyridin-5-yl)benzene-1,2-diamine

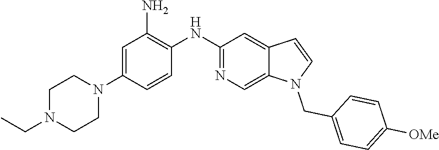 [SMILES: CCN1CCN(c2ccc(Nc3cc4ccn(Cc5ccc(OC)cc5)c4cn3)c(N)c2)CC1]

To a stirred solution of Step2 (0.5 g, 1.0 mmol) in EtOH/H₂O (6 ml/2 ml), Fe (0.56 mg, 10 mmol) and NH₄Cl (0.56 g, 10 mmol) were added. The resulting mixture was stirred at refluxing forl h, then cooled to room temperature and filtered. The solution was quenched with water and extracted with EA. The organic layer was washed with brine and dried over Na₂SO₄. The solution was filtered and concentrated to give 0.43 g of the title compound which was used in the next step without further purification (yield 91%). MS (ES+): 457 [M +1]⁺

### Step 4 N-(5-(4-ethylpiperazin-1-yl)-2-(1-(4-methoxybenzyl)-1H-pyrrolo[2,3-c]pyridin-5-ylamino)phenyl)acrylamide

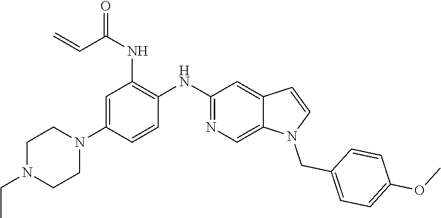 [SMILES: C=CC(=O)Nc1cc(N2CCN(CC)CC2)ccc1Nc1cc2ccn(Cc3ccc(OC)cc3)c2cn1]

To a solution of Step 3 (150 mg, 0.33 mmol) in DCM (6 ml) at 5° C. was added acrylic acid (19.5 mg, 0.27 mmol), DIPEA (0.3 ml 1.68 mmol)), then T3P (1 g 1.57 mmol) was added drop wise. After addition, the reaction mixture was stirred for 0.5 h. The reaction mixture was quenched with water and extracted with EA. The organic phase was washed with brine and dried over Na₂SO₄. The solution was filtered and concentrated. The residue was purified by column photography (MeOH/DCM=4%) to give 30 mg of the title product as a gray solid. MS (ES+): 511.2[M+1]⁺.

¹HNMR: (400 MHz, CDCl₃) δ 1.14-1.17 (t, J=7.2 Hz, 3H), 2.47-2.51 (m, 2H), 2.62-2.64 (t, J=4.8,4H), 3.8 (s, 6H), 5.25 (s, 2H), 5.63-5.67 (m, 2H), 6.1-6.4(m, 3H), 6.42 (s, 1H), 6.67-6.69 (m, 1H), 6.85-6.88(d, J=8.8,1H), 7.11-7.17(m, 3H), 8.29(s, 1H), 8.44(s,1H).

### EXAMPLE 19

### N-(2-((2-(2,6-difluoro-3,5-dimethoxybenzoyl)-1-methyl-1H-pyrrolo[2,3-c]pyridin-5-yl)amino)-5-(4-ethylpiperazin-1-yl)phenyl)acrylamide

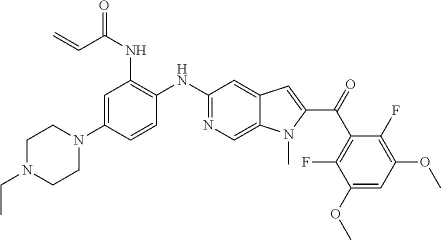 [SMILES: C=CC(=O)Nc1cc(N2CCN(CC)CC2)ccc1Nc1cc2cc(C(=O)c3c(F)c(OC)cc(OC)c3F)n(C)c2cn1]

### Step 1 (5-chloro-1-(phenylsulfonyl)-1H-pyrrolo[2,3-c]pyridin-2-yl)(2,6-difluoro-3,5-dimethoxyphenyl)methanone

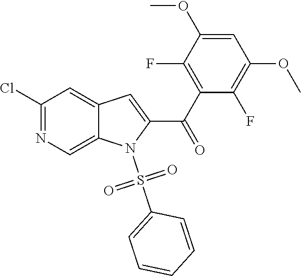 [SMILES: COc1cc(OC)c(F)c(C(=O)c2cc3cc(Cl)ncc3n2S(=O)(=O)c2ccccc2)c1F]

The mixture of (5-chloro-1-(phenylsulfonyl)-1H-pyrrolo[2,3-c]pyridin-2-yl)(2,6-difluoro-3,5-dimethoxyphenyl)methanol (4 g, 8.1 mmol) in acetone (100 ml) was cooled down to 5° C., then Jone's (4 ml) was added drop wise. After addition, the mixture was stirred at 5° C. for 30 min. The reaction mixture was quenched with water and extracted with EA. The organic layer was washed with brine and dried over Na₂SO₄. The solution was filtered and concentrated to give 3.5 g of the crude title product as an off-white solid which was used in the next step without further purification. Yield: 87%. MS (ES+):493 [M +1]⁺.

### Step 2 (5-chloro-1H-pyrrolo[2,3-c]pyridin-2-yl)(2,6-difluoro-3,5-dimethoxyphenyl)methanone

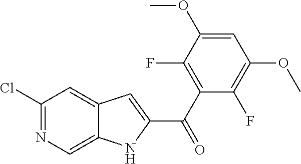 [SMILES: COc1cc(OC)c(F)c(C(=O)c2cc3cc(Cl)ncc3[nH]2)c1F]

To a solution of CH₃ONa (0.77 g, 14.2 mmol) in THF/MeOH (35 ml/35 ml) was added the product of Step 1 (3.5 g, 7.1 mmol) at RT. The reaction mixture was stirred for 0.5 h. The reaction mixture was quenched with water and stirred for 10 min, filtered and dried in vacuum to give 2 g of the title product as an off-white solid. Yield: 80%. MS (ES+):353 [M+1]⁺.

### Step 3 (5-chloro-1-methyl-1H-pyrrolo[2,3-c]pyridin-2-yl)(2,6-difluoro-3,5-dimethoxyphenyl)methanone

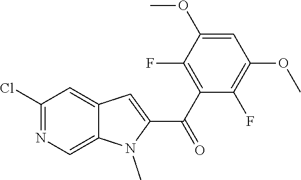 [SMILES: COc1cc(OC)c(F)c(C(=O)c2cc3cc(Cl)ncc3n2C)c1F]

To a solution of Step 2 (0.9 g, 0.255 mmol) in DMF (18 ml) was added Cs₂CO₃ (0.1.66 g, 7.67 mmol) and Mel (1.09 g, 7.67 mmol). The resulting mixture was stirred at 40° C. for 2 h, then cooled to room temperature, quenched with water and stirred for 30 minutes, filtrated and washed with water. The solid was dried in vacuum to give 0.8 g of the title product as an off-white solid. Yield 86%. MS (ES+): 367 [M +1]⁺

### Step 4 (2,6-difluoro-3,5-dimethoxyphenyl) (5-(4-(4-ethylpiperazin-1-yl)-2-nitrophenylamino)-1-methyl-1H-pyrrolo[2,3-c]pyridin-2-yl) methanone

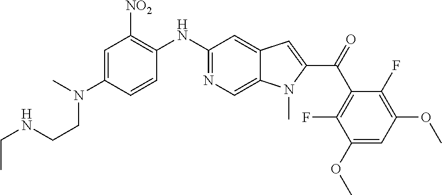 [SMILES: CCNCCN(C)c1ccc(Nc2cc3cc(C(=O)c4c(F)c(OC)cc(OC)c4F)n(C)c3cn2)c([N+](=O)[O-])c1]

To a solution of the product of Step3 (0.84 g, 2.23 mmol) and 4-(4-ethylpiperazin-1-yl)-2-nitroaniline (0.5 g, 2 mmol) in toluene (25 ml) was added X-phos (0.35 g, 0.73 mmol), Cs₂CO₃ (1.5 g, 4.6 mmol). Argon was bubbled through the mixture for 10 min and Pd₂ (dba) (0.35 g, 0.41 mmol) was added. Argon was bubbled through the mixture again for 10 min. The reaction mixture was heated to 115° C. and stirred overnight. After cooling to room temperature, the reaction was partitioned between EA and water. The aqueous phase was extracted with EA. The combined organic phases were washed with brine, dried over Na₂SO₄. The solution was filtered and concentrated. The residue was purified by column photography (MeOH/DCM=4%) to give 0.85 g of pure product as a dark brown solid. Yield: 64%. MS (ES+): 581 [M +1]⁺

### Step 5 (5-(2-amino-4-(4-ethylpiperazin-1-yl)phenylamino)-1-methyl-1H-pyrrolo[2,3-c]pyridin-2-yl)(2,6-difluoro-3,5-dimethoxyphenyl)methanone

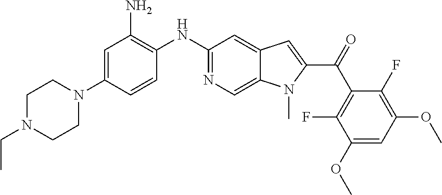 [SMILES: CCN1CCN(c2ccc(Nc3cc4cc(C(=O)c5c(F)c(OC)cc(OC)c5F)n(C)c4cn3)c(N)c2)CC1]

Pd/C (10%, 0.15 g) was added to a solution of the product of Step 4 (0.3 g, 0.53 mmol) in MeOH(10 ml). The reaction was stirred for 16 hours under hydrogen (1 atm, balloon) at room temperature. The reaction mixture was filtered through a layer of Celite. The filtrate was concentrated to afford 0.25 of crude title product as dark foam which was used in the next step without further purification. Yield: 90%.

MS (ES+): 551 [M]⁺

### Step 6 N-(2-(2-(2,6-difluoro-3,5-dimethoxybenzoyl)-1-methyl-1H-pyrrolo[2,3-c]pyridin-5-ylamino)-5-(4-ethylpiperazin-1-yl)phenyl)acrylamide

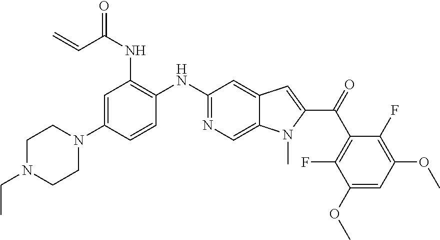 [SMILES: C=CC(=O)Nc1cc(N2CCN(CC)CC2)ccc1Nc1cc2cc(C(=O)c3c(F)c(OC)cc(OC)c3F)n(C)c2cn1]

To a solution of Step 5 (150 mg, 0.26 mmol) in DCM (6 ml) at 5° C. was added acrylic acid (19.5mg, 0.2 mmol), DIPEA (0.3 ml 1.68 mmol)), then T3P (1 g 1.57 mmol) was added dropwise. After addition, the reaction mixture was stirred for 0.5 h. The reaction mixture was quenched with water and extracted with EA. The organic phase was washed with brine and dried over Na₂SO₄. The solution was filtered and concentrated. The residue was purified by silica gel chromatography (MeOH/DCM=4%) to give 25 mg of the title product as a red solid. Yield: 15%.

MS (ES+): 605.2[M+1]⁺.

¹HNMR: (400 MHz, CDCl₃) δ 1.14-1.176 (t, J=7.2 Hz, 3H), 2.47-(m, 2H), 2.61-2.64 (t, J=4.8,4H) 3.29-3.32 (t, J=4.8, 4H), 3.92 (s, 6H), 4.23(s,3H),5.65-5.68(d, J=10 Hz, 1H),5.75(s,1H), 6.14-6.16 (m, 1H), 6.31-6.35 (m, 1H), 6.66-6.77 (m,3H), 7.11-7.13(d, J=8.8,1H), 8.22-8.26(d J=14.4, 1H), 8.6(s, 1H).

### EXAMPLE 20

### N-(2-((6-(2,6-difluoro-3,5-dimethoxybenzoyl)furo[3,2-d]pyrimidin-2-yl)amino)-5-(4-ethylpiperazin-1-yl)phenyl)acrylamide

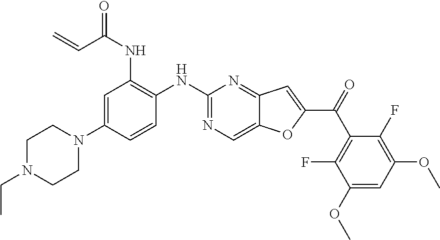 [SMILES: C=CC(=O)Nc1cc(N2CCN(CC)CC2)ccc1Nc1ncc2oc(C(=O)c3c(F)c(OC)cc(OC)c3F)cc2n1]

### Step 1 (2,4-dichlorofuro[3,2-d]pyrimidin-6-yl)(2,6-difluoro-3,5-dimethoxyphenyl)methanol

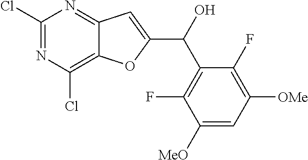 [SMILES: COc1cc(OC)c(F)c(C(O)c2cc3nc(Cl)nc(Cl)c3o2)c1F]

To a stirred solution of 2,4-dichlorofuro[3,2-d]pyrimidine (12.0 g, 63.5 mmol) in anhydrous THF (650 ml), LDA (47.3 ml, 2M in THF) was added drop wise at −60° C. and stirred for 1 h. 2,6-difluoro-3,5-dimethoxybenzaldehyde (16.68 g, 82.5 mmol) in anhydrous THF (105 ml) was then added drop wise. The resulting mixture was stirred at −60° C. for 2 h. The solution was quenched with sat. NH₄Cl and extracted with EA. The organic layer was washed with brine and dried over Na₂SO₄. The solution was filtered, concentrated and purified by column photography (PE:EA=3:1) to give 21.5 g of the title product as a yellow solid. Yield: 86%. MS (ES+): 391 [M +1]⁺.

### Step 2 (2-chlorofuro[3,2-d]pyrimidin-6-yl)(2,6-difluoro-3,5-dimethoxyphenyl)methanol

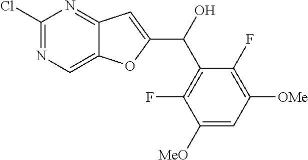 [SMILES: COc1cc(OC)c(F)c(C(O)c2cc3nc(Cl)ncc3o2)c1F]

To a stirred solution of (2,4-dichlorofuro[3,2-d]pyrimidin-6-yl)(2,6-difluoro-3,5-dimethoxyphenyl)methanol (1.0 g, 2.6 mmol) in MeOH (15 ml), Zn (0.67 g, 10.3 mmol) and AcOH (0.9 ml, 15.6 mmol) were added. The resulting mixture was stirred at 70° C. for 4 h, then cooled to room temperature and filtered. The solution was quenched with water and extracted with EA. The organic layer was washed with brine and dried over Na₂SO₄. The solution was filtered and concentrated to give 0.72 g of the title product as a yellow solid. Yield: 79.1%.

### Step 3 (2-chlorofuro[3,2-d]pyrimidin-6-yl)(2,6-difluoro-3,5-dimethoxyphenyl)methanone

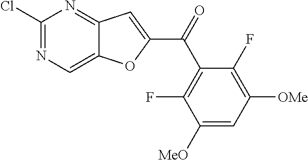 [SMILES: COc1cc(OC)c(F)c(C(=O)c2cc3nc(Cl)ncc3o2)c1F]

To a stirred solution of (2-chlorofuro[3,2-d]pyrimidin-6-yl)(2,6-difluoro-3,5-dimethoxyphenyl)methanol (2.0 g) in acetone (20 ml), Jone's reagent (12 ml) was added dropwise at 0-5° C. and stirred for 2.5 h. The solution was quenched with water and extracted with EA. The organic layer was washed with sat.NaHCO3, brine and dried over Na₂SO₄. The solution was filtered and concentrated to give 0.986 g of the title product as a yellow solid. Yield: 49.6%. MS (ES+): 355 [M +1]⁺.

### Step 4 (2,6-difluoro-3,5-dimethoxyphenyl)(2-(4-(4-ethylpiperazin-1-yl)-2-nitrophenylamino)furo[3,2-d]pyrimidin-6-yl)methanone

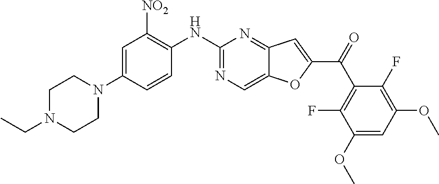 [SMILES: CCN1CCN(c2ccc(Nc3ncc4oc(C(=O)c5c(F)c(OC)cc(OC)c5F)cc4n3)c([N+](=O)[O-])c2)CC1]

A mixture of (2-chlorofuro[3,2-d]pyrimidin-6-yl)(2,6-difluoro-3,5-dimethoxyphenyl)methanone (0.986 g, 2.79 mmol), 4-(4-ethylpiperazin-1-yl)-2-nitroaniline (0.7 g, 2.79 mmol), Pd₂(dba)₃ (0.51 g, 0.558 mmol), X-phos (0.53 g, 1.116 mmol) and Cs₂CO₃ (1.82 g, 5.58 mmol) in toluene (20 ml) was heated at 120° C. under Are for 5-6 h. The reaction mixture was cooled to room temperature, filtered and concentrated to dryness. The residue was purified by column photography (MeOH/DCM=4%) to give 1.0 g of the title product as a dark brown solid. Yield: 63.3%.

### Step 5 (2-(2-amino-4-(4-ethylpiperazin-1-yl)phenylamino)furo[3,2-d]pyrimidin-6-yl)(2,6-difluoro-3,5-dimethoxyphenyl)methanone

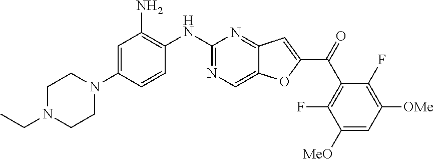 [SMILES: CCN1CCN(c2ccc(Nc3ncc4oc(C(=O)c5c(F)c(OC)cc(OC)c5F)cc4n3)c(N)c2)CC1]

To a stirred solution of (2,6-difluoro-3,5-dimethoxyphenyl)(2-(4-(4-ethylpiperazin-1-yl)-2-nitrophenylamino)furo[3,2-d]pyrimidin-6-yl)methanone (200 mg) in EtOH/H20 (80 ml/20 ml), Fe (200 mg) and NH₄C1 (200 mg) were added. The resulting mixture was stirred at 70° C. for 2.5 h, then cooled to room temperature and filtered. The solution was quenched with water and extracted with EA. The organic layer was washed with brine and dried over Na₂SO₄. The solution was filtered and concentrated to give 205 mg of the crude title compound which was used in the next step without further purification. MS (ES+): 539.2 [M +1]⁺.

### Step 6 N-(2-(6-(2,6-difluoro-3,5-dimethoxybenzoyl)furo[3,2-d]pyrimidin-2-ylamino)-5-(4-ethylpiperazin-1-yl)phenyl)acrylamide

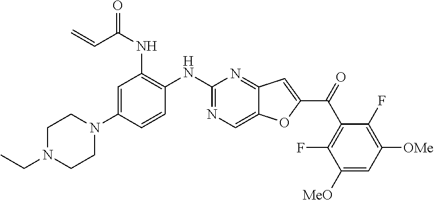 [SMILES: C=CC(=O)Nc1cc(N2CCN(CC)CC2)ccc1Nc1ncc2oc(C(=O)c3c(F)c(OC)cc(OC)c3F)cc2n1]

The product of Step 5 (200 mg, 0.37 mmol) and Et3N (56 mg, 0.555 mmol) were dissolved in DCM (4 ml). At 0-10° C., acrylyl chloride (34 mg, 0.37 mmol) was added to the reaction mixture, and then stirred for 0.5 h. After the reaction was complete, the reaction mixture was concentrated and purified by column photography (MeOH/DCM=6%) to get 117 mg of the title product as a red solid. Yield: 53.2%. MS (ES+): 593.3 [M+1]⁺.

¹HNMR: (400 MHz, CDCl₃) 1.20-1.24 (t, J=16 Hz, 3H), 2.84-2.90 (m, 2H), 2.96-3.02 (m, 4H), 3.44-3.46 (d, J=8 Hz, 4H), 3.95 (s, 6H), 5.71-5.74 (d, J=12 Hz, 1H), 6.20-6.27 (m, 1H), 6.37-6.41 (d, J=16 Hz, 1H), 6.72-6.75 (t, J=12 Hz, 1H), 6.81-6.85 (t, J=16 Hz, 1H), 7.26-7.40(m, 3H), 7.64-7.66(s, 1H), 8.47-8.48(s, 1H), 8.77(s, 1H).

### EXAMPLE 21

### N-(2-((2-(2,6-difluoro-3,5-dimethoxybenzoyl)-1H-pyrrolo[2,3-c]pyridin-5 -yl)amino)-5-(4-ethylpiperazin-1-yl)phenyl)acrylamide

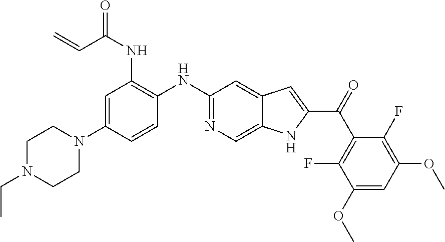 [SMILES: C=CC(=O)Nc1cc(N2CCN(CC)CC2)ccc1Nc1cc2cc(C(=O)c3c(F)c(OC)cc(OC)c3F)[nH]c2cn1]

### Step 1 (5-chloro-1-(4-methoxybenzyl)-1H-pyrrolo[2,3-c]pyridin-2-yl)(2,6-difluoro-3,5-dimethoxyphenyl)methanone

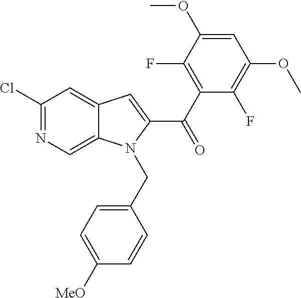 [SMILES: COc1ccc(Cn2c(C(=O)c3c(F)c(OC)cc(OC)c3F)cc3cc(Cl)ncc32)cc1]

To a solution of (5-chloro-1H-pyrrolo[2,3-c]pyridin-2-yl)(2,6-difluoro-3,5-dimethoxyphenyl)methanone (0.85 g,2.27 mmol) in DMF (10 ml) was added Cs₂CO₃ (0.1.6 g,4.9 mmol) and PMBCl (0.45 g, 2.9 mmol). The resulting mixture was stirred at 40° C. for 2 h, then cooled to room temperature and quenched with water and stirred for 30 minutes, filtrated and washed with water. The solid was dried in vacuum to give 0.85 g of the title product as an off-white solid. Yield: 70.8%. MS (ES+): 473 [M+1]⁺

### Step 2 (2,6-difluoro-3,5-dimethoxyphenyl)(5-(4-(4-ethylpiperazin-1-yl)-2-nitrophenylamino)-1-(4-methoxybenzyl)-1H-pyrrolo[2,3-c]pyridin-2-yl)methanone

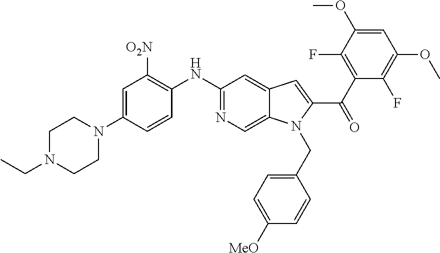 [SMILES: CCN1CCN(c2ccc(Nc3cc4cc(C(=O)c5c(F)c(OC)cc(OC)c5F)n(Cc5ccc(OC)cc5)c4cn3)c([N+](=O)[O-])c2)CC1]

To a solution of the product of Step 1 (0.84 g, 1.78 mmol) and 4-(4-ethylpiperazin-1-yl)-2-nitroaniline (0.37 g, 1.5 mmol) in toluene (25 ml) was added X-phos (0.27 g, 0.57 mmol), Cs₂CO₃ (1.0 g, 3.06 mmol). Argon was bubbled through the mixture for 10 min and Pd₂(dba)3(0.27 g,0.3 mmol) was added. Argon was bubbled through the mixture again for 10 min. The reaction mixture was heated to 115° C. and stirred overnight. After cooling to room temperature, the reaction was partitioned between EA and water. The aqueous phase was extracted with EA. The combined organic phases were washed with brine, dried with Na₂SO₄, filtered and concentrated. The residue was purified by column photography (MeOH/DCM=4%) to give 0.8 g of the title product as a dark brown solid. Yield: 66.7%. MS (ES+): 687 [M +1]⁺

### Step 3 (2,6-difluoro-3,5-dimethoxyphenyl)(5-(4-(4-ethylpiperazin-1-yl)-2-nitrophenylamino)-1H-pyrrolo[2,3 -c]pyridin-2-yl)methanone

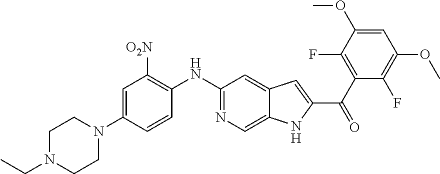 [SMILES: CCN1CCN(c2ccc(Nc3cc4cc(C(=O)c5c(F)c(OC)cc(OC)c5F)[nH]c4cn3)c([N+](=O)[O-])c2)CC1]

To a solution of the product of Step 2 (0.595 g, 0.866 mmol) in TFA (15 ml) was added trifluoromethanesulfonic acid (1.5 ml). The resulting mixture was stirred at room temperature for 2 hours. The reaction mixture was concentrated . The residue was dissolved in water, then adjust ph>8 with solid Na₂CO₃, The aqueous phase was extracted with EA. The combined organic phases were washed with brine, dried with Na₂SO₄, filtered and concentrated. The residue was purified by column photography (MeOH/DCM=4%) to give 0.44 g of the title product as a dark brown solid. Yield: 90%. MS (ES+): 567 [M +1]⁺

### Step 4 (5-(2-amino-4-(4-ethylpiperazin-1-yl)phenylamino)-1H-pyrrolo[2,3 -c]pyridin-2-yl)(2,6-difluoro-3,5-dimethoxyphenyl)methanone

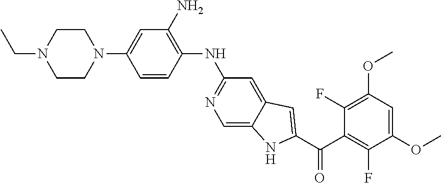 [SMILES: CCN1CCN(c2ccc(Nc3cc4cc(C(=O)c5c(F)c(OC)cc(OC)c5F)[nH]c4cn3)c(N)c2)CC1]

Pd/C (10%, 0.15 g) was added to a solution of the product of Step 3 (0.3 g, 0.53 mmol) in MeOH(10 ml). The reaction was stirred for 16 hours under hydrogen (1 atm, ballon) at room temperature. The reaction mixture was filtered through a layer of Celite. The filtrate was concentrate to afford 0.25 of the title product as dark foam which was used in the next without further purification. Yiled: 90%. MS (ES+): 537 [M +1].

### Step 5 N-(2-(2-(2,6-difluoro-3,5-dimethoxybenzoyl)-1H-pyrrolo[2,3-c]pyridin-5-ylamino)-5-(4-ethylpiperazin-1-yl)phenyl)acrylamide

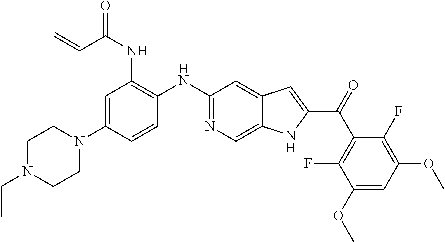 [SMILES: C=CC(=O)Nc1cc(N2CCN(CC)CC2)ccc1Nc1cc2cc(C(=O)c3c(F)c(OC)cc(OC)c3F)[nH]c2cn1]

To a solution of the product of Step 4 (250 mg, 0.46 mmol) in DCM (10 ml) at 5° C. was added acrylic acid (33.5 mg, 0.51), DIPEA (0.25 ml 1.4 mmol)), then T3P (1.8 g 2.8 mmol) was added drop wise. After addition, the reaction mixture was stirred for 0.5 h. The reaction mixture was quenched with water and extracted with EA. The organic phase was washed with brine and dried over Na₂SO₄. The solution was filtered and concentrated. The residue was purified by column photography (MeOH/DCM=4%) to give 20 mg of the title product as a red solid. Yield: 7.3%. MS (ES+): 591.2 [M+1]⁺.

¹HNMR: (400 MHz, CDCl₃) δ 1.15-1.20 (t, J=16 Hz, 3H), 2.5-2.51 (m, 2H), 2.56 (m, J=4.4,4H), 3.3-3.35 (d, J=4.4,4H), 3.95 (s, 6H), 5.63-5.65 (d, J=10 Hz, 1H), 6.16-6.348 (m, 3H), 6.449 (s, 1H), 8.264-8.311 (t, J=18.8,1H), 8.735 (s, 1H), 7.26-7.40(m, 3H), 10.145(s, 1H).

### EXAMPLE 22

### N-(2-((3 -(2,6-difluoro-3,5-dimethoxybenzoyl)-1H-pyrrolo[2,3-c]pyridin-5-yl)amino)-5-(4-ethylpiperazin-1-yl)phenyl)acrylamide

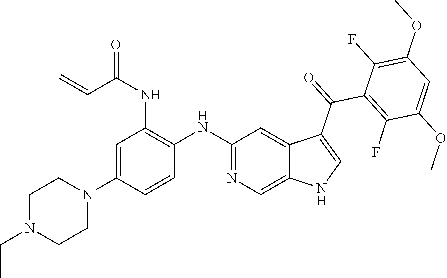 [SMILES: C=CC(=O)Nc1cc(N2CCN(CC)CC2)ccc1Nc1cc2c(C(=O)c3c(F)c(OC)cc(OC)c3F)c[nH]c2cn1]

### Step 1 (5-chloro-1H-pyrrolo[2,3-c]pyridin-3-yl)(2,6-difluoro-3,5-dimethoxyphenyl)methanone

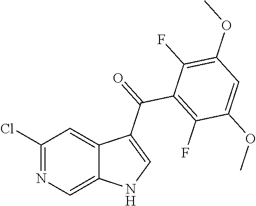 [SMILES: COc1cc(OC)c(F)c(C(=O)c2c[nH]c3cnc(Cl)cc23)c1F]

To a stirred solution of 2,6-difluoro-3,5-dimethoxybenzoyl chloride (543 mg, 2.29 mmol) in DCE (6 ml) was added AlCl₃ (611 mg, 4.58 mmol). After stirring for 30 min, 5-chloro-1H-pyrrolo[2,3-c]pyridine (350 mg, 152.58 mmol) was added and the mixture was stirred at RT for 2 h. The solution was quenched with saturated Na₂CO₃ and extracted with DCM. The organic layer was washed with brine, dried and evaporated to give 160 mg of the title product. Yield: 19.8%.

### Step 2 (5-chloro-1-(4-methoxybenzyl)-1H-pyrrolo[2,3-c]pyridin-3-yl)(2,6-difluoro-3,5-dimethoxyphenyl)methanone

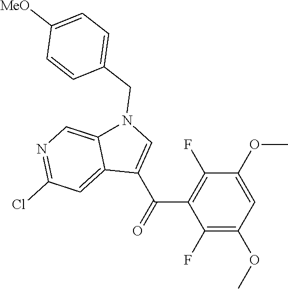 [SMILES: COc1ccc(Cn2cc(C(=O)c3c(F)c(OC)cc(OC)c3F)c3cc(Cl)ncc32)cc1]

To a mixture of (5-chloro-1H-pyrrolo[2,3-c]pyridin-3-yl)(2,6-difluoro-3,5-dimethoxyphenyl)methanone (180 mg, 0.51 mmol), Cs₂CO₃ (332.5 mg, 1.02 mmol) in THF (6 ml) was added PMBCl (96 mg, 0.61 mmol). The mixture was stirred at 40° C. overnight. The mixture was diluted with water, extracted with EA. The organic phase was washed with brine, dried and evaporated to give the crude product which purified by column chromatography to give 80 mg of the title product. Yield: 33.2%.

### Step 3 (2,6-difluoro-3,5-dimethoxyphenyl)(5-((4-(4-ethylpiperazin-1-yl)-2-nitrophenyl)amino)-1-(4-methoxybenzyl)-1H-pyrrolo[2,3-c]pyridin-3-yl)methanone

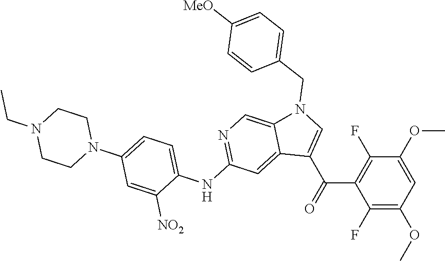 [SMILES: CCN1CCN(c2ccc(Nc3cc4c(C(=O)c5c(F)c(OC)cc(OC)c5F)cn(Cc5ccc(OC)cc5)c4cn3)c([N+](=O)[O-])c2)CC1]

The mixture of (5-chloro-1-(4-methoxybenzyl)-1H-pyrrolo[2,3-c]pyridin-3-yl)(2,6-difluoro-3,5-dimethoxyphenyl)methanone (1 g, 2.11 mmol), 4-(4-ethylpiperazin-1-yl)-2-nitroaniline (530 mg, 2.11 mmol), Pd₂(dba)₃ (400 mg, 0.423 mmol), X-phos (405 mg, 0.846 mmol) and Cs₂CO₃ (1.38 g, 4.23 mmol) in toluene (10 ml) was heated at 110° C. under Ar₂ and stirred overnight. The reaction mixture was cooled to room temperature, filtered and concentrated to dryness. The residue was purified by column photography to give 0.9 g of the title product as a dark brown solid. Yield: 62%.

### Step 4 (2,6-difluoro-3,5-dimethoxyphenyl)(5-((4-(4-ethylpiperazin-1-yl)-2-nitrophenyl)amino)-1H-pyrrolo[2,3-c]pyridin-3-yl)methanone

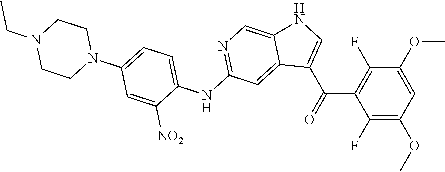 [SMILES: CCN1CCN(c2ccc(Nc3cc4c(C(=O)c5c(F)c(OC)cc(OC)c5F)c[nH]c4cn3)c([N+](=O)[O-])c2)CC1]

To a stirred solution of (2,6-difluoro-3,5-dimethoxyphenyl)(5-((4-(4-ethylpiperazin-1-yl)-2-nitrophenyl)amino)-1-(4-methoxybenzyl)-1H-pyrrolo[2,3-c]pyridin-3-yl)methanone (500 mg, 0.728 mmol) in TFA (10 ml) was added 98% H₂SO₄ (1 ml). The mixture was stirred at 40° C. for 5 h, then quenched with aqueous Na₂CO₃, extracted with DCM. The organic layer washed with brine, dried and evaporated. The residue was purified by column chromatography to give 180 mg of the title product. Yield: 43.6%.

### Step 5 (5-((2-amino-4-(4-ethylpiperazin-1-yl)phenyl)amino)-1H-pyrrolo[2,3-c]pyridin-3-yl)(2,6-difluoro-3,5-dimethoxyphenyl)methanone

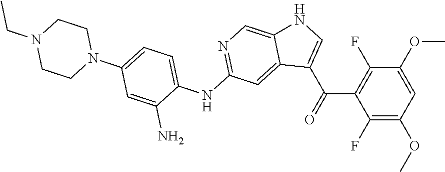 [SMILES: CCN1CCN(c2ccc(Nc3cc4c(C(=O)c5c(F)c(OC)cc(OC)c5F)c[nH]c4cn3)c(N)c2)CC1]

To a solution of (2,6-difluoro-3,5-dimethoxyphenyl)(5-((4-(4-ethylpiperazin-1-yl)-2-nitrophenyl)amino)-1H-pyrrolo[2,3-c]pyridin-3-yl)methanone (150 mg, 0.265 mmol) in MeOH (10 ml) was added 10% Pd/C (45 mg, 33% in H₂O). The mixture was stirred under 1 atmosphereof H₂ at room temperature overnight. The mixture was filtered and the filtrate was evaporated to give 120 mg of the title product. Yield: 84.5%.

### Step 6 N-(2-((3-(2,6-difluoro-3,5-dimethoxybenzoyl)-1H-pyrrolo[2,3-c]pyridin-5-yl)amino)-5-(4-ethylpiperazin-1-yl)phenyl)acrylamide

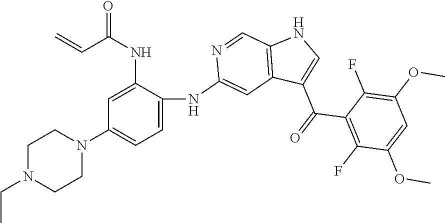 [SMILES: C=CC(=O)Nc1cc(N2CCN(CC)CC2)ccc1Nc1cc2c(C(=O)c3c(F)c(OC)cc(OC)c3F)c[nH]c2cn1]

To a solution of (5-((2-amino-4-(4-ethylpiperazin-1-yl)phenyl)amino)-1H-pyrrolo[2,3-c]pyridin-3-yl)(2,6-difluoro-3,5-dimethoxyphenyl)methanone (120 mg, 0.224 mmol) in DCM (3 ml) was added T3P (50% in DMF, 854 mg, 1.342 mmol), DIEA (173.4 mg, 1.342 mmol) and acrylic acid (16.1 mg, 0.224 mmol) at 0° C. The mixture was stirred at 0° C. for 1 h, then quenched with saturated aqueous Na₂CO₃, extracted with EA. The organic layer was washed with brine, dried and evaporated. The residue was purified by pre-TLC to give 5 mg of the title product as a yellow solid. Yield: 3.8%. MS (ES+): 591.2 [M+1]⁺.

¹HNMR: (400 MHz, CDCl₃) δ 1.53-1.58 (m, 2H), 2.70-2.72 (m, 3H), 2.84 (s, 4H), 3.45 (s, 4H), 3.95 (s, 6H), 5.69-5.72 (d, J=10.8 Hz, 1H), 6.21-6.42 (m, 5H), 6.68-7.20 (m, 4H), 7.64-8.22(s, 1H), 8.37(s, 1H), 9.40(s, 1H).

### EXAMPLE 23

### N-(2-((6-(2,6-difluoro-3,5-dimethoxybenzoyl)furo[3,2-d]pyrimidin-2-yl)amino)-3 -methylphenyl)acrylamide

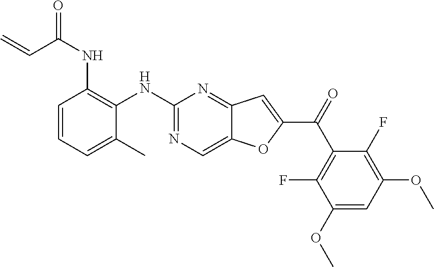 [SMILES: C=CC(=O)Nc1cccc(C)c1Nc1ncc2oc(C(=O)c3c(F)c(OC)cc(OC)c3F)cc2n1]

### Step 1 (2,6-difluoro-3,5-dimethoxyphenyl)(2-(2-methyl-6-nitrophenylamino)furo[3,2-d]pyrimidin-6-yl)methanone

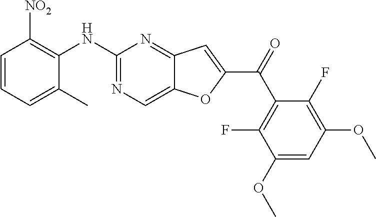 [SMILES: COc1cc(OC)c(F)c(C(=O)c2cc3nc(Nc4c(C)cccc4[N+](=O)[O-])ncc3o2)c1F]

To a solution of (2-chlorofuro[3,2-d]pyrimidin-6-yl)(2,6-difluoro-3,5-dimethoxyphenyl)methanone (0.4 g, 1.13mmol) and 2-methyl-6-nitroaniline (0.16 g, 1.05 mmol) in toluene (20 ml) was added X-phos (0.2 g, 0.42 mmol), Cs₂CO₃ (0.72 g, 2.2 mmol). Argon was bubbled through the mixture for 10 min and Pd₂(dba)₃ (0.2 g,0.23 mmol) was added. Argon was bubbled through the mixture again for 10 min. The reaction mixture was heated to 115° C. and stirred overnight. After cooling to room temperature, the reaction was partitioned between EA and water. The aqueous phase was extracted with EA. The combined organic phases were washed with brine and dried over Na₂SO₄. The solution was filtered and concentrated. The residue was purified by column photography (EA/PE=40%) to give 0.2 g of pure product as an off-white solid. Yield: 38%. MS (ES+): 471 [M +1]⁺

### Step 2 (2-(2-amino-6-methylphenylamino)furo[3,2-d]pyrimidin-6-yl)(2,6-difluoro-3,5-dimethoxyphenyl)methanol

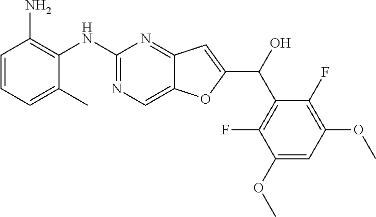 [SMILES: COc1cc(OC)c(F)c(C(O)c2cc3nc(Nc4c(C)cccc4N)ncc3o2)c1F]

To a stirred solution of the product of Step 1 (0.16 g, 0.34 mmol) in EtOH/H₂O (8 ml/2 ml), Fe (200 mg, 3.4 mmol) and NH₄Cl (0.18 g, 0.34 mmol) were added. The resulting mixture was stirred at refluxing for 2 h, then cooled to room temperature and filtered. The solution was quenched with water and extracted with EA. The organic layer was washed with brine and dried over Na₂SO₄. The solution was filtered and concentrated to give 0.43 mg of crude title compound which was used in the next step without further purification. Yield: 91%. MS (ES+): 447 [M+1]⁺.

### Step 3 N-(2-(6-((2,6-difluoro-3,5-dimethoxyphenyl)(hydroxy)methyl)furo[3,2-d]pyrimidin-2-ylamino)-3-methylphenyl)acrylamide

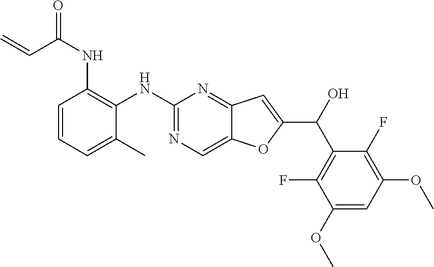 [SMILES: C=CC(=O)Nc1cccc(C)c1Nc1ncc2oc(C(O)c3c(F)c(OC)cc(OC)c3F)cc2n1]

To a solution of the product of Step 2 (130 mg, 0.29 mmol) in DCM (5 ml) at 5° C. was added acrylic acid (100 mg, 1.38 mmol), DIPEA (0.3 ml 1.68 mmol)), then T3P (1.2 g 1.8 mmol) was added drop wise. After addition, the reaction mixture was stirred for 2 h. The reaction mixture was quenched with water and extracted with EA. The organic phase was washed with brine and dried over Na₂SO₄. The solution was filtered and concentrated. The residue was purified by column photography (EA/PE=1) to give 80 mg of pure product as a yellow solid. Yield: 53%. MS (ES+): 497.1[M+1]⁺

### Step 4 N-(2-(6-(2,6-difluoro-3,5-dimethoxybenzoyl)furo[3,2-d]pyrimidin-2-ylamino)-3-methylphenyl) acrylamide

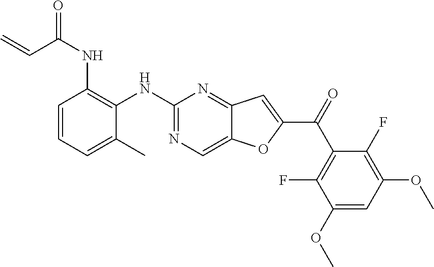 [SMILES: C=CC(=O)Nc1cccc(C)c1Nc1ncc2oc(C(=O)c3c(F)c(OC)cc(OC)c3F)cc2n1]

To a solution of the product of Step 3 (80 mg, 0.16 mmol) in DCM(7 ml) at room temperature was added MnO₂ (285 mg,3.3 mmol). The reaction was stirred at room temperature for 16 hours. The reaction mixture was filtered through a layer of Celite. The filtrate was concentrate and purified by pre-TLC (PE:EA=1:1) to give 10 mg of product as a yellow solid. Yield 13%. MS (ES+): 495.1 [M+1]⁺.

¹HNMR: (400 MHZ, DMSO-d₆) δ 2.14 (s, 3H), 3.9 (s, 6H), 5.66-5.69 (d, J=10,1H), 6.18-6.22 (d, J=15.6, 1H), 7.0-7.26(m, 3H), 7.68-7.74(m, 2H), 7.79(s,1H), 7.11-7.17(m, 3H), 8.56(s, 1H),9.02(s,1H).

### EXAMPLE 24

### N-(2-((2-(2,6-difluoro-3,5-dimethoxybenzoyl)thieno[2,3-c]pyridin-5-yl)amino)-3-methylphenyl)acrylamide

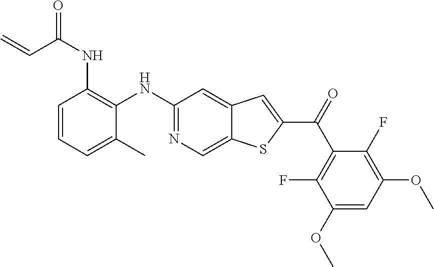 [SMILES: C=CC(=O)Nc1cccc(C)c1Nc1cc2cc(C(=O)c3c(F)c(OC)cc(OC)c3F)sc2cn1]

Step 1 (5-chlorothieno[2,3-c]pyridin-2-yl)(2,6-difluoro-3,5-dimethoxyphenyl)methanone

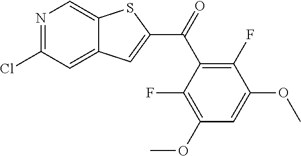 [SMILES: COc1cc(OC)c(F)c(C(=O)c2cc3cc(Cl)ncc3s2)c1F]

To a stirred solution of (5-chlorothieno[2,3-c]pyridin-2-yl)(2,6-difluoro-3,5-dimethoxyphenyl)methanol (400 mg, 1.1 mmol) in acetone (16 ml), Jones reagent (0.53 ml) was added drop wise at 0° C. and stirred for 1 h. The solution was quenched with water and extracted with EA. The organic layer was washed with brine and dried over Na₂SO₄. The solution was filtered and concentrated to give 370 mg of crude product. Yield: 93%.

### Step 2 (2,6-difluoro-3,5-dimethoxyphenyl)(5-(2-methyl-6-nitrophenylamino)thieno[2,3-c]pyridin-2-yl)methanone

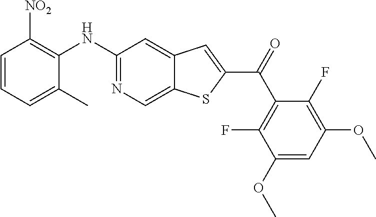 [SMILES: COc1cc(OC)c(F)c(C(=O)c2cc3cc(Nc4c(C)cccc4[N+](=O)[O-])ncc3s2)c1F]

A mixture of (5-chlorothieno[2,3-c]pyridin-2-yl)(2,6-difluoro-3,5-dimethoxyphenyl)methanone (370 mg, 1 mmol), 2-methyl-6-nitroaniline (228.4 mg, 1.5 mmol), Pd₂(dba)₃ (183.2 mg, 0.2 mmol), X-phos (190.7 mg, 0.4 mmol) and Cs₂CO₃ (652 mg, 2 mmol) in toluene (10 ml) was heated at 115° C. under Are overnight. The reaction mixture was concentrated. The residue was purified by silica gel chromatography (PE:EA=1:1) to give 200 mg of pure product. Yield: 41.2%.

### Step 3 (5-(2-amino-6-methylphenylamino)thieno[2,3-c]pyridin-2-yl)(2,6-difluoro-3,5-dimethoxyphenyl)methanone

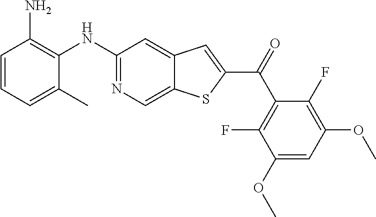 [SMILES: COc1cc(OC)c(F)c(C(=O)c2cc3cc(Nc4c(C)cccc4N)ncc3s2)c1F]

To a stirred solution of (2,6-difluoro-3,5-dimethoxyphenyl)(5-(2-methyl-6-nitrophenylamino)thieno[2,3-c]pyridin-2-yl)methanone (180 mg, 0.371 mmol) in i-PrOH /H₂O (3 ml/3 ml), Fe (207.1 mg, 3.71 mmol) and NH₄Cl (198.3 mg, 3.71 mmol) were added. The resulting mixture was stirred at 100° C. for 2 h, then cooled to room temperature and filtered. The solution was quenched with water and extracted with EA. The organic layer was washed with brine and dried over Na₂SO₄. The solution was filtered and concentrated to give 140 mg of crude title compound which was used in the next step without further purification. Yield: 82.9%.

### Step 4 N-(2-(2-(2,6-difluoro-3,5-dimethoxybenzoyl)thieno[2,3-c]pyridin-5-ylamino)-3-methylphenyl)acrylamide

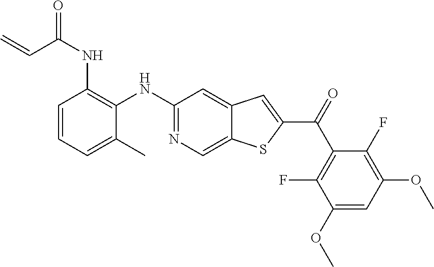 [SMILES: C=CC(=O)Nc1cccc(C)c1Nc1cc2cc(C(=O)c3c(F)c(OC)cc(OC)c3F)sc2cn1]

The product of Step 3 (140 mg, 0.307 mmol) was dissolved in DCM (3 ml). At RT, T3P (50% in DMF, 1.2 g, 1.844 mmol) and acrylic acid (133 mg, 1.844 mmol) were added to the reaction mixture, and then stirred for 1 h. After the reaction was complete, the reaction mixture was quenched with water, extracted with EA. The organic layer was washed with brine and dried over Na₂SO₄. The solution was filtered, concentrated and purified by silica gel chromatography (PE:EA=1:1) to get 30 mg of the title product. Yield: 19.2%. MS (ES+): 510 [M+1]⁺.

¹HNMR: (400 MHz, CDCl₃) δ 2.20 (s , 3H), 3.92 (s, 6H), 5.66-5.69 (d, J=12 Hz,1H), 5.99 (s, 1H), 6.13-6.15 (m, 1H), 6.30-6.34 (d, J=16 Hz, 1H), 6.39 (s, 1H),6.75-6.79 (t, J=16 Hz ,1H), 7.06-7.08 (d, J=8 Hz, 1H), 7.29-7.32 (d, J=12 Hz, 1H), 7.45(s, 1H), 8.14(s, 1H), 8.37-8.40(d, J=12 Hz, 1H), 8.87(s, 1H).

### EXAMPLE 25

### N-(2-((2-(2,6-difluoro-3,5-dimethoxybenzoyl)furo[2,3-c]pyridin-5-yl)amino)-3-(trifluoromethyl)phenyl)acrylamide

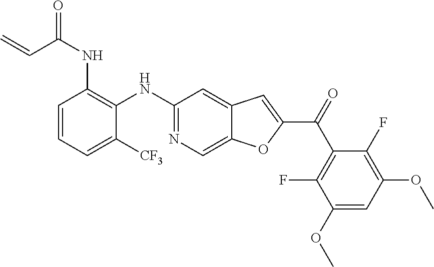 [SMILES: C=CC(=O)Nc1cccc(C(F)(F)F)c1Nc1cc2cc(C(=O)c3c(F)c(OC)cc(OC)c3F)oc2cn1]

### Step 1 (2,6-difluoro-3,5-dimethoxyphenyl)(5-(2-nitro-6-(trifluoromethyl)phenylamino)furo[2,3-c]pyridin-2-yl)methanone

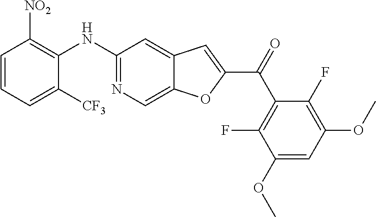 [SMILES: COc1cc(OC)c(F)c(C(=O)c2cc3cc(Nc4c([N+](=O)[O-])cccc4C(F)(F)F)ncc3o2)c1F]

A mixture of (5-chlorofuro[2,3-c]pyridin-2-yl)(2,6-difluoro-3,5-dimethoxyphenyl)methanone (700 mg, 1.98 mmol), 2-nitro-6-(trifluoromethyl)aniline (420 mg, 2.04 mmol), Pd₂(dba)₃ (366 mg, 0.40 mmol), X-phos (380 mg, 0.80 mmol) and Cs₂CO₃ (1.3 g, 3.99 mmol) in toluene (10 ml) was heated at 115° C. under Ar₂ for 15 h. The reaction mixture was cooled to room temperature, quenched with water and extracted with EA. The organic layer was washed with brine and dried over Na₂SO₄. The solution was filtered and concentrated to dryness. The residue was purified by silica gel chromatography(EA:PE=1:2)to give 150 mg of pure product as a yellow solid. MS (ES+): 524 [M +1]⁺¹.

### Step 2 tert-butyl 2-(2,6-difluoro-3,5-dimethoxybenzoyl)furo[2,3-c]pyridin-5-yl(2-nitro-6-(trifluoromethyl)phenyl)carbamate

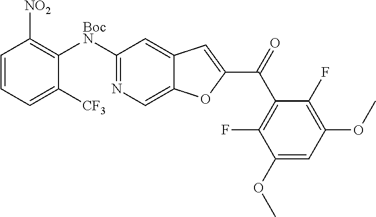 [SMILES: COc1cc(OC)c(F)c(C(=O)c2cc3cc(N(C(=O)OC(C)(C)C)c4c([N+](=O)[O-])cccc4C(F)(F)F)ncc3o2)c1F]

To a stirred solution of (2,6-difluoro-3,5-dimethoxyphenyl)(5-(2-nitro-6-(trifluoromethyl)phenylamino)furo[2,3-c]pyridin-2-yl)methanone (150 mg, 0.28 mmol) in 1,4-dioxane (3 ml), DMAP (4 mg, 0.03 mmol) and (Boc)₂O (94 mg, 0.43 mmol) were added. The resulting mixture was stirred at 70° C. for 2 h, then cooled to room temperature. The solution was quenched with water and extracted with EA. The organic layer was washed with brine and dried over Na₂SO₄. The solution was filtered and concentrated to give 160 mg of crude product as a yellow solid. MS (ES+): 624 [M +1]⁺¹.

### Step 3 tert-butyl 2-amino-6-(trifluoromethyl)phenyl(2-(2,6-difluoro-3,5-dimethoxybenzoyl)furo[2,3-c]pyridin-5-yl)carbamate

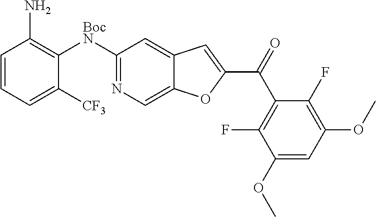 [SMILES: COc1cc(OC)c(F)c(C(=O)c2cc3cc(N(C(=O)OC(C)(C)C)c4c(N)cccc4C(F)(F)F)ncc3o2)c1F]

To a stirred solution of tert-butyl 2-(2,6-difluoro-3,5-dimethoxybenzoyl)furo[2,3-c]pyridin-5-yl(2-nitro-6-(trifluoromethyl)phenyl)carbamate (150 mg, 0.24 mmol) in EtOH/H₂O (4 ml/1 ml), Fe (67 mg, 1.2 mmol) and NH₄Cl (64 mg, 1.2 mmol) were added. The resulting mixture was stirred at 90° C. for 2 h, then cooled to room temperature and filtered. The solution was quenched with saturated Na₂CO₃ and extracted with DCM. The organic layer was washed with brine and dried over Na₂SO₄. The solution was filtered and concentrated to give 160 mg of crude title compound which was used in the next step without further purification. MS (ES+): 594 [M +1]⁺¹.

### Step 4 tert-butyl 2-acrylamido-6-(trifluoromethyl)phenyl(2-(2,6-difluoro-3,5-dimethoxybenzoyl)furo[2,3-c]pyridin-5-yl)carbamate

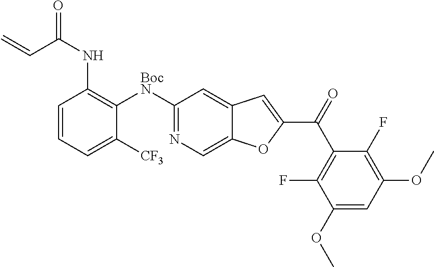 [SMILES: C=CC(=O)Nc1cccc(C(F)(F)F)c1N(C(=O)OC(C)(C)C)c1cc2cc(C(=O)c3c(F)c(OC)cc(OC)c3F)oc2cn1]

The product of Step3 (110 mg, 0.18 mmol) and Et3N (37 mg, 0.36 mmol) were dissolved in DCM (4 ml). At RT, acrylyl chloride (94 mg, 1.04 mmol) was added to the reaction mixture, and then stirred for 2 h. After the reaction was complete, the reaction mixture was concentrated and purified by silica gel chromatography (EA:PE=1:2) to get 90 mg of pure product as a yellow solid. MS (ES+): 648[M+1]+1.

### Step 5 N-(2-(2-(2,6-difluoro-3,5-dimethoxybenzoyl)furo[2,3-c]pyridin-5-ylamino)-3-(trifluoromethyl)phenyl)acrylamide

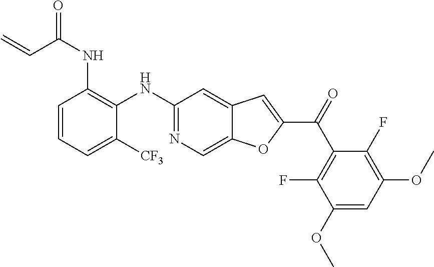 [SMILES: C=CC(=O)Nc1cccc(C(F)(F)F)c1Nc1cc2cc(C(=O)c3c(F)c(OC)cc(OC)c3F)oc2cn1]

To a stirred solution of the product of Step 4 (90 mg) in DCM (5 ml), TFA (1 ml) was added at rt. The resulting mixture was stirred at RT for 2 h, then concentrated in vacuo and the residue was recrystallized from MTBE/PE(1 ml/2 ml) to give 45 mg of pure product as a yellow solid. MS (ES+): 548.1 [M +1]⁺¹.

¹HNMR: (400 MHz, DMSO-d₆) δ 3.94 (s, 6H), 5.61-5.64 (d, J=12 Hz, 1H), 6.09-6.14 (m, 1H), 6.32-6.39 (m, 1H), 6.77 (s, 1H), 7.24-7.28 (m, 1H), 7.50-7.59 (m, 1H), 7.76 (s, 1H), 7.90 (s, 1H), 8.21-8.22 (d, J=4 Hz, 1H), 8.58(s, 1H), 9.44(s, 1H).

### EXAMPLE 26

### N-(3-chloro-2-((2-(2,6-difluoro-3,5-dimethoxybenzoyl)furo[2,3-c]pyridin-5-yl)amino)phenyl)acrylamide

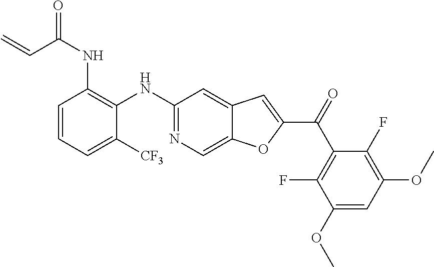 [SMILES: C=CC(=O)Nc1cccc(C(F)(F)F)c1Nc1cc2cc(C(=O)c3c(F)c(OC)cc(OC)c3F)oc2cn1]

### Step 1 tert-butyl 2-(2,6-difluoro-3,5-dimethoxybenzoyl)furo[2,3-c]pyridin-5-ylcarbamate

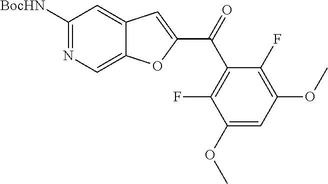 [SMILES: COc1cc(OC)c(F)c(C(=O)c2cc3cc(NC(=O)OC(C)(C)C)ncc3o2)c1F]

A mixture of (5-chlorofuro[2,3-c]pyridin-2-yl)(2,6-difluoro-3,5-dimethoxyphenyl)methanone (1 g, 2.83 mmol), NH₂Boc (1 g, 8.53 mmol), Pd₂(dba)₃ (0.515 g, 0.558 mmol), X-phos (0.535 g, 1.116 mmol) and Cs₂CO₃ (1.84 g, 5.64 mmol) in toluene (15 ml) was heated at 115° C. under N₂ for 15 h. The reaction mixture was cooled to room temperature, filtered and concentrated to dryness. The residue was purified by silica gel chromatography (EA/PE=1:2) to give 500 mg of the title product as a yellow solid. Yield: 42%. MS (ES+): 435 [M+1]⁺.

### Step 2 (5-aminofuro[2,3-c]pyridin-2-yl)(2,6-difluoro-3,5-dimethoxyphenyl)methanone hydrochloride

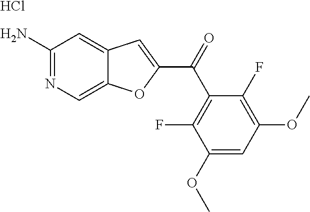 [SMILES: Cl ; COc1cc(OC)c(F)c(C(=O)c2cc3cc(N)ncc3o2)c1F]

The product of Step 1 (500 mg, 1.15 mmol) was added to HCl/EA (4N, 15 ml). The resulting mixture was stirred at 25° C. for 15 h. The mixture was filtered and dried to give 300 mg of the title product as a yellow solid. Yield: 42%. MS (ES+): 335 [M+1]⁺.

### Step 3 (5-(2-chloro-6-nitrophenylamino)furo[2,3-c]pyridin-2-yl)(2,6-difluoro-3,5-dimethoxyphenyl)methanone

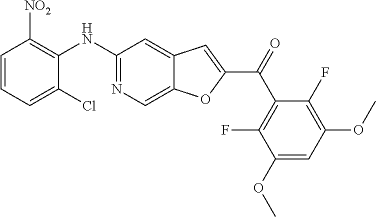 [SMILES: COc1cc(OC)c(F)c(C(=O)c2cc3cc(Nc4c(Cl)cccc4[N+](=O)[O-])ncc3o2)c1F]

A mixture of the product of Step 2 (350 mg, 0.94 mmol), 2-bromo-1-chloro-3-nitrobenzene (270 mg, 1.24 mmol), Pd₂(dba)₃ (170 mg, 0.186 mmol), X-phos (180 mg, 0.378 mmol) and Cs₂CO₃ (1.20 g, 3.68 mmol) in toluene (5 ml) was heated at 115° C. under N₂ for 15 h. The reaction mixture was cooled to room temperature. The solution was quenched with water and extracted with EA. The organic layer was washed with brine and dried over Na₂SO₄. The solution was filtered, concentrated and purified by silica gel chromatography (EA/PE=1:2) to give 200 mg of pure product as a yellow solid. Yield: 43%. MS (ES+): 490 [M+1]⁺.

### Step 4 tert-butyl 2-chloro-6-nitrophenyl(2-(2,6-difluoro-3,5-dimethoxybenzoyl)furo[2,3-c]pyridin-5-yl)carbamate

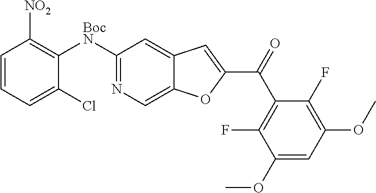 [SMILES: COc1cc(OC)c(F)c(C(=O)c2cc3cc(N(C(=O)OC(C)(C)C)c4c(Cl)cccc4[N+](=O)[O-])ncc3o2)c1F]

A mixture of the product of Step 3 (220 mg, 0.45 mmol), DMAP (6 mg, 0.05 mmol), (Boc)₂₀ (147 mg, 0.67 mmol) in 1,4-dioxane (4 ml) was heated at 70° C. for 2 h. The reaction mixture was cooled to room temperature. The solution was quenched with water and extracted with EA. The organic layer was washed with brine and dried over Na₂SO₄. The solution was filtered, concentrated to give 300 mg of crude title compound which was used in the next step without further purification. MS (ES+): 590 [M +1]⁺.

### Step 5 tert-butyl 2-amino-6-chlorophenyl(2-(2,6-difluoro-3,5-dimethoxybenzoyl)furo[2,3-c]pyridin-5-yl)carbamate

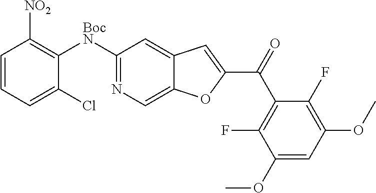 [SMILES: COc1cc(OC)c(F)c(C(=O)c2cc3cc(N(C(=O)OC(C)(C)C)c4c(Cl)cccc4[N+](=O)[O-])ncc3o2)c1F]

To a stirred solution of the product of Step 4 (200 mg, 0.34 mmol) in EtOH/H20 (4 ml/1 ml), Fe (200 mg, 3.57 mmol) and NH₄Cl (190 mg, 3.55 mmol) were added. The resulting mixture was stirred at 90° C. for 2 h, then cooled to room temperature and filtered. The solution was quenched with water and extracted with DCM. The organic layer was washed with brine and dried over Na₂SO₄. The solution was filtered and concentrated to give 250 mg of crude title compound which was used in the next step without further purification. MS (ES+): 560 [M +1]⁺.

### Step 6 tert-butyl 2-acrylamido-6-chlorophenyl(2-(2,6-difluoro-3,5-dimethoxybenzoyl)furo[2,3-c]pyridin-5-yl)carbamate

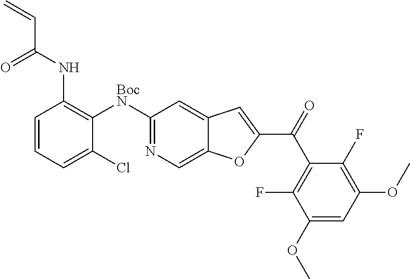 [SMILES: C=CC(=O)Nc1cccc(Cl)c1N(C(=O)OC(C)(C)C)c1cc2cc(C(=O)c3c(F)c(OC)cc(OC)c3F)oc2cn1]

The product of Step 5 (200 mg, 0.36 mmol) and Et₃N (72 mg, 0.71 mmol) were dissolved in DCM (4 ml). At 25° C., acrylyl chloride (200 mg, 2.22 mmol) was added to the reaction mixture, and then stirred for 0.5 h. After the reaction was complete, the reaction mixture was concentrated and purified by silica gel chromatography (EA/PE=1:1) to get 200 mg of pure product as a yellow solid. Yield: 90%. MS (ES+): 614 [M+1]⁺¹.

### Step 7 N-(3-chloro-2-(2-(2,6-difluoro-3,5-dimethoxybenzoyl)furo[2,3-c]pyridin-5-ylamino)phenyl)acrylamide

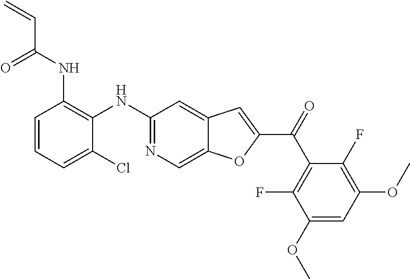 [SMILES: C=CC(=O)Nc1cccc(Cl)c1Nc1cc2cc(C(=O)c3c(F)c(OC)cc(OC)c3F)oc2cn1]

To a stirred solution of the product of Step 6 (180 mg, 0.29 mmol) in DCM (4 ml), TFA (1 ml) were added. The resulting mixture was stirred at room temperature for 2 h, the reaction mixture was concentrated and neutralized with Na₂CO₃ (1 ml 10%), and extracted with EA. The organic layer was washed with brine and dried over Na₂SO₄. The solution was filtered and concentrated, the residue was recrystallized from (MTBE/PE=1:1) to give 60 mg of the title compound as a yellow solid. Yield: 40%. MS (ES+): 514 [M +1]⁺.

¹HNMR: (400 MHz, DMSO-d₆) δ 3.94 (s, 6H), 5.68-5.70 (d, J=8 Hz, 1H), 6.18-6.23 (d, J=20 Hz, 1H), 6.47-6.54 (q, 1H), 6.72 (s, 1H), 7.25-7.34 (m, 3H), 7.76 (s, 1H), 7.95-7.97(d, J=72 Hz, 1H), 8.12(s, 1H), 8.65(s, 1H), 9.58(s, 1H).

### EXAMPLE 27

### N-(2-((2-(2,6-difluoro-3,5-dimethoxybenzoyl)-1H-pyrrolo[2,3-c]pyridin-5-yl)amino)-3-methylphenyl)acrylamide

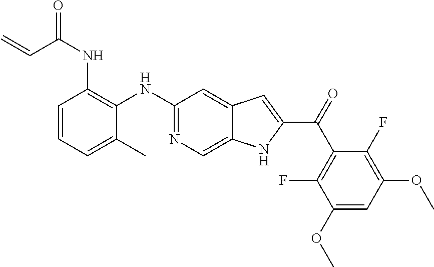 [SMILES: C=CC(=O)Nc1cccc(C)c1Nc1cc2cc(C(=O)c3c(F)c(OC)cc(OC)c3F)[nH]c2cn1]

### Step 1 5-chloro-1-(phenylsulfonyl)-1H-pyrrolo[2,3-c]pyridine

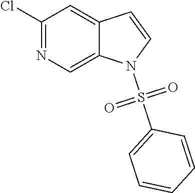 [SMILES: O=S(=O)(c1ccccc1)n1ccc2cc(Cl)ncc21]

To a stirred solution of 5-chloro-1H-pyrrolo[2,3-c]pyridine (20 g, 131 mmol) in toluene (200 ml), NaH (31 g ,1.2916 mol) was added at 0° C. in portions. Benzenesulfonyl chloride (100 ml, 0.7813 mol) was added at 0° C., and then the reaction mixture was stirred for 5 h at 15° C. The solution was quenched with water and extracted with EA. The organic layer was washed with brine and dried over Na₂SO₄. The solution was filtered, concentrated and purified by silica gel chromatography (PE:EA=6:1) to give 23 g of pure product. Yield: 60%.

### Step 2 (5-chloro-1-(phenylsulfonyl)-1H-pyrrolo[2,3-c]pyridin-2-yl)(2,6-difluoro-3,5-dimethoxyphenyl)methanol

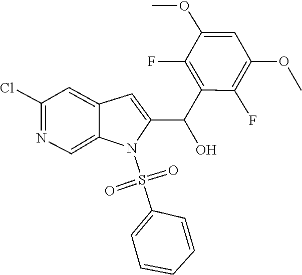 [SMILES: COc1cc(OC)c(F)c(C(O)c2cc3cc(Cl)ncc3n2S(=O)(=O)c2ccccc2)c1F]

To a stirred solution of the product of Step 1 (9.4 g ,32.1 mmol) in anhydrous THF (94 ml), LDA (24 ml , 182.1 mmol, 2M in THF) was added dropwise at −60° C. and stirred for 1 h. 2,6-Difluoro-3,5-dimethoxybenzaldehyde (8.45 g ,202.15 mmol) was then added in portions. The resulting mixture was stirred at −60° C. for 2 h. The solution was quenched with sat.NH₄Cl and extracted with EA. The organic layer was washed with brine and dried over Na₂SO₄. The solution was filtered, concentrated. PE was added to the residue and stirred at RT overnight, filtered and dried to give 9 g of the title product. Yield: 87%.

### Step 3 (5-chloro-1H-pyrrolo[2,3-c]pyridin-2-yl)(2,6-difluoro-3,5-dimethoxyphenyl)methanol

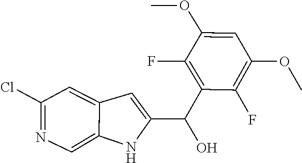 [SMILES: COc1cc(OC)c(F)c(C(O)c2cc3cc(Cl)ncc3[nH]2)c1F]

To a solution of CH₃ONa (5.46 g,101.1 mmol) in THF/MeOH (50 m/50 ml) was added the product of Step 2 (5 g, 10.1 mmol) at RT. The reaction mixture was stirred for 1 h. The reaction mixture was quenched with water and extracted with EA. The organic phase was washed with brine and dried over Na₂SO₄. The solution was filtered and concentrated. The residue was purified by silica gel chromatography (PE:EA=1:1) to give 3.5 g of the title product. Yield: 95%.

### Step 4 (5-chloro-1H-pyrrolo[2,3-c]pyridin-2-yl)(2,6-difluoro-3,5-dimethoxyphenyl)methanone

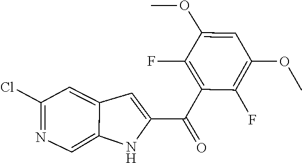 [SMILES: COc1cc(OC)c(F)c(C(=O)c2cc3cc(Cl)ncc3[nH]2)c1F]

A mixture of the product of Step 3 (756 mg, 2.13 mmol) in acetone (14 ml) was cooled down to 5° C., then Jone's reagent (1.5 ml) was added drop wise. After addition, the mixture was stirred at 5° C. for 4 h. The reaction mixture was quenched with water and extracted with EA. The organic layer was washed with brine and dried over Na₂SO₄. The solution was filtered and concentrated to give 285 mg of pure product. Yield: 37%.

### Step 5 (5-chloro-1-(4-methoxybenzyl)-1H-pyrrolo[2,3-c]pyridin-2-yl)(2,6-difluoro-3,5-dimethoxyphenyl)methanone

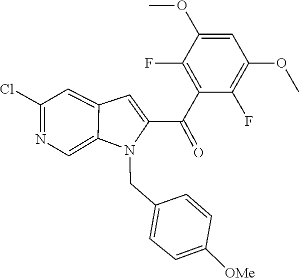 [SMILES: COc1ccc(Cn2c(C(=O)c3c(F)c(OC)cc(OC)c3F)cc3cc(Cl)ncc32)cc1]

To a solution of the product of Step 4 (100 mg, 0.28 mmol) in DMF (8 ml) were added Cs₂CO₃ (370 mg, 1.12 mmol) and PMBCl (0.08 ml, 0.56 mmol). The resulting mixture was stirred at 40° C. for 2 h. The reaction mixture was quenched with water and extracted with EA. The organic layer was washed with brine and dried over Na₂SO₄. The solution was filtered, concentrated and purified by silica gel chromatography (PE:EA=3: 1) to give 65 mg of the title product. Yield: 48%.

### Step 6 (2,6-difluoro-3,5-dimethoxyphenyl)(1-(4-methoxybenzyl)-5-(2-methyl-6-nitrophenylamino)-1H-pyrrolo[2,3-c]pyridin-2-yl)methanone

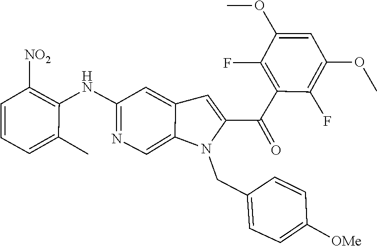 [SMILES: COc1ccc(Cn2c(C(=O)c3c(F)c(OC)cc(OC)c3F)cc3cc(Nc4c(C)cccc4[N+](=O)[O-])ncc32)cc1]

To a solution of the product of Step 5 (65 mg, 0.14 mmol) and 2-methyl-6-nitroaniline (21 mg, 0.14 mmol) in toluene (10 ml) was added X-phos (26 mg, 0.056 mmol), Cs₂CO₃ (90 mg, 0.28 mmol). Argon was bubbled through the mixture for 10 min and Pd₂(dba)₃ (25 mg, 0.028 mmol) was added. Argon was bubbled through the mixture again for 10 min. The reaction mixture was heated to 115° C. and stirred overnight. After cooling to room temperature, the reaction was partitioned between EA and water. The aqueous phase was extracted with EA. The combined organic phases were washed with brine and dried over Na₂SO₄. The solution was filtered and concentrated. The residue was purified by silica gel chromatography (MeOH/DCM=10%) to give 25 mg of the title product. Yield: 30%.

### Step 7 (2,6-difluoro-3,5-dimethoxyphenyl)(5-(2-methyl-6-nitrophenylamino)-1H-pyrrolo[2,3-c]pyridin-2-yl)methanone

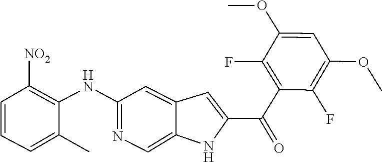 [SMILES: COc1cc(OC)c(F)c(C(=O)c2cc3cc(Nc4c(C)cccc4[N+](=O)[O-])ncc3[nH]2)c1F]

To a solution of the product of Step 6 (17 mg, 0.029 mmol) in TFA (1 ml) was added trifluoromethanesulfonic acid (0.1 ml, 1.13 mmol). The resulting mixture was stirred at room temperature for 20 min. The reaction mixture was concentrated . The residue was dissolved in water, then adjust ph>8 with solid Na₂CO₃. The aqueous phase was extracted with EA. The combined organic phases were washed with brine, dried with Na₂SO₄, filtered and concentrated. The residue was purified by prep-TLC (MeOH/DCM=10%) to give 11 mg of the title product. Yield: 80%.

### Step 8 tert-butyl 5-((tert-butoxycarbonyl)(2-methyl-6-nitrophenyl)amino)-2-(2,6-difluoro-3,5-dimethoxybenzoyl)-1H-pyrrolo[2,3-c]pyridine-1-carboxylate

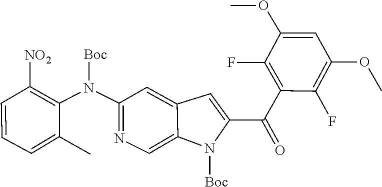 [SMILES: COc1cc(OC)c(F)c(C(=O)c2cc3cc(N(C(=O)OC(C)(C)C)c4c(C)cccc4[N+](=O)[O-])ncc3n2C(=O)OC(C)(C)C)c1F]

To a stirred solution of the product of Step 7 (11 mg ,0.02 mmol) in 1,4-Dioxane (4 ml), DMAP (30 mg ,0.41 mmol) and (Boc)₂O (50 mg ,0.23 mmol) were added. The resulting mixture was stirred at RT for 2 h. The solution was quenched with water and extracted with DCM. The organic layer was washed with brine and dried over Na₂SO₄. The solution was filtered, concentrated and purified by prep-TLC (DCM:MeOH=9:1) to give 6 mg of the title product. Yield: 40%.

### Step 9 tert-butyl 5-((2-amino-6-methylphenyl)(tert-butoxycarbonyl)amino)-2-(2,6-difluoro-3,5-dimethoxybenzoyl)-1H-pyrrolo[2,3-c]pyridine-1-carboxylate

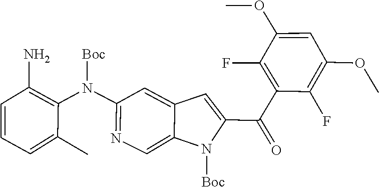 [SMILES: COc1cc(OC)c(F)c(C(=O)c2cc3cc(N(C(=O)OC(C)(C)C)c4c(C)cccc4N)ncc3n2C(=O)OC(C)(C)C)c1F]

To a stirred solution of the product of Step 8 (90 mg ,0.13 mmol) in i-PrOH/H₂O (18 ml/1.5 ml), Fe (300 mg ,5.36 mmol) and NH₄Cl (300 mg ,5.56 mmol) were added. The resulting mixture was stirred at 80° C. for 1 h, then cooled to room temperature and filtered. The solution was quenched with saturated Na₂CO₃ and extracted with EA. The organic layer was washed with brine and dried over Na₂SO₄. The solution was filtered, concentrated and purified by prep-TLC (DCM:MeOH=9:1) to give 48 mg of the title product. Yield: 56%.

### Step 10 tert-butyl 5-((2-acrylamido-6-methylphenyl)(tert-butoxycarbonyl)amino)-2-(2,6-difluoro-3,5-dimethoxybenzoyl)-1H-pyrrolo[2,3-c]pyridine-1-carboxylate

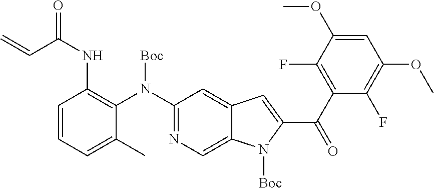 [SMILES: C=CC(=O)Nc1cccc(C)c1N(C(=O)OC(C)(C)C)c1cc2cc(C(=O)c3c(F)c(OC)cc(OC)c3F)n(C(=O)OC(C)(C)C)c2cn1]

The product of Step 9 (48 mg, 0.075 mmol) and Et₃N (0.08 ml, 0.58 mmol) were dissolved in DCM (7 ml). At RT, acrylyl chloride (3.5 ml, 51.1 mmol) was added drop wise to the reaction mixture, and then stirred for 1 h. After the reaction was complete, the reaction mixture was concentrated and purified by prep-TLC (MeOH/DCM=10%) to give 26 mg of the title product. Yield: 50%.

### Step 11 N-(2-(2-(2,6-difluoro-3,5-dimethoxybenzoyl)-1H-pyrrolo[2,3-c]pyridin-5-ylamino)-3-methylphenyl)acrylamide

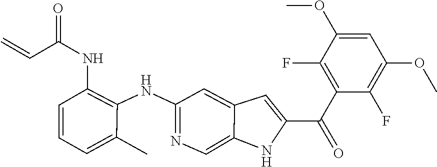 [SMILES: C=CC(=O)Nc1cccc(C)c1Nc1cc2cc(C(=O)c3c(F)c(OC)cc(OC)c3F)[nH]c2cn1]

To a stirred solution of the product of Step 10 (26 mg , 0.04 mmol) in DCM (4 ml), TFA (4 ml) was added at 0° C. The resulting mixture was stirred for 3 h. The solution was quenched with saturated Na2CO3 and extracted with DCM. The organic layer was washed with brine and dried over Na₂SO₄. The solution was filtered, concentrated and purified by prep-TLC (DCM:MeOH=9:1) to give 5 mg of pure product as a yellow solid. MS (ES+): 493.1[M+1]⁺.

¹HNMR: (400 MHz, CDCl₃) δ 2.21 (s, 3H), 3.92 (s, 6H), 5.62-5.65(d, J=12 Hz, 1H), 6.04-6.31(m,4H), 6.73-7.28 (m, 3H), 8.39-8.43 (m, 2H), 8.54(s, 1H), 8.72(s, 1H).

### EXAMPLE 28

### (±)-N-(2-((2-((2,6-difluoro-3,5-dimethoxyphenyl)(hydroxy)methyl)thieno[2,3-c]pyridin-5-yl)amino)-3-methylphenyl)acrylamide

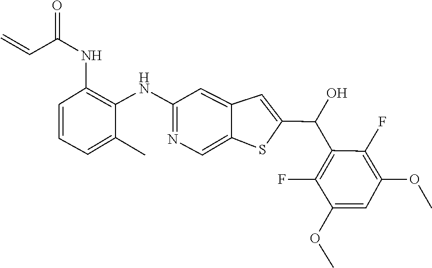 [SMILES: C=CC(=O)Nc1cccc(C)c1Nc1cc2cc(C(O)c3c(F)c(OC)cc(OC)c3F)sc2cn1]

To a stirred solution of N-(2-(2-(2,6-difluoro-3,5-dimethoxybenzoyl)thieno[2,3-c]pyridin-5-ylamino)-3-methylphenyl)acrylamide (Example 26, 7 mg, 0.014 mmol) in MeOH/THF (0.5 ml/2 ml), NaBH₄ (0.5 mg, 0.014 mmol) was added at 0° C. and stirred for 1 h. The solution was quenched with brine and extracted with EA. The organic layer was washed with brine and dried over Na₂SO₄. The solution was filtered, concentrated and recrystallized from MTBE:PE(1:1) to give 3 mg of the title product as an off-white solid. MS (ES+): 512.2 [M+1]⁺.

¹HNMR: (400 MHz, CDCl₃) δ 2.19 (s, 3H), 3.88 (s, 6H), 5.64-5.67 (d, J=12 Hz,1H), 5.91 (s, 1H), 6.13-615 (q, 1H), 6.26-6.38 (m, 1H), 6.63-6.67 (t, J=16 Hz, 1H), 6.73 (s, 1H), 7.04-7.06 (d, J=8 Hz, 1H), 7.26-7.30(t, J=16 Hz, 1H), 8.26 (s, 1H), 8.39-8.41(d, J=8 Hz, 1H), 8.64(s, 1H).

### EXAMPLE 29

### (±)-N-(3-chloro-2-((2-((2,6-difluoro-3,5-dimethoxyphenyl)(hydroxy)methyl)furo[2,3-c]pyridin-5-yl)amino)phenyl)acrylamide

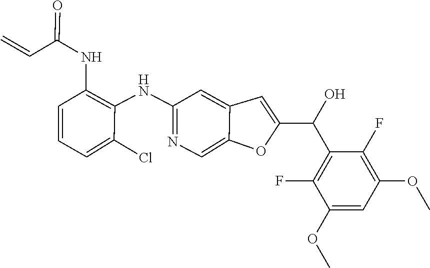 [SMILES: C=CC(=O)Nc1cccc(Cl)c1Nc1cc2cc(C(O)c3c(F)c(OC)cc(OC)c3F)oc2cn1]

To a stirred solution of N-(3-chloro-2-(2-(2,6-difluoro-3,5-dimethoxybenzoyl)furo[2,3-c]pyridin-5-ylamino)phenyl)acrylamide (Example 28) (10 mg, 0.019 mole ) in MeOH/THF (0.5 ml/2 ml), NaBH₄ (0.7 mg, 0.019 mol) was added at 0° C. and stirred for 1 h. The solution was quenched with water and extracted with EA. The organic layer was washed with brine and dried over Na₂SO₄. The solution was filtered, concentrated and purified by prep-TLC (EA:PE=1:1)to give 6 mg of the title product as an off-white solid. Yield: 60%. MS (ES+): 560 [M +1]⁺.

¹HNMR: (400 MHz, CDCl₃) 3.90 (s, 6H), 5.66-5.67 (d, J=4 Hz, 1H), 6.08-6.15 (m, 2H), 6.27-6.33 (m, 2H), 6.55 (s, 1H), 6.65-6.69 (t, J=20 Hz, 1H), 7.24-7.30 (m, 2H), 8.40-8.42(d, J=8 Hz, 1H), 8.66(s, 1H).

### EXAMPLE 30

### (±)-N-(2-((2-((2,6-difluoro-3,5-dimethoxyphenyl)(hydroxy)methyl)furo[2,3-c]pyridin-5-yl)amino)-3-(trifluoromethyl)phenyl)acrylamide

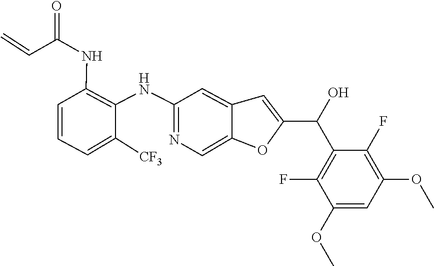 [SMILES: C=CC(=O)Nc1cccc(C(F)(F)F)c1Nc1cc2cc(C(O)c3c(F)c(OC)cc(OC)c3F)oc2cn1]

To a stirred solution of N-(2-(2-(2,6-difluoro-3,5-dimethoxybenzoyl)furo[2,3-c]pyridin-5-ylamino)-3-(trifluoromethyl)phenyl)acrylamide (Example 27, 10 mg, 0.018 mmol) in MeOH/THF (0.5 ml/2 ml), NaBH₄ (0.7 mg, 0.018 mmol) was added at ° C. and stirred for 1 h. The solution was quenched with brine and extracted with EA. The organic layer was washed with brine and dried over Na₂SO₄. The solution was filtered, concentrated and purified by prep-TLC (EA:PE=1:1) to give 6 mg of the title product as an off-white solid. MS (ES+):550 [M+1]⁺¹.

¹HNMR: (400 MHz, CDCl₃) δ 3.90 (s, 6H), 5.64-5.67 (d, J=12 Hz, 1H), 6.02-6.09 (m, 1H), 6.20-6.27 (m, 3H), 6.38 (s, 1H), 6.55 (s, 1H), 6.65-6.69 (t, J=16 Hz,1H), 744-7.49 (m, 2H), 8.40 (s, 1H), 8.51(s, 1H), 8.64-8.66 (m,1H).

### EXAMPLE 31

### (±)- N-(2-((6-((2,6-difluoro-3 ,5-dimethoxyphenyl)(hydroxy)methyl)furo[3,2-d]pyrimidin-2-yl)amino)-5-(4-ethylpiperazin-1-yl)phenyl)acrylamide

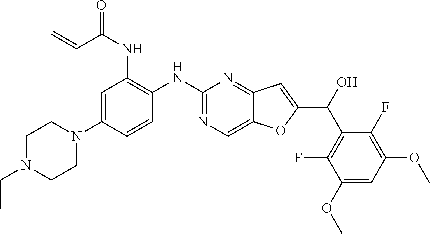 [SMILES: C=CC(=O)Nc1cc(N2CCN(CC)CC2)ccc1Nc1ncc2oc(C(O)c3c(F)c(OC)cc(OC)c3F)cc2n1]

### Step 1 (±)-(2-(2-amino-4-(4-ethylpiperazin-1-yl)phenylamino)furo[3,2-d]pyrimidin-6-yl)(2,6-difluoro-3,5-dimethoxyphenyl)methanol

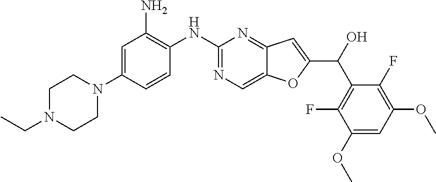 [SMILES: CCN1CCN(c2ccc(Nc3ncc4oc(C(O)c5c(F)c(OC)cc(OC)c5F)cc4n3)c(N)c2)CC1]

To a stirred solution of (2,6-difluoro-3,5-dimethoxyphenyl)(2-(4-(4-ethylpiperazin-1-yl)-2-nitrophenylamino)furo[3,2-d]pyrimidin-6-yl)methanone (the product of Step 4 of Example 22) (400 mg) in EtOH/H₂O (16 ml/8 ml), Fe (400 mg) and NH₄Cl (400 mg) were added. The resulting mixture was stirred at 70° C. for 2.5 h, then cooled to room temperature and filtered. The solution was quenched with water and extracted with EA. The organic layer was washed with brine and dried over Na₂SO₄. The solution was filtered and concentrated to give 348 mg of crude title compound which was used in the next step without further purification. MS (ES+): 541.3 [M +1]⁺.

### Step 2 (±)-N-(2-((6-((2,6-difluoro-3,5-dimethoxyphenyl)(hydroxy)methyl)furo[3,2-d]pyrimidin-2-yl)amino)-5-(4-ethylpiperazin-1-yl)phenyl)acrylamide

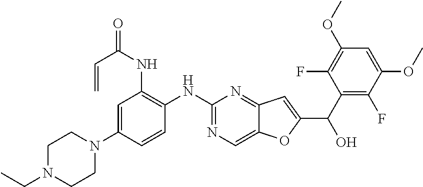 [SMILES: C=CC(=O)Nc1cc(N2CCN(CC)CC2)ccc1Nc1ncc2oc(C(O)c3c(F)c(OC)cc(OC)c3F)cc2n1]

The product of Step 1 (348 mg, 0.64 mmol) and Et₃N (0.13 ml, 0.96 mmol) were dissolved in DCM (4 ml). At 0-10° C., acrylyl chloride (57.92 mg, 0.64 mmol) was added to the reaction mixture, and then stirred for 0.5 h. After the reaction was complete, the resulting mixture was washed with sat. NaHCO₃, brine and dried over Na₂SO₄. The solution was filtered and concentrated. THF (4 ml) and 1.5 N NaOH (4 ml) were added to the residue and stirred at RT. The solution was quenched with water and extracted with EA. The organic layer was washed with brine and dried over Na₂SO₄. The solution was filtered, concentrated and purified by silica gel chromatography (MeOH/DCM=6%) to get 75 mg of pure product as a yellow solid. Yield: 19.6%. MS (ES+): 595.3 [M+1]⁺.

¹HNMR: (400 MHz, MeOH-d4) δ 1.17-1.20 (t, J=12 Hz, 3H), 2.55-2.60 (q, 2H), 2.70-2.74 (m, 4H), 3.25-3.27 (m, 4H), 3.86 (s, 6H), 5.73-5.76 (d, J=12 Hz, 1H), 6.24 (s, 1H), 6.32-6.45 (m, 2H), 6.71 (s, 1H), 6.90-6.94 (m, 2H), 7.28(s, 1H), 7.52-7.54(d, J=8 Hz, 1H), 8.44 (s, 1H).

### EXAMPLE 32

### 5-((2-acrylamido-4-(4-ethylpiperazin-1-yl)phenyl)amino)-N-(2,6-difluoro-3,5-dimethoxyphenyl)furo[2,3-c]pyridine-2-carboxamide

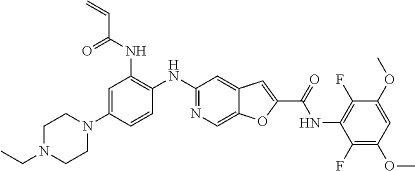 [SMILES: C=CC(=O)Nc1cc(N2CCN(CC)CC2)ccc1Nc1cc2cc(C(=O)Nc3c(F)c(OC)cc(OC)c3F)oc2cn1]

### Step 1 methyl 2,6-difluoro-3,5-dimethoxybenzoate

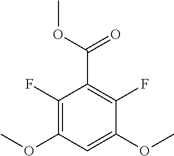

To a stirred solution of methyl 3, 5-dimethoxybenzoate (80.0 g, 408 mmol) in acetonitrile (150 ml), a solution of Selectfluor (200 g, 560 mmol) in acetonitrile (7 L) was added drop wise at 0° C. and stirred at room temperature for 15 h. The mixture was concentrated. The residue was quenched with citric acid (aq) and extracted with MTBE. The organic layer was washed with brine and dried over Na₂SO₄. The solution was filtered, concentrated and purified by silica gel chromatography (PE:EA=5:1) to give 17.5 g of the title product as a light yellow solid. Yield: 18.5%.

### Step 2 2,6-difluoro-3,5-dimethoxybenzoic acid

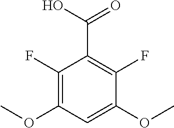

To a stirred solution of methyl 2,6-difluoro-3,5-dimethoxybenzoate (30.0 g, 129 mmol) in THF/H₂O (150 ml/150 ml), NaOH (20 g. 500 mmol) was added and stirred at room temperature for 6 h. The resulting mixture was acidified with 4N HCl, and then THF was evaporated. The resulting precipitate was collected by filtration and dried to give 26.9 g of the title product as a white solid. Yield: 95%.

### Step 3 tert-butyl 2,6-difluoro-3,5-dimethoxyphenylcarbamate

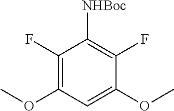

To a stirred solution of methyl 2,6-difluoro-3,5-dimethoxybenzoic acid (8.0 g, 36.7 mmol) in toluene (80 ml), t-BuOH (3.7 ml. 44 mmol), Et₃N (6.1 ml. 44 mmol) and DPPA (11.1 g, 40.3 mmol) was added and stirred at 70° C. for 2 h. The solution was quenched with water and extracted with EA. The organic layer was washed with brine and dried over Na₂SO₄. The solution was filtered and concentrated to give 8.0 g of the title product as a white solid. Yield: 75.5%.

### Step 4 2,6-difluoro-3,5-dimethoxyaniline

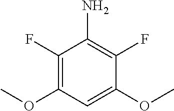

To a stirred solution of tert-butyl 2,6-difluoro-3,5-dimethoxyphenylcarbamate (8.0 g, 27.6 mmol) in 4N HCl in EA (250 ml) and stirred at room temperature for 15 h. The solution was quenched with Na₂CO₃ and extracted with EA. The organic layer was washed with brine and dried over Na₂SO₄. The solution was filtered and concentrated to give 3.2 g of pure product as an off-white solid. Yield: 61.5%.

### Step 5 2-chloro-5-(methoxymethoxy)isonicotinaldehyde

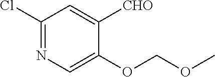

To a stirred solution of 2-chloro-5-(methoxymethoxy)pyridine (50.0 g, 289 mmol) in THF (250 ml), n-Butyllithium (125 ml ,318 mmol) was added drop wise at −60° C. and stirred for 2 h. Then DMF (24 ml, 318 mmol) was added drop wise and stirred for 1 h. Then the reaction mixture was stirred at 20° C. for 1 h. The solution was quenched with sat.NH₄Cl and extracted with EA. The organic layer was washed with brine and dried over Na₂SO₄. The solution was filtered, concentrated and purified by silica gel chromatography (PE:EA=6:1) to give 42 g of the title product. Yield: 72%.

### Step 6 2-chloro-5-hydroxyisonicotinaldehyde

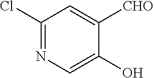

To a stirred solution of 2-chloro-5-(methoxymethoxy)isonicotinaldehyde (48.0 g, 238 mmol) in THF (250 ml), 3N HCl (600 ml) was added and stirred at 60° C. for 4 h and stirred at room temperature for 15 h. The solution was quenched with Na₂CO₃ and extracted with EA. The organic layer was washed with brine and dried over Na₂SO₄. The solution was filtered, concentrated and purified by silica gel chromatography (PE:EA=6:1) to give 23.6 g of the title product. Yield: 63%.

### Step 7 methyl 5-chlorofuro[2,3-c]pyridine-2-carboxylate

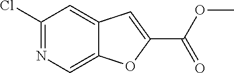

To a stirred solution of 2-chloro-5-hydroxyisonicotinaldehyde (23.6 g, 150 mmol) in THF/DMF (500/60 ml), methyl bromoacetate (23.6 ml, 225 mmol) and K₂CO₃ (46 g ,300 mmol) were added and stirred at 50° C. for 1 h and stirred at room temperature for 15 h. The solution was quenched with water and extracted with EA. The organic layer was washed with brine and dried over Na₂SO₄. The solution was filtered, concentrated and purified by silica gel chromatography (PE:EA=9:1) to give 11.3 g of the title product. Yield: 36%.

### Step 8 methyl 5-(4-(4-ethylpiperazin-1-yl)-2-nitrophenylamino)furo[2,3-c]pyridine-2-carboxylate

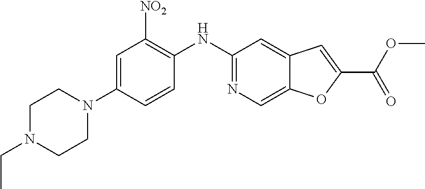 [SMILES: CCN1CCN(c2ccc(Nc3cc4cc(C(=O)OC)oc4cn3)c([N+](=O)[O-])c2)CC1]

A mixture of methyl 5-chlorofuro[2,3-c]pyridine-2-carboxylate (1.0 g, 4.72 mmol), 4-(4-ethylpiperazin-1-yl)-2-nitroaniline (1.18 g, 4.72 mmol), Pd₂(dba)₃ (0.87 g, 0.95 mmol), X-phos (0.90 g, 1.89 mmol) and Cs₂CO₃ (3.08 g, 9.45 mmol) in toluene (10 ml) was heated at 110° C. under N₂ for 15 h. The reaction mixture was cooled to room temperature, filtered and concentrated to dryness. The residue was purified by silica gel chromatography (MeOH/3:97) to give 1.3 g of the title product as a dark brown solid. Yield: 65%. MS (ES+): 426 [M +1]⁺

### Step 9 5-(4-(4-ethylpiperazin-1-yl)-2-nitrophenylamino)furo[2,3-c]pyridine-2-carboxylic acid

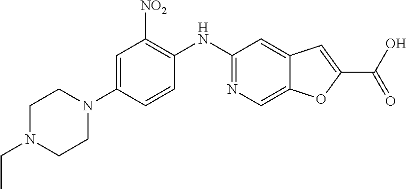 [SMILES: CCN1CCN(c2ccc(Nc3cc4cc(C(=O)O)oc4cn3)c([N+](=O)[O-])c2)CC1]

To a stirred solution of methyl 5-(4-(4-ethylpiperazin-1-yl)-2-nitrophenylamino)furo[2,3-c]pyridine-2-carboxylate (500 mg, 1.17 mmol) in THF/H₂O (9 ml/3 ml), LiOH H₂O (500 mg, 11.9 mmol) was added and stirred at room temperature for 1 h. The solution was quenched with 1N HCl and extracted with EA. The organic layer was washed with brine and dried over Na₂SO₄. The solution was filtered, concentrated and purified by silica gel chromatography (CH₃CN:H₂O=1:4) to give 290 mg of pure product. Yield: 60%. MS (ES+): 412 [M +1]⁺.

### Step 10 N-(2,6-difluoro-3,5-dimethoxyphenyl)-5-(4-(4-ethylpiperazin-1-yl)-2-nitrophenylamino)furo[2,3-c]pyridine-2-carboxamide

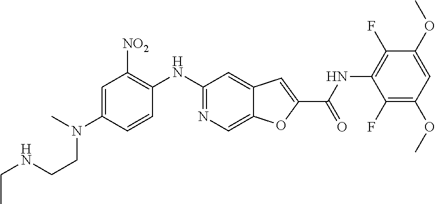 [SMILES: CCNCCN(C)c1ccc(Nc2cc3cc(C(=O)Nc4c(F)c(OC)cc(OC)c4F)oc3cn2)c([N+](=O)[O-])c1]

To a stirred solution of 5-(4-(4-ethylpiperazin-1-yl)-2-nitrophenylamino)furo[2,3-c]pyridine-2-carboxylic acid (250 mg, 0.61 mmol) in DMF (5 ml), DMAP (446 ml, 3.65 mmol) and HATU (924 mg, 2.43 mmol) were added. The resulting mixture was stirred at room temperature for 15 h. The solution was quenched with water and extracted with EA. The organic layer was washed with brine and dried over Na₂SO₄. The solution was filtered, concentrated and purified by silica gel chromatography (MeOH/DCM=3:97) to give 200 mg of the title product as a dark-red solid. Yield: 62%.

### Step 11 5-(2-amino-4-(4-ethylpiperazin-1-yl)phenylamino)-N-(2,6-difluoro-3,5-dimethoxyphenyl)furo[2,3-c]pyridine-2-carboxamide

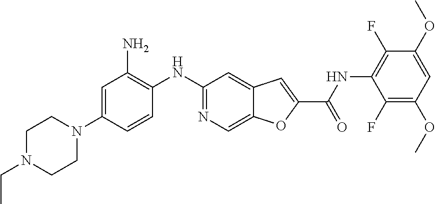 [SMILES: CCN1CCN(c2ccc(Nc3cc4cc(C(=O)Nc5c(F)c(OC)cc(OC)c5F)oc4cn3)c(N)c2)CC1]

To a stirred solution of the product of Step 10 (50 mg, 0.095 mmol) in EtOH/H₂O (2 ml/0.5 ml), Fe (48 mg, 0.95 mmol) and NH₄Cl (46 mg, 0.95 mmol) were added. The resulting mixture was stirred at 85° C. for 2 h, then cooled to room temperature and filtered. The solution was quenched with water and extracted with DCM. The organic layer was washed with brine and dried over Na₂SO₄. The solution was filtered and concentrated to give 50 mg of crude title compound which was used in the next step without further purification. MS (ES+): 553 [M +1]⁺.

### Step 12 5-(2-acrylamido-4-(4-ethylpiperazin-1-yl)phenylamino)-N-(2,6-difluoro-3,5-dimethoxyphenyl)furo[2,3-c]pyridine-2-carboxamide

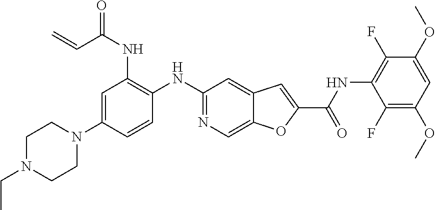 [SMILES: C=CC(=O)Nc1cc(N2CCN(CC)CC2)ccc1Nc1cc2cc(C(=O)Nc3c(F)c(OC)cc(OC)c3F)oc2cn1]

The product of Step 11 (30 mg, 0.05 mmol) and Et₃N (54 mg, 0.55 mmol) were dissolved in DCM (1 ml). At 0-10° C., 3-chloropropionyl chloride (10 mg, 0.08 mmol) was added to the reaction mixture, and then stirred for 1 h. After the reaction was complete, the reaction mixture was concentrated and purified by prep-TLC (MeOH:DCM=5:95) to get 10 mg of the title product as a yellow solid. Yield: 33%. MS (ES+): 607.7 [M+1]+.

¹HNMR: (400 MHz, DMSO-d₆) δ 1.16-1.24 (t, J=32 Hz, 3H), 2.51-2.90 (m, 8H), 3.06-3.08 (m, 1H), 3.90 (s, 6H), 6.21-6.25 (d, J=16 Hz, 1H), 6.47-6.51 (m, 1H), 6.81-6.90 (m, 2H), 7.01-7.04 (t, J=16 Hz, 1H), 7.36-7.37 (t, J=4 Hz, 2H), 7.59(s, 1H), 7.84(s, 1H), 8.59(s, 1H), 9.68(s, 1H) , 10.63(s, 1H).

### EXAMPLE 33

### (Z)-3-chloro-N-(2-((2-(2,6-difluoro-3,5-dimethoxybenzoyl)furo[2,3-c]pyridin-5-yl)amino)-5-(4-ethylpiperazin-1-yl)phenyl)acrylamide

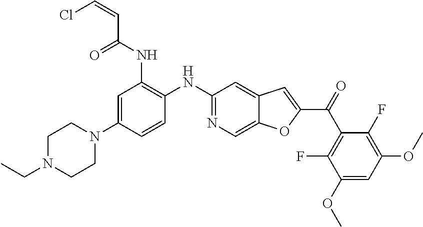 [SMILES: CCN1CCN(c2ccc(Nc3cc4cc(C(=O)c5c(F)c(OC)cc(OC)c5F)oc4cn3)c(NC(=O)/C=C\Cl)c2)CC1]

### Step 1 (2,6-difluoro-3,5-dimethoxyphenyl)(5-(4-(4-ethylpiperazin-1-yl)-2-nitrophenylamino)furo[2,3-c]pyridin-2-yl)methanone

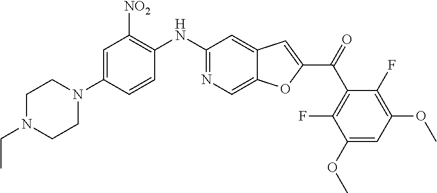 [SMILES: CCN1CCN(c2ccc(Nc3cc4cc(C(=O)c5c(F)c(OC)cc(OC)c5F)oc4cn3)c([N+](=O)[O-])c2)CC1]

A mixture of (5-chlorofuro[2,3-c]pyridin-2-yl)(2,6-difluoro-3,5 -dimethoxyphenyl)methanone (300 mg, 0.848 mmol), 4-(4-ethylpiperazin-1-yl)-2-nitroaniline (212 mg, 0.848 mmol), Pd₂(dba)₃ (156 mg, 0.170 mmol), X-phos (162 mg, 0.339 mmol) and Cs₂CO₃ (553 mg, 1.696 mmol) in toluene (9 ml) was heated at 115° C. under Are overnight. The reaction mixture was cooled to room temperature, filtered and concentrated to dryness. The residue was purified by silica gel chromatography (DCM:MeOH=97:3) to give 300 mg of the title product. Yield: 62.3%.

### Step 2 (5-(2-amino-4-(4-ethylpiperazin-1-yl)phenylamino)furo[2,3-c]pyridin-2-yl)(2,6-difluoro-3,5-dimethoxyphenyl)methanone

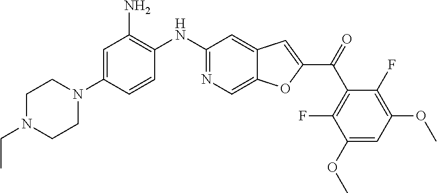 [SMILES: CCN1CCN(c2ccc(Nc3cc4cc(C(=O)c5c(F)c(OC)cc(OC)c5F)oc4cn3)c(N)c2)CC1]

To a stirred solution of the product of Step 1 (100 mg, 0.176 mmol) in i-PrOH /H₂O (9 ml/3 ml), Fe (100 mg, 1.76 mmol) and NH₄Cl (100 mg, 1.76 mmol) were added. The resulting mixture was stirred at 80° C. for 1 h, then cooled to room temperature and filtered. The solution was quenched with water and extracted with EA. The organic layer was washed with brine and dried over Na₂SO₄. The solution was filtered and concentrated to give 100 mg of crude title compound which was used in the next step without further purification.

### Step 3 (Z)-3-chloro-N-(2-(2-(2,6-difluoro-3,5-dimethoxybenzoyl)furo[2,3-c]pyridin-5-ylamino)-5-(4-ethylpiperazin-1-yl)phenyl)acrylamide

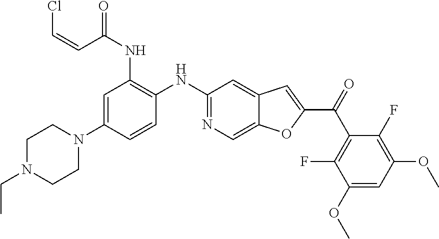 [SMILES: CCN1CCN(c2ccc(Nc3cc4cc(C(=O)c5c(F)c(OC)cc(OC)c5F)oc4cn3)c(NC(=O)/C=C\Cl)c2)CC1]

The product of Step 2 (100 mg, 0.176 mmol) was dissolved in DCM (9 ml). At 0° C., T3P (50% in DMF, 336 mg , 1.056 mmol), Et₃N (0.25 ml, 1.76 mmol) and cis-3-Chloroacrylic acid (56 mg, 0.528 mmol) were added to the reaction mixture, and then stirred for 1 h. After the reaction was complete, the reaction mixture was quenched with saturated aqueous Na₂CO₃, extracted with EA. The organic layer was washed with brine and dried over Na₂SO₄. The solution was filtered, concentrated and purified by prep-TLC (DCM:MeOH=95:5) to give 10 mg of the title product. Yield: 9.1%. MS (ES+): 626 [M+1]⁺.

¹HNMR: (400 MHz, CDCl₃) δ 1.28 (s , 3H), 2.85 (s, 2H), 2.86 (s, 4H), 3.45 (s, 5H), 3.98(s, 6H), 6.21 (m, 1H), 6.43 (s, 1H),6.50-6.52 (d, J=8 Hz ,1H), 6.75 (m, 1H), 6.81(m, 1H), 7.17-7.19(m, 1H), 7.22(s, 1H), 8.20(s, 1H), 8.60(s, 1H), 8.84(s, 1H).

### EXAMPLE 34

### N-((3S,4S)-3-((6-((2,6-difluoro-3,5-dimethoxyphenyl)(hydroxy)methyl)furo[3,2-d]pyrimidin-2-yl)amino)tetrahydro-2H-pyran-4-yl)acrylamide

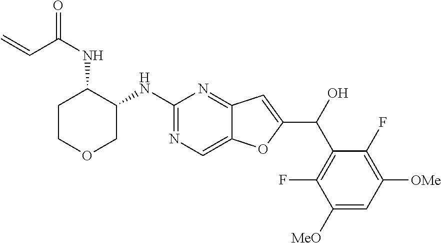 [SMILES: C=CC(=O)N[C@H]1CCOC[C@H]1Nc1ncc2oc(C(O)c3c(F)c(OC)cc(OC)c3F)cc2n1]

### Step 1 (2-((3S,4S)-4-aminotetrahydro-2H-pyran-3-ylamino)furo[3,2-d]pyrimidin-6-yl)(2,6-difluoro-3,5-dimethoxyphenyl)methanol

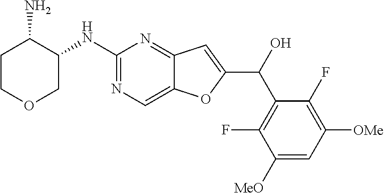 [SMILES: COc1cc(OC)c(F)c(C(O)c2cc3nc(N[C@@H]4COCC[C@@H]4N)ncc3o2)c1F]

Pd/C (10%, 80 mg) was added to a solution of (2-((3S,4S)-4-azidotetrahydro-2H-pyran-3-ylamino)furo[3,2-d]pyrimidin-6-yl)(2,6-difluoro-3,5-dimethoxyphenyl)methanone (80 mg) in THF (32 ml). The reaction mixture was stirred for 2 h under H₂ (1 atm, balloon) at RT. The reaction mixture was filtered through a layer of Celite®. The filtrate was concentrated and purified by prep-TLC (10% MeOH/DCM) to give 10 mg of the title product. Yield: 13.2%. MS (ES+): 437.1 [M+H]⁺.

### Step 2 N-((3S,4S)-3-((6-((2,6-difluoro-3,5-dimethoxyphenyl)(hydroxy)methyl)furo[3,2-d]pyrimidin-2-yl)amino)tetrahydro-2H-pyran-4-yl)acrylamide

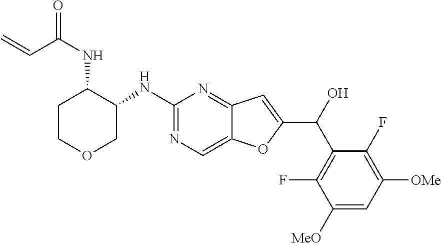 [SMILES: C=CC(=O)N[C@H]1CCOC[C@H]1Nc1ncc2oc(C(O)c3c(F)c(OC)cc(OC)c3F)cc2n1]

The product of Step 1 (10 mg) and Et₃N (30 mg) were dissolved in DCM (2 ml). At 0-10° C., acrylyl chloride (10 mg) was added to the reaction mixture, and then stirred for 0.5 h. After the reaction was complete, the resulting mixture was washed with sat. NaHCO3, brine and dried over Na₂SO₄. The solution was filtered and concentrated. THF (1 ml) and 1.5 N NaOH (1 ml) were added to the residue and stirred at RT. The solution was quenched with water and extracted with EA. The organic layer was washed with brine and dried over Na₂SO₄. The solution was filtered, concentrated and purified by prep-TLC (EA) to give 5 mg of the title product as a white solid. Yield: 44.6%. MS (ES+): 491.2 [M+1]⁺.

¹HNMR: (400 MHz, CDCl₃) δ 2.07 (m, 2H), 3.38 (bs, 1H), 3.55-3.61 (t, J=24 Hz, 1H), 3.68-3.71 (d, J=12 Hz, 1H), 3.91-3.94 (m, 9H), 4.22 (m, 1H), 4.34 (m, 1H), 5.55 (s, 1H), 5.58 (d, J=0.8 Hz, 1H), 5.72-5.74 (d, J=8 Hz, 1H), 5.93-6.00 (m, 1H), 6.16-6.20 (d, J=16 Hz, 1H), 6.29 (s, 1H), 6.64-6.71 (m, 2H),7.05(s, 1H), 8.39 (s, 1H).

### EXAMPLE 35

### N-((3S,4S)-3-((6-(2,6-difluoro-3,5-dimethoxybenzoyl)furo[3,2-d]pyrimidin-2-yl)amino)tetrahydro-2H-pyran-4-yl)acrylamide

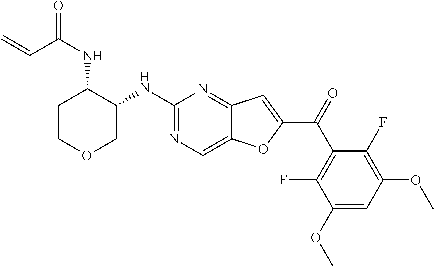 [SMILES: C=CC(=O)N[C@H]1CCOC[C@H]1Nc1ncc2oc(C(=O)c3c(F)c(OC)cc(OC)c3F)cc2n1]

### Step 1 (2-((3S,4S)-4-azidotetrahydro-2H-pyran-3-ylamino)furo[3,2-d]pyrimidin-6-yl)(2,6-difluoro-3,5-dimethoxyphenyl)methanone

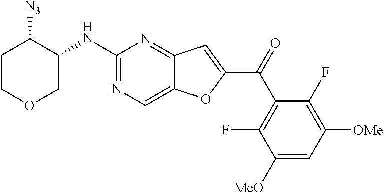 [SMILES: COc1cc(OC)c(F)c(C(=O)c2cc3nc(N[C@@H]4COCC[C@@H]4N=[N+]=[N-])ncc3o2)c1F]

A mixture of (2-chlorofuro[3,2-d]pyrimidin-6-yl)(2,6-difluoro-3,5-dimethoxyphenyl)methanone (250 mg, 0.70 mmol), (3S,4S)-4-azidotetrahydro-2H-pyran-3-amine (120 mg, 0.85 mmol), Pd₂(dba)₃ (128 mg, 0.14 mmol), Xantphos (162 mg, 0.28 mmol) and Cs₂CO₃ (684 mg, 2.1 mmol) in toluene (25 ml) was heated at 95° C. under Are and stirred overnight. The reaction mixture was cooled to room temperature, filtered and concentrated to dryness. The residue was purified by prep-TLC (40% EA/PE) to give 100 mg of the title product as a yellow solid. Yield: 30.9%. MS (ES+): 461.1 [M +1]^{+.}

### Step 2 (2-((3S,4S)-4-aminotetrahydro-2H-pyran-3-ylamino)furo[3,2-d]pyrimidin-6-yl)(2,6-difluoro-3,5-dimethoxyphenyl)methanone

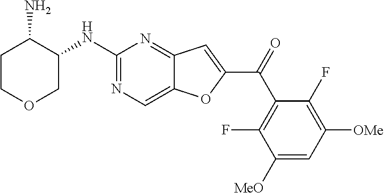 [SMILES: COc1cc(OC)c(F)c(C(=O)c2cc3nc(N[C@@H]4COCC[C@@H]4N)ncc3o2)c1F]

To a stirred solution of the product of Step 1 (90 mg) in EtOH/H₂O (36 ml/9 ml), Fe (90 mg) and NH₄Cl (90 mg) were added. The resulting mixture was stirred at 70° C. for 8 h, then cooled to room temperature and filtered. The solution was quenched with water and extracted with EA. The organic layer was washed with brine and dried over Na₂SO₄. The solution was filtered, concentrated and purified by prep-TLC (10% MeOH/DCM) to give 8 mg of the title product as a yellow solid. Yield: 9.4%.MS (ES+): 435.1 [M +1]⁺.

### Step 3 N-((3S,4S)-3-((6-(2,6-difluoro-3,5-dimethoxybenzoyl)furo[3,2-d]pyrimidin-2-yl)amino)tetrahydro-2H-pyran-4-yl)acrylamide

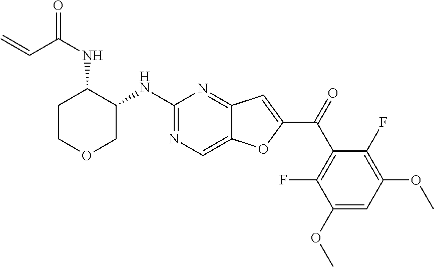 [SMILES: C=CC(=O)N[C@H]1CCOC[C@H]1Nc1ncc2oc(C(=O)c3c(F)c(OC)cc(OC)c3F)cc2n1]

The product of Step 2 (8 mg) and Et₃N (20 mg) were dissolved in DCM (2 ml). At 0-10° C., acrylyl chloride (8 mg) was added to the reaction mixture, and then stirred for 0.5 h. After the reaction was complete, the reaction mixture was concentrated and purified by prep-TLC (EA) to give 5 mg of the title product as a yellow solid. Yield: 55.6%. MS (ES+): 489.1 [M+1]⁺.

¹HNMR: (400 MHz, CDCl₃) δ 2.09-2.11 (t, J=8 Hz, 2H), 3.57-3.63 (t, J=24 Hz,1H), 3.70-3.77 (t, J=28 Hz,1H), 3.96-4.05 (m, 8H), 4.28 (m, 1H), 4.41-4.44 (d, J=12 Hz, 1H), 5.57-5.60 (m, J=12 Hz, 1H), 5.88-5.99 (m, 2H), 6.17-6.22 (d, J=20 Hz, 1H), 6.68 (s, 1H), 6.82-6.88(t, J=24 Hz,1H), 7.30(s, 1H), 8.70(s, 1H).

### EXAMPLE 36

### N-(2-((2-(2,6-difluoro-3,5-dimethoxybenzoyl)-3-hydroxyfuro[2,3-c]pyridin-5-yl)amino)-5-(4-ethylpiperazin-1-yl)phenyl)acrylamide

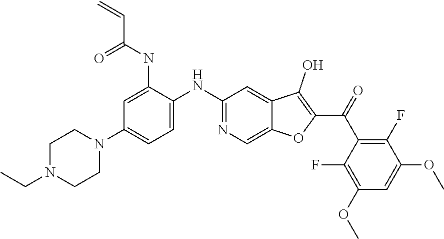 [SMILES: C=CC(=O)Nc1cc(N2CCN(CC)CC2)ccc1Nc1cc2c(O)c(C(=O)c3c(F)c(OC)cc(OC)c3F)oc2cn1]

### Step 1 (5-chloro-3-hydroxyfuro[2,3-c]pyridin-2-yl)(2,6-difluoro-3,5-dimethoxyphenyl)methanone

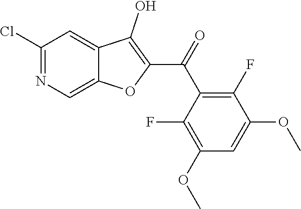 [SMILES: COc1cc(OC)c(F)c(C(=O)c2oc3cnc(Cl)cc3c2O)c1F]

To a stirred solution of methyl 2-chloro-5-hydroxyisonicotinate (1.27 g, 6.79 mmol) in DMF (12 ml), K₂CO₃ (1.88 g, 13.6 mmol), 2-bromo-1-(2,6-difluoro-3,5-dimethoxyphenyl)ethanone (2 g, 6.80 mmol) were added. The resulting mixture was stirred at room temperature for 15 h. The solution was quenched with (1N HCl) and stirred for 1 h. The resulting precipitate was collected by filtration and dried to give 1.4 g of the title product as a white solid. Yield: 88.1%. MS (ES+): 370 [M +1]⁺.

### Step 2 (5-chloro-3-(3,4,5-trimethoxybenzyloxy)furo[2,3-c]pyridin-2-yl)(2,6-difluoro-3,5-dimethoxyphenyl)methanone

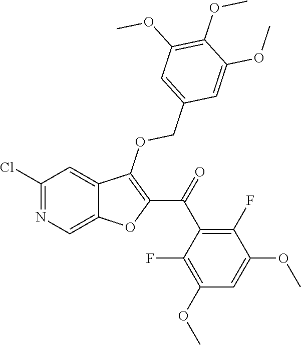 [SMILES: COc1cc(OC)c(F)c(C(=O)c2oc3cnc(Cl)cc3c2OCc2cc(OC)c(OC)c(OC)c2)c1F]

To a stirred solution of (5-chloro-3-hydroxyfuro[2,3-c]pyridin-2-yl)(2,6-difluoro-3,5-dimethoxyphenyl)methanone (1.4 g, 3.78 mmol) in THF (32 ml), (3,4,5-trimethoxyphenyl)methanol (0.91 g, 4.59 mmol), PPh₃ (1.49 g, 5.68 mmol) were added. The mixture cooled to 0° C. then DEAD (1 g, 5.75 mmol) was added drop wise. The resulting mixture was stirred at 0° C. for 1 h. The solution was quenched with water and extracted with EA. The organic layer was washed with brine and dried over Na₂SO₄. The solution was filtered and concentrated. The residue was purified by silica gel chromatography (EA/PE=1:1) to give 1.2 g of the title product as a white solid. Yield: 59% . MS (ES+): 535 (M+1)⁺.

### Step 3 (2,6-difluoro-3,5-dimethoxyphenyl)(5-(4-(4-ethylpiperazin-1-yl)-2-nitrophenylamino)-3-(3,4,5-trimethoxybenzyloxy)furo[2,3-c]pyridin-2-yl)methanone

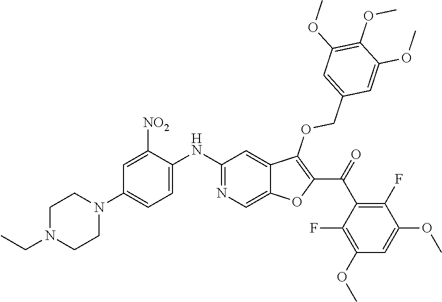 [SMILES: CCN1CCN(c2ccc(Nc3cc4c(OCc5cc(OC)c(OC)c(OC)c5)c(C(=O)c5c(F)c(OC)cc(OC)c5F)oc4cn3)c([N+](=O)[O-])c2)CC1]

The mixture of the product of Step 2 (600 mg, 1.12 mmol), 4-(4-ethylpiperazin-1-yl)-2-nitroaniline (274 mg, 1.1 mmol), Pd₂(dba)₃ (200 mg, 0.22 mmol), X-phos (208 mg, 0.44 mmol) and Cs₂CO₃ (712 mg, 2.2 mmol) in toluene (36 ml) was heated at 110° C. under N₂ for 4 h. The reaction mixture was cooled to room temperature, filtered and concentrated to dryness. The residue was purified by silica gel chromatography (MeOH/DCM=2:98) to give 200 mg of pure product as a red solid. Yield: 42%. MS (ES+): 764 (M+1)⁺.

### Step 4 (5-(2-amino-4-(4-ethylpiperazin-1-yl)phenylamino)-3-(3,4,5-trimethoxybenzyloxy)furo[2,3-c]pyridin-2-yl)(2,6-difluoro-3,5-dimethoxyphenyl)methanone

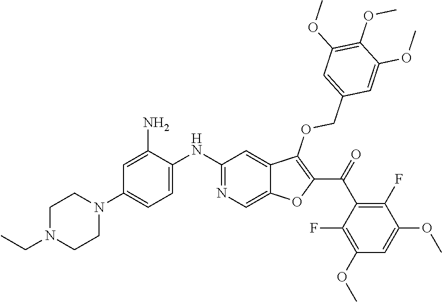 [SMILES: CCN1CCN(c2ccc(Nc3cc4c(OCc5cc(OC)c(OC)c(OC)c5)c(C(=O)c5c(F)c(OC)cc(OC)c5F)oc4cn3)c(N)c2)CC1]

To a stirred solution of the product of Step 3 (100 mg) in EtOH (20 ml), Pd/C (200 mg) were added. The resulting mixture was stirred at RT for 10 min under hydrogen atmosphere. Then the solution was filtered and concentrated to give 80 mg of crude title compound which was used in the next step without further purification. MS (ES+): 563 [M +1]⁺.

### Step 5 N-(2-(2-(2,6-difluoro-3,5-dimethoxybenzoyl)-3-(3,4,5-trimethoxybenzyloxy)furo[2,3-c]pyridin-5-ylamino)-5-(4-ethylpiperazin-1-yl)phenyl)acrylamide

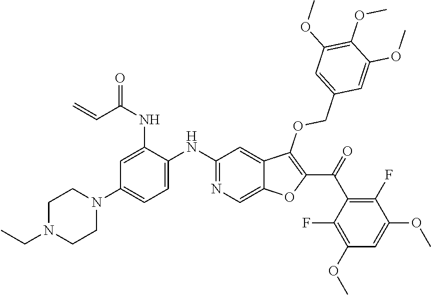 [SMILES: C=CC(=O)Nc1cc(N2CCN(CC)CC2)ccc1Nc1cc2c(OCc3cc(OC)c(OC)c(OC)c3)c(C(=O)c3c(F)c(OC)cc(OC)c3F)oc2cn1]

The product of Step 4 (80 mg, 0.11 mmol) and Et₃N (110 mg, 1.1 mmol) were dissolved in DCM (1 ml). At 0-10° C., 3-chloropropionyl chloride (28 mg, 0.22 mmol) was added to the reaction mixture, and then stirred for 1 h. After the reaction was complete, the reaction mixture was concentrated and purified by prep-TLC (MeOH:DCM=5:95) to get 20 mg of the title product as a yellow solid. Yield: 90%. MS (ES+): 788 [M+1]⁺.

### Step 6 N-(2-(2-(2,6-difluoro-3,5-dimethoxybenzoyl)-3-hydroxyfuro[2,3-c]pyridin-5-ylamino)-5-(4-ethylpiperazin-1-yl)phenyl)acrylamide

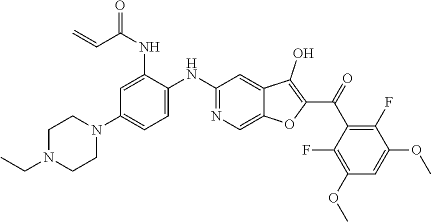 [SMILES: C=CC(=O)Nc1cc(N2CCN(CC)CC2)ccc1Nc1cc2c(O)c(C(=O)c3c(F)c(OC)cc(OC)c3F)oc2cn1]

To a stirred solution of the product of Step 5 (20 mg 0.025 mmol) in TFA (1 ml). The resulting mixture was stirred at room temperature for 0.5 h, the reaction mixture was concentrated and neutralized with Na₂CO₃, and extracted with DCM, washed with brine . The organic layer was washed with brine and dried over Na₂SO₄. The solution was filtered and concentrated. The residue was purified by silica gel chromatography (CH₃CN:H₂O=1:5) to give 6.8 mg of pure product as a yellow solid. Yield: 45%. MS (ES+): 608 [M +1]⁺.

¹HNMR: (400 MHz, MeOD) δ 1.48-1.42 (m, 3H),3.22 (s, 8H), 3.70-3.71 (m, 2H),3.94 (s, 6H), 5.36-5.50 (m, 1H), 5.73-6.43 (m, 2H), 7.07-7.40 (m, 2H), 7.41-7.42 (d, J =8 Hz, 1H), 7.51-7.52 (d, J=4 Hz, 1H), 8.46(d, J=16 Hz, 1H).

### EXAMPLE 37

### N-((3S,4S)-3-((2-(2,6-dichloro-3,5-dimethoxyphenyl)furo[2,3-c]pyridin-5-yl)amino)tetrahydro-2H-pyran-4-yl)acrylamide

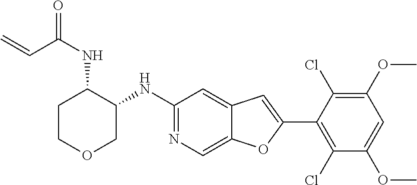 [SMILES: C=CC(=O)N[C@H]1CCOC[C@H]1Nc1cc2cc(-c3c(Cl)c(OC)cc(OC)c3Cl)oc2cn1]

### Step 1 2,4-dichloro-3-ethynyl-1,5-dimethoxybenzene

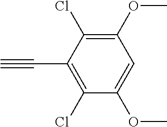

To a stirred solution of 2,6-dichloro-3,5-dimethoxybenzaldehyde (5.0 g, 21.4 mmol) and dimethyl 1-diazo-2-oxopropylphosphonate (4.9 g, 25.5 mmol) in MeOH (100 ml), K₂CO₃ (4.4 g, 31.9 mmol) was added and stirred overnight. The solution was quenched with water and extracted with EA. The organic layer was washed with brine and dried over Na₂SO₄. The solution was filtered, concentrated to give 4.6 g of the title product as a yellow solid. Yield: 93%. MS (ES+): 231 [M+1]⁺.

### Step 2 5-chloro-2-(2,6-dichloro-3,5-dimethoxyphenyl)furo[2,3 -c]pyridine

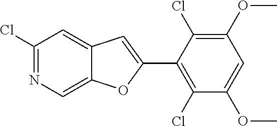 [SMILES: COc1cc(OC)c(Cl)c(-c2cc3cc(Cl)ncc3o2)c1Cl]

A mixture of 2,4-dichloro-3-ethynyl-1,5-dimethoxybenzene (1.68 g, 7.3 mmol), 6-chloro-4-iodopyridin-3-ol (1.86 g, 7.3 mmol), CuI (0.278 g, 1.45 mmol), DIEA (4.7 g, 36.4 mmol), Pd₂(pph₃)₂C₂ (0.51 g, 0.7 mmol) in DMF (16 ml) was heated at 70° C. under N₂ and stirred overnight. The reaction mixture was cooled to RT, quenched with sat.NH₄Cl and extracted with EA. The organic layer was washed with brine and dried over Na₂SO₄. The solution was filtered, concentrated and purified by silica gel chromatography (PE:EA=4:1) to give 1.4 g of the title product as a yellow solid. Yield: 53.8%. MS (ES+): 358 [M +1]⁺.

### Step 3 N-((3S,4S)-4-azidotetrahydro-2H-pyran-3-yl)-2-(2,6-dichloro-3,5-dimethoxyphenyl)furo[2.3-c]pyridin-5-amine

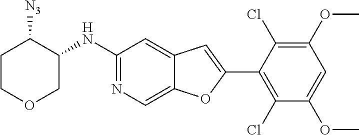 [SMILES: COc1cc(OC)c(Cl)c(-c2cc3cc(N[C@@H]4COCC[C@@H]4N=[N+]=[N-])ncc3o2)c1Cl]

A mixture of 5-chloro-2-(2,6-dichloro-3,5-dimethoxyphenyl)furo[2,3-c]pyridine (240 mg, 0.67 mmol), (3S,4S)-4-azidotetrahydro-2H-pyran-3-amine (107 mg, 0.75 mmol), t-BuOK (264 mg, 2.36 mmol), Binap (120 mg, 0.19 mmol), Pd₂(dba)₃ (120 mg, 0.13 mmol) in toluene (5 ml) was heated at 110° C. under N₂ and stirred overnight. The reaction mixture was cooled to RT, quenched with water and extracted with EA. The organic layer was washed with brine and dried over Na₂SO₄. The solution was filtered, concentrated and purified by silica gel chromagraphy (PE:EA=2:1) to give 50 mg of the title product as a yellow solid. Yield: 16%. MS (ES+): 464 [M +1]⁺.

### Step 4 (3S ,4S)-N3-(2-(2,6-dichloro-3,5-dimethoxyphenyl)furo[2,3-c]pyridin-5-yl)tetrahydro-2H-pyran-3,4-diamine

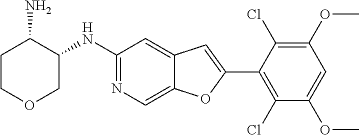 [SMILES: COc1cc(OC)c(Cl)c(-c2cc3cc(N[C@@H]4COCC[C@@H]4N)ncc3o2)c1Cl]

To a stirred solution of the product of Step 3 (20 mg, 0.043 mmol) in MeOH (1 ml), NaBH₄(10 mg, 0.26 mmol) was added and the resulting mixture was stirred at RT for 20 minutes. The solution was quenched with water and extracted with EA. The organic layer was washed with brine and dried over Na₂SO₄. The solution was filtered and concentrated to give 20 mg of crude title compound as black foam which was used in the next step without further purification. MS (ES+): 438 [M +1]⁺.

### Step 5 N-((3S ,4S)-3-(2-(2,6-dichloro-3,5-dimethoxyphenyl)furo[2,3-c]pyridin-5-ylamino)tetrahydro-2H-pyran-4-yl)acrylamide

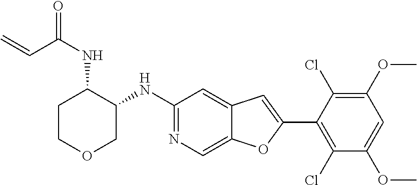 [SMILES: C=CC(=O)N[C@H]1CCOC[C@H]1Nc1cc2cc(-c3c(Cl)c(OC)cc(OC)c3Cl)oc2cn1]

To a stirred solution of the product of Step 4 (20 mg, 0.045 mmol) in DCM (2 ml), Et₃N (0.01 ml), acrylic acid (3 mg, 0.04 mmol), HATU (20 mg, 0.053 mmol) were added at 0-5° C. and stirred for 1 h. The solution was quenched with water and extracted with EA. The organic layer was washed with brine and dried over Na₂SO₄. The solution was filtered and concentrated and purified by prep-TLC (PE:EA=1:2) to give 12 mg of pure product as a yellow solid. MS (ES+): 491.9 [M+1]⁺.

¹HNMR: (400 MHz, CDCl₃) δ 2.02 (m, 2H), 3.48-3.99 (m, 12H), 5.33-5.38 (m, 1H), 5.58-5.61 (d, J=12 Hz, 1H), 6.07 (m,1H), 6.21-6.22 (d, J=4 Hz, 1H), 6.70-6.73 (m, 3H), 7.30 (s, 1H), 8.04 (s, 1H).

### EXAMPLE 38

Inhibitory Activity

Compounds' inhibitory activities against FGFR-4 or FGFR-1 were measured in BaF3 cells expressing Tel-FGFR-4 and luciferase or Tel-FGFR-1 and luciferase, whose growth was dependent on FGFR-4 or FGFR-1 kinase but independent of IL-3. The cells were plated in 384-well plates in RPMI1640 with 10% FBS. Compounds were added as 11-point dilutions. Cell viability was determined by Bright-Glo luciferase assay (Promega) after cells were incubated with compounds for 2 days. The IC₅₀ value was determined as the concentration for 50% growth inhibition compared to DMSO treated cells (A: IC₅₀<0.1 μM; B: IC₅₀ between 0.1 μM and 1 μM; C: IC₅₀ between 1 μM and 10 μM).

TABLE 1
IC₅₀ in BaF3 cellular assays

| | FGFR-4 | FGFR-1 |
|---|---|---|
| Compound | IC₅₀ (μM) | IC₅₀ (μM) |
| Example 1 | B | >3.3 |
| Example 3 | A | >10 |
| Example 5 | A | >10 |
| Example 7 | B | >10 |
| Example 8 | B | >10 |
| Example 10 | B | >3.3 |
| Example 11 | B | C |
| Example 12 | B | >10 |
| Example 13A | A | >10 |
| Example 13B | A | >10 |
| Example 14 | B | >10 |
| Example 16 | A | C |
| Example 17 | A | >10 |
| Example 19 | B | C |
| Example 20 | A | C |
| Example 21 | B | >3.3 |
| Example 22 | B | C |
| Example 23 | A | >6.65 |
| Example 24 | A | >6.65 |
| Example 25 | B | >3.3 |
| Example 26 | A | >5.1 |
| Example 28 | A | >10 |
| Example 29 | A | >10 |
| Example 30 | B | >10 |
| Example 31 | A | C |
| Example 32 | B | >3.3 |
| Example 33 | A | >3.3 |
| Example 34 | C | >10 |
| Example 35 | A | >10 |
| Example 36 | C | >10 |
| Example 37 | B | >10 |

The results suggest that the compounds of invention are specific inhibitors for FGFR-4 because they exhibit superior inhibitory activity against FGFR-4 but poor inhibitory activity against FGFR-1.

What is claimed is:

1. A compound of Formula I or a pharmaceutically acceptable salt thereof, 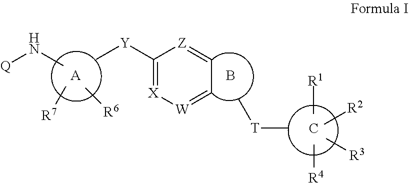
wherein
ring C is a 6-10 membered aryl or 5-12 membered heteroaryl;
each of R¹, R², R³ and R⁴ is independently selected from the group consisting of halo, cyano, C₁₋₆-alkoxy, hydroxy, amino, C(O)NH₂, C(O)NHC₁₋₆alkyl, C(O)N(C₁₋₆alkyl)₂, C₁₋₆alkylsulfonyl, S(O)₂NH₂, S(O)₂NHC₁₋₆alkyl, NHC(O)NH₂, NHC(O)NHC₁₋₆alkyl, C₁₋₆alkyl, NHC(O)OC₁₋₆alkyl, C(O)—C₁₋₆alkyl, —C(O)C₁₋₆alkylamino, C₁₋₆heteroalkyl, heterocyclyl, and heterocyclylalkyl, wherein each of R¹, R², R³ and R⁴ is independently substituted with 0-5 R¹⁰; or each of R¹, R², R³ and R⁴ together with the neighboring group can form a substituted or unsubstituted 5-12 membered carbocyclyl, or substituted or unsubstituted 5-12 membered heterocyclyl;
each R¹⁰ is, independently, selected from C₁₋₆ alkyl, C₁₋₆ alkoxy, halo, hydroxy, oxo, amino, cyano, 5-12 membered cycloalkyl or 5-12 membered heterocyclyl;
Q is a moiety capable of forming a covalent bond with a nucleophile and is one of: 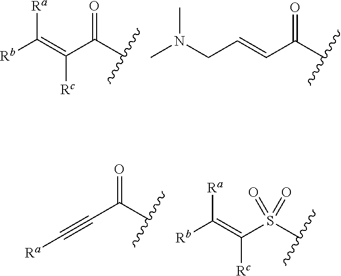
each R^{a}, R^{b}, or R^{c} is, independently, H, halogen, substituted or unsubstituted C₁₋₄alkyl, substituted or unsubstituted C₁₋₄cycloalkyl, or cyano;
ring A is a substituted or unsubstituted 5-10 membered aryl, substituted or unsubstituted 5-12 membered heteroaryl, substituted or unsubstituted 3-7 membered heterocyclyl or substituted or unsubstituted 3-12 membered cyclyloalkyl group; R⁶ and R⁷ are each independently selected from H, halogen, C₁₋₆ alkyl, C₁₋₆ halogenated alkyl, C₁₋₆ cycloalkyl, C₁₋₆ halogenated cycloalkyl, C₃₋₁₀ heterocyclic ring, or R⁸;
R⁸ is selected from H, halogen, C₁₋₆ alkyl, C₁₋₆ alkoxy, C₁₋₆halogenated alkyl, C₁₋₆cycloalkyl, C₁₋₆cycloalkoxy, C₁₋₆halogenated cycloalkyl, C₃₋₁₀heterocyclic ring, C₆₋₁₀aryl, or C₃₋₁₀heteroaryl, or R⁸ is a group selected from the following: 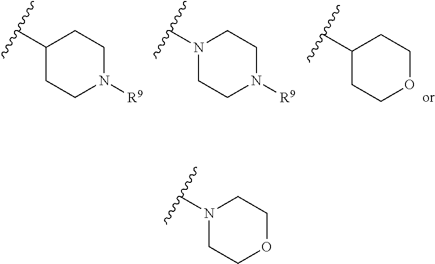
R⁹ is C₁₋₆ alkyl, C(O)R, (C(O)N(R)₂, C(S)R, C(S)N(R)₂, S(O)₂R, S(O)₂N(R)₂, R is selected from H, halogen, C₁₋₆ alkyl, C₁₋₆ alkoxy, 1-6halogenated alkyl, 1-6halogenated alkyl, C₁₋₆cycloalkyl, C₁₋₆cycloalkoxy, C₁₋₆halogenated cycloalkyl, C₃₋₁₀heterocyclic ring, C₆₋₁₀aryl, or C₃₋₁₀heteroaryl;
Y is NH, O, S, CH₂ or Y is a nullity;
each of X, W and Z is, independently, N or CR⁵; R⁵ is H, halogen, C₁₋₆ alkyl, C₁₋₆ halogenated alkyl;
Ring B is a 6-membered aryl, 5-membered heteroaryl, 5-7 membered heterocyclyl or 3-7 membered cyclyloalkyl groups, and Ring B is unsubstituted or substituted by 1 to 3 R^{d};
R^{d} is halogen, cyano, C₁₋₆ alkyl; C₁₋₆ halogenated alkyl, C₁₋₆ alkoxy, or R^{e};
T is C(O), C(S), C(O)NR^{e}, C(S)NR^{e}, NR^{e}C(O)NR^{e}, NR^{e}C(S)NR^{e}, S(O)₂, S(O)₂NR^{e}, [C(R^{e})₂]_{q} or NR^{e}; q is 1 to 3;
and R^{e} is, independently, H, halogen, C₁₋₆ alkyl and C₁₋₆ halogenated alky, OH, OC₁₋₈ alkyl, OC₁₋₈ cycloalkyl, O-aryl, O-heteroaryl; alternatively, together with the carbon atom they are attached to, two R^{e} forms a 3 to 6-membered carbocyclic ring or heterocyclic ring in which one or more than one carbon atom can be replaced with a heteroatom such as O, S, S(O)₂ or NR^{e};
with the proviso R^{e} is not halogen when R^{e} is on a nitrogen atom.

2. The compound of Formula I or pharmaceutically acceptable salt thereof according to claim 1, wherein ring A is substituted or unsubstituted cyclobutyl, substituted or unsubstituted cyclopentyl, substituted or unsubstituted cyclohexyl, substituted or unsubstituted pyrrolidinyl, substituted or unsubstituted piperidinyl, substituted or unsubstituted tetrahydrofuranyl, substituted or unsubstituted tetrohydropiranyl, substituted or unsubstituted phenyl, substituted or unsubstituted pyridyl, or substituted or unsubstituted pyrazolyl.

3. The compound of Formula I or pharmaceutically acceptable salt thereof according to claim 1, wherein the ring A is of a structure selected from the group consisting of: 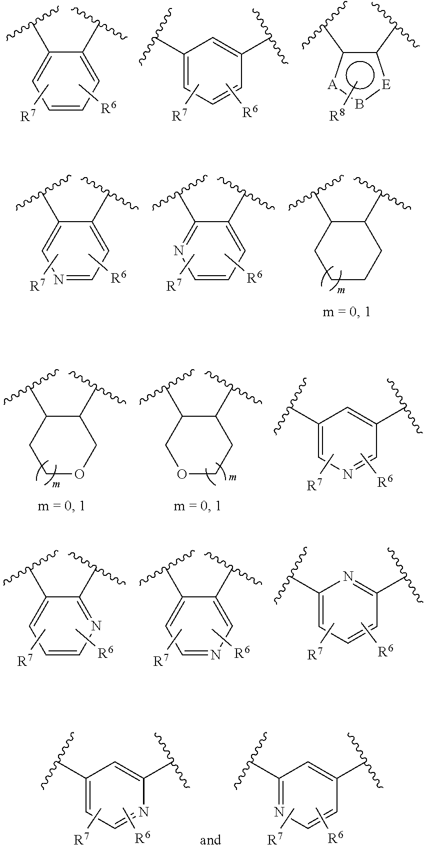
in which two of A, B, and E are N, and the other is C.

4. The compound of Formula I or pharmaceutically acceptable salt thereof according to claim 1, wherein structures of 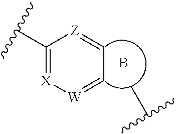
are selected from the group consisting of: 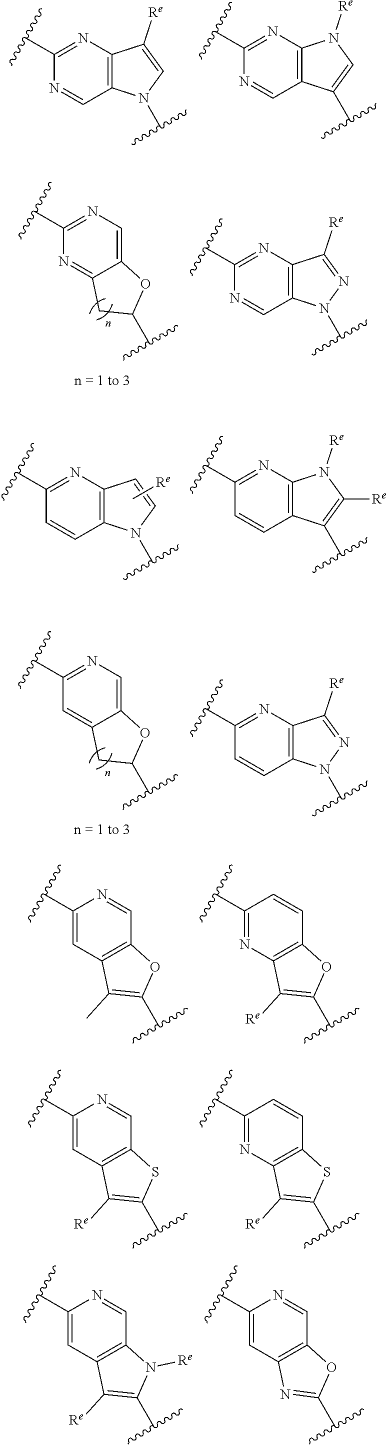 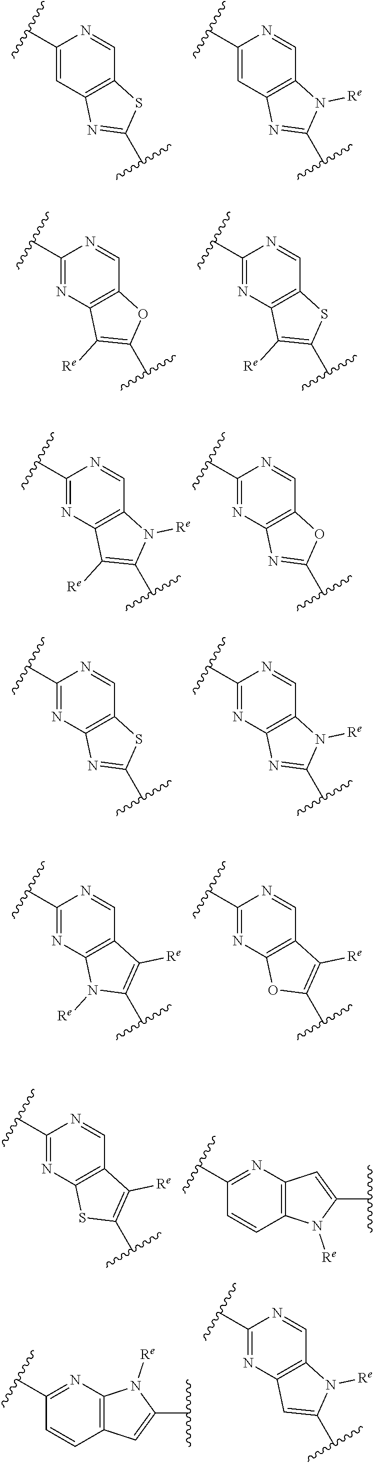 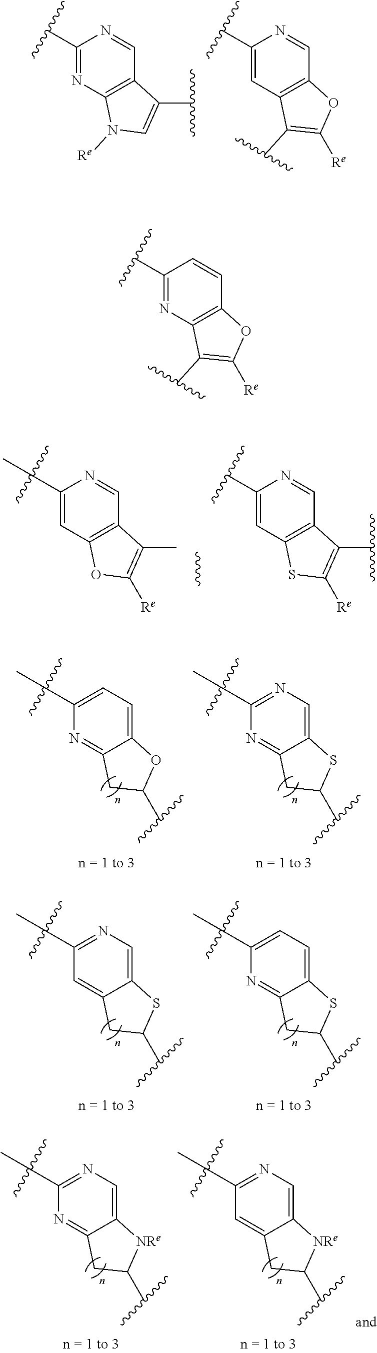 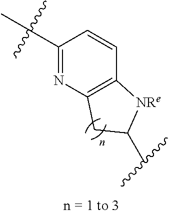
Ring B is substituted with 1 to 3 R^{e}, with the proviso R^{e} is not halogen when R^{e} is on a nitrogen atom.

5. The compound of Formula I or pharmaceutically acceptable salt thereof according to claim 1, wherein 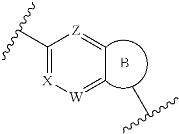
is selected from the group consisting of: 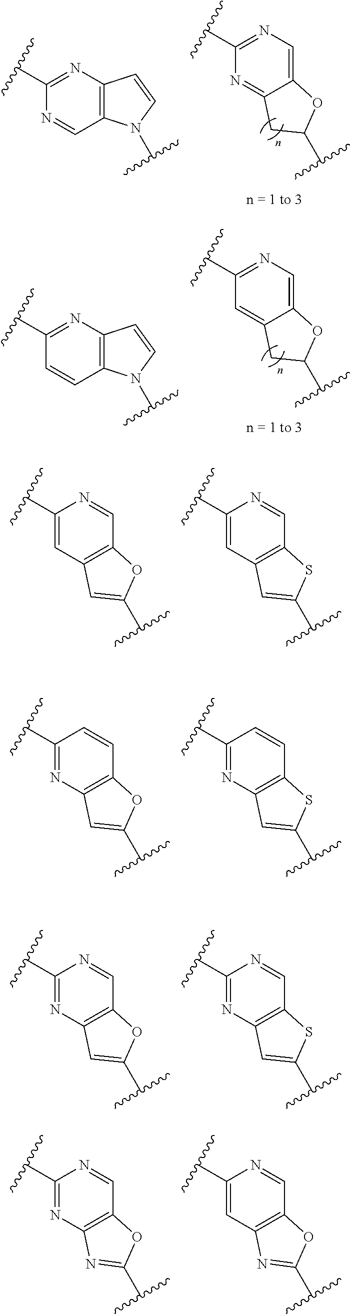 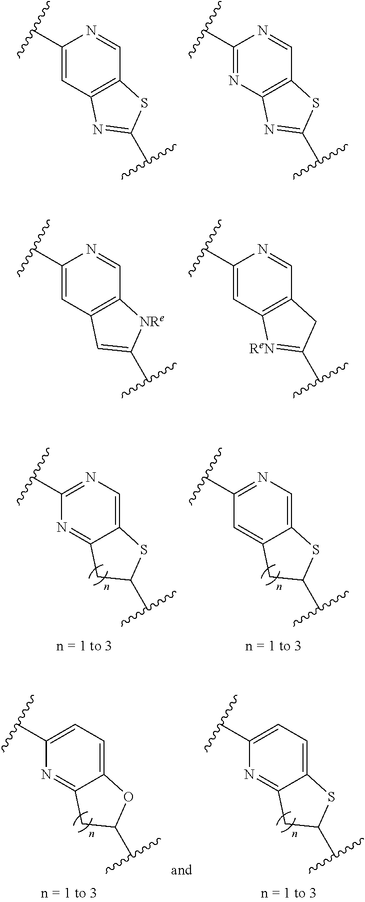
Ring B is substituted with 1 to 3 R^{e}, with the proviso R^{e} is not halogen when R^{e} is on a nitrogen atom.

6. The compound of Formula I or pharmaceutically acceptable salt thereof according to claim 1, wherein 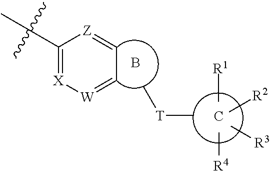
is shown as below: 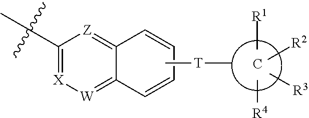
T is C(O), C(S), C(O)NR^{e}, C(S)NR^{e}, NR^{e}C(O)NR^{e}, NR^{e}C(S)NR^{e}, S(O)₂, S(O)₂NR^{e}, [C(R^{e})₂]_{q}, with the proviso that T is not a bond; q is 1 to 3; X, W, Z and R^{e} is defined as in claim 1; Ring C is a 6-10 membered aryl or 5-12 membered heteroaryl;
each of R¹, R², R³ and R⁴ is, independently, halo, cyano, C₁₋₆ alkoxy, hydroxy, amino, C(O)NH₂, C(O)NHC₁₋₆ alkyl, C(O)N(C₁₋₆ alkyl)₂, C₁₋₆ alkylsulfonyl, S(O)₂NH₂, S(O)₂NHC₁₋₆ alkyl, NHC(O)NH₂, NHC(O)NHC₁₋₆alkyl, C₁₋₆alkyl, NHC(O)OC₁₋₆alkyl, C(O)-C₁₋₆-alkyl, —C(O)C₁₋₆ alkylamino, C₁₋₆ heteroalkyl, heterocyclyl, or heterocyclylalkyl, wherein each of R¹, R², R³ and R⁴ is independently substituted with 0-5 R¹⁰; or each of R¹, R², R³ and R⁴ together with the neighboring group can form a 5-12 membered carbocyclyl, 5-12 membered heterocyclyl;
each R¹⁰ is, independently, selected from C₁₋₆ alkyl, C₁₋₆-alkoxy, halo, hydroxy, oxo, amino, cyano, cycloalkyl and heterocyclyl.

7. The compound of Formula I or pharmaceutically acceptable salt thereof according to claim 1, wherein the compound selected from the group consisting of the compounds below:

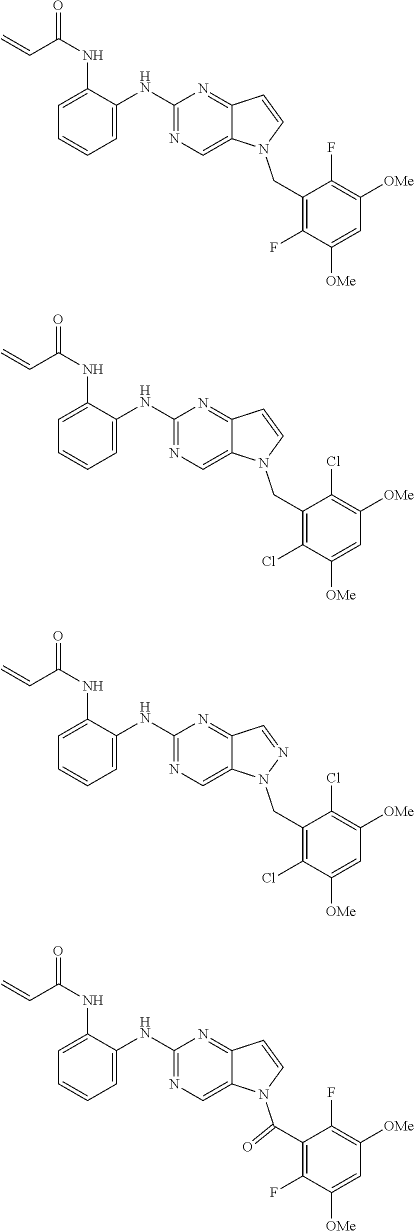 [SMILES: C=CC(=O)Nc1ccccc1Nc1ncc2c(ccn2Cc2c(F)c(OC)cc(OC)c2F)n1 ; C=CC(=O)Nc1ccccc1Nc1ncc2c(ccn2Cc2c(Cl)c(OC)cc(OC)c2Cl)n1 ; C=CC(=O)Nc1ccccc1Nc1ncc2c(cnn2Cc2c(Cl)c(OC)cc(OC)c2Cl)n1 ; C=CC(=O)Nc1ccccc1Nc1ncc2c(ccn2C(=O)c2c(F)c(OC)cc(OC)c2F)n1]

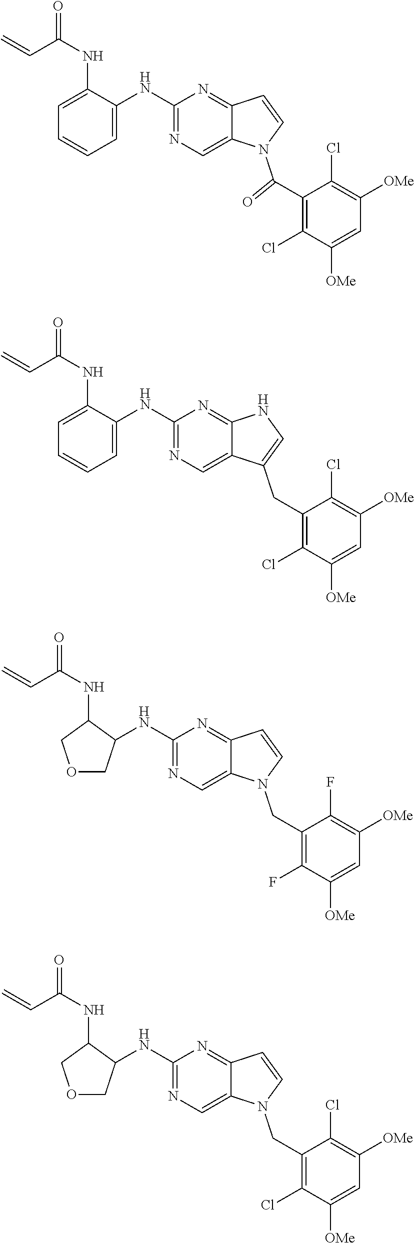 [SMILES: C=CC(=O)Nc1ccccc1Nc1ncc2c(ccn2C(=O)c2c(Cl)c(OC)cc(OC)c2Cl)n1 ; C=CC(=O)Nc1ccccc1Nc1ncc2c(Cc3c(Cl)c(OC)cc(OC)c3Cl)c[nH]c2n1 ; C=CC(=O)NC1COCC1Nc1ncc2c(ccn2Cc2c(F)c(OC)cc(OC)c2F)n1 ; C=CC(=O)NC1COCC1Nc1ncc2c(ccn2Cc2c(Cl)c(OC)cc(OC)c2Cl)n1]

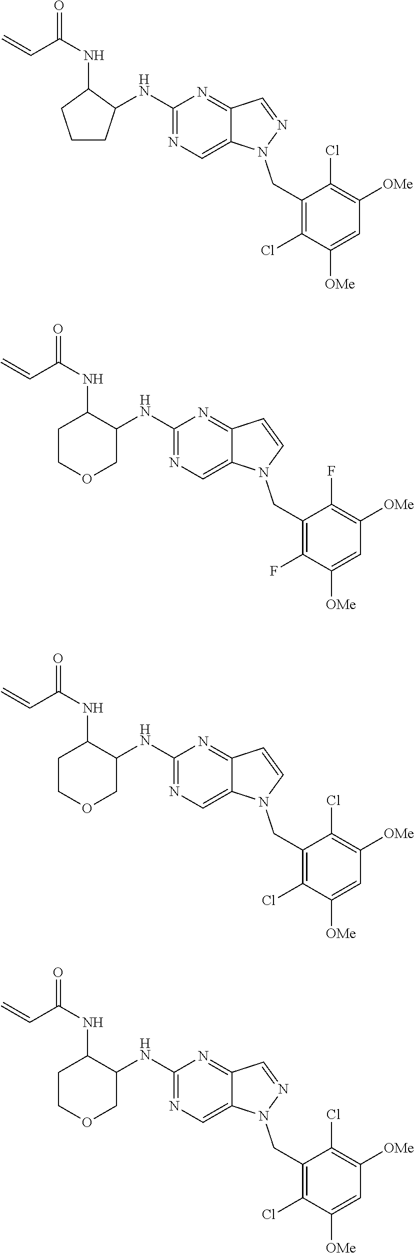 [SMILES: C=CC(=O)NC1CCCC1Nc1ncc2c(cnn2Cc2c(Cl)c(OC)cc(OC)c2Cl)n1 ; C=CC(=O)NC1CCOCC1Nc1ncc2c(ccn2Cc2c(F)c(OC)cc(OC)c2F)n1 ; C=CC(=O)NC1CCOCC1Nc1ncc2c(ccn2Cc2c(Cl)c(OC)cc(OC)c2Cl)n1 ; C=CC(=O)NC1CCOCC1Nc1ncc2c(cnn2Cc2c(Cl)c(OC)cc(OC)c2Cl)n1]

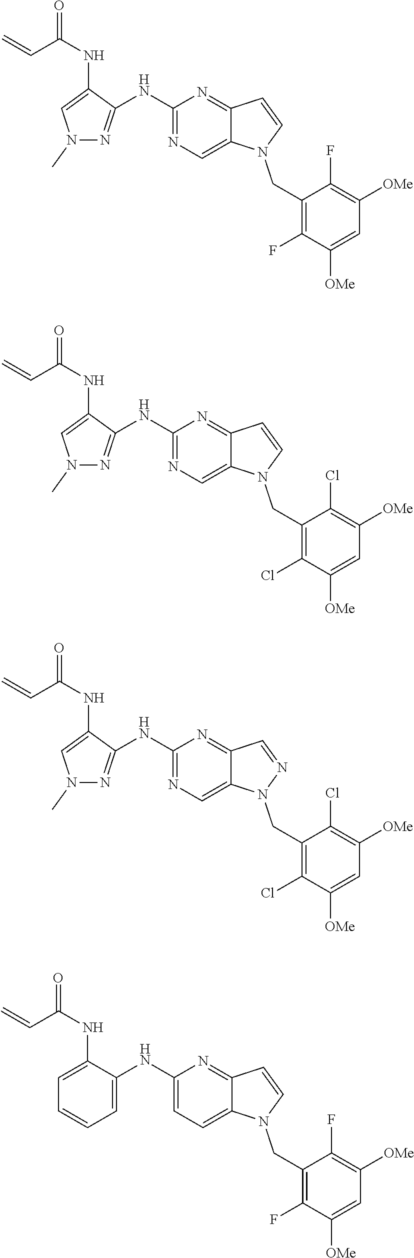 [SMILES: C=CC(=O)Nc1cn(C)nc1Nc1ncc2c(ccn2Cc2c(F)c(OC)cc(OC)c2F)n1 ; C=CC(=O)Nc1cn(C)nc1Nc1ncc2c(ccn2Cc2c(Cl)c(OC)cc(OC)c2Cl)n1 ; C=CC(=O)Nc1cn(C)nc1Nc1ncc2c(cnn2Cc2c(Cl)c(OC)cc(OC)c2Cl)n1 ; C=CC(=O)Nc1ccccc1Nc1ccc2c(ccn2Cc2c(F)c(OC)cc(OC)c2F)n1]

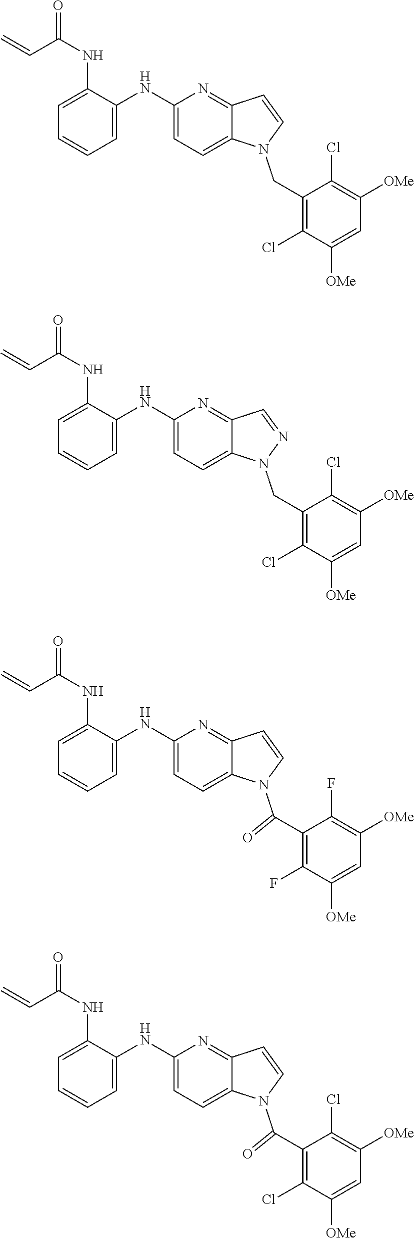 [SMILES: C=CC(=O)Nc1ccccc1Nc1ccc2c(ccn2Cc2c(Cl)c(OC)cc(OC)c2Cl)n1 ; C=CC(=O)Nc1ccccc1Nc1ccc2c(cnn2Cc2c(Cl)c(OC)cc(OC)c2Cl)n1 ; C=CC(=O)Nc1ccccc1Nc1ccc2c(ccn2C(=O)c2c(F)c(OC)cc(OC)c2F)n1 ; C=CC(=O)Nc1ccccc1Nc1ccc2c(ccn2C(=O)c2c(Cl)c(OC)cc(OC)c2Cl)n1]

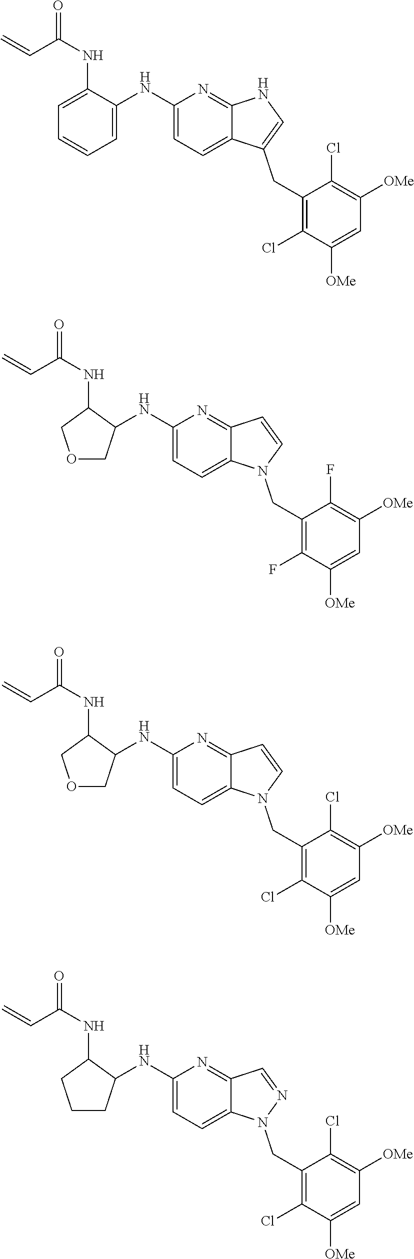 [SMILES: C=CC(=O)Nc1ccccc1Nc1ccc2c(Cc3c(Cl)c(OC)cc(OC)c3Cl)c[nH]c2n1 ; C=CC(=O)NC1COCC1Nc1ccc2c(ccn2Cc2c(F)c(OC)cc(OC)c2F)n1 ; C=CC(=O)NC1COCC1Nc1ccc2c(ccn2Cc2c(Cl)c(OC)cc(OC)c2Cl)n1 ; C=CC(=O)NC1CCCC1Nc1ccc2c(cnn2Cc2c(Cl)c(OC)cc(OC)c2Cl)n1]

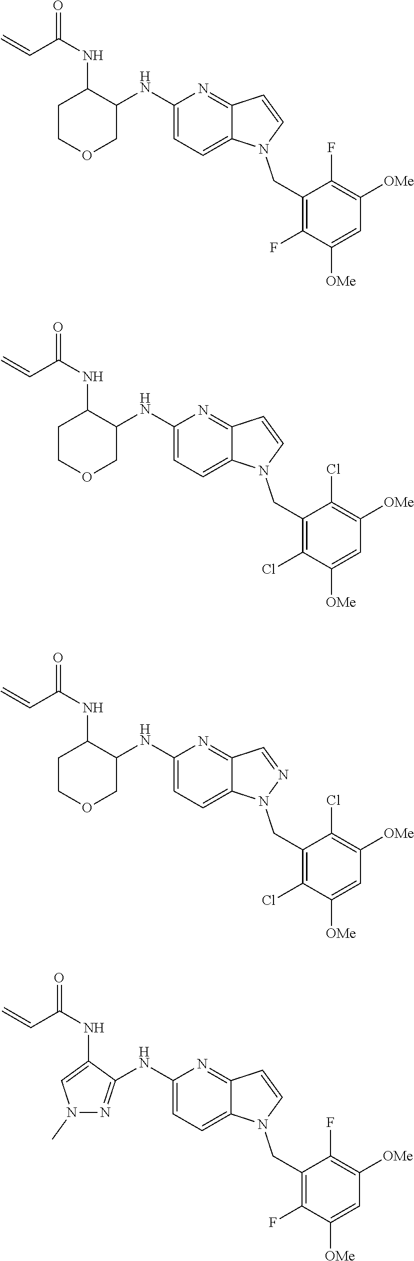 [SMILES: C=CC(=O)NC1CCOCC1Nc1ccc2c(ccn2Cc2c(F)c(OC)cc(OC)c2F)n1 ; C=CC(=O)NC1CCOCC1Nc1ccc2c(ccn2Cc2c(Cl)c(OC)cc(OC)c2Cl)n1 ; C=CC(=O)NC1CCOCC1Nc1ccc2c(cnn2Cc2c(Cl)c(OC)cc(OC)c2Cl)n1 ; C=CC(=O)Nc1cn(C)nc1Nc1ccc2c(ccn2Cc2c(F)c(OC)cc(OC)c2F)n1]

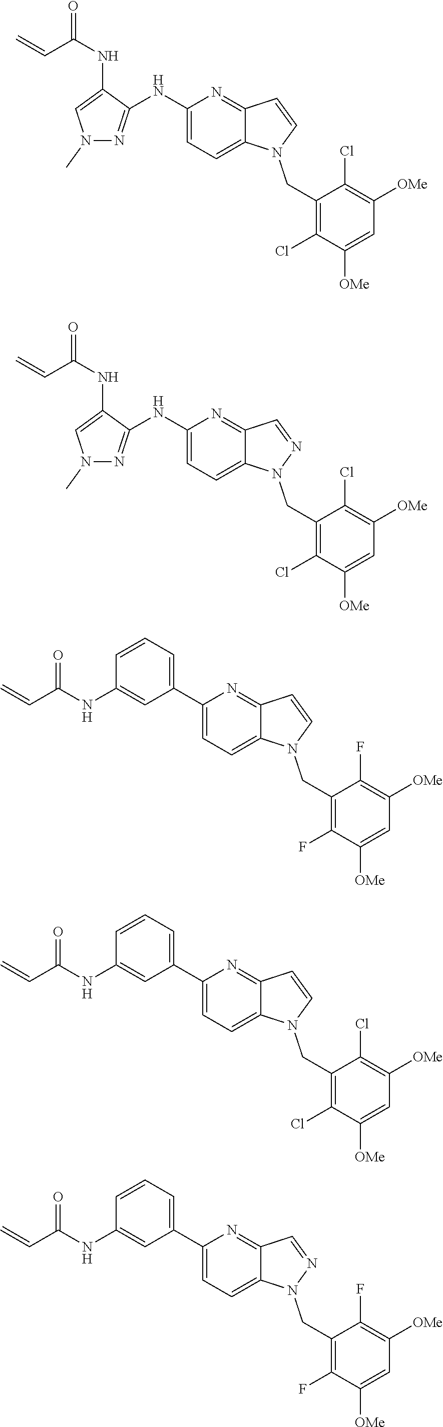 [SMILES: C=CC(=O)Nc1cn(C)nc1Nc1ccc2c(ccn2Cc2c(Cl)c(OC)cc(OC)c2Cl)n1 ; C=CC(=O)Nc1cn(C)nc1Nc1ccc2c(cnn2Cc2c(Cl)c(OC)cc(OC)c2Cl)n1 ; C=CC(=O)Nc1cccc(-c2ccc3c(ccn3Cc3c(F)c(OC)cc(OC)c3F)n2)c1 ; C=CC(=O)Nc1cccc(-c2ccc3c(ccn3Cc3c(Cl)c(OC)cc(OC)c3Cl)n2)c1 ; C=CC(=O)Nc1cccc(-c2ccc3c(cnn3Cc3c(F)c(OC)cc(OC)c3F)n2)c1]

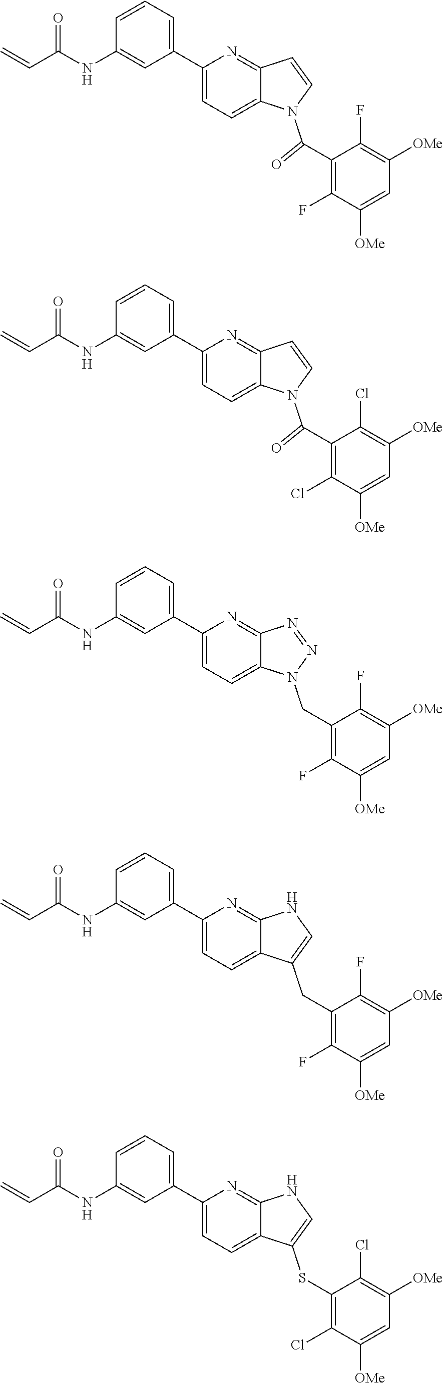 [SMILES: C=CC(=O)Nc1cccc(-c2ccc3c(ccn3C(=O)c3c(F)c(OC)cc(OC)c3F)n2)c1 ; C=CC(=O)Nc1cccc(-c2ccc3c(ccn3C(=O)c3c(Cl)c(OC)cc(OC)c3Cl)n2)c1 ; C=CC(=O)Nc1cccc(-c2ccc3c(nnn3Cc3c(F)c(OC)cc(OC)c3F)n2)c1 ; C=CC(=O)Nc1cccc(-c2ccc3c(Cc4c(F)c(OC)cc(OC)c4F)c[nH]c3n2)c1 ; C=CC(=O)Nc1cccc(-c2ccc3c(Sc4c(Cl)c(OC)cc(OC)c4Cl)c[nH]c3n2)c1]

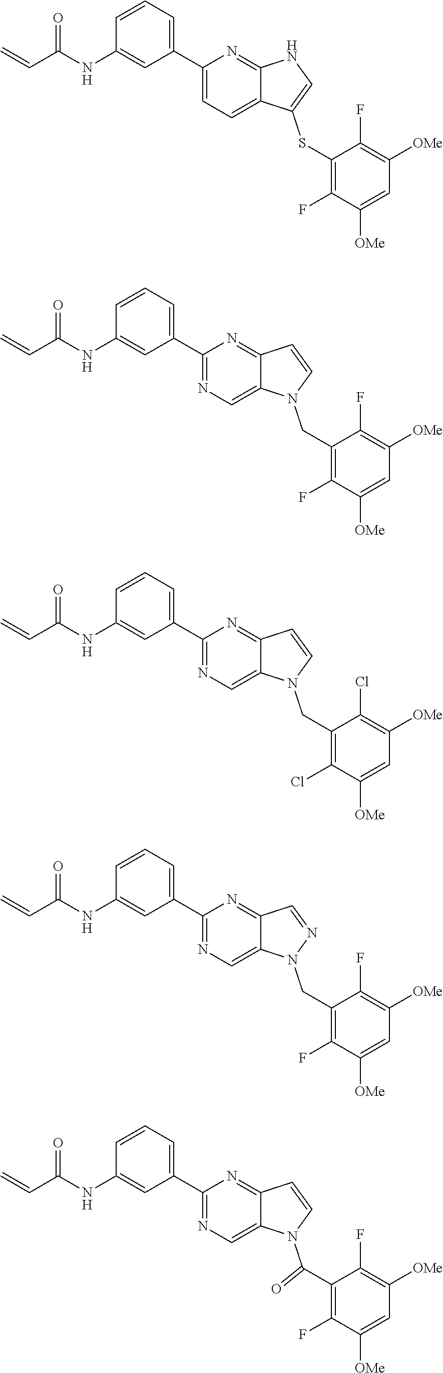 [SMILES: C=CC(=O)Nc1cccc(-c2ccc3c(Sc4c(F)c(OC)cc(OC)c4F)c[nH]c3n2)c1 ; C=CC(=O)Nc1cccc(-c2ncc3c(ccn3Cc3c(F)c(OC)cc(OC)c3F)n2)c1 ; C=CC(=O)Nc1cccc(-c2ncc3c(ccn3Cc3c(Cl)c(OC)cc(OC)c3Cl)n2)c1 ; C=CC(=O)Nc1cccc(-c2ncc3c(cnn3Cc3c(F)c(OC)cc(OC)c3F)n2)c1 ; C=CC(=O)Nc1cccc(-c2ncc3c(ccn3C(=O)c3c(F)c(OC)cc(OC)c3F)n2)c1]

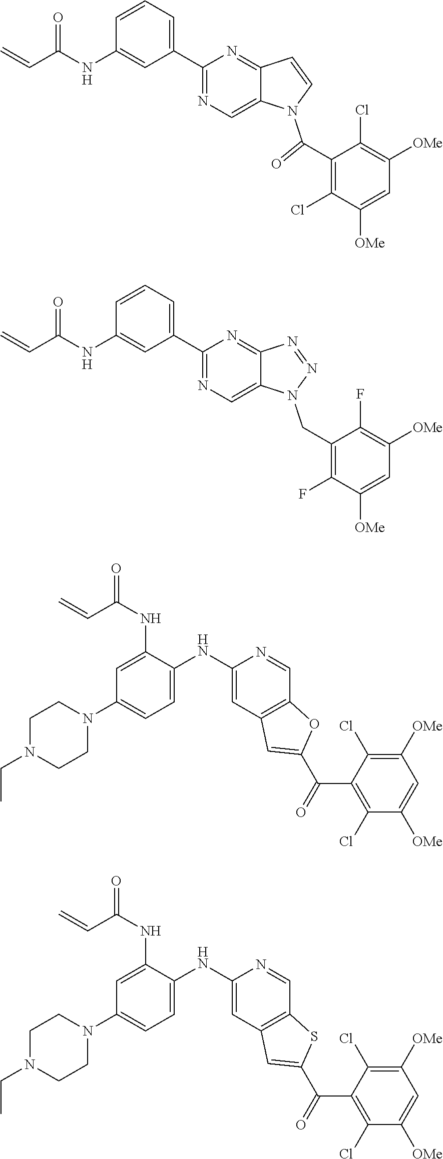 [SMILES: C=CC(=O)Nc1cccc(-c2ncc3c(ccn3C(=O)c3c(Cl)c(OC)cc(OC)c3Cl)n2)c1 ; C=CC(=O)Nc1cccc(-c2ncc3c(nnn3Cc3c(F)c(OC)cc(OC)c3F)n2)c1 ; C=CC(=O)Nc1cc(N2CCN(CC)CC2)ccc1Nc1cc2cc(C(=O)c3c(Cl)c(OC)cc(OC)c3Cl)oc2cn1 ; C=CC(=O)Nc1cc(N2CCN(CC)CC2)ccc1Nc1cc2cc(C(=O)c3c(Cl)c(OC)cc(OC)c3Cl)sc2cn1]

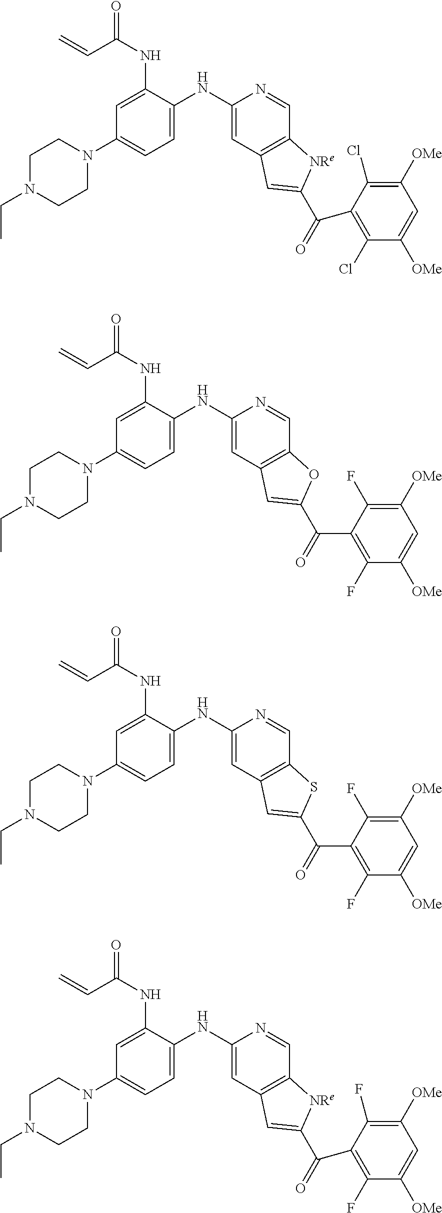

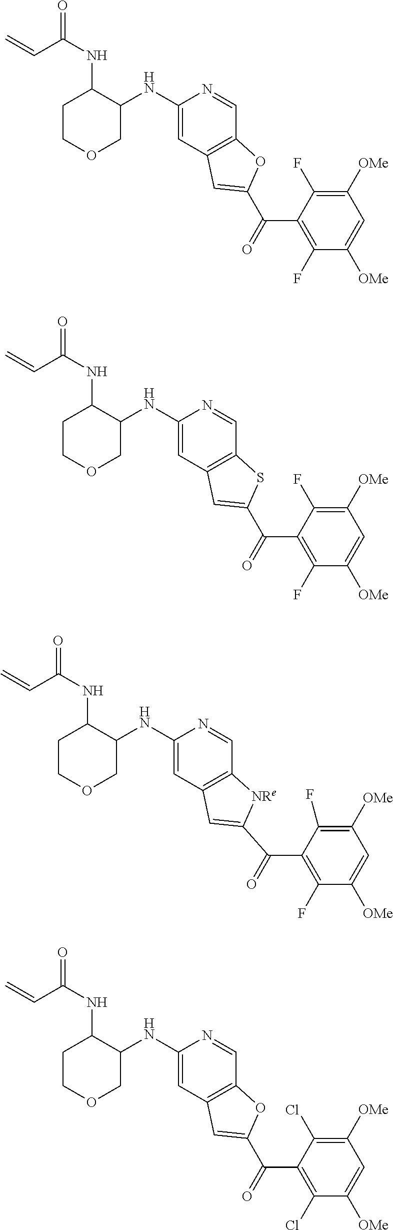

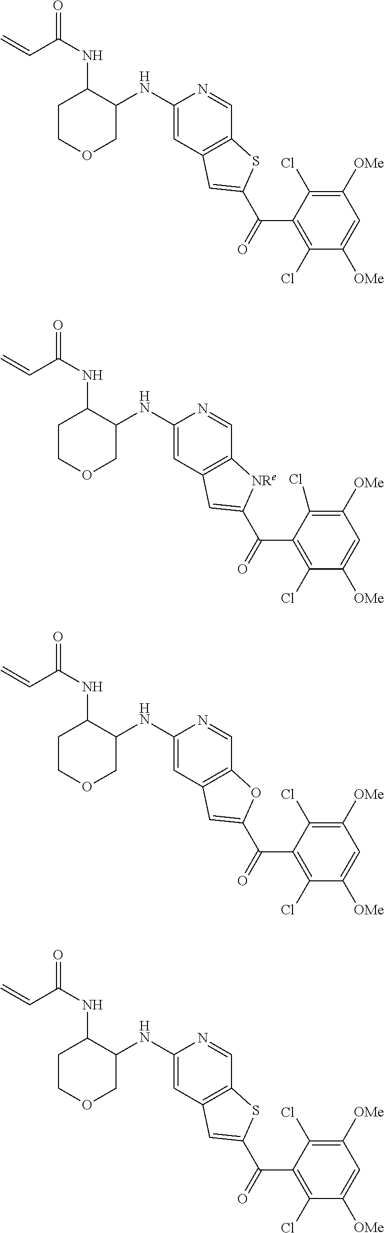

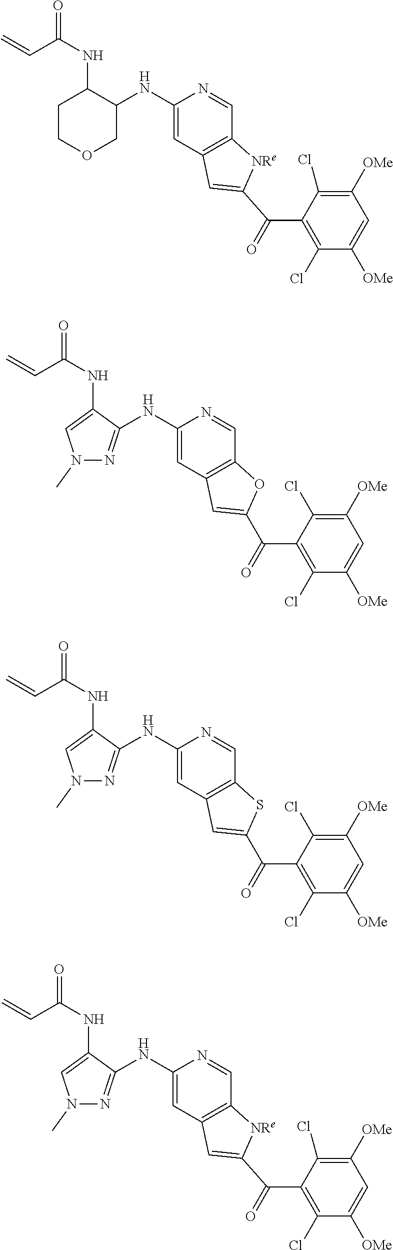

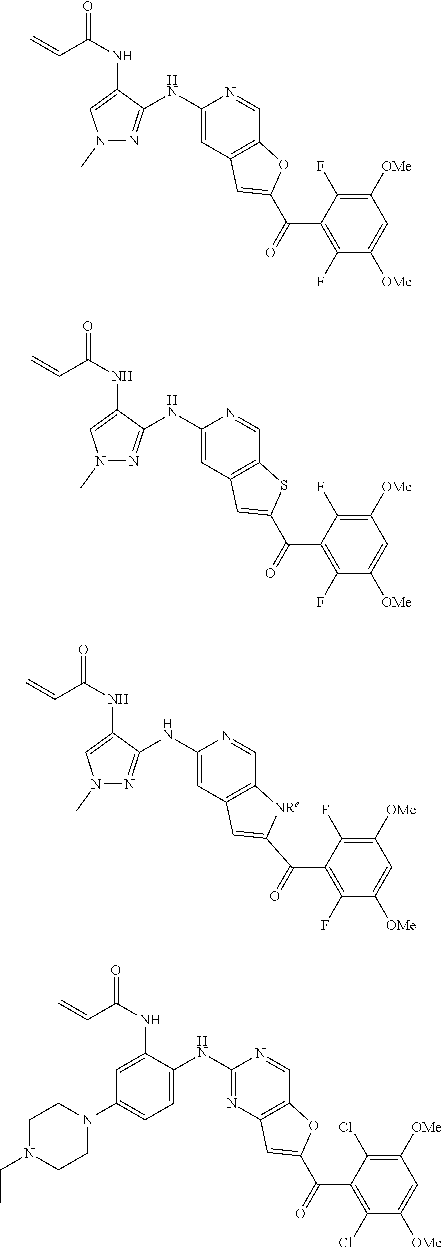

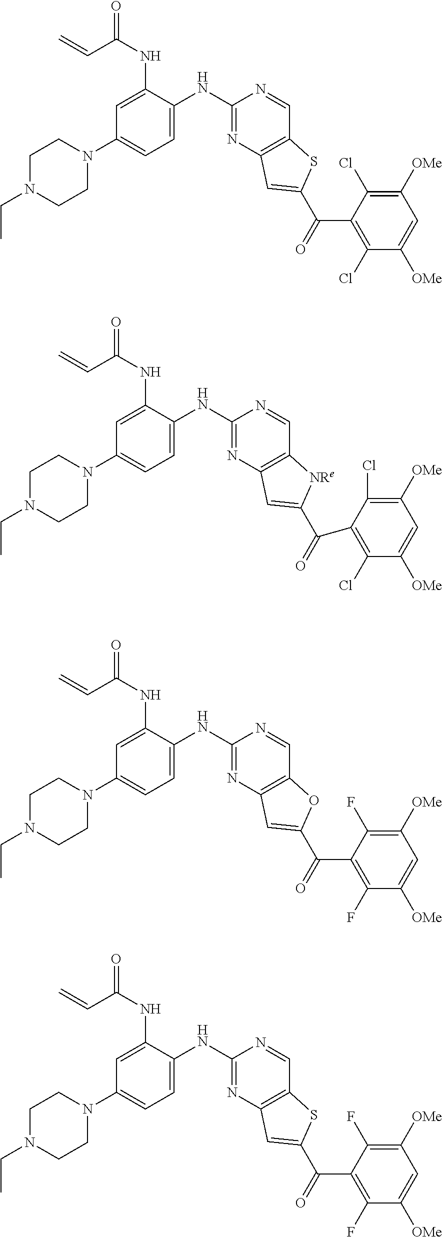

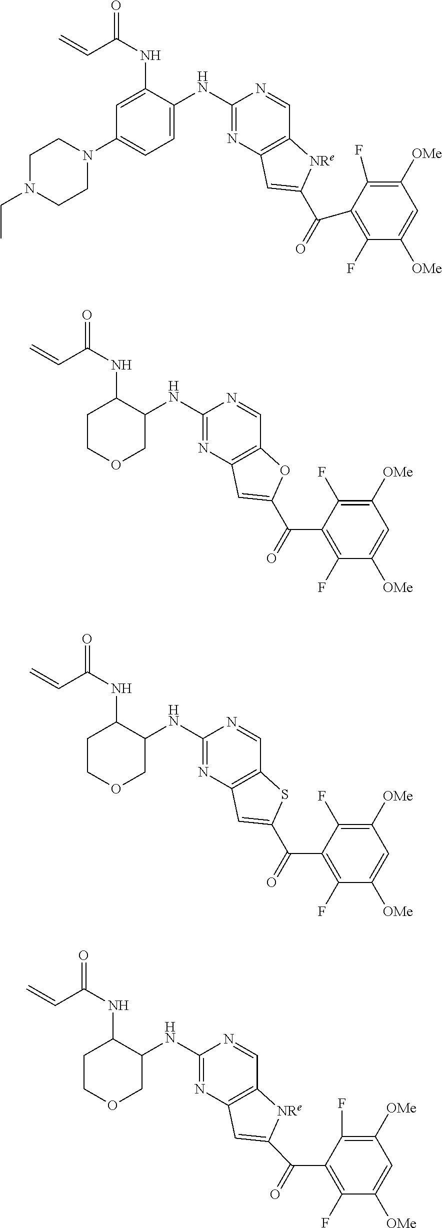

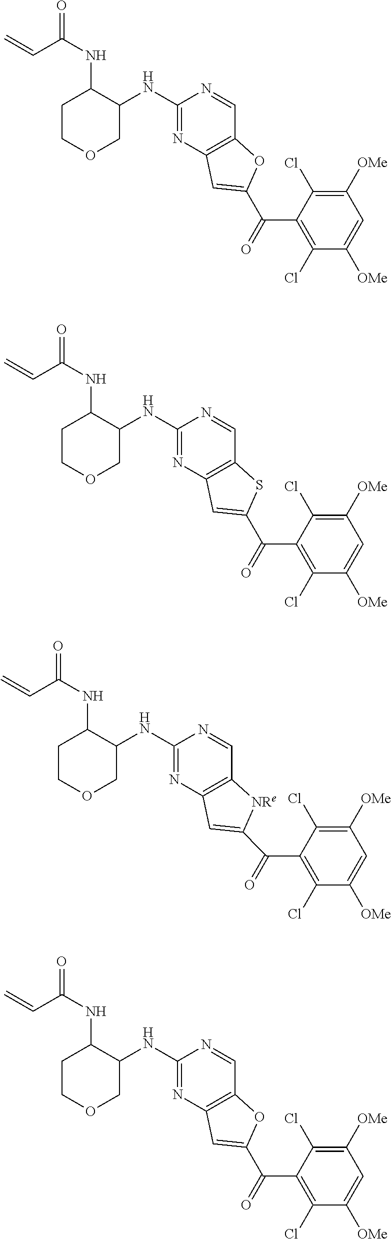

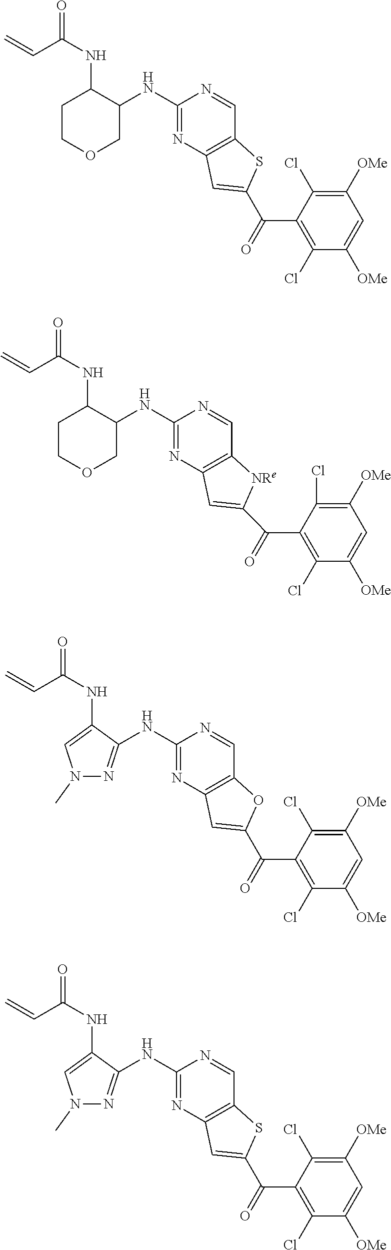

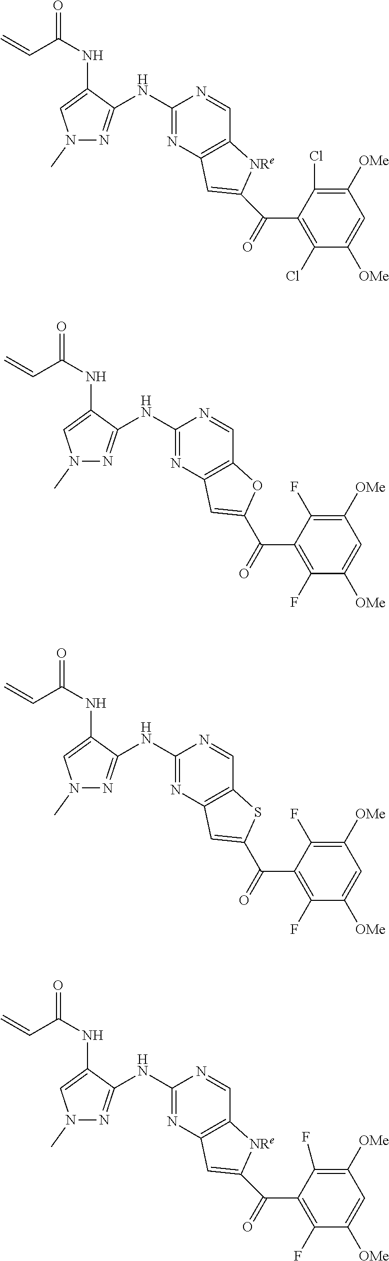

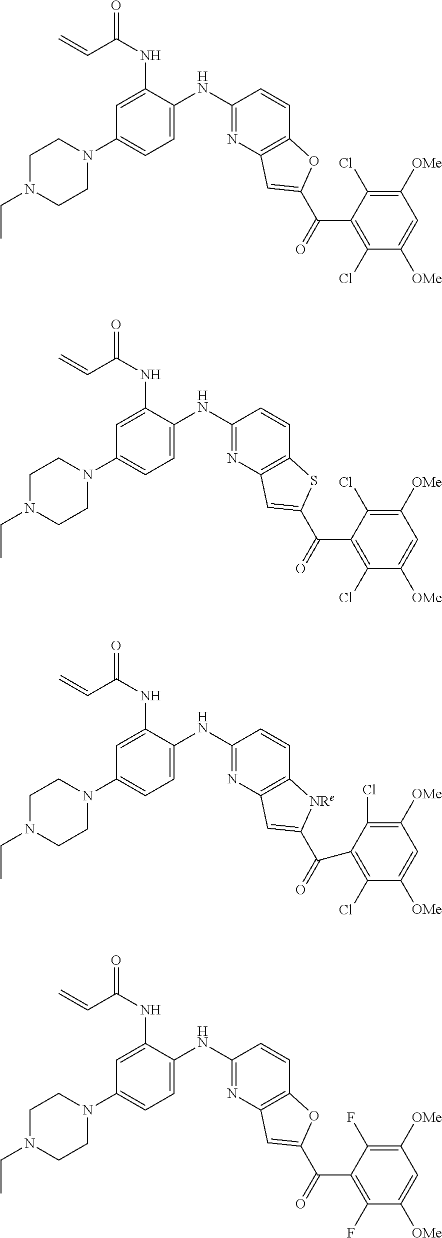

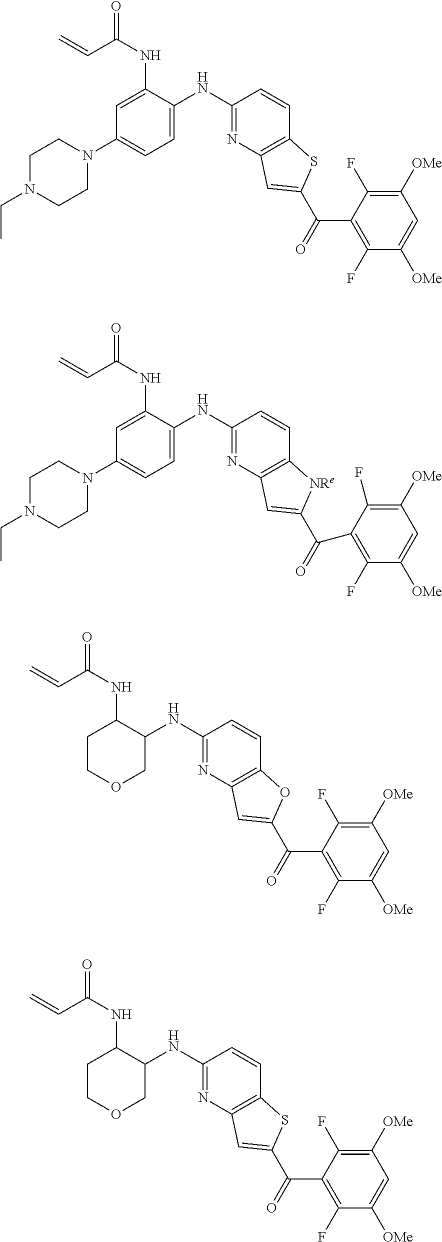

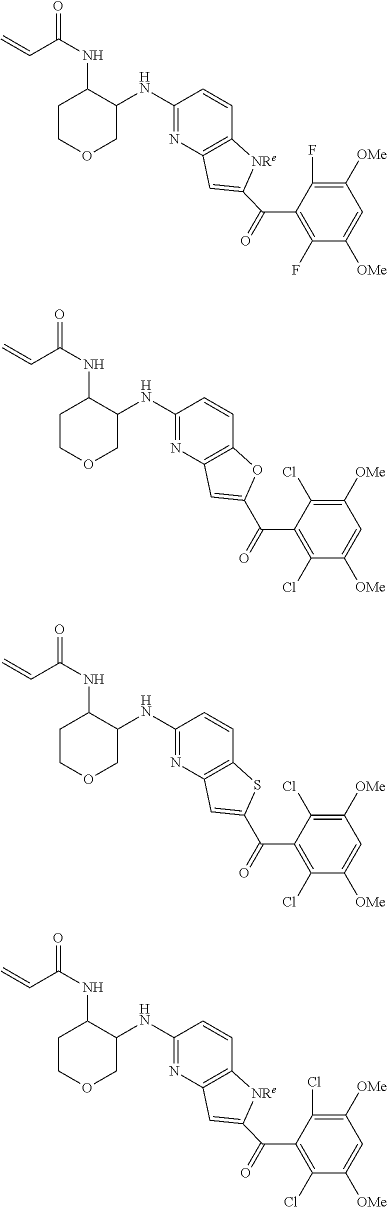

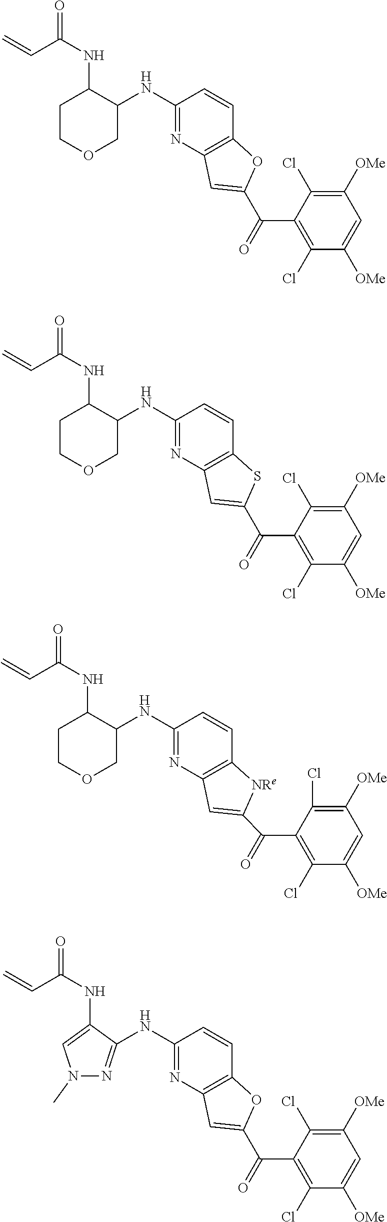

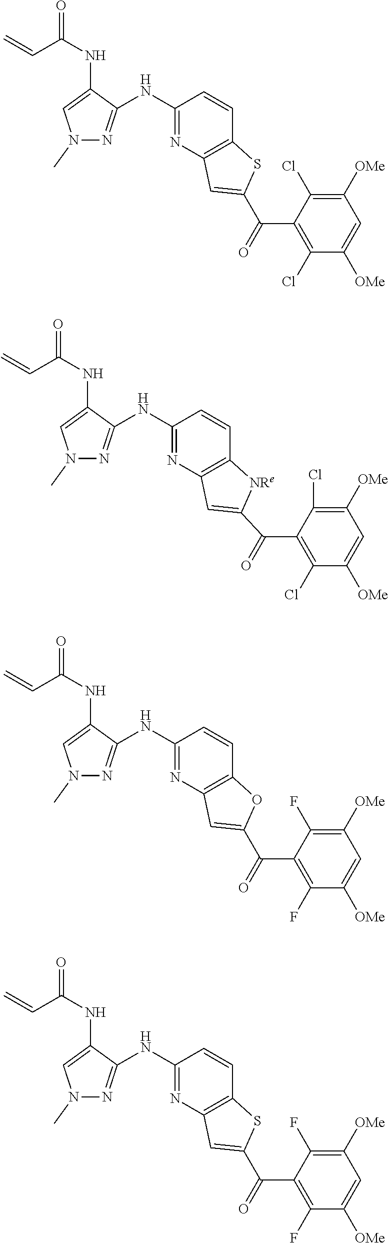

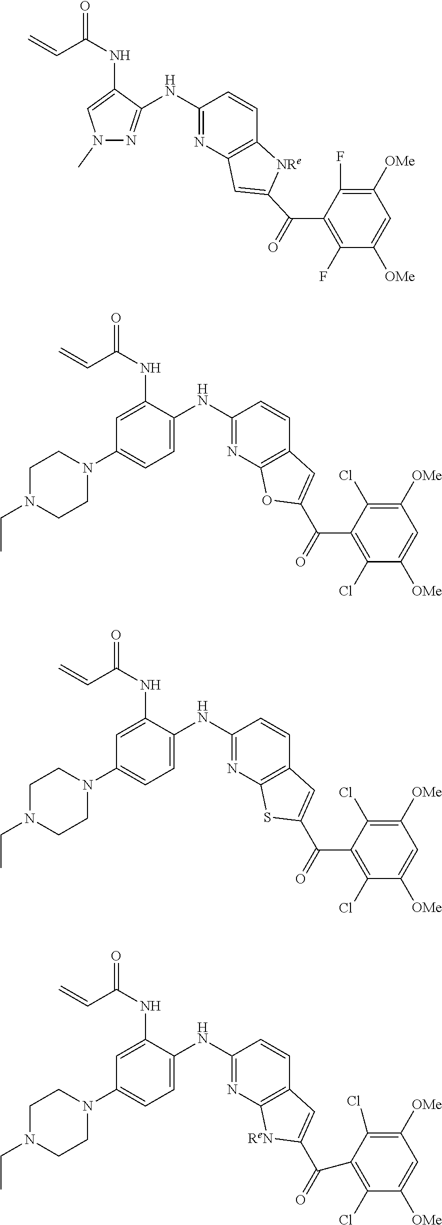

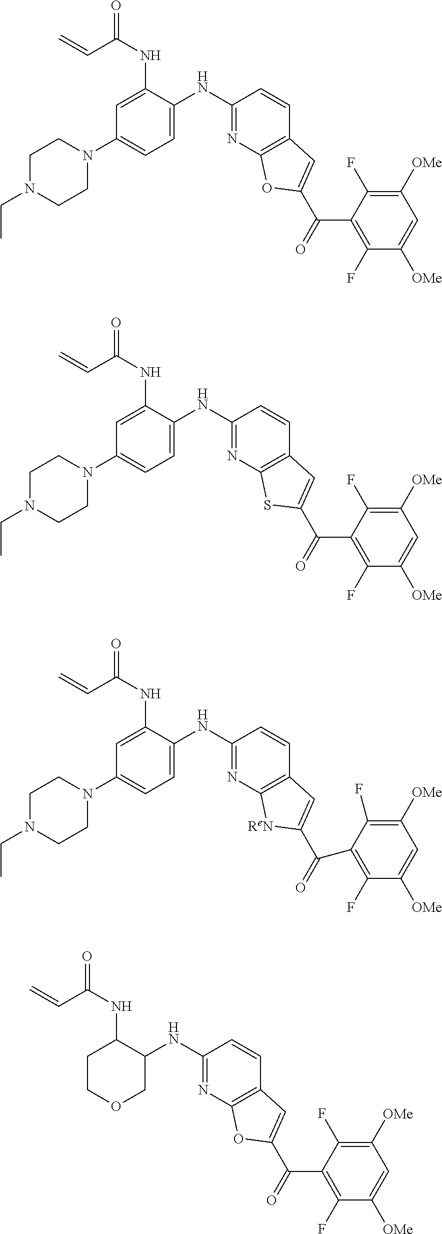

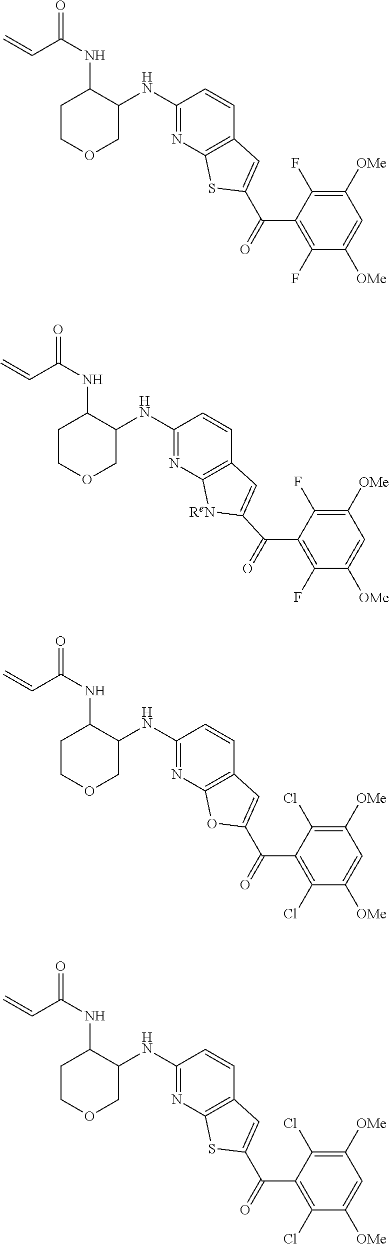

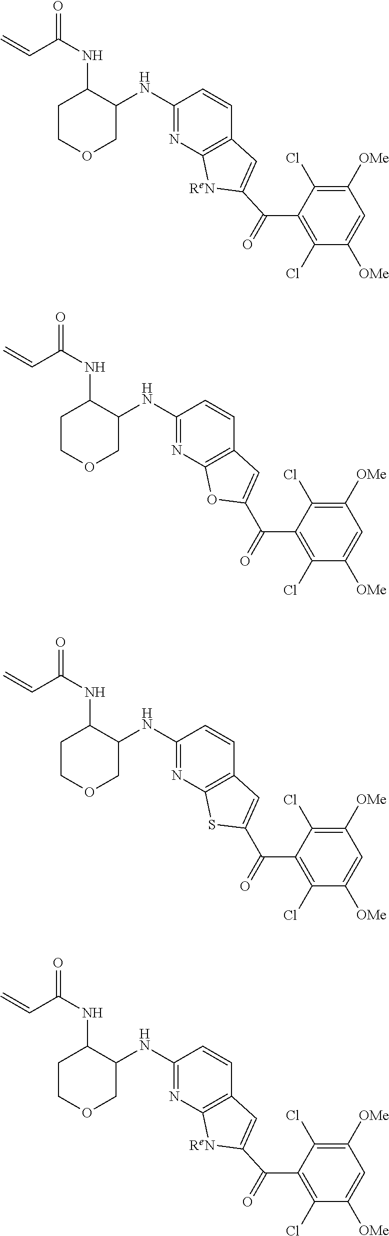

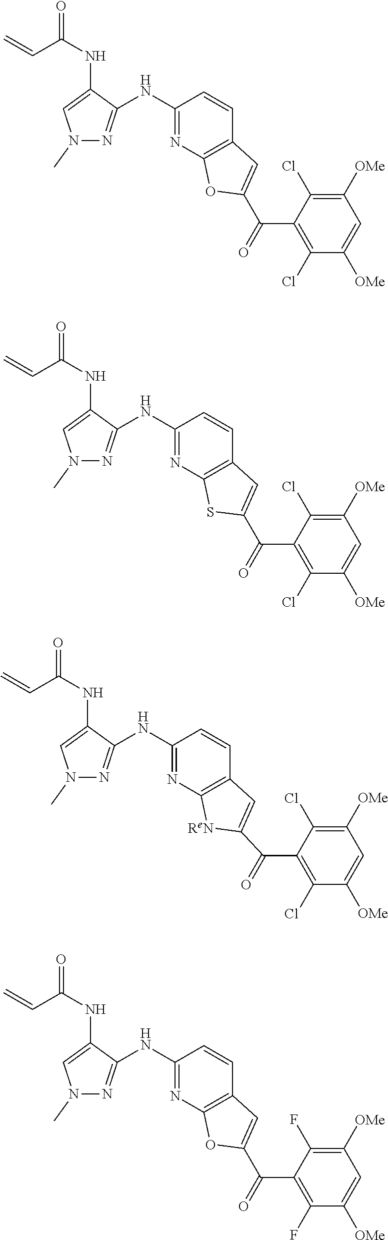

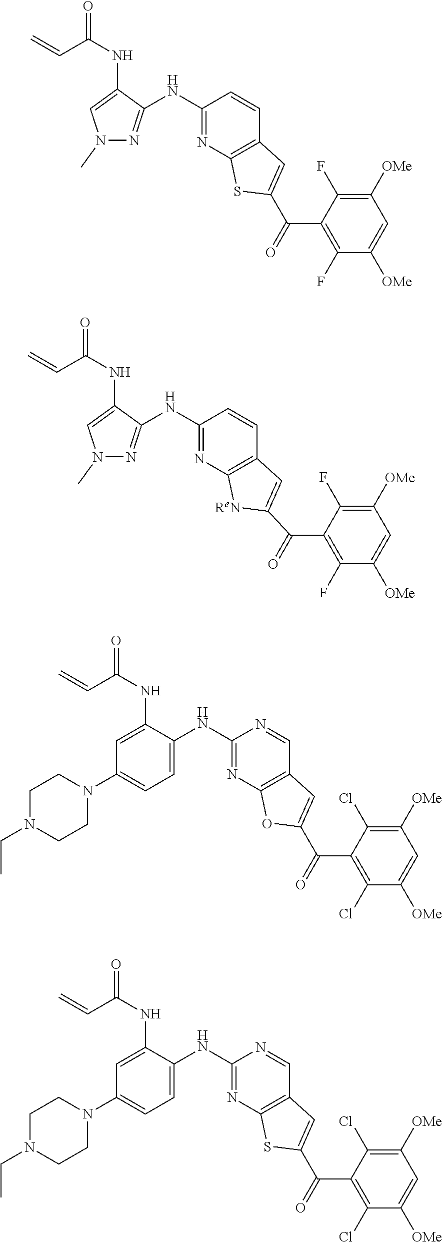

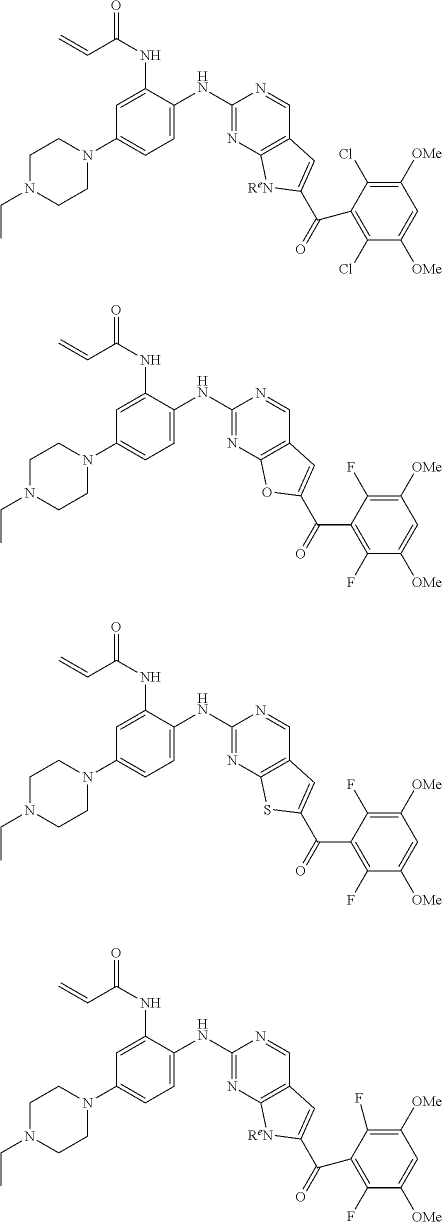

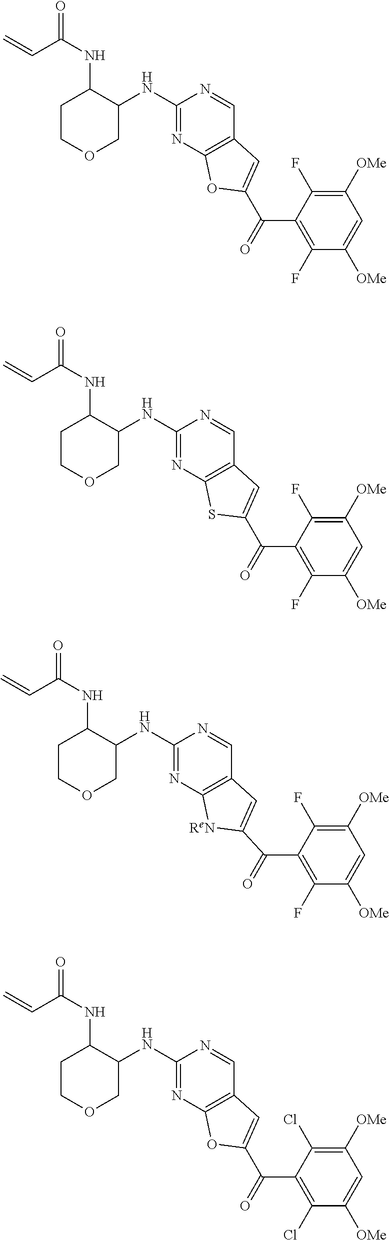

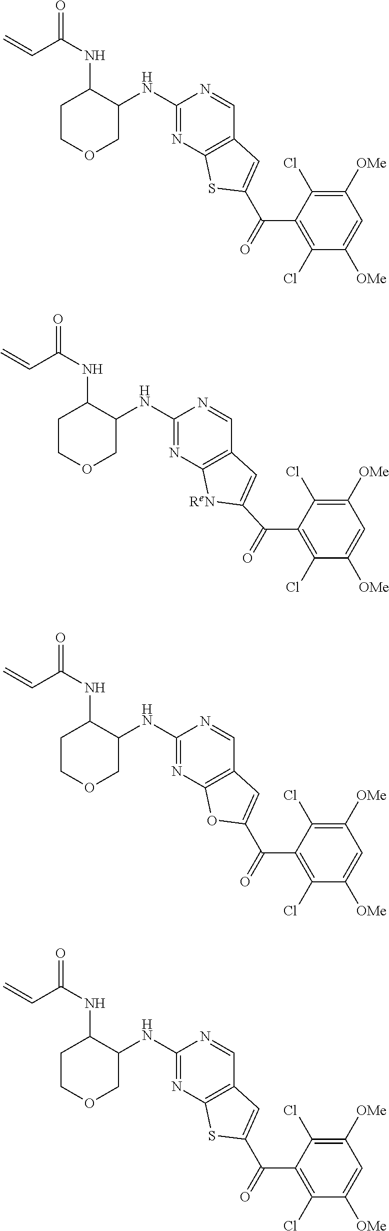

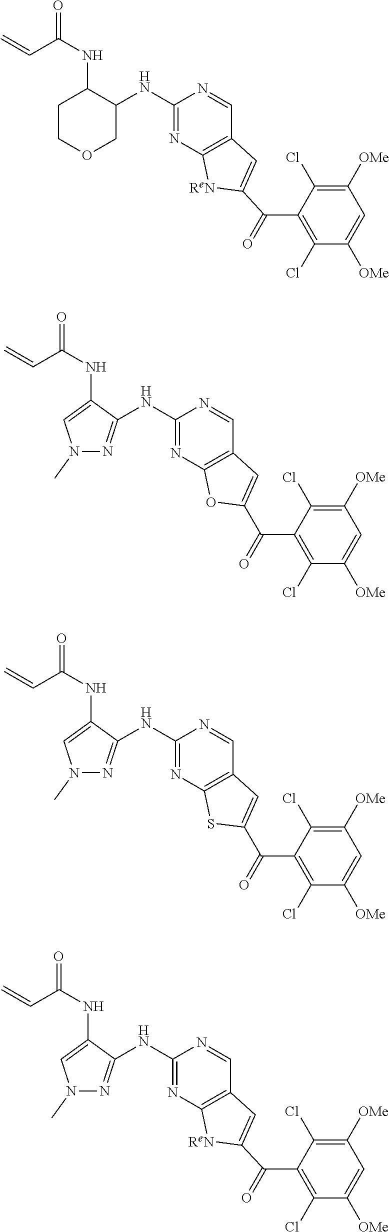

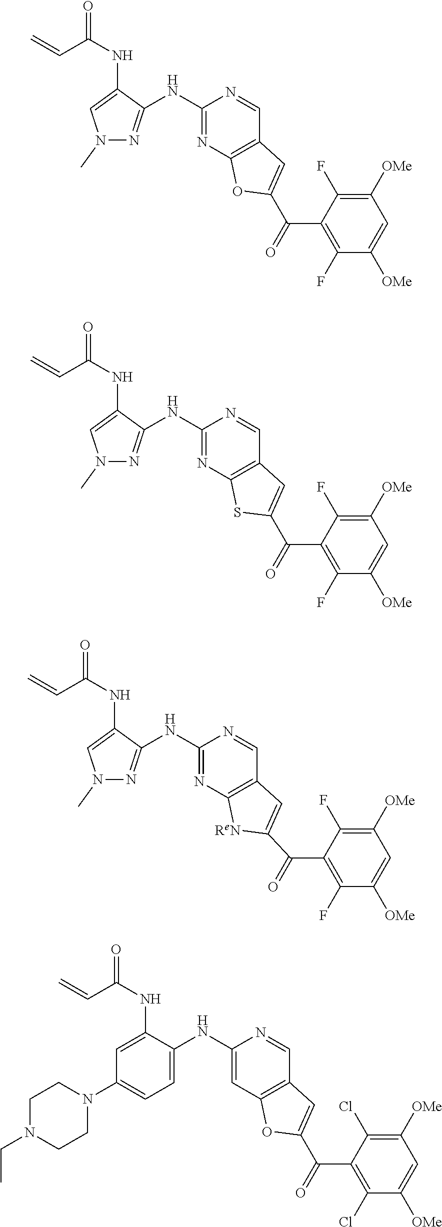

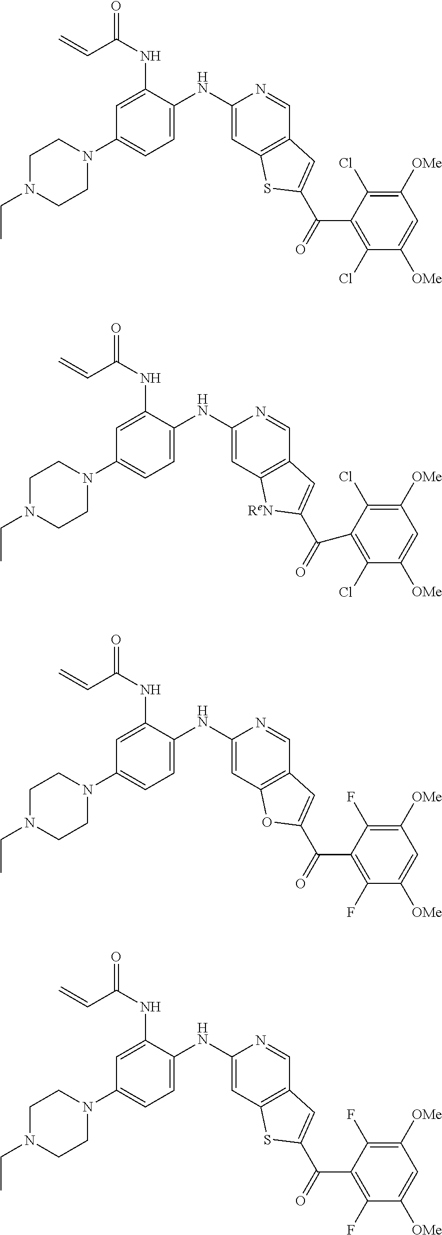

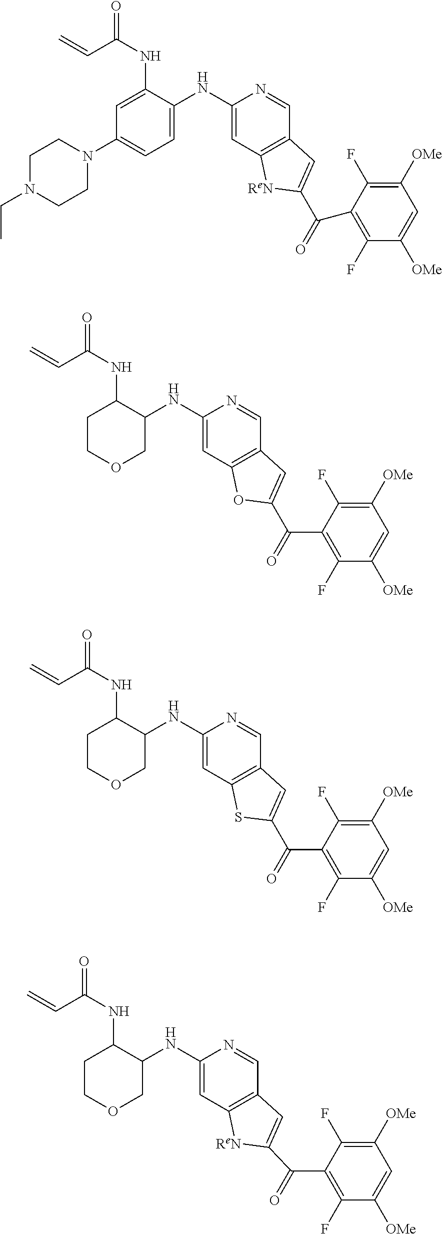

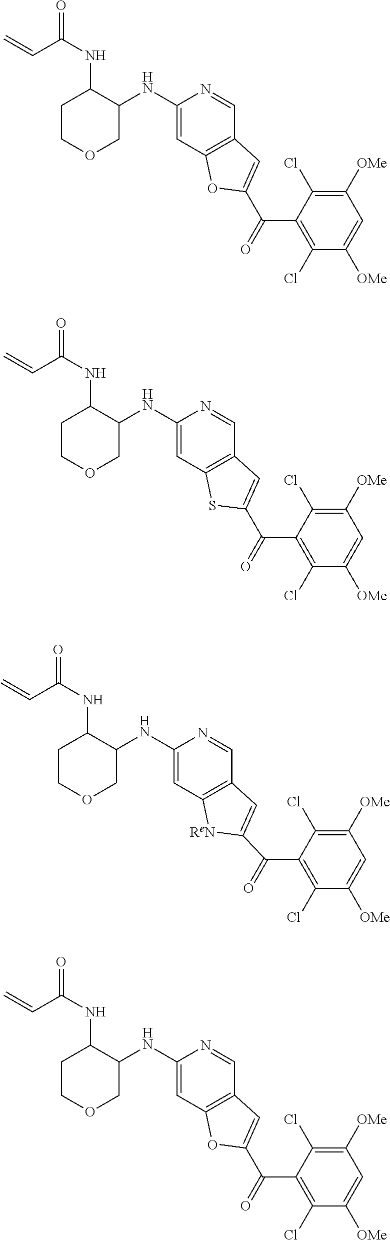

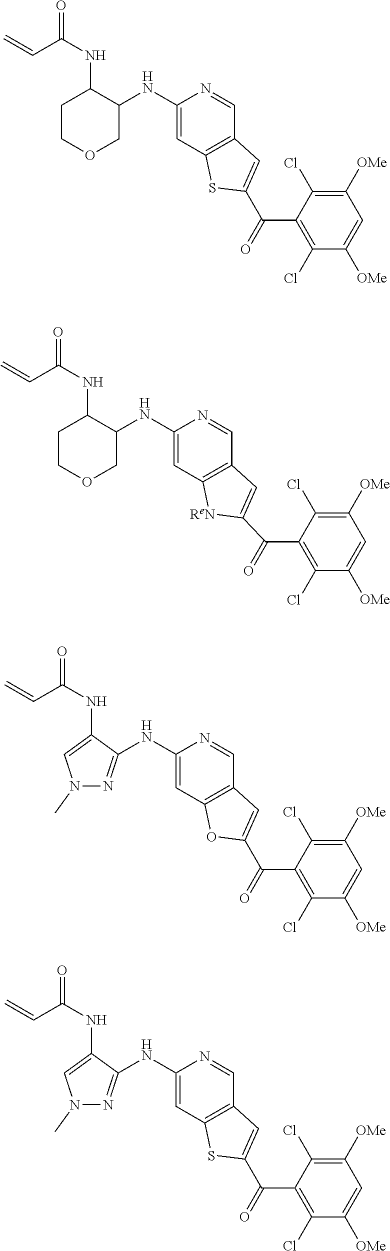

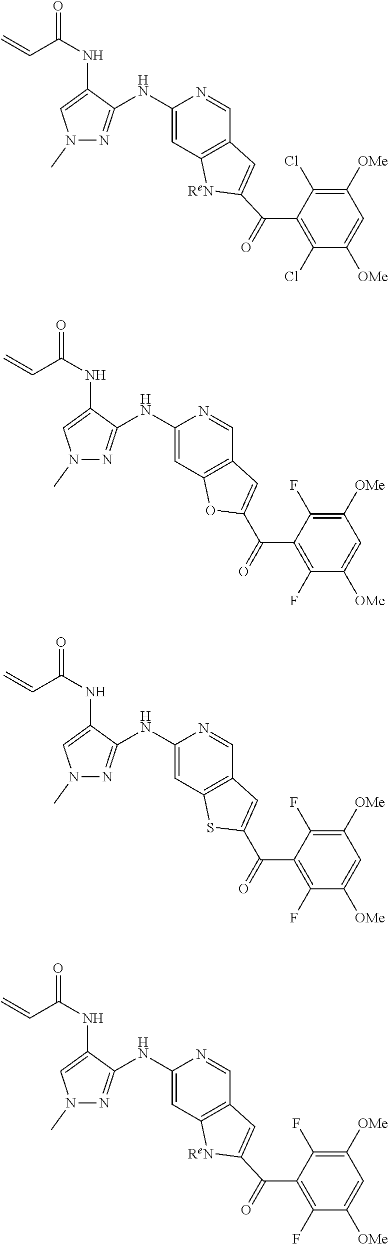

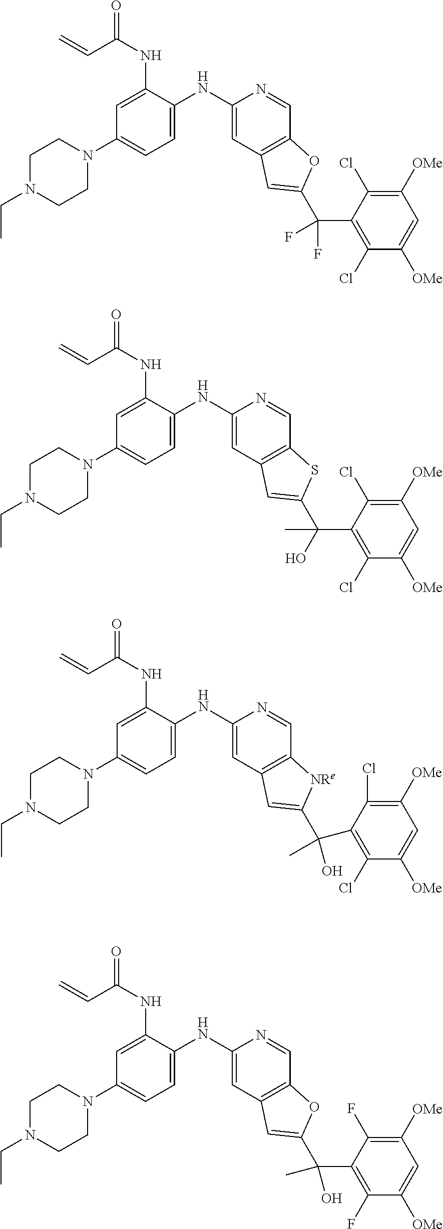

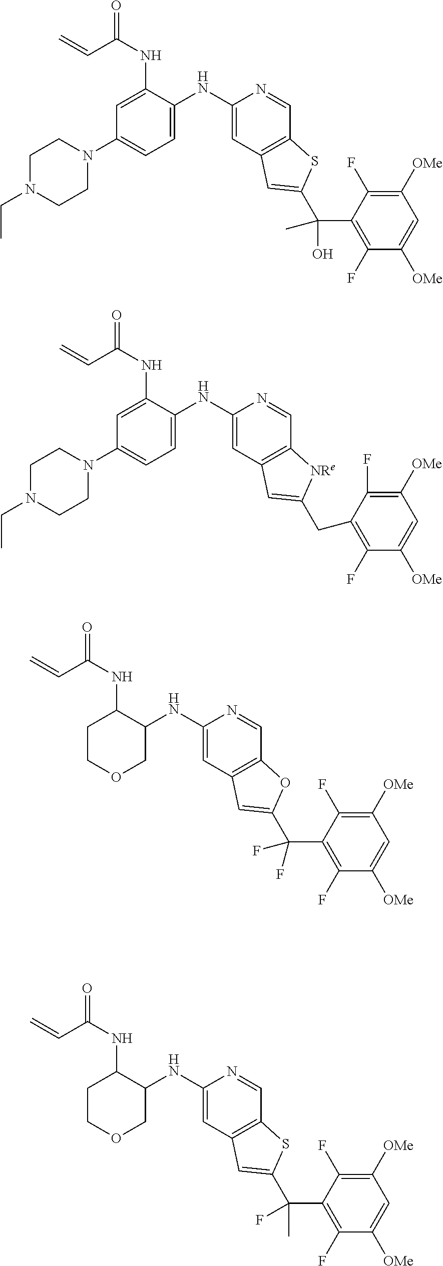

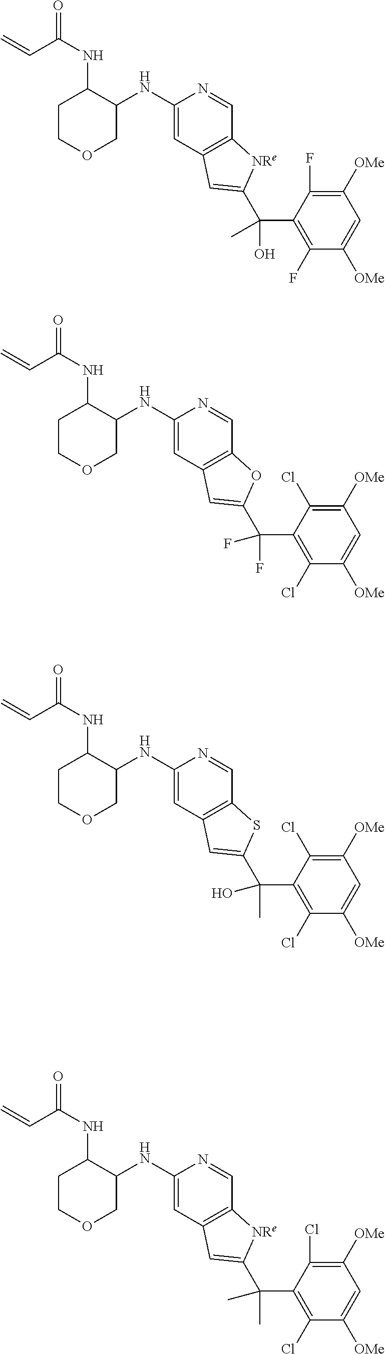

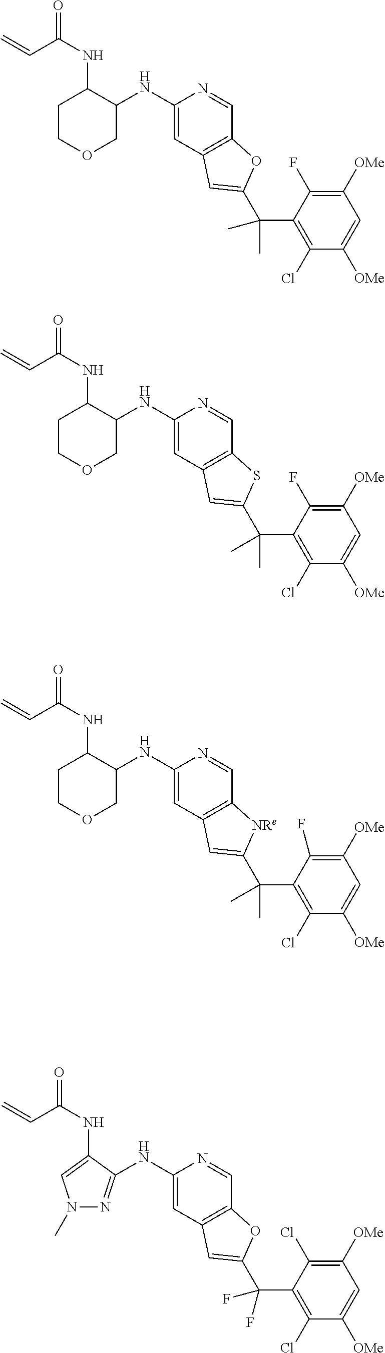

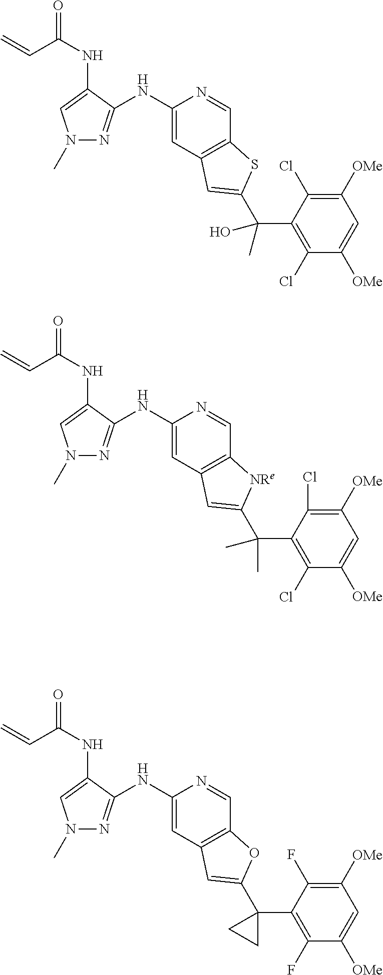

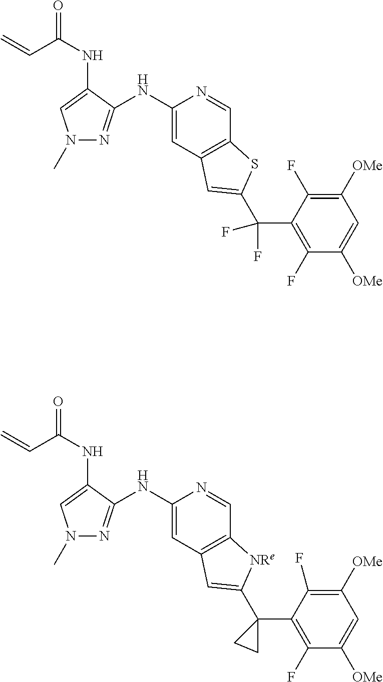

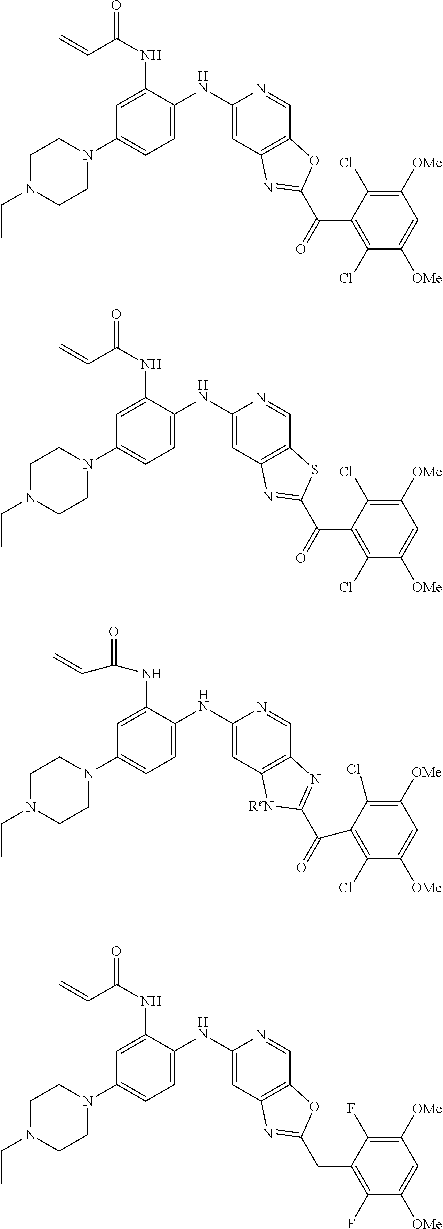

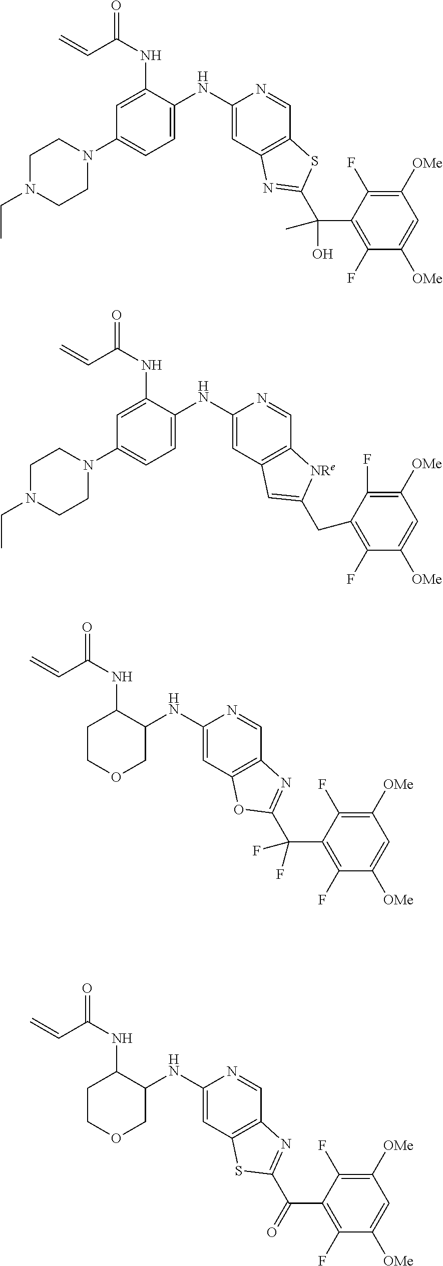

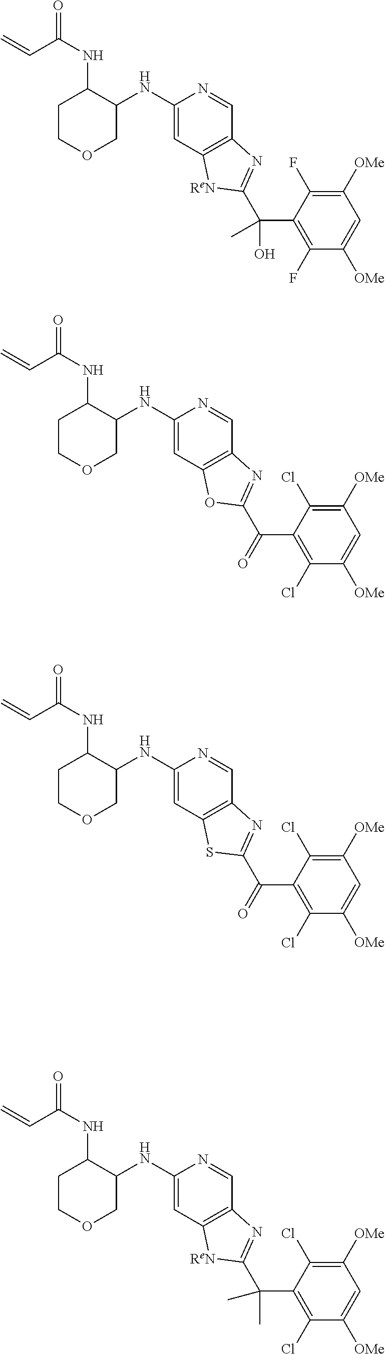

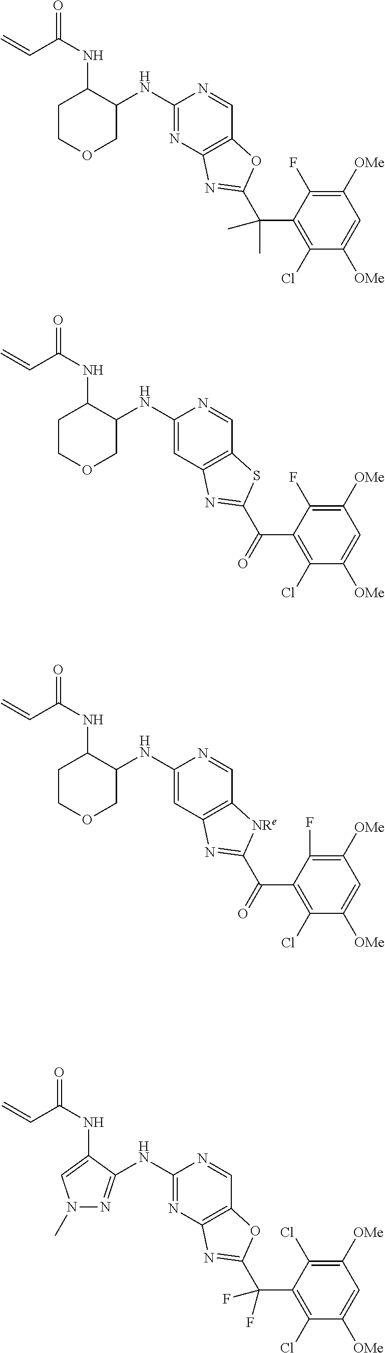

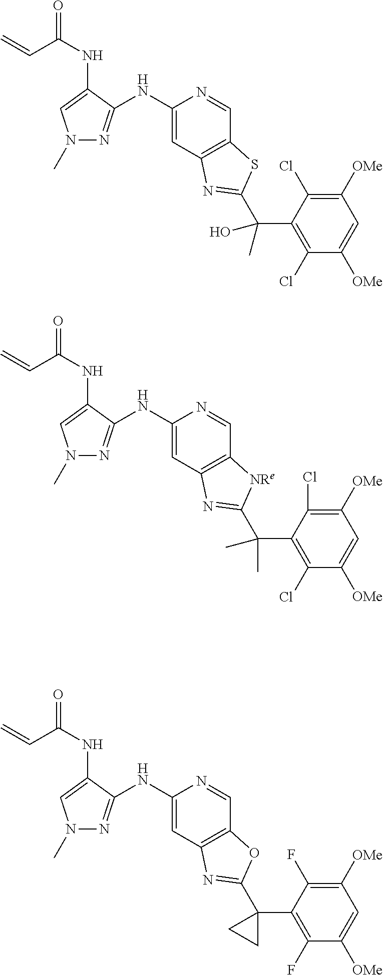

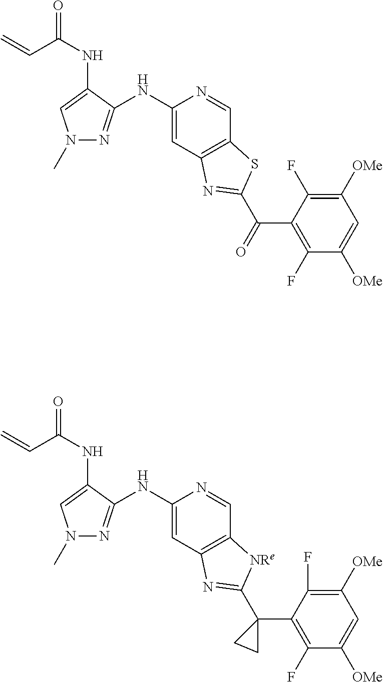

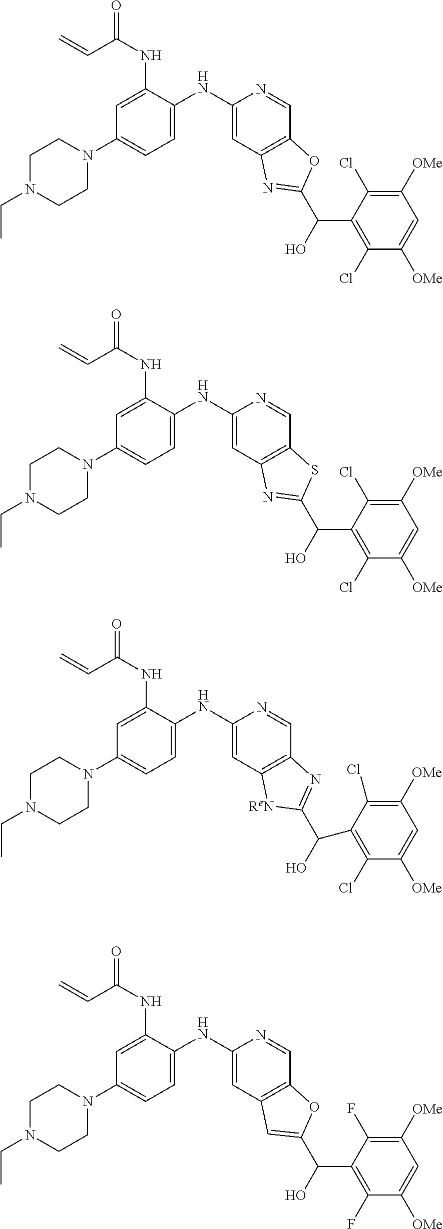

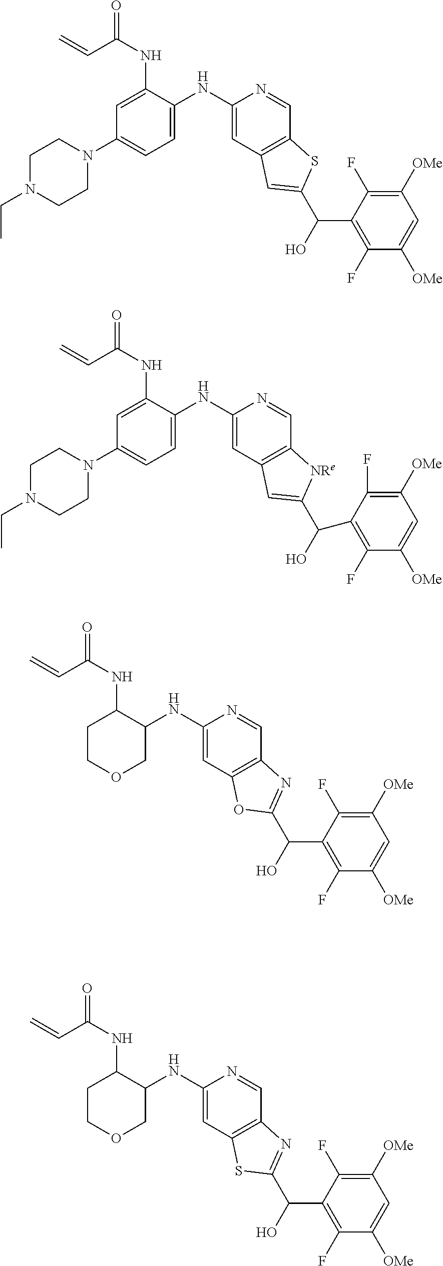

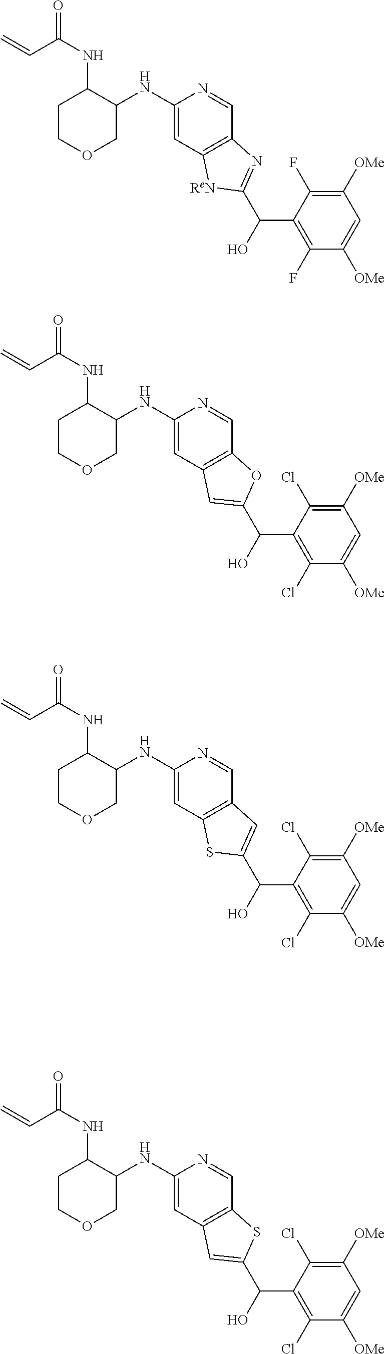

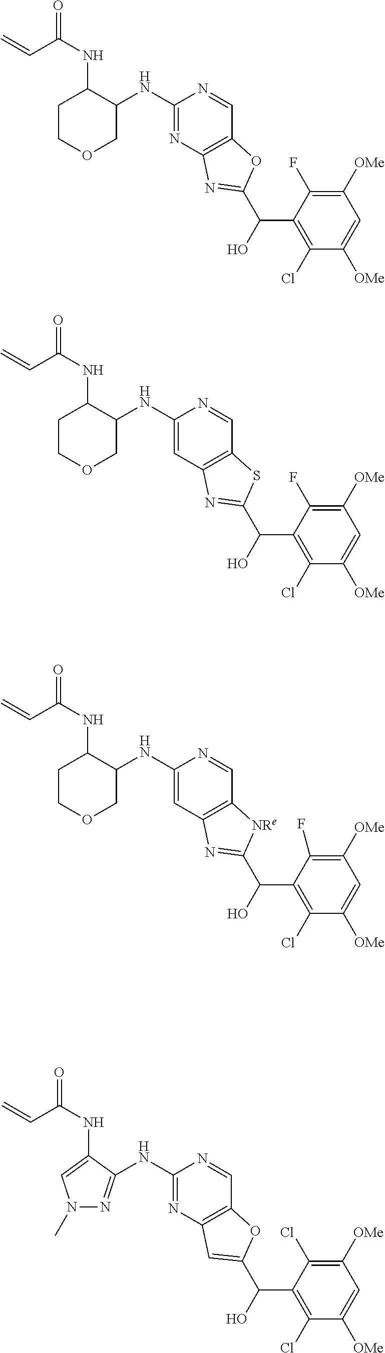

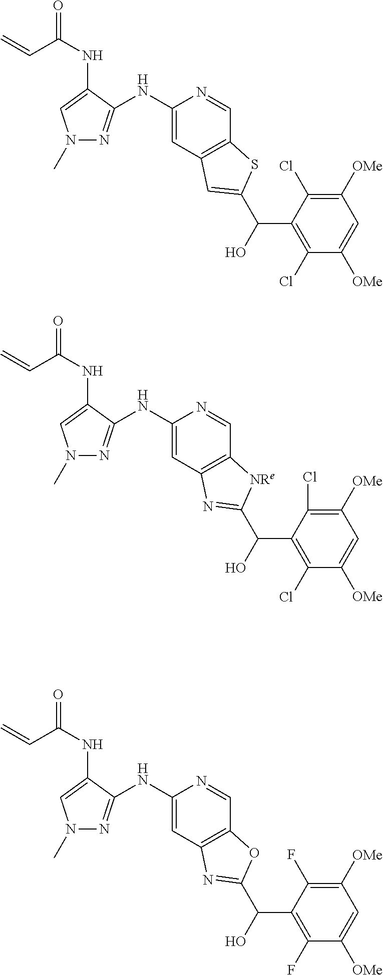

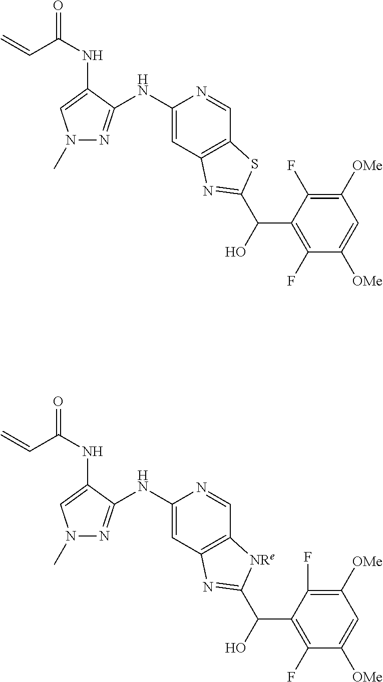

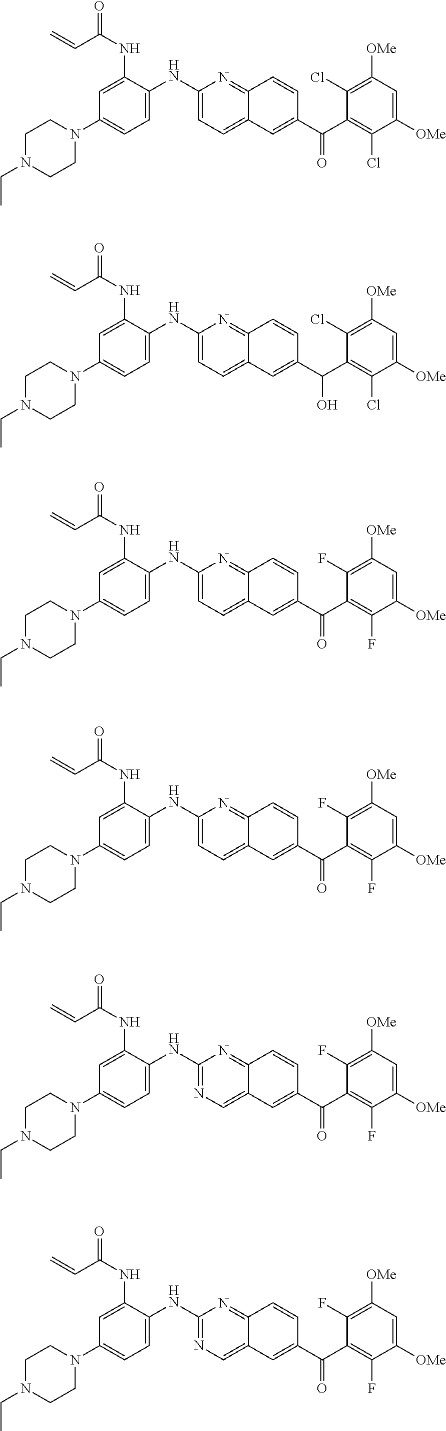 [SMILES: C=CC(=O)Nc1cc(N2CCN(CC)CC2)ccc1Nc1ccc2cc(C(=O)c3c(Cl)c(OC)cc(OC)c3Cl)ccc2n1 ; C=CC(=O)Nc1cc(N2CCN(CC)CC2)ccc1Nc1ccc2cc(C(O)c3c(Cl)c(OC)cc(OC)c3Cl)ccc2n1 ; C=CC(=O)Nc1cc(N2CCN(CC)CC2)ccc1Nc1ccc2cc(C(=O)c3c(F)c(OC)cc(OC)c3F)ccc2n1 ; C=CC(=O)Nc1cc(N2CCN(CC)CC2)ccc1Nc1ccc2cc(C(=O)c3c(F)c(OC)cc(OC)c3F)ccc2n1 ; C=CC(=O)Nc1cc(N2CCN(CC)CC2)ccc1Nc1ncc2cc(C(=O)c3c(F)c(OC)cc(OC)c3F)ccc2n1 ; C=CC(=O)Nc1cc(N2CCN(CC)CC2)ccc1Nc1ncc2cc(C(=O)c3c(F)c(OC)cc(OC)c3F)ccc2n1]

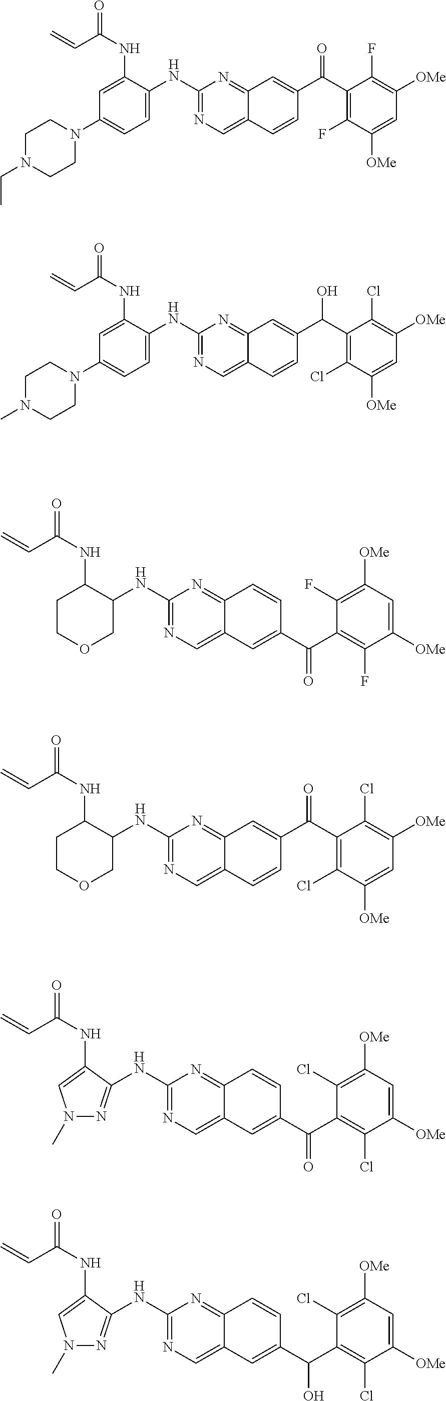 [SMILES: C=CC(=O)Nc1cc(N2CCN(CC)CC2)ccc1Nc1ncc2ccc(C(=O)c3c(F)c(OC)cc(OC)c3F)cc2n1 ; C=CC(=O)Nc1cc(N2CCN(C)CC2)ccc1Nc1ncc2ccc(C(O)c3c(Cl)c(OC)cc(OC)c3Cl)cc2n1 ; C=CC(=O)NC1CCOCC1Nc1ncc2cc(C(=O)c3c(F)c(OC)cc(OC)c3F)ccc2n1 ; C=CC(=O)NC1CCOCC1Nc1ncc2ccc(C(=O)c3c(Cl)c(OC)cc(OC)c3Cl)cc2n1 ; C=CC(=O)Nc1cn(C)nc1Nc1ncc2cc(C(=O)c3c(Cl)c(OC)cc(OC)c3Cl)ccc2n1 ; C=CC(=O)Nc1cn(C)nc1Nc1ncc2cc(C(O)c3c(Cl)c(OC)cc(OC)c3Cl)ccc2n1]

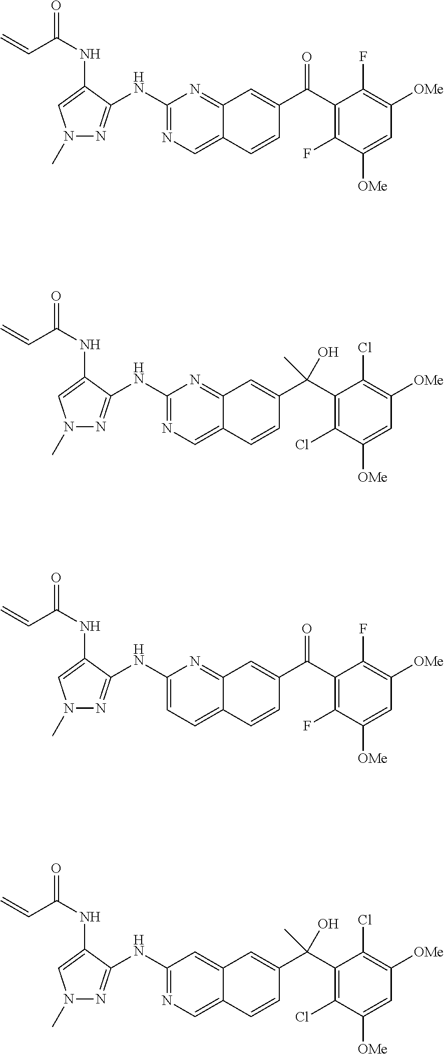 [SMILES: C=CC(=O)Nc1cn(C)nc1Nc1ncc2ccc(C(=O)c3c(F)c(OC)cc(OC)c3F)cc2n1 ; C=CC(=O)Nc1cn(C)nc1Nc1ncc2ccc(C(C)(O)c3c(Cl)c(OC)cc(OC)c3Cl)cc2n1 ; C=CC(=O)Nc1cn(C)nc1Nc1ccc2ccc(C(=O)c3c(F)c(OC)cc(OC)c3F)cc2n1 ; C=CC(=O)Nc1cn(C)nc1Nc1cc2cc(C(C)(O)c3c(Cl)c(OC)cc(OC)c3Cl)ccc2cn1]

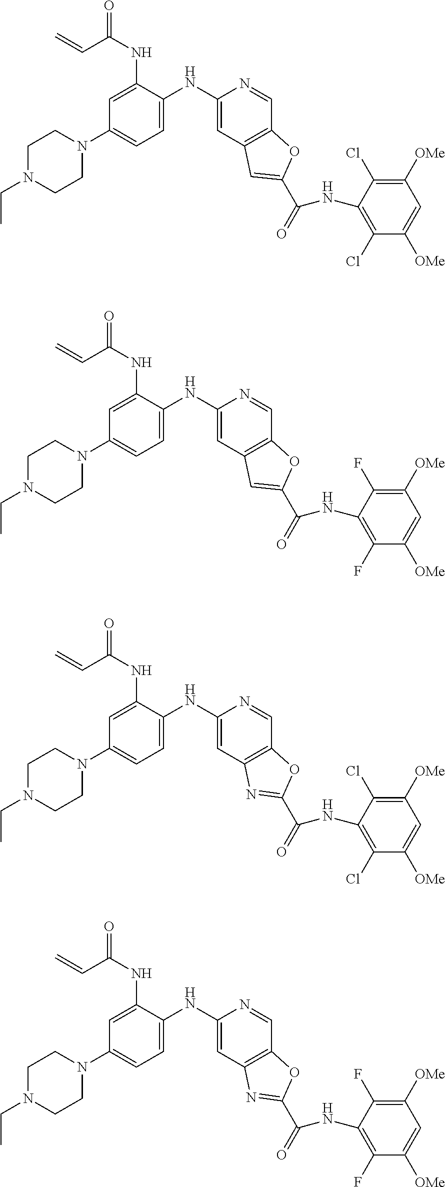 [SMILES: C=CC(=O)Nc1cc(N2CCN(CC)CC2)ccc1Nc1cc2cc(C(=O)Nc3c(Cl)c(OC)cc(OC)c3Cl)oc2cn1 ; C=CC(=O)Nc1cc(N2CCN(CC)CC2)ccc1Nc1cc2cc(C(=O)Nc3c(F)c(OC)cc(OC)c3F)oc2cn1 ; C=CC(=O)Nc1cc(N2CCN(CC)CC2)ccc1Nc1cc2nc(C(=O)Nc3c(Cl)c(OC)cc(OC)c3Cl)oc2cn1 ; C=CC(=O)Nc1cc(N2CCN(CC)CC2)ccc1Nc1cc2nc(C(=O)Nc3c(F)c(OC)cc(OC)c3F)oc2cn1]

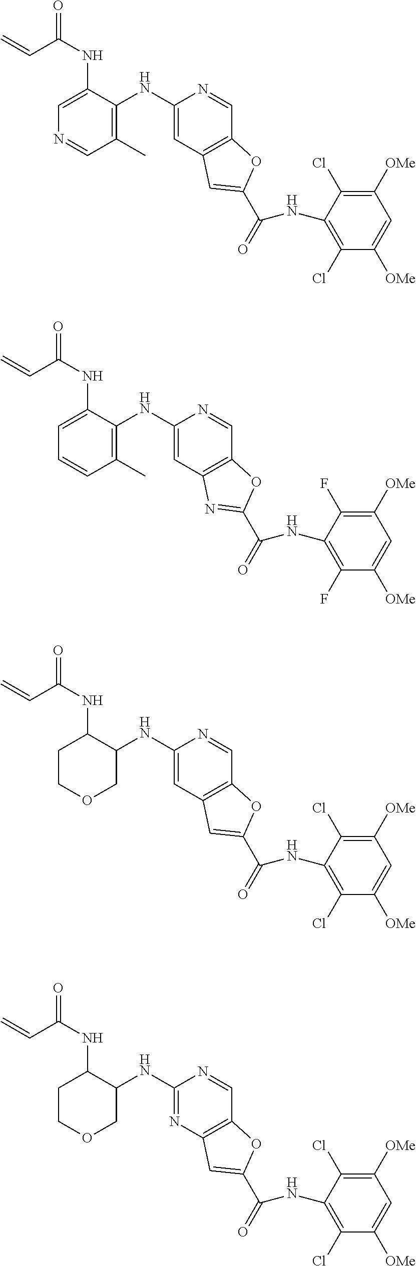 [SMILES: C=CC(=O)Nc1cncc(C)c1Nc1cc2cc(C(=O)Nc3c(Cl)c(OC)cc(OC)c3Cl)oc2cn1 ; C=CC(=O)Nc1cccc(C)c1Nc1cc2nc(C(=O)Nc3c(F)c(OC)cc(OC)c3F)oc2cn1 ; C=CC(=O)NC1CCOCC1Nc1cc2cc(C(=O)Nc3c(Cl)c(OC)cc(OC)c3Cl)oc2cn1 ; C=CC(=O)NC1CCOCC1Nc1ncc2oc(C(=O)Nc3c(Cl)c(OC)cc(OC)c3Cl)cc2n1]

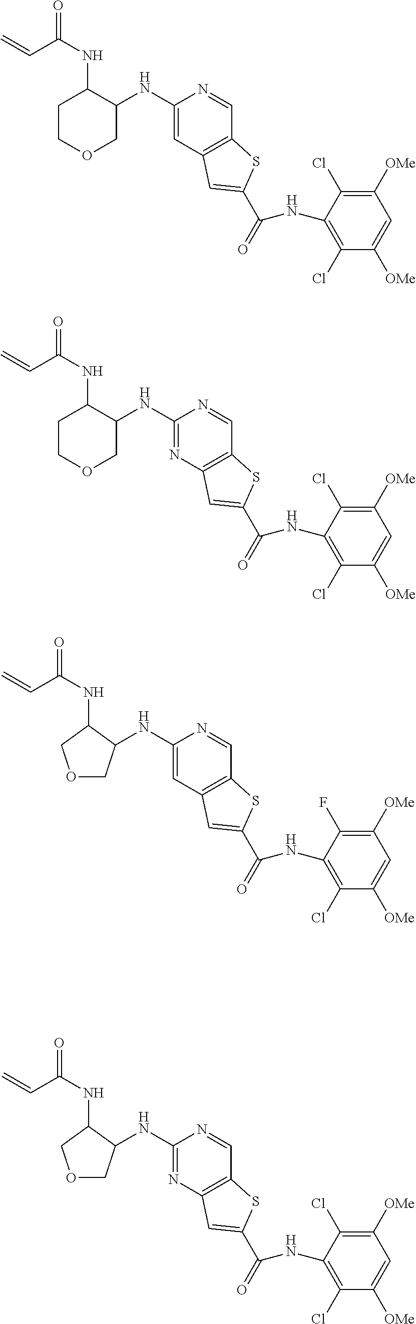 [SMILES: C=CC(=O)NC1CCOCC1Nc1cc2cc(C(=O)Nc3c(Cl)c(OC)cc(OC)c3Cl)sc2cn1 ; C=CC(=O)NC1CCOCC1Nc1ncc2sc(C(=O)Nc3c(Cl)c(OC)cc(OC)c3Cl)cc2n1 ; C=CC(=O)NC1COCC1Nc1cc2cc(C(=O)Nc3c(F)c(OC)cc(OC)c3Cl)sc2cn1 ; C=CC(=O)NC1COCC1Nc1ncc2sc(C(=O)Nc3c(Cl)c(OC)cc(OC)c3Cl)cc2n1]

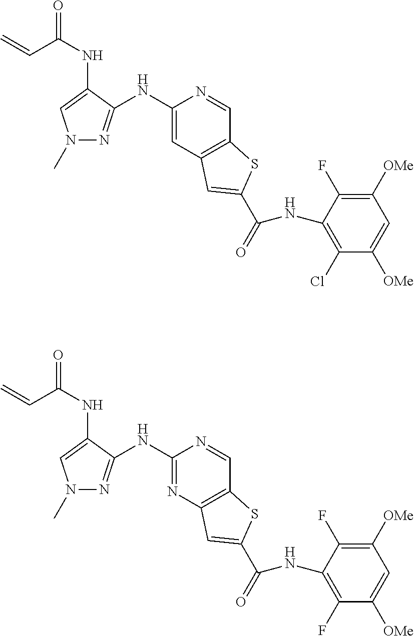 [SMILES: C=CC(=O)Nc1cn(C)nc1Nc1cc2cc(C(=O)Nc3c(F)c(OC)cc(OC)c3Cl)sc2cn1 ; C=CC(=O)Nc1cn(C)nc1Nc1ncc2sc(C(=O)Nc3c(F)c(OC)cc(OC)c3F)cc2n1]

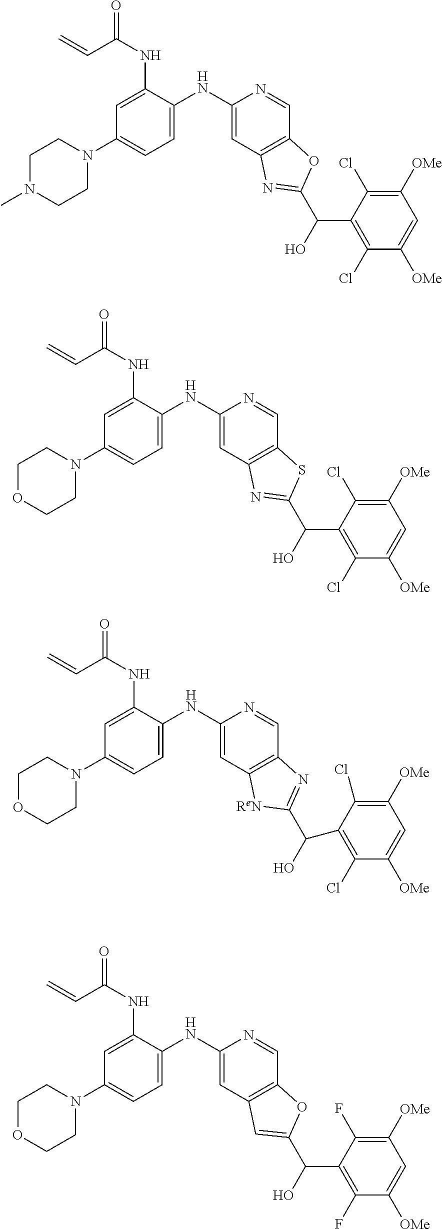

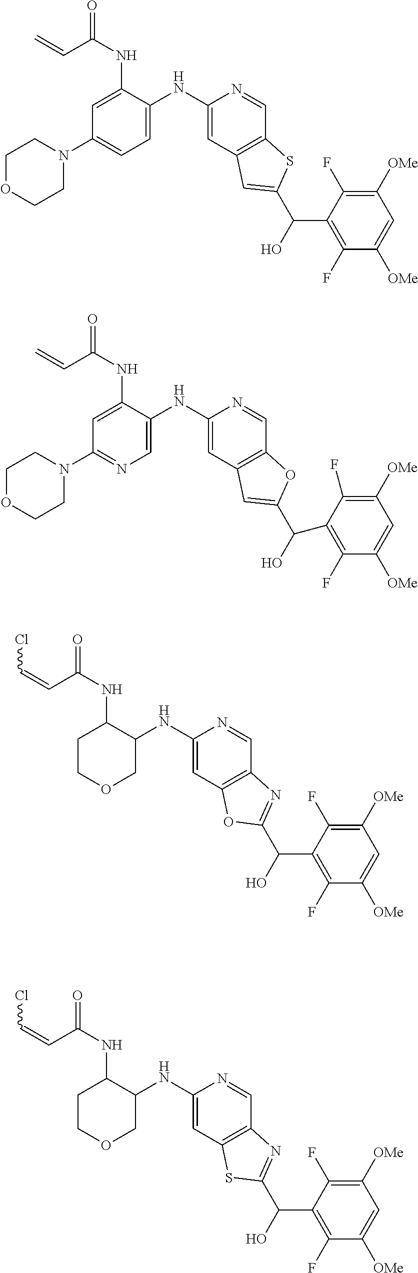 [SMILES: C=CC(=O)Nc1cc(N2CCOCC2)ccc1Nc1cc2cc(C(O)c3c(F)c(OC)cc(OC)c3F)sc2cn1 ; C=CC(=O)Nc1cc(N2CCOCC2)ncc1Nc1cc2cc(C(O)c3c(F)c(OC)cc(OC)c3F)oc2cn1 ; COc1cc(OC)c(F)c(C(O)c2nc3cnc(NC4COCCC4NC(=O)C=CCl)cc3o2)c1F ; COc1cc(OC)c(F)c(C(O)c2nc3cnc(NC4COCCC4NC(=O)C=CCl)cc3s2)c1F]

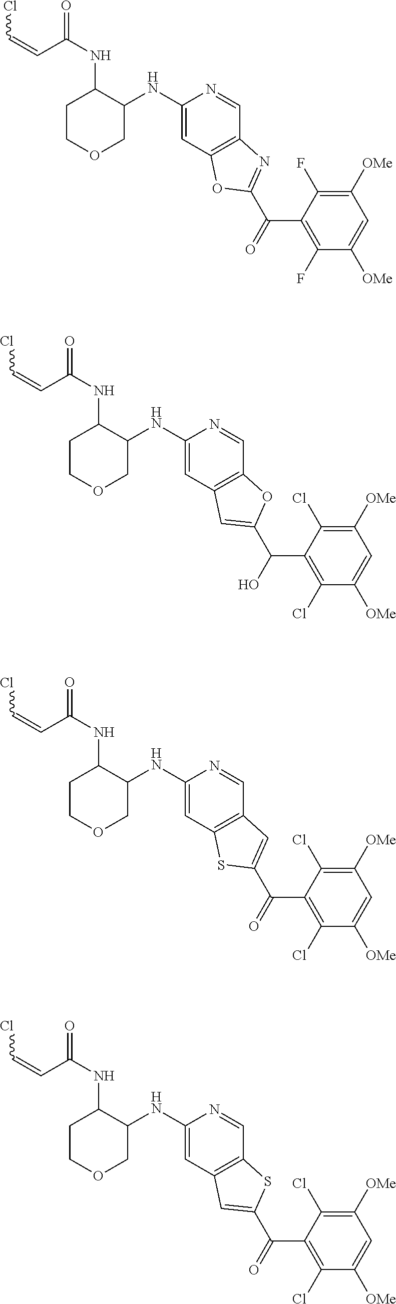 [SMILES: COc1cc(OC)c(F)c(C(=O)c2nc3cnc(NC4COCCC4NC(=O)C=CCl)cc3o2)c1F ; COc1cc(OC)c(Cl)c(C(O)c2cc3cc(NC4COCCC4NC(=O)C=CCl)ncc3o2)c1Cl ; COc1cc(OC)c(Cl)c(C(=O)c2cc3cnc(NC4COCCC4NC(=O)C=CCl)cc3s2)c1Cl ; COc1cc(OC)c(Cl)c(C(=O)c2cc3cc(NC4COCCC4NC(=O)C=CCl)ncc3s2)c1Cl]

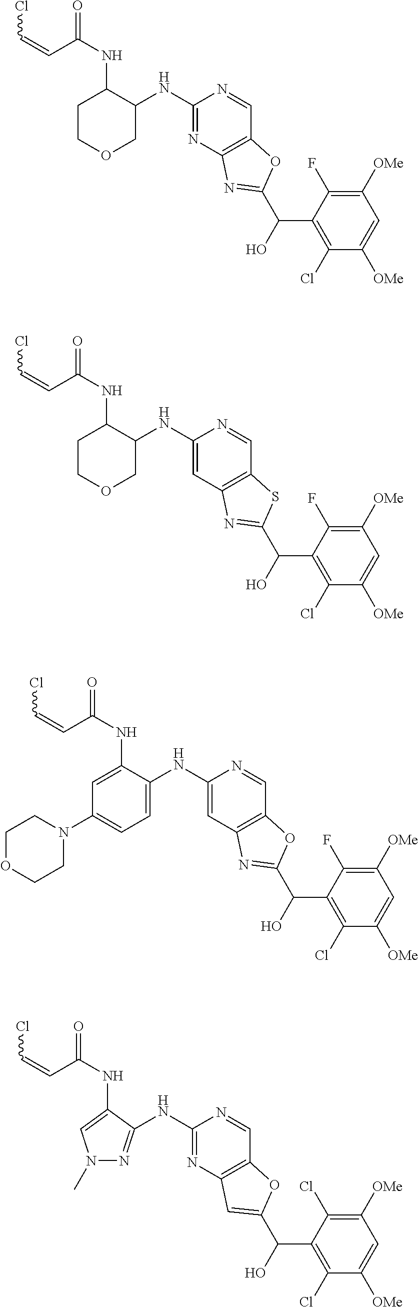 [SMILES: COc1cc(OC)c(Cl)c(C(O)c2nc3nc(NC4COCCC4NC(=O)C=CCl)ncc3o2)c1F ; COc1cc(OC)c(Cl)c(C(O)c2nc3cc(NC4COCCC4NC(=O)C=CCl)ncc3s2)c1F ; COc1cc(OC)c(Cl)c(C(O)c2nc3cc(Nc4ccc(N5CCOCC5)cc4NC(=O)C=CCl)ncc3o2)c1F ; COc1cc(OC)c(Cl)c(C(O)c2cc3nc(Nc4nn(C)cc4NC(=O)C=CCl)ncc3o2)c1Cl]

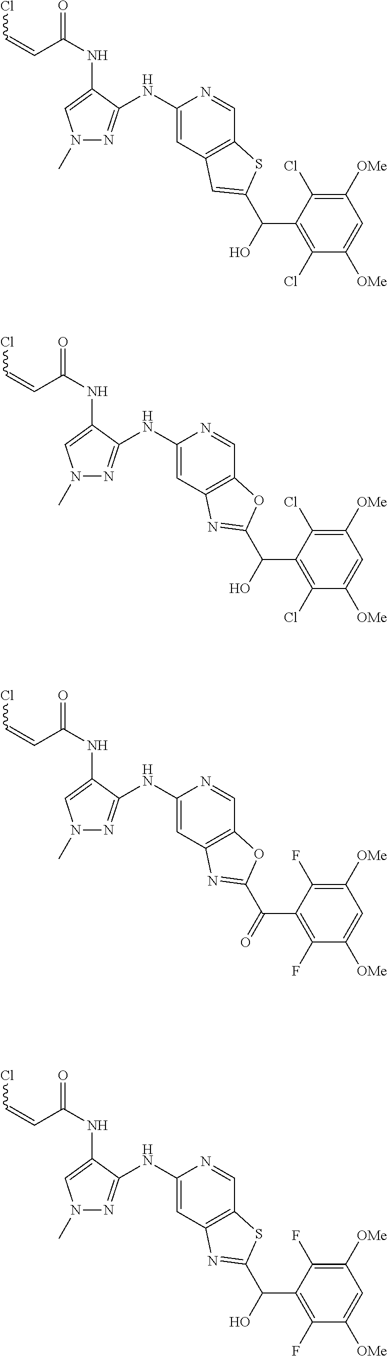 [SMILES: COc1cc(OC)c(Cl)c(C(O)c2cc3cc(Nc4nn(C)cc4NC(=O)C=CCl)ncc3s2)c1Cl ; COc1cc(OC)c(Cl)c(C(O)c2nc3cc(Nc4nn(C)cc4NC(=O)C=CCl)ncc3o2)c1Cl ; COc1cc(OC)c(F)c(C(=O)c2nc3cc(Nc4nn(C)cc4NC(=O)C=CCl)ncc3o2)c1F ; COc1cc(OC)c(F)c(C(O)c2nc3cc(Nc4nn(C)cc4NC(=O)C=CCl)ncc3s2)c1F]

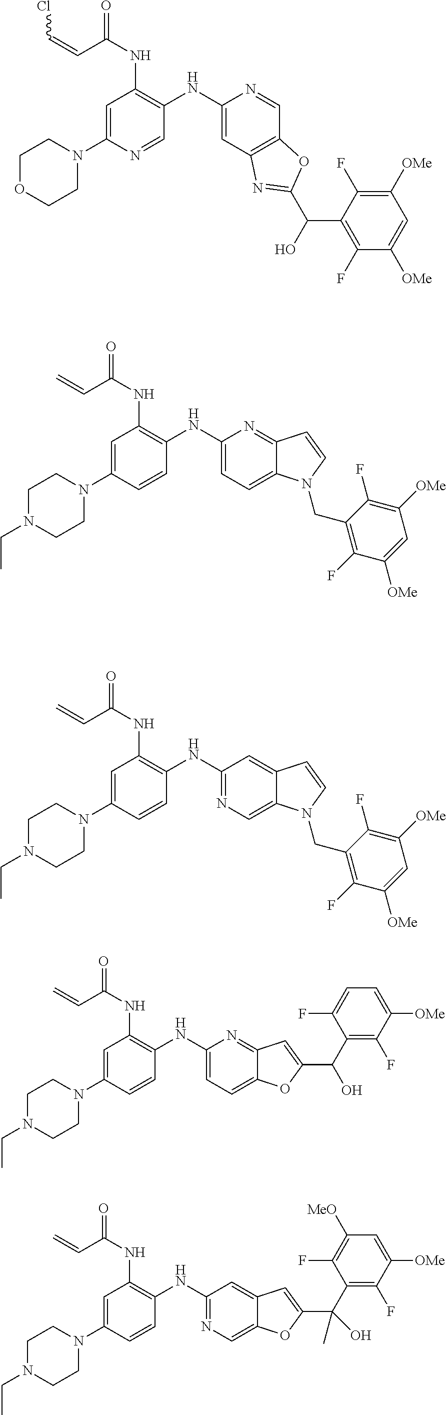 [SMILES: COc1cc(OC)c(F)c(C(O)c2nc3cc(Nc4cnc(N5CCOCC5)cc4NC(=O)C=CCl)ncc3o2)c1F ; C=CC(=O)Nc1cc(N2CCN(CC)CC2)ccc1Nc1ccc2c(ccn2Cc2c(F)c(OC)cc(OC)c2F)n1 ; C=CC(=O)Nc1cc(N2CCN(CC)CC2)ccc1Nc1cc2ccn(Cc3c(F)c(OC)cc(OC)c3F)c2cn1 ; C=CC(=O)Nc1cc(N2CCN(CC)CC2)ccc1Nc1ccc2oc(C(O)c3c(F)ccc(OC)c3F)cc2n1 ; C=CC(=O)Nc1cc(N2CCN(CC)CC2)ccc1Nc1cc2cc(C(C)(O)c3c(F)c(OC)cc(OC)c3F)oc2cn1]

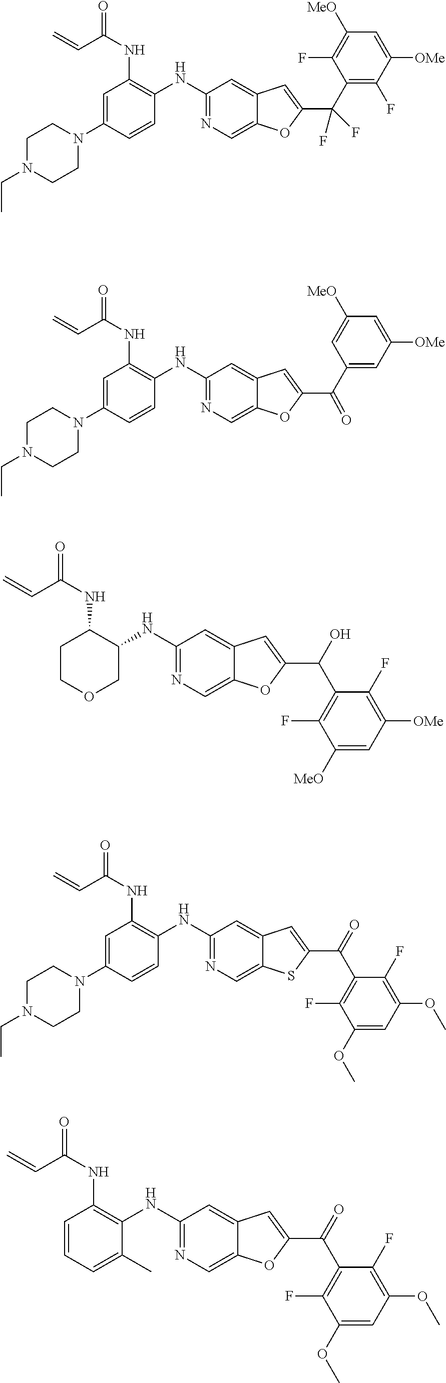

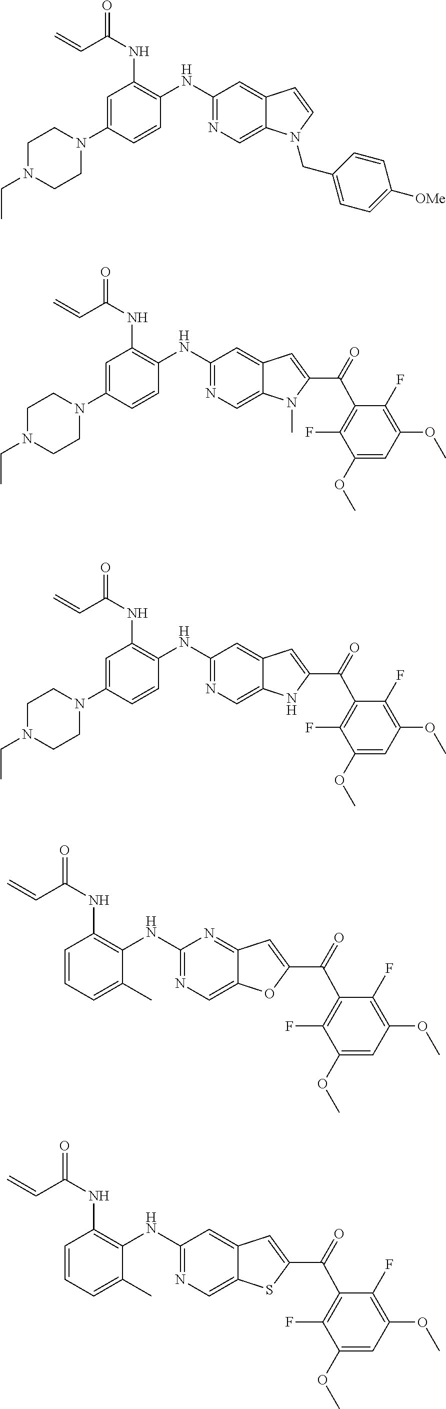

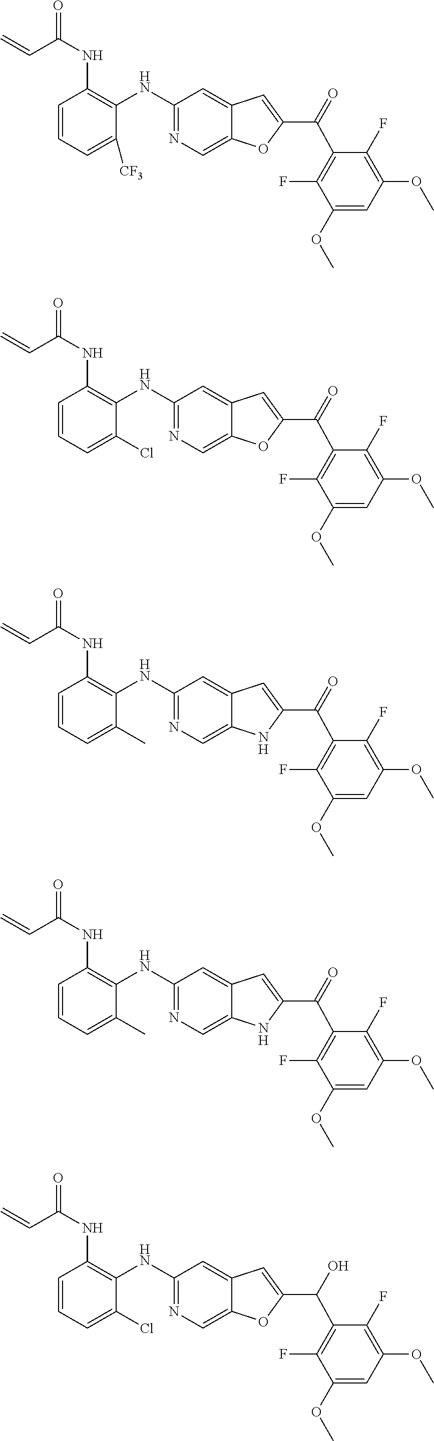

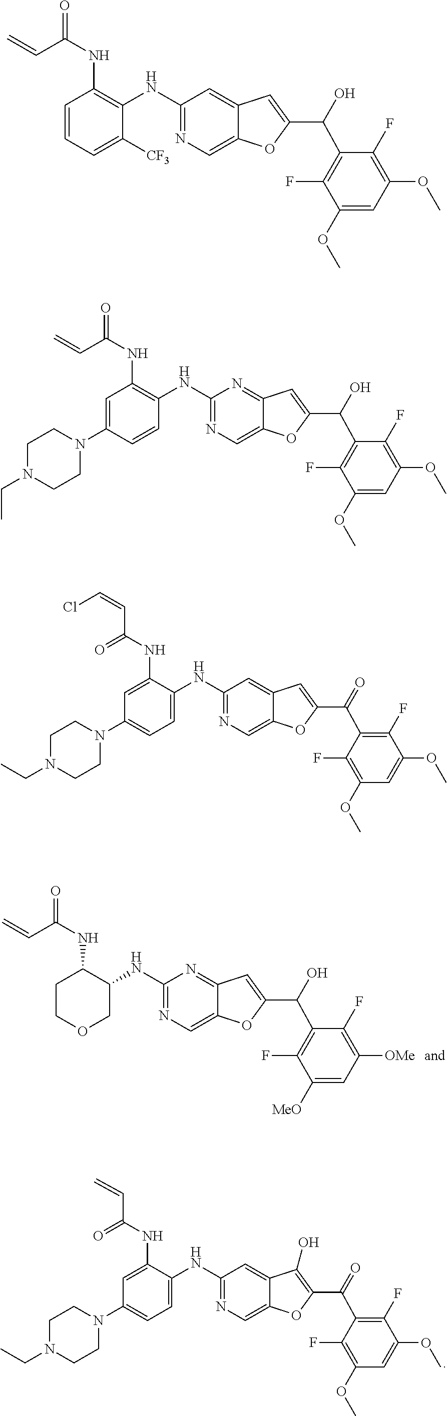

8. A method for treating a condition mediated by FGFR-4 or overexpressed FGFR-4, comprising administering a therapeutically effective amount of the compound or pharmaceutically acceptable salt thereof according to claim 1.

9. A method for treating a condition characterized by amplified FGF19 or overexpressed FGF19 or cancer, comprising administering a therapeutically effective amount of the compound or pharmaceutically acceptable salt thereof according to claim 1 wherein if the cancer is being treated, the cancer is selected from the group consisting of liver cancer, breast cancer, lung cancer, ovarian cancer, and a sarcoma.

10. The method of claim 8 wherein the cancer is the liver cancer and is a hepatocellular carcinoma.

11. The method of claim 8 further comprising co-administering with with one or more of other anti-cancer drugs to hepatocellular carcinoma, liver cancer, breast cancer, lung cancer, ovarian cancer, or a sarcoma.
